SEQUENCE LISTING (171 sequences)

SEQ ID NO: 1 - DNA, 19 bp, Artificial Sequence
  Description of Artificial Sequence Synthetic oligonucleotide
atcgtgggcgtccgcaaga

SEQ ID NO: 2 - DNA, 24 bp, Artificial Sequence
  Description of Artificial Sequence Synthetic oligonucleotide
agatgctgaccaactcgtgtgtga

SEQ ID NO: 3 - DNA, 21 bp, Artificial Sequence
  Description of Artificial Sequence Synthetic oligonucleotide
tggaggaggtgggcatctacc

SEQ ID NO: 4 - DNA, 25 bp, Artificial Sequence
  Description of Artificial Sequence Synthetic oligonucleotide
tgaggctcaaagtcagatgctactg

SEQ ID NO: 5 - DNA, 13 bp, Artificial Sequence
  Description of Artificial Sequence Synthetic oligonucleotide
acgcagaagccct

SEQ ID NO: 6 - DNA, 13 bp, Artificial Sequence
  Description of Artificial Sequence Synthetic oligonucleotide
aaggaagaagccc

SEQ ID NO: 7 - DNA, 13 bp, Artificial Sequence
  Description of Artificial Sequence Synthetic oligonucleotide
agttcaaaagccc

SEQ ID NO: 8 - DNA, 14 bp, Artificial Sequence
  Description of Artificial Sequence Synthetic oligonucleotide
acgtcaaagccctt

SEQ ID NO: 9 - DNA, 24 bp, Artificial Sequence
  Description of Artificial Sequence Synthetic oligonucleotide
tcccacctagaatctgctggctac

SEQ ID NO: 10 - DNA, 25 bp, Artificial Sequence
  Description of Artificial Sequence Synthetic oligonucleotide
gctgttcaaacagatcacatccaca

SEQ ID NO: 11 - DNA, 25 bp, Artificial Sequence
  Description of Artificial Sequence Synthetic oligonucleotide
atcgctcacaccaaatccttggacc

SEQ ID NO: 12 - DNA, 24 bp, Artificial Sequence
  Description of Artificial Sequence Synthetic oligonucleotide
gtgcgtgagagtgagagcagtcct

SEQ ID NO: 13 - DNA, 21 bp, Artificial Sequence
  Description of Artificial Sequence Synthetic oligonucleotide
cagggtgttgaagcggctctc

SEQ ID NO: 14 - DNA, 26 bp, Artificial Sequence
  Description of Artificial Sequence Synthetic oligonucleotide
ccatctcgctgagatacgaagggagg

SEQ ID NO: 15 - DNA, 20 bp, Artificial Sequence
  Description of Artificial Sequence Synthetic oligonucleotide
gcgatgccttccatcagtcg

SEQ ID NO: 16 - DNA, 22 bp, Artificial Sequence
  Description of Artificial Sequence Synthetic oligonucleotide
tgaaggtgttttcgggcagaag

SEQ ID NO: 17 - DNA, 22 bp, Artificial Sequence
  Description of Artificial Sequence Synthetic oligonucleotide
catgggtgcatcgggtgacctg

SEQ ID NO: 18 - DNA, 17 bp, Artificial Sequence
  Description of Artificial Sequence Synthetic oligonucleotide
tgggcgtccgcaagacc

SEQ ID NO: 19 - DNA, 20 bp, Artificial Sequence
  Description of Artificial Sequence Synthetic oligonucleotide
gtcgtgtccgaggccaccat

SEQ ID NO: 20 - DNA, 19 bp, Artificial Sequence
  Description of Artificial Sequence Synthetic oligonucleotide
cggttgtcgtgtccgaggc

SEQ ID NO: 21 - DNA, 19 bp, Artificial Sequence
  Description of Artificial Sequence Synthetic oligonucleotide
cacgccgcagtgccataag

SEQ ID NO: 22 - DNA, 23 bp, Artificial Sequence
  Description of Artificial Sequence Synthetic oligonucleotide
acagtccttcgacagcagcagtc

SEQ ID NO: 23 - DNA, 22 bp, Artificial Sequence
  Description of Artificial Sequence Synthetic oligonucleotide
cttctccctgacatccgtggag

SEQ ID NO: 24 - DNA, 21 bp, Artificial Sequence
  Description of Artificial Sequence Synthetic oligonucleotide
ggaggaggtgggcatctaccg

SEQ ID NO: 25 - DNA, 21 bp, Artificial Sequence
  Description of Artificial Sequence Synthetic oligonucleotide
gagttccaacgagcggcttca

SEQ ID NO: 26 - DNA, 24 bp, Artificial Sequence
  Description of Artificial Sequence Synthetic oligonucleotide
acccggagcttttcacctttagtt

SEQ ID NO: 27 - DNA, 24 bp, Artificial Sequence
  Description of Artificial Sequence Synthetic oligonucleotide
agacccggagcttttcacctttag

SEQ ID NO: 28 - DNA, 15 bp, Artificial Sequence
  Description of Artificial Sequence Synthetic oligonucleotide
aagggcttctgcgtc

SEQ ID NO: 29 - DNA, 13 bp, Artificial Sequence
  Description of Artificial Sequence Synthetic oligonucleotide
gacgcagaagccc

SEQ ID NO: 30 - DNA, 14 bp, Artificial Sequence
  Description of Artificial Sequence Synthetic oligonucleotide
gacgcagaagccct

SEQ ID NO: 31 - DNA, 13 bp, Artificial Sequence
  Description of Artificial Sequence Synthetic oligonucleotide
aagggcttcttcc

SEQ ID NO: 32 - DNA, 14 bp, Artificial Sequence
  Description of Artificial Sequence Synthetic oligonucleotide
agggcttcttcctt

SEQ ID NO: 33 - DNA, 14 bp, Artificial Sequence
  Description of Artificial Sequence Synthetic oligonucleotide
agagttcaaaagcc

SEQ ID NO: 34 - DNA, 16 bp, Artificial Sequence
  Description of Artificial Sequence Synthetic oligonucleotide
aagggcttttgaactc

SEQ ID NO: 35 - DNA, 16 bp, Artificial Sequence
  Description of Artificial Sequence Synthetic oligonucleotide
aagggcttttgaactc

SEQ ID NO: 36 - DNA, 15 bp, Artificial Sequence
  Description of Artificial Sequence Synthetic oligonucleotide
aagggctttgacgtc

SEQ ID NO: 37 - DNA, 153 bp, Homo sapiens
ttcgcttggcgcaaaatgttggagatctgcctgaagctggtgggctgcaaatccaagaaggggctgtcctcctcctccagctgttatctggaagaagcccttcagcggccagtagcatctgactttgagcctcagggtctgagtcaacccgct

SEQ ID NO: 38 - DNA, 532 bp, Homo sapiens
tttcgacatcccctgtgaatatttctgtttagggctttcttcttgaaaagaaattgttattcagcccgtttaaaacaaatcaagaaacttttgggtaacattgcaattacatgaaattgataaccgcgaaaataattgggactcctgcttgcaagtgtcaacctaaaaaaagtgcttccttttgttatggaagatgtctttctgtgattgacttcaattgctgacttgtggagatgcagcgaatgtgaaatcccacgtatatgccatttccctctacgctcgctgaccgttctggaagatcttgaacccctctctggaaaggggtacctattattactttatggggcagcagcctggaaaagtacttggggaccaaagaaggccaagcttgcctgccctgcattttatcaaaggagcagggaagaaggaatcatcgaggcatgggggtccacactgcaatgtttttgtggaacaagcccttcagcggccagtagcatctgactttgagcctcagggtctgagtgaagccgct

SEQ ID NO: 39 - DNA, 6267 bp, Homo sapiens
agctgcaagtggcgggcgcccaggcagatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 40 - DNA, 90 bp, Homo sapiens
atgatgagtctccggggctctatgggtttctgaatgtcatcgtccactcagccactggatttaagcagagttcaagtaagtactggtttg

SEQ ID NO: 41 - DNA, 204 bp, Homo sapiens
ccctttctcttccagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtaaaagggttgtgg

SEQ ID NO: 42 - DNA, 468 bp, Homo sapiens
gtccacagcattccgctgaccatcaataaggaagatgatgagtctccggggctctatgggtttctgaatgtcatcgtccactcagccactggatttaagcagagttcaaaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaat

SEQ ID NO: 43 - DNA, 90 bp, Homo sapiens; identical to SEQ ID NO: 40

SEQ ID NO: 44 - DNA, 6927 bp, Homo sapiens
ggggggagggtggcggctcgatgggggagccgcctccagggggcccccccgccctgtgcccacggcgcggcccctttaagaggcccgcctggctccgtcatccgcgccgcggccacctccccccggccctccccttcctgcggcgcagagtgcgggccgggcgggagtgcggcgagagccggctggctgagcttagcgtccgaggaggcggcggcggcggcggcggcacggcggcggcggggctgtggggcggtgcggaagcgagaggcgaggagcgcgcgggccgtggccagagtctggcggcggcctggcggagcggagagcagcgcccgcgcctcgccgtgcggaggagccccgcacacaatagcggcgcgcgcagcccgcgcccttccccccggcgcgccccgccccgcgcgccgagcgccccgctccgcctcacctgccaccagggagtgggcgggcattgttcgccgccgccgccgccgcgcgggccatgggggccgcccggcgcccggggccgggctggcgaggcgccgcgccgccgctgagacgggccccgcgcgcagcccggcggcgcaggtaaggccggccgcgccatggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagaatgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaaatctgtactgcaccctggaggtggattcctttgggtattttgtgaataaagcaaagacgcgcgtctacagggacacagctgagccaaactggaacgaggaatttgagatagagctggagggctcccagaccctgaggatactgtgctatgaaaagtgttacaacaagacgaagatccccaaggaggacggcgagagcacggacagactcatggggaagggccaggtccagctggacccgcaggccctgcaggacagagactggcagcgcaccgtcatcgccatgaatgggatcgaagtaaagctctcggtcaagttcaacagcagggagttcagcttgaagaggatgccgtcccgaaaacagacaggggtcttcggagtcaagattgctgtggtcaccaagagagagaggtccaaggtgccctacatcgtgcgccagtgcgtggaggagatcgagcgccgaggcatggaggaggtgggcatctaccgcgtgtccggtgtggccacggacatccaggcactgaaggcagccttcgacgtcaataacaaggacgtgtcggtgatgatgagcgagatggacgtgaacgccatcgcaggcacgctgaagctgtacttccgtgagctgcccgagcccctcttcactgacgagttctaccccaacttcgcagagggcatcgctctttcagacccggttgcaaaggagagctgcatgctcaacctgctgctgtccctgccggaggccaacctgctcaccttccttttccttctggaccacctgaaaagggtggcagagaaggaggcagtcaataagatgtccctgcacaacctcgccacggtctttggccccacgctgctccggccctccgagaaggagagcaagctccctgccaaccccagccagcctatcaccatgactgacagctggtccttggaggtcatgtcccaggtccaggtgctgctgtacttcctgcagctggaggccatccctgccccggacagcaagagacagagcatcctgttctccaccgaagtctaaaggtcccagtccatctcctggaggcggacagatggcctggaaacctctggctaatcgggccatccgtagagcgggaaccttcctgaggtgtccttgggccacccccaagtgttgggccatctgccaagagacagcgacccaaagccgaaggacaggtggcctggaaagatcccgcccaggtctgggagccccaggctggcctcagactgtggttttttatgtggccacctgagggcgccccaagccagttcatctcggagtccaggcctggccctgggagacagggtgaagggagtggtttttatgaacttaacttagagtctaaaagatttctactggatcacttgtcaagatgcgccctctctggggagaagggaacgtgactggattccctcactgttgtatcttgaataaacgctgctgcttcatcctgtgggggccgtggccctgtccctgtgtgggtggggcctcttccatttccctgacttagaaaccacactccacttctaacagggtttgagaggcttagtcagcactgggtagcgttttgactccattcttggctttctttttctttccagaaggatttttgtgcagaaatgggtcttttgttgccatgttagtcctccttggaaggcagctcagaaggcctgtgaaatgtcaggggacaggacccccagggagggaaccccaggctacgcactttagggttcgttctccagggagagcgacctcgtcccccgatcctgaccgcccttccggcccacgctctcctgtttggcttccacaggcctggacttctctggcttctctgcccacacactccctgcccccagtgtccctgcccctgccccagcacaggtgacttcatttctgtcctctcagctcagtggactcgctcaacttttgtataagtctccacttggtggcagcagcttgctgatgacttgttttaaaactttcatcctaaataaccttttgatacttgaatatttgtaagttttatacatagtttctaatttttttccgaacagatccagatacctaataagatgctggaatgtaatccctggacaatccgtgtcctggcagcatttggtcttcctctaagcgcctggctccgctgttctcaggagtgggttctgaagtctctggagaacaggatacgtggagggttaggaaggggccaggcctagagacgggagactccctcccggagcaggtggaggcacaggaccattcgctaccccatctgccgacacctgcgggggagcccaggcattctttgtaaaccctcctgaccacctggctcaaagaaaacagaagcatggagaccgccaagtattttcaagaaataaccccatgaatattccatcacttttttagaaagaggggcttggggcaggcagaggagagaagggagagcaaactgagagccaagtttccacacggtcctgcaggaggagaggatgcagctgcccagagggaagcaggatcacatttaaggaagtgtgtggggtccctggatgacaccagcacccagtgcggctctgtctggcacccgctcccaaggtgggaggagtgggtgtcccctgtgtgtcagtgggcagctcctgctgaacccgcagctcactagggagcctgacagtggggccatgcgcctgacactcctctctgcttgtggacctggcaaggcagggagcagaaaacagccacttgaaggctttctgtctgcgtctgtgtgcagtgtggatttagttgtgcttttttcttgctgggagagcacagccaccatttacaagcagtgtcaccgtcgtgggtggcgaggacagaacaggagcctctgctctctgtacctatctgggcccggtgggctcccttgtcctggcttccatctctgtctcagcgaccattcagccctgcgcaggaacacgtgttgcttagaaaagccaaatccagccttgtctctgcctcctctggtctcatgatgtgcatctgttaccttgaaactggaaaccagtctatcaatgtctgtgccaattttttattccctccccaacctccttccccatacgactttttatttatgtaggatgtgtgctgtctaatgatgggatgaccacacttttccatgttctaaaagtgctcctctcccgcagggtcccagggctggtggttgctttgggtctacagctacgtcttacccacctcctgcctcaacagcctgtgtggtggcaaagccggtgtggggctggggaacgcagcgttctccaggagggggacctggctctccttctgcaatgcaggcgaaggcctagatgccagtgtgacctcccacaaggcgtggcttccagactccccggccggaagtgatgcttttttgccttgggccctgggtttgaagcagcctggctttctcttggtaagtggctggtgtcttagcagctgcaatctgagctcagccacctacacaccaccgtggccgacactttcattaaaaagtttcctgagacga

SEQ ID NO: 45 - DNA, 6795 bp, Homo sapiens
ggggggagggtggcggctcgatgggggagccgcctccagggggcccccccgccctgtgcccacggcgcggcccctttaagaggcccgcctggctccgtcatccgcgccgcggccacctccccccggccctccccttcctgcggcgcagagtgcgggccgggcgggagtgcggcgagagccggctggctgagcttagcgtccgaggaggcggcggcggcggcggcggcacggcggcggcggggctgtggggcggtgcggaagcgagaggcgaggagcgcgcgggccgtggccagagtctggcggcggcctggcggagcggagagcagcgcccgcgcctcgccgtgcggaggagccccgcacacaatagcggcgcgcgcagcccgcgcccttccccccggcgcgccccgccccgcgcgccgagcgccccgctccgcctcacctgccaccagggagtgggcgggcattgttcgccgccgccgccgccgcgcgggccatgggggccgcccggcgcccggggccgggctggcgaggcgccgcgccgccgctgagacgggccccgcgcgcagcccggcggcgcaggtaaggccggccgcgccatggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagaatgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaaatctgtactgcaccctggaggtggattcctttgggtattttgtgaataaagcaaagacgcgcgtctacagggacacagctgagccaaactggaacgagctggacccgcaggccctgcaggacagagactggcagcgcaccgtcatcgccatgaatgggatcgaagtaaagctctcggtcaagttcaacagcagggagttcagcttgaagaggatgccgtcccgaaaacagacaggggtcttcggagtcaagattgctgtggtcaccaagagagagaggtccaaggtgccctacatcgtgcgccagtgcgtggaggagatcgagcgccgaggcatggaggaggtgggcatctaccgcgtgtccggtgtggccacggacatccaggcactgaaggcagccttcgacgtcaataacaaggacgtgtcggtgatgatgagcgagatggacgtgaacgccatcgcaggcacgctgaagctgtacttccgtgagctgcccgagcccctcttcactgacgagttctaccccaacttcgcagagggcatcgctctttcagacccggttgcaaaggagagctgcatgctcaacctgctgctgtccctgccggaggccaacctgctcaccttccttttccttctggaccacctgaaaagggtggcagagaaggaggcagtcaataagatgtccctgcacaacctcgccacggtctttggccccacgctgctccggccctccgagaaggagagcaagctccctgccaaccccagccagcctatcaccatgactgacagctggtccttggaggtcatgtcccaggtccaggtgctgctgtacttcctgcagctggaggccatccctgccccggacagcaagagacagagcatcctgttctccaccgaagtctaaaggtcccagtccatctcctggaggcggacagatggcctggaaacctctggctaatcgggccatccgtagagcgggaaccttcctgaggtgtccttgggccacccccaagtgttgggccatctgccaagagacagcgacccaaagccgaaggacaggtggcctggaaagatcccgcccaggtctgggagccccaggctggcctcagactgtggttttttatgtggccacctgagggcgccccaagccagttcatctcggagtccaggcctggccctgggagacagggtgaagggagtggtttttatgaacttaacttagagtctaaaagatttctactggatcacttgtcaagatgcgccctctctggggagaagggaacgtgactggattccctcactgttgtatcttgaataaacgctgctgcttcatcctgtgggggccgtggccctgtccctgtgtgggtggggcctcttccatttccctgacttagaaaccacactccacttctaacagggtttgagaggcttagtcagcactgggtagcgttttgactccattcttggctttctttttctttccagaaggatttttgtgcagaaatgggtcttttgttgccatgttagtcctccttggaaggcagctcagaaggcctgtgaaatgtcaggggacaggacccccagggagggaaccccaggctacgcactttagggttcgttctccagggagagcgacctcgtcccccgatcctgaccgcccttccggcccacgctctcctgtttggcttccacaggcctggacttctctggcttctctgcccacacactccctgcccccagtgtccctgcccctgccccagcacaggtgacttcatttctgtcctctcagctcagtggactcgctcaacttttgtataagtctccacttggtggcagcagcttgctgatgacttgttttaaaactttcatcctaaataaccttttgatacttgaatatttgtaagttttatacatagtttctaatttttttccgaacagatccagatacctaataagatgctggaatgtaatccctggacaatccgtgtcctggcagcatttggtcttcctctaagcgcctggctccgctgttctcaggagtgggttctgaagtctctggagaacaggatacgtggagggttaggaaggggccaggcctagagacgggagactccctcccggagcaggtggaggcacaggaccattcgctaccccatctgccgacacctgcgggggagcccaggcattctttgtaaaccctcctgaccacctggctcaaagaaaacagaagcatggagaccgccaagtattttcaagaaataaccccatgaatattccatcacttttttagaaagaggggcttggggcaggcagaggagagaagggagagcaaactgagagccaagtttccacacggtcctgcaggaggagaggatgcagctgcccagagggaagcaggatcacatttaaggaagtgtgtggggtccctggatgacaccagcacccagtgcggctctgtctggcacccgctcccaaggtgggaggagtgggtgtcccctgtgtgtcagtgggcagctcctgctgaacccgcagctcactagggagcctgacagtggggccatgcgcctgacactcctctctgcttgtggacctggcaaggcagggagcagaaaacagccacttgaaggctttctgtctgcgtctgtgtgcagtgtggatttagttgtgcttttttcttgctgggagagcacagccaccatttacaagcagtgtcaccgtcgtgggtggcgaggacagaacaggagcctctgctctctgtacctatctgggcccggtgggctcccttgtcctggcttccatctctgtctcagcgaccattcagccctgcgcaggaacacgtgttgcttagaaaagccaaatccagccttgtctctgcctcctctggtctcatgatgtgcatctgttaccttgaaactggaaaccagtctatcaatgtctgtgccaattttttattccctccccaacctccttccccatacgactttttatttatgtaggatgtgtgctgtctaatgatgggatgaccacacttttccatgttctaaaagtgctcctctcccgcagggtcccagggctggtggttgctttgggtctacagctacgtcttacccacctcctgcctcaacagcctgtgtggtggcaaagccggtgtggggctggggaacgcagcgttctccaggagggggacctggctctccttctgcaatgcaggcgaaggcctagatgccagtgtgacctcccacaaggcgtggcttccagactccccggccggaagtgatgcttttttgccttgggccctgggtttgaagcagcctggctttctcttggtaagtggctggtgtcttagcagctgcaatctgagctcagccacctacacaccaccgtggccgacactttcattaaaaagtttcctgagacga

SEQ ID NO: 46 - DNA, 63 bp, Homo sapiens
gatggcgagggcgccttccatggagacgcagatggctcgttcggaacaccacctggatacggc

SEQ ID NO: 47 - DNA, 63 bp, Homo sapiens
gatggcgagggcgccttccatggagacgcagaagcccttcagcggccagtagcatctgacttt

SEQ ID NO: 48 - DNA, 491 bp, Homo sapiens
ggaaggggagggcaagggcccgctcctgcgcacgcagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagaagcccttcagcggccagtagcatctgactttgagcctcagggtctgagtgaagccgctcgttggaactccaagg

SEQ ID NO: 49 - DNA, 663 bp, Homo sapiens
tggagctgcagatgctgaccaactcgtgtgtgaaactccagactgtccacagcattccgctgaccatcaataaggaaggtgggcccccccgtttccgtgtacagggcacctgcagggagggcaggcagctagcctgaaggctgatccccccatgcccggctaaagcaagaaattttcattgcatattttaagcaaaggcaaatgcatatgtggatagactgttttaatttgactaaagtcatattgaatccatgaattttagaagctcaaactattggggaacaataattaccaccttggagtgaaaatacttaatttccacaagatttagtaaaggaagagttttttaaaaaccaccttaatgataatagtatgtacagatgttaagaaatgaaataggaatgtgtaatgttggaaacacaaatatttttgcttctgagaataaaactaattttttctcccaattttctcttcctttttcttttttctgttcccccctttctcttccagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccaagtggagat

SEQ ID NO: 50 - DNA, 238 bp, Homo sapiens
tctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcccctttctcttccagaagcccttcagcggccagtagcatctgactttgagcctcagggtctgagtgaagcc

SEQ ID NO: 51 - DNA, 486 bp, Homo sapiens
ctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcagttccaacgagctccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcc

SEQ ID NO: 52 - DNA, 163 bp, Homo sapiens
gaagtaaagctctcggtcaagttcaacagcagggagttcagcttgaagaggatgccgtcccgaaaacagacaggggtcttcggagtcccagcttgtgcatgtgtgtttttaagaaaacgtccacaagaagcccttcagcggccagtagcatctgactttgagc

SEQ ID NO: 53 - DNA, 297 bp, Homo sapiens
acgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaagtgaaaagctccgggtcttaggctataatcacaatggggaatggtgt

SEQ ID NO: 54 - DNA, 1365 bp, Homo sapiens
gccccgcgcgcagcccggcggcgcaggtaaggccggccgcgccatggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaactaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagaagcccttcagcggccagtagcatctgactttcagcctcaggg

SEQ ID NO: 55 - DNA, 468 bp, Homo sapiens; identical to SEQ ID NO: 42

SEQ ID NO: 56 - DNA, 1545 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagataacactctaa

SEQ ID NO: 57 - DNA, 4902 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccggcggccactcaggacttcagcaagctcctcagttcggtgaaggaaatcagtgacatagtgcagaggtag

SEQ ID NO: 58 - DNA, 1290 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggtggatga

SEQ ID NO: 59 - DNA, 853 bp, Homo sapiens
cacagcattccgctgaccatcaacaaggaaggtgggccccccccgtctccgtgtacagggcacctgcagggagggcaggcagctagcctgaaggctgatccccccttcctgttagcacttttgatgggactagtggactttggttcagaaggaagagctatgcttgttagggcctcttgtctcctcccaggagtggacaaggtgggttaggagcagtttctccctgagtggctgctgctgggtggttgaggagatgcacggcttctgttcctagtcacaattgcggtttgacctaccaccctttgctcgttaaaggagcagctttgaaatctggactgcagggatatccaaaacaacaactgcatgtttctaagggagtcgactctccttagaggagttcttgtacaatagccctgggcaaaaacagaacttgccctattttttatactgaaaaggacagctggacaaaatactgaacgcaatttttcccctaagaaaaagcattatttccctaaaatgtcttatattaggaacagagcacttgaataaacataattgatttataaaaactgaggctatacacttacctatctgttcagtacaaacaggaagcttcaaatgtaaacgtgaattctcatacacttataaatgcatatctttatgtggacttgttaaaatgaaattggtatttaggaatttggagatttttagtagttacacaagaatcaatgaaaaagaacgaagctggtttccaaagctgatatgtctgatttggttcctttcttctcaggtgaaaagctccgggtcttaggctataatcacaatggggaatggtgtgaagcccaaaccaaaa

SEQ ID NO: 60 - DNA, 253 bp, Homo sapiens
gaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaagaggacaccatggaggtggaagagttcttgaaagaagctgcagtcatgaaagagatcaaacaccctaa

SEQ ID NO: 61 - DNA, 234 bp, Homo sapiens
cagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcaggaggacaccatggaggtggaagagttcttgaaagaagctgcagtcatgaaagagatcaaacaccctaa

SEQ ID NO: 62 - DNA, 216 bp, Homo sapiens
cagatgctgaccaactcgtgtgtgaaactccagactgtccacagcattccgctgaccatcaataaggaagatgatgagtctccggggctctatgggtttctgaatgtcatcgtccactcagccactggatttaagcagagttcaactctacgtctcctccgagagccgcttcaacaccctggccgagttggttcatcatcattcaacggtggccga

SEQ ID NO: 63 - DNA, 237 bp, Homo sapiens
cagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagctctacgtctcctccgagagccgcttcaacaccctggccgagttggttcatcatcattcaacggtggccga

SEQ ID NO: 64 - DNA, 178 bp, Homo sapiens
gaagtgtttcagaagcttctccctgacatccgtggagctgcagatgctgaccaactcgtgtgtgaaactccagactgtccacagcattccgctgaccatcaataaggaaggaggacaccatggaggtggaagagttcttgaaagaagctgcagtcatgaaagagatcaaacaccctaa

SEQ ID NO: 65 - DNA, 141 bp, Homo sapiens
cagatgctgaccaactcgtgtgtgaaactccagactgtccacagcattccgctgaccatcaataaggaagctctacgtctcctccgagagccgcttcaacaccctggccgagttggttcatcatcattcaacggtggccga

SEQ ID NO: 66 - DNA, 293 bp, Homo sapiens
cagatgctgaccaactcgtgtgtgaaactccagactgtccacagcattccgctgaccatcaataaggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtga

SEQ ID NO: 67 - DNA, 393 bp, Homo sapiens
ctctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagaggctggtctcgaactcctgacctcagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaag

SEQ ID NO: 68 - DNA, 5373 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagagtgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaacaaggaagatgatgagtctccggggctctatgggtttctgaatgtcatcgtccactcagccactggatttaagcagagttcaaaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccggcggccactcaggacttcagcaagctcctcagttcggtgaaggaaatcagtgacatagtgcagaggtag

SEQ ID NO: 69 - DNA, 1404 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagttctgaagactgaagtcccacatcaaggtgtccgcagggccaggctgtctctggaggctctaggggaggatccttcctttctgttcccagcatctgatggcccacggcattcctcgacctggtga

SEQ ID NO: 70 - DNA, 1377 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatactgctgcttatgtctcccagcatggccttcagggtgcacagccgcaacggcaagagttacacgttcctga

SEQ ID NO: 71 - DNA, 1398 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagaacatccgggagcagcagaagaagtgtttcagaagcttctccctgacatccgtggagctgcagatgctgaccaactcgtgtgtgaaactccagactgtccacagcattccgctgaccatcaataa

SEQ ID NO: 72 - DNA, 1326 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttggaactccaaggaaaaccttctcgctggacccagtga

SEQ ID NO: 73 - DNA, 1665 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgatctcctctga

SEQ ID NO: 74 - DNA, 1674 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaaagcccttcagcggccagtagcatctga

SEQ ID NO: 75 - DNA, 1221 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccagcggccagtag

SEQ ID NO: 76 - DNA, 1542 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacccagtgaaaatgaccccaaccttttcgttgcactgtatgattttgtggccagtggagataacactctaa

SEQ ID NO: 77 - DNA, 1365 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcaggacgccctccgcatctcacagaacttcctgtccagcatcaatga

SEQ ID NO: 78 - DNA, 4935 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctgcagatgctgaccaactcgtgtgtgaaactccagactgtccacagcattccgctgaccatcaataaggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccggcggccactcaggacttcagcaagctcctcagttcggtgaaggaaatcagtgacatagtgcagaggtag

SEQ ID NO: 79 - DNA, 1575 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggacccagtgaaaatgaccccaaccttttcgttgcactgtatgattttgtggccagtggagataacactctaa

SEQ ID NO: 80 - DNA, 997 bp, Homo sapiens
gcgaacaagggcagcaaagctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaaaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccctaactacgacaagt

SEQ ID NO: 81 - DNA, 922 bp, Homo sapiens
gcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagt

SEQ ID NO: 82 - DNA, 1079 bp, Homo sapiens
gtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagt

SEQ ID NO: 83 - DNA, 1496 bp, Homo sapiens
cccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgccgaccaactcgtgtgtgaaactccagactgtccacagcattccgctgaccatcaataaggaagatgatgagtctccggggctctatgggtttctgaatgtcatcgtccactcagccactggatttaagcagagttcaaatctgtactgcaccctggaggtggattcctttgggtattttgtgaataaagcaaagacgcgcgtctacagggacacagctgagccaaactggaacgaggaatttgagatagagctggagggctcccagaccctgaggatactgtgctatgaaaagtgttacaacaagacgaagatccccaaggaggacggcgagagcacggacagactcatggggaagggccaggtccagctggacccgcaggccctgcaggacagagactggcagcgcaccgtcatcgccatgaatgggatcgaagtaaagctctcggtcaagttcaacagcagggagttcagcttgaagaggatgccgtcccgaaaacagacaggggtcctcggagtcaagattgctgtggtcaccaagagagagaggtccaaggtgccctacatcgtgcgccagtgcgtggaggagatcgagcgccgaggcatggaggaggtgggcatctaccgcgtgtccggtgtggccacggacatccaggcactgaaggcagccttcgacgtcaaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagcatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagccctctgtctatggtgtgtcccccaactacgacaagtgggagatggaacgcacggacatcaccatgaagcacaa

SEQ ID NO: 84 - DNA, 942 bp, Homo sapiens
gtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaa

SEQ ID NO: 85 - DNA, 707 bp, Homo sapiens
cccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaacaaggaaggtgaaaagctccgggtcttaggctataatcacaatggggaatggtgtgaagcccaaaccaaaaatggccaaggctgggtcccaagcaactacatcacgccagccaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaa

SEQ ID NO: 86 - DNA, 1758 bp, Homo sapiens
gtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggcctccgtggacaactacggagttgccatggaaatggctgagaagtgctgtcaggccaatgctcagtttgcagaaatctccgagaacctgagagccagaagcaacaaagatgccaaggatccaacgaccaagaactctctggaaaaagcccttcagcggccagtagcatctgactttgagcctcagggtctgagtgaagccgctcgttggaactccaaggaagaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaa

SEQ ID NO: 87 - DNA, 782 bp, Homo sapiens
cccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaagtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaa

SEQ ID NO: 88 - DNA, 742 bp, Homo sapiens
gtggagacactgtgtgtatctctagataagaagatatgcaccacgttgaaaatactcagtgtagatctctatgtgtataggtatctgtatatctttccttttgtttacaactgttaaaaaacctcaaaatagttctcttcaaaagaagagagattccaagcaacccatctttcttcagtatgtatgttctgtacatacttatcggagcgcgccagtaagtatcaggcatatatatctgtctgttagcaatgattattacatcatcagatcagcatgtgctatactccctgcaagaaatatactgacatgaacaggcagttcttggagaagaaagagcatttctttaagtacctggggaatacagctctcagtgatcagcagggagtttatttgaggacatcagtcacctttggggttgccatgtacaatgagatttataatcatgatactcttcggtggtagtttcaaaagacactactaatacgcaggaagcgttccagctatttaatgctggcaactactgtttaatggtcagttaaatctgtgataatggttggaagtgggtggggttatgaaattgtagatgtttttagaaaaacttgtgaatgaaaatgaatccaagtgtttcatgtgaagatgttgagccattgctatcatgcattcctgtctcatggcagaaaattttgaagattaaaaaataaaataatcaaaatgtttcctctttctaaaaaaaaaaaaaaaa

SEQ ID NO: 89 - DNA, 5565 bp, Homo sapiens
ggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcacatcacgaaactttattttacgaggaacatttaatgatggcttccaaatagaagtacagggtcatacagatgagctttggggtcttgccacacatcccttcaaagatttgctcttgacatgtgctcaggacaggcaggtgtgcctgtggaactcaatggaacacaggctggaatggaccaggctggtagatgaaccaggacactgtgcagattttcatccaagtggcacagtggtggccataggaacgcactcaggcaggtggtttgttctggatgcagaaaccagagatctagtttctatccacacagacgggaatgaacagctctctgtgatgcgctactcaatagatggtaccttcctggctgtaggatctcatgacaactttatttacctctatgtagtctctgaaaatggaagaaaatatagcagatatggaaggtgcactggacattccagctacatcacacaccttgactggtccccagacaacaagtatataatgtctaactcgggagactatgaaatattgtactgggacattccaaatggctgcaaactaatcaggaatcgatcggattgtaaggacattgattggacgacatatacctgtgtgctaggatttcaagtatttggtgtctggccagaaggatctgatgggacagatatcaatgcactggtgcgatcccacaatagaaaggtgatagctgttgccgatgacttttgtaaagtccatctgtttcagtatccctgctccaaagcaaaggctcccagtcacaagtacagtgcccacagcagccatgtcaccaatgtcagttttactcacaatgacagtcacctgatatcaactggtggaaaagacatgagcatcattcagtggaaacttgtggaaaagttatctttgcctcagaatgagactgtagcggatactactctaaccaaagcccccgtctcttccactgaaagtgtcatccaatctaatactcccacaccgcctccttctcagcccttaaatgagacagctgaagaggaaagtagaataagcagttctcccacacttctggagaacagcctggaacaaactgtggagccaagtgaagaccacagcgaggaggagagtgaagagggcagcggagaccttggtgagcctctttatgaagagccatgcaacgagataagcaaggagcaggccaaagccacccttctggaggaccagcaagacccttcgccctcgtcctaacaccctggcttcagtgcaactcttttccttcagctgcatgtgattttgtgataaagttcaggtaacaggatgggcagtgatggagaatcactgttgattgagattttggtttccatgtgatttgttttcttcaatagtcttattttcagtctctcaaatacagccaacttaaagttttagtttggtgtttattgaaaattaaccaaacttaatactaggagaagactgaatcattaatgatgtctcacaaattactgtgtacctaagtggtgtgatgtaaatactggaaacaaaaacagcagttgcattgattttgaaaacaaacccccttgttatctgaacatgttttcttcaggaacaaccagaggtatcacaaacactgttactcatctactggctcagactgtactacttttttttttttttttcctgaaaaagaaaccagaaaaaaatgtactcttactgagataccctctcaccccaaatgtgtaatggaaaatttttaattaagaaaaacttcagttttgccaagtgcaatggtgttgccttctttaaaaaatgccgttttcttacactaccagtggatgtccagacatgctcttagtctactagagaggtgctgccttttctaagtcataatgaggaacagtcccttaatttcttgtgtgcaactctgttttatcctagaactaagagagcattggtttgttaaagagctttcaatgtatattaaaaccttcaatactcagaaatgatggattcctccaaggagtcctttactagcctaaacattctcaaatgtttgagattcaagtgaatggaaggaaaaccacatgcctttaaaactaaactgtaataattacctggctaatttcagctaagccttcatcataatttgttccctcagtaataggagaaatataaatacagtaagtttagattattgaattggtgcttgaaatttattggttttgttgtaattttatacagattatatgagggataagatactcatcaaattgcaaattcttttttttacagaagtgtgggtaacagtcacagcagttttttttaccaacagcatacttaacagacttgctgtgtagcagtttttttctggtggagttgctgtaagtcttgtaagtctaatgtggctatcctactcttttgggcaatgcatgtattatgcattggaaaggtattttttttaagttctgttggctagctatggttttcagtacatttcctactttaagagtaattactgacaaatatgtatttcctatatgtttatactttgattataaaaaagtattttgttttgattttttaacttgctgcattgttttgatactttctatttttttggtcaaatcatgtttagaaactttggatgagttaagaagtcttaagtatgcaggcgtttacgtgattgtgccattccaaagtgcatcagaactgtcattcccttctaatatcttctcaggagtaatacaaatcaggtatttcatcatcatttggtaatatgaaaactccagtgaactcccaaggacatttacaacatttatattcacacgctgtatggaagggtgtgggtgtgtgtgaaggggcgagtggagacactgtgtgtatctctagataagaagatatgcaccacgttgaaaatactcagtgtagatctctatgtgtataggtatctgtatatctttccttttgtttacaactgttaaaaaacctcaaaatagttctcttcaaaagaagagagattccaagcaacccatctttcttcagtatgtatgttctgtacatacttatcggagcgcgccagtaagtatcaggcatatatatctgtctgttagcaatgattattacatcatcagatcagcatgtgctatactccctgcaagaaatatactgacatgaacaggcagttcttggagaagaaagagcatttctttaagtacctggggaatacagctctcagtgatcagcagggagtttatttgaggacatcagtcacctttggggttgccatgtacaatgagatttataatcatgatactcttcggtggtagtttcaaaagacactactaatacgcaggaagcgttccagctatttaatgctggcaactactgtttaatggtcagttaaatctgtgataatggttggaagtgggtggggttatgaaattgtagatgtttttagaaaaacttgtgaatgaaaatgaatccaagtgtttcatgtgaagatgttgagccattgctatcatgcattcctgtctcatggcagaaaattttgaagattaaaaaataaaataatcaaaatgtttcctctttctaaaaaaaaaaaaaaaaaa

SEQ ID NO: 90 - DNA, 5391 bp, Homo sapiens
ggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcacatcacgaaactttattttacgaggaacatttaatgatggcttccaaatagaagtacagggtcatacagatgagctttggggtcttgccacacatcccttcaaagatttgctcttgacatgtgctcaggacaggcaggtgtgcctgtggaactcaatggaacacaggctggaatggaccaggctggtagatgaaccaggacactgtgcagattttcatccaagtggcacagtggtggccataggaacgcactcaggcaggtggtttgttctggatgcagaaaccagagatctagtttctatccacacagacgggaatgaacagctctctgtgatgcgctactcaatagatggtaccttcctggctgtaggatctcatgacaactttatttacctctatgtagtctctgaaaatggaagaaaatatagcagatatggaaggtgcactggacattccagctacatcacacaccttgactggtccccagacaacaagtatataatgtctaactcgggagactatgaaatattgtactgggacattccaaatggctgcaaactaatcaggaatcgatcggattgtaaggacattgattggacgacatatacctgtgtgctaggatttcaagtatttggtgtctggccagaaggatctgatgggacagatatcaatgcactggtgcgatcccacaatagaaaggtgatagctgttgccgatgacttttgtaaagtccatctgtttcagtatccctgctccaaagcaaaggctcccagtcacaagtacagtgcccacagcagccatgtcaccaatgtcagttttactcacaatgacagtcacctgatatcaactggtggaaaagacatgagcatcattcagtggaaacttgtggaaaagttatctttgcctcagaatgagactgtagcggatactactctaaccaaagcccccgtctcttccactgaaagtgtcatccaatctaatactcccacaccgcctccttctcagcccttaaatgagacagctgaagaggaaagtagaataagcagttctcccacacttctggagaacagcctggaacaaactgtggagccaagtgaagaccacagcgaggaggagagtgaagagggcagcggagaccttggtgagcctctttatgaagagccatgcaacgagataagcaaggagcaggccaaagccacccttctggaggaccagcaagacccttcgccctcgtcctaacaccctggcttcagtgcaactcttttccttcagctgcatgtgattttgtgataaagttcaggtaacaggatgggcagtgatggagaatcactgttgattgagattttggtttccatgtgatttgttttcttcaatagtcttattttcagtctctcaaatacagccaacttaaagttttagtttggtgtttattgaaaattaaccaaacttaatactaggagaagactgaatcattaatgatgtctcacaaattactgtgtacctaagtggtgtgatgtaaatactggaaacaaaaacagcagttgcattgattttgaaaacaaacccccttgttatctgaacatgttttcttcaggaacaaccagaggtatcacaaacactgttactcatctactggctcagactgtactacttttttttttttttttcctgaaaaagaaaccagaaaaaaatgtactcttactgagataccctctcaccccaaatgtgtaatggaaaatttttaattaagaaaaacttcagttttgccaagtgcaatggtgttgccttctttaaaaaatgccgttttcttacactaccagtggatgtccagacatgctcttagtctactagagaggtgctgccttttctaagtcataatgaggaacagtcccttaatttcttgtgtgcaactctgttttatcctagaactaagagagcattggtttgttaaagagctttcaatgtatattaaaaccttcaatactcagaaatgatggattcctccaaggagtcctttactagcctaaacattctcaaatgtttgagattcaagtgaatggaaggaaaaccacatgcctttaaaactaaactgtaataattacctggctaatttcagctaagccttcatcataatttgttccctcagtaataggagaaatataaatacagtaagtttagattattgaattggtgcttgaaatttattggttttgttgtaattttatacagattatatgagggataagatactcatcaaattgcaaattcttttttttacagaagtgtgggtaacagtcacagcagttttttttaccaacagcatacttaacagacttgctgtgtagcagtttttttctggtggagttgctgtaagtcttgtaagtctaatgtggctatcctactcttttgggcaatgcatgtattatgcattggaaaggtattttttttaagttctgttggctagctatggttttcagtacatttcctactttaagagtaattactgacaaatatgtatttcctatatgtttatactttgattataaaaaagtattttgttttgattttttaacttgctgcattgttttgatactttctatttttttggtcaaatcatgtttagaaactttggatgagttaagaagtcttaagtatgcaggcgtttacgtgattgtgccattccaaagtgcatcagaactgtcattcccttctaatatcttctcaggagtaatacaaatcaggtatttcatcatcatttggtaatatgaaaactccagtgaactcccaaggacatttacaacatttatattcacacgctgtatggaagggtgtgggtgtgtgtgaaggggcgagtggagacactgtgtgtatctctagataagaagatatgcaccacgttgaaaatactcagtgtagatctctatgtgtataggtatctgtatatctttccttttgtttacaactgttaaaaaacctcaaaatagttctcttcaaaagaagagagattccaagcaacccatctttcttcagtatgtatgttctgtacatacttatcggagcgcgccagtaagtatcaggcatatatatctgtctgttagcaatgattattacatcatcagatcagcatgtgctatactccctgcaagaaatatactgacatgaacaggcagttcttggagaagaaagagcatttctttaagtacctggggaatacagctctcagtgatcagcagggagtttatttgaggacatcagtcacctttggggttgccatgtacaatgagatttataatcatgatactcttcggtggtagtttcaaaagacactactaatacgcaggaagcgttccagctatttaatgctggcaactactgtttaatggtcagttaaatctgtgataatggttggaagtgggtggggttatgaaattgtagatgtttttagaaaaacttgtgaatgaaaatgaatccaagtgtttcatgtgaagatgttgagccattgctatcatgcattcctgtctcatggcagaaaattttgaagattaaaaaataaaataatcaaaatgtttcctctttctaaaaaaaaaaaaaaaaaa

SEQ ID NO: 91 - DNA, 3421 bp, Homo sapiens
gctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaggaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagagtttagtgcttcaagggccaggctgccaggccatgttgcagctgaccacccacctgcagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactca

SEQ ID NO: 92 - DNA, 2082 bp, Homo sapiens
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtgcttcaagggccaggctgccaggccatgttgcagctgaccacccacctgcagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 93 - DNA, 2014 bp, Homo sapiens
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 94 - DNA, 2131 bp, Homo sapiens
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggttcagagctcaggggaggatatggagatccagggaggcttcctgtaggaagtggcctgtgtagtgcttcaagggccaggctgccaggccatgttgcagctgaccacccacctgcagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 95 - DNA, 3409 bp, Homo sapiens
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaggaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagagatatgctggatgagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaagaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 96 - DNA, 2506 bp, Homo sapiens
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagcaaaaatgtcaactcgcgaaaaaaacagccaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 97 - DNA, 2473 bp, Homo sapiens
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 98 - DNA, 3926 bp, Homo sapiens
ggcggcgcggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctgagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcacacactcacttctcttccttgggatccctaagaccgtggaggagagagaggcaatcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttcttttaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgaaaaaaaaaaaaaaaaaaaaaaaaaaa

SEQ ID NO: 99 - DNA, 4679 bp, Homo sapiens
ggcggcgcggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctgagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcacatcacgaaactttattttacgaggaacatttaatgatggcttccaaatagaagtacagggtcatacagatgagctttggggtcttgccacacatcccttcaaagatttgctcttgacatgtgctcaggacaggcaggtgtgcctgtggaactcaatggaacacaggctggaatggaccaggctggtagatgaaccaggacactgtgcagattttcatccaagtggcacagtggtggccataggaacgcactcaggcaggtggtttgttctggatgcagaaaccagagatctagtttctatccacacagacgggaatgaacagctctctgtgatgcgctactcaatagatggtaccttcctggctgtaggatctcatgacaactttatttacctctatgtagtctctgaaaatggaagaaaatatagcagatatggaaggtgcactggacattccagctacatcacacaccttgactggtccccagacaacaagtatataatgtctaactcgggagactatgaaatattgtacttgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcacacactcacttctcttccttgggatccctaagaccgtggaggagagagaggcaatcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttcttttaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgaaaaaaaaaaaaaaaaaaaaaaaaaaa

SEQ ID NO: 100 - DNA, 5905 bp, Homo sapiens
ctcctcccgcaccgccctgtcgcccaacggcggcctcaggagtgatcgggcagcagtcggccggccagcggacggcagagcgggcggacgggtaggcccggcctgctcttcgcgaggaggaagaaggtggccactctcccggtccccagaacctccccagcccccgcagtccgcccagaccgtaaagggggacgctgaggagccgcggacgctctccccggtgccgccgccgctgccgccgccatggctgccatgatggatcggaagtgagcattagggttaacggctgccggcgccggctcttcaagtcccggctccccggccgcctccacccggggaagcgcagcgcggcgcagctgactgctgcctctcacggccctcgcgaccacaagccctcaggtccggcgcgttccctgcaagactgagcggcggggagtggctcccggccgccggccccggctgcgagaaagatggcggacctggccgagtgcaacatcaaagtgatgtgtcgcttcagacctctcaacgagtctgaagtgaaccgcggcgacaagtacatcgccaagtttcagggagaagacacggtcgtgatcgcgtccaagccttatgcatttgatcgggtgttccagtcaagcacatctcaagagcaagtgtataatgactgtgcaaagaagattgttaaagatgtacttgaaggatataatggaacaatatttgcatatggacaaacatcctctgggaagacacacacaatggagggtaaacttcatgatccagaaggcatgggaattattccaagaatagtgcaagatatttttaattatatttactccatggatgaaaatttggaatttcatattaaggtttcatattttgaaatatatttggataagataagggacctgttagatgtttcaaagaccaacctttcagttcatgaagacaaaaaccgagttccctatgtaaaggggtgcacagagcgttttgtatgtagtccagatgaagttatggataccatagatgaaggaaaatccaacagacatgtagcagttacaaatatgaatgaacatagctctaggagtcacagtatatttcttattaatgtcaaacaagagaacacacaaacggaacaaaagctgagtggaaaactttatctggttgatttagctggtagtgaaaaggttagtaaaactggagctgaaggtgctgtgctggatgaagctaaaaacatcaacaagtcactttctgctcttggaaatgttatttctgctttggctgagggtagtacatatgttccatatcgagatagtaaaatgacaagaatccttcaagattcattaggtggcaactgtagaaccactattgtaatttgctgctctccatcatcatacaatgagtctgaaacaaaatctacactcttatttggccaaagggccaaaacaattaagaacacagtttgtgtcaatgtggagttaactgcagaacagtggaaaaagaagtatgaaaaagaaaaagaaaaaaataagatcctgcggaacactattcagtggcttgaaaatgagctcaacagatggcgtaatggggagacggtgcctattgatgaacagtttgacaaagagaaagccaacttggaagctttcacagtggataaagatattactcttaccaatgataaaccagcaaccgcaattggagttataggaaattttactgatgctgaaagaagaaagtgtgaagaagaaattgctaaattatacaaacagcttgatgacaaggatgaagaaattaaccagcaaagtcaactggtagagaaactgaagacgcaaatgttggatcaggaggagcttttggcatctaccagaagggatcaagacaatatgcaagctgagctgaatcgccttcaagcagaaaatgatgcctctaaagaagaagtgaaagaagttttacaggccctagaagaacttgctgtcaattatgatcagaagtctcaggaagttgaagacaaaactaaggaatatgaattgcttagtgatgaattgaatcagaaatcggcaactttagcgagtatagatgctgagcttcagaaacttaaggaaatgaccaaccaccagaaaaaacgagcagctgagatgatggcatctttactaaaagaccttgcagaaataggaattgctgtgggaaataatgatgtaaagcagcctgagggaactggcatgatagatgaagagttcactgttgcaagactctacattagcaaaatgaagtcagaagtaaaaaccatggtgaaacgttgcaagcagttagaaagcacacaaactgagagcaacaaaaaaatggaagaaaatgaaaaggagttagcagcatgtcagcttcgtatctctcaacatgaagccaaaatcaagtcattgactgaataccttcaaaatgtggaacaaaagaaaagacagttggaggaatctgtcgatgccctcagtgaagaactagtccagcttcgagcacaagagaaagtccatgaaatggaaaaggagcacttaaataaggttcagactgcaaatgaagttaagcaagctgttgaacagcagatccagagccatagagaaactcatcaaaaacagatcagtagtttgagagatgaagtagaagcaaaagcaaaacttattactgatcttcaagaccaaaaccagaaaatgatgttagagcaggaacgtctaagagtagaacatgagaagttgaaagccacagatcaggaaaagagcagaaaactacatgaacttacggttatgcaagatagacgagaacaagcaagacaagacttgaagggtttggaagagacagtggcaaaagaacttcagactttacacaacctgcgcaaactctttgttcaggacctggctacaagagttaaaaagagtgctgagattgattctgatgacaccggaggcagcgctgctcagaagcaaaaaatctcctttcttgaaaataatcttgaacagctcactaaagtgcacaaacagttggtacgtgataatgcagatctccgctgtgaacttcctaagttggaaaagcgacttcgagctacagctgagagagtgaaagctttggaatcagcactgaaagaagctaaagaaaatgcatctcgtgatcgcaaacgctatcagcaagaagtagatcgcataaaggaagcagtcaggtcaaagaatatggccagaagagggcattctgcacagattgctaaacctattcgtcccgggcaacatccagcagcttctccaactcacccaagtgcaattcgtggaggaggtgcatttgttcagaacagccagccagtggcagtgcgaggtggaggaggcaaacaagtgtaatcgtttatacatacccacaggtgttaaaaagtaatcgaagtacgaagaggacatggtatcaagcagtcattcaatgactataacctctactcccttgggattgtagaattataacttttaaaaaaaatgtataaattatacctggcctgtacagctgtttcctacctactcttcttgtaaactctgctgcttcccaacacaactagagtgcaattttggcatcttaggagggaaaaaggacagtttacaactgtggccctatttattacacagtttgtctatcgtgtcttaaatttagtctttactgtgccaagctaactgtaccttataggactgtactttttgtattttttgtgtatgtttattttttaatctcagtttaaattacctagctgctactgcttcttgtttttcttttcctattaaaacgtcttcctttttttttcttaagagaaaatggaacatttaggttaaatgtctttaaattttaccacttaacaacactacatgcccataaaatatatccagtcagtactgtattttaaaatcccttgaaatgatgatatcagggttaaaattacttgtattgtttctgaagtttgctcctgaaaactactgtttgagcactgaaacgttacaaatgcctaataggcatttgagactgagcaaggctacttgttatctcatgaaatgcctgttgccgagttattttgaatagaaatattttaaagtatcaaaagcagatcttagtttaagggagtttggaaaaggaattatatttctctttttcctgattctgtactcaacaagtcttgatggaattaaaatactctgctttattctggtgagcctgctagctaatataagtattggacaggtaataatttgtcatctttaatattagtaaaatgaattaagatattataggattaaacataattttatacggttagtactttattggccgacctaaatttatagcgtgtggaaattgagaaaaatgaagaaacaggacagatatatgatgaattaaaaatatatataggtcaattttggtctgaaatccctgaggtgtttttaacctgctacactaatttgtacactaatttatttctttagtctagaaatagtaaattgtttgcaagtcactaataatcattagataaattattttcttggccatagccgataattttgtaatcagtactaagtgtatacgtatttttgccactttttcctcagatgattaaagtaagtcaacagcttattttaggaaactgtaaaagtaatagggaaagagatttcactatttgcttcatcagtggtaggggggcggtgactgcaactgtgttagcagaaattcacagagaatggggatttaaggttagcagagaaacttggaaagttctgtgttaggatcttgctggcagaattaactttttgcaaaagttttatacacagatatttgtattaaatttggagccatagtcagaagactcagatcataattggcttatttttctatttccgtaactattgtaatttccacttttgtaataattttgatttaaaatataaatttatttatttatttttttaatagtcaaaaatctttgctgttgtagtctgcaacctctaaaatgattgtgttgcttttaggattgatcagaagaaacactccaaaaattgagatgaaatgttggtgcagccagttataagtaatatagttaacaagcaaaaaaagtgctgccaccttttatgatgattttctaaatggagaaacatttggctgcatccacatagacctttatgttttgttttcagttgaaaacttgcctcctttggcaacattcgtaaatgaagcagaatttttttttctcttttttccaaatatgttagttttgttcttgtaagatgtatcatgggtattggtgctgtgtaatgaacaacgaattttaattagcatgtggttcagaatatacaatgttaggtttttaaaaagtatcttgatggttcttttctatttataatttcagactttcataaagtgtaccaagaatttcataaatttgttttcagtgaactgctttttgctatggtaggtcattaaacacagcacttactcttaaaaatgaaaatttctgatcatctaggatattgacacatttcaatttgcagtgtctttttgactggatatattaacgttcctctgaatggcattgatagatggttcagaagagaaactcaatgaaataaagagaatatttattcatggcgattaattaaattatttgcctaacttaagaaaactactgtgcgtaactctcagtttgtgcttaactccatttgacatgaggtgacagaagagagtctgagtctacctgtggaatatgttggtttattttcagtgcttgaagatacattcacaaatacttggtttgggaagacaccgtttaattttaagttaacttgcatgttgtaaatgcgttttatgtttaaataaagaggaaaattttttgaaatgtaaaaaaaaaaaaaaaaaa

SEQ ID NO: 101 - DNA, 3096 bp, Homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcaggtagggagaggaacgcgttgtggtgatcagcagctcggaagactcagatgccgaaaactcgtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccaggaacatcctgcccagctgcaaaggggcatcagcccaccccaccggatacgaggggctgtgcgatcccgcagccgctccctccggggctcctcccatttatcccagtggctcaacaacttttttgccctccccttctcctccatggcttcccagcttgacatgtcttccgtggtgggggcaggggaaagcagagcccagactcttggagcaggtgttccccctggggactctgtcagaggctccatggaggcctctcaagtccaagtgcctctggaagcctctccaattacattcccaccaccctgtgccccagaaaggccccccatcagcccagtcccaggcgcccgtcaagcaggcctctgagagtgctacccttctcttgtaaccttgcagccaacacccctgcccggcccctgagctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtattctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaa

SEQ ID NO: 102 - DNA, 2944 bp, Homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccaggaacatcctgcccagctgcaaaggggcatcagcccaccccaccggatacgaggggctgtgcgatcccgcagccgctccctccggggctcctcccatttatcccagtggctcaacaacttttttgccctccccttctcctccatggcttcccagcttgacatgtcttccgtggtgggggcaggggaaagcagagcccagactcttggagcaggtgttccccctggggactctgtcagaggctccatggaggcctctcaagtccaagtgcctctggaagcctctccaattacattcccaccaccctgtgccccagaaaggccccccatcagcccagtcccaggcgcccgtcaagcaggcctctgagagtgctacccttctcttgtaaccttgcagccaacacccctgcccggcccctgagctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtattctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaa

SEQ ID NO: 103 - DNA, 3088 bp, Homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccaggaacatcctgcccagctgcaaaggggcatcagcccaccccaccggatacgaggggctgtgcgatcccgcagccgctccctccggggctcctcccatttatcccagtggctcaacaacttttttgccctccccttctcctccatggcttcccagcttgacatgtcttccgtggtgggggcaggggaaagcagagcccagactcttggagcaggtgttccccctggggactctgtcagaggctccatggaggcctctcaagtccaagtgcctctggaagcctctccaattacattcccaccaccctgtgccccagaaaggccccccatcagcccagtcccaggcgcccgtcaagcaggcctctgagagtgctacccttctcttgtaaccttgcagccaacacccctgcccggcccctgagctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtattctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaa

SEQ ID NO: 104 - DNA, 2254 bp, Homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaagtgggttctcctggggctacccccacccctttctaatttagtctctgagtcccaaaaagaagtgcaggcagagccatctgccaggcccaggagagctctgagctctggccaacaactgcagccaggctgggcagagcactccggctcacctgggctcctggcgtgtcatttgctggcttgaataaagatgtccgccttatccagtgcctgagtgtgcgagagaggcagatgcctccaaaaaaaaaaaaaaaaa

SEQ ID NO: 105 - DNA, 2110 bp, Homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaagtgggttctcctggggctacccccacccctttctaatttagtctctgagtcccaaaaagaagtgcaggcagagccatctgccaggcccaggagagctctgagctctggccaacaactgcagccaggctgggcagagcactccggctcacctgggctcctggcgtgtcatttgctggcttgaataaagatgtccgccttatccagtgcctgagtgtgcgagagaggcagatgcctccaaaaaaaaaaaaaaaaa

SEQ ID NO: 106 - DNA, 3751 bp, Homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcggtgagtggcccagaagttcagcccaggactcctgcctccccccatttcaggtcccagggggcacagccacagcaggtgactctcagacttgccttgcgcctggggaattttccagtgaggcattgagtcccaagctgtgctgaggacagtctccaaatgacagctgcatgcctggaccaccccagccctcccactacaccagggccaggagctcttctgaaatgctgatagcatgtgtccagagccctgtctcttttctggaatgtccaagacctttgtctggagcttccttatgcattttctgtcctctaagtcctcagtgaggagggctggacaagaatccattccttggtttattacagccagtgggggccagtgaaggggtgtcaggccacagggcagctatataggggccaaacaagtgaggtttgactccatccatgtagaaagatatataaatccattccacagtgaaacaggtggcctcgtgggtagtgacccttctgtccctagaggtttattactagaggctggactatcacctgtccaggggaaaaggggatctgaatagagtgaaaggttggactgggtggcccctgaggtctcttccagccctcagtctgaggttctgtattggaaagtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccaggaacatcctgcccagctgcaaaggggcatcagcccaccccaccggatacgaggggctgtgcgatcccgcagccgctccctccggggctcctcccatttatcccagtggctcaacaacttttttgccctccccttctcctccatggcttcccagcttgacatgtcttccgtggtgggggcaggggaaagcagagcccagactcttggagcaggtgttccccctggggactctgtcagaggctccatggaggcctctcaagtccaagtgcctctggaagcctctccaattacattcccaccaccctgtgccccagaaaggccccccatcagcccagtcccaggcgcccgtcaagcaggcctctgagagtgctacccttctcttgtaaccttgcagccaacacccctgcccggcccctgagctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtattctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaaaaaaaaaaaaaaaaaaaaaaaa

SEQ ID NO: 107 - DNA, 1797 bp, Homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtattctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaa

SEQ ID NO: 108 - DNA, 1851 bp, Homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaagtgggttctcctggggctacccccacccctttctaatttagtctctgagtcccaaaaagaagtgcaggcagagccatctgccaggcccaggagagctctgagctctggccaacaactgcagccaggctgggcagagcactccggctcacctgggctcctggcgtgtcatttgctggcttgaataaagatgtccgccttatccagtgcctgagtgtgcgagagaggcagatgcctccaaaaaaaaaaaaaaaaa

SEQ ID NO: 109 - DNA, 5600 bp, Homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaacccagaagattagccagctggctgcggtgaaccgggaaagcaagttccgcgtggtcatccagcctgaagccttcttcagcatctactccaaggccgtgtccctggaggtggggctgcagcacttcctcagctttctgagctccatgcgccgccctatcttggcctgctacaagctgtgggggcctggcctcccaaacttcttccgggccctggaggacattaacaggctgtgggaattccaggaggccatctcgggcttcctggctgccctgcctctcatccgggagcgtgtgcccggggccagcagcttcaaactcaagaacctggcccagacctacctggcgagaaacatgagcgagcgcagcgccatggctgccgtgctggccatgcgtgacctgtgccgcctcctcgaggtctccccgggcccccagctggcccagcatgtctaccccttcagtagcctgcagtgctttgcctccctgcagcccctggtgcaggcagctgtgctgccccgggctgaggcccgcctcctggccctacacaacgtgagcttcatggagctgctgagtgcacaccgccgtgaccggcaggggggcctgaagaagtacagccgctatctaagcctgcagaccaccacgttgccccctgcccagcctgctttcaacctgcaggctctgggcacctactttgaaggcctgttggagggtccggcgctggcacgggcagaaggagtctccaccccacttgctggccgtggcttggcagagagggcctcccagcagagctgagaggagggggtgaccagcttggagtctctggtgggcagagagggatggggtccctgagccaggccccacccatcacagcattcccaggtcctggtacccagccctcagttgtcatttggttcagaatcagttccctttctctgggaccaaatttcccttctctaaacatcctacagagaaggttccaaagctgagcacccatcaccctaggtgtgcaccagactcctattagcccctccttccaggagctagaaccagggaggtcctgagtgaggaaggcatgacctctgggctctctaggtggccctggtcttgccccatccatcccacctgccactaccttgggtgtccttacagctctgccctgaccccagcccctgcccctggacacctgctgacagctcccacacagaacagtctgaggtgatgctggctacagccctggcagcccatggcaactcaaagtgccttccaaaccaaactgcccctcatgaggttagggtcccccaccctccacctgcccagaggccaactctgttcccttctcctttcatcccagaggggcctcatcagcaaagacagaaactgatgttccatgcatgtccctgcttccaaagcctgaccttccaaagcttgcttccttcctcctggctgcacagacacctctgcttggccacagctttgctctttggtcctcaggccaccagacccctcacagcatggcccttggctcttcctgcactgccctcaccctcagccgtcccaaggagtgcccagcactactcagcatgtagtccaggacctgcggcatcagcatcccctgggagcttcttagaaatgcagacctgtgggctctaccacaggcctctggaatcagaatcttcagggtggggcccagcagtctgtgttttaacaggttctcccggtgatgctggtgcatgctcaagtttgagaaccgctgctctcatagactgctcctccaaggggaagcagtgtggaacagcagagaaagtcccgtccctatctctgactgtgcagtgagtgtggcctaggaacaggtcccttcctaagctctagtgtccccatctgtaaaatgggctgattggcctccacggtcagagcggccatctggctgtgccatcctgcatttttaggaatggaaagcaggcctctgaggcagtggacaggaagacttctcatccttgaattctagctcccattccaaactgttagtccccaaccttgtggtcacatcagaggtcaacagcagaacagaggcaggtcagggttgtccgctctgattagcagaacgacagccccttcctcctcctccctccttcccatccctactcattagctccctgctcctgggatgcttcccaagcagcccaggcctctagcctccatggctgatgacctcagctttcacactggtgaagtctgtgtacccatactgcagccccatcccatggggcccctgaagacccactgaggaattccgtagggtcttgttcccacgaccggagtgctggctctcacagtgaattttgatgcatttaaaataagattctgatgccagactgttaaaacaggcgctggttctgaagcaccgatggaaacagattgatgcagatggaaggaggaagggagactgggcccactgatttccagccccaccatgtgtgctgttttcagatgtgattgaggggtgttctgccctgcctccactgtcacagcctttaataaagatgtgaatcttgaaagcctgatgctccaatcacagactctgctcagcatccccagaggaaccactgagaagccttttgcctggataggagggggctctcccaggcataggcttgtggccacttgcccagcacaactgtgttttgtggggcgtcttttaggacttagcagagctgagagccctttgttgctcaatgctgtgagccttaagcatgtgaatccccccacatgacaagtggggagacccagggtaggctttcctttggatcatctccaaatgggagtgccatgtggcaggaaagaagcaccgatttcaagaattacttcctagagaaagtcaggaactagagaccagagtctgcaggaactttgccactgcccttcactggctaatcctgtacttctctctgtgttccttgagtgacaggtggtaaaacccttaaaaagggaggtgtgggaggcccaggactttggtaacaggtgatgaatgtgttgtcttggccatcacaggtggagcttcagttagtacccgagggattcccctggcacaagcattataggaattagtccaccactggcgtgggtgagcagccaggtaatgggaattgatcagtgccaccacccttgaccccaactaccatcccagatgcccagattccagtcctggagttttattctctcagcttgatcctttgaccaagaagatccagtcccaatatgtcacctgtgcttcatctttggactatatctcaggtgtttacgtgtcaactaatgggagcttactgggttagaagtcaggacactgagtttcaatcctcacttagtagtctgtggccctctttggcacattaacctccctccttgatcctcaggctgcacctctggcaaatgggagaatgcggctccttttgacttcaagggatctttgtgggaaccaatgagaaaatgtgtgtgaagatagctttttggtatattttaaaagtgcacaaatttaagatcttactgctttataaagtgctttatgaattataggaaaataaacacattttagatcttagaaaaaaa

SEQ ID NO: 110 - DNA, 2872 bp, Homo sapiens
cttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgatctcaagtgcgacatcagcgcagagatccagcagcgacaggaggagctggacgccatgacgcaggcgctgcaggagcaggatagtgcctttggcgcggttcacgcgcagatgcacgcggctgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccaggaacatcctgcccagctgcaaaggggcatcagcccaccccaccggatacgaggggctgtgcgatcccgcagccgctccctccggggctcctcccatttatcccagtggctcaacaacttttttgccctccccttctcctccatggcttcccagcttgacatgtcttccgtggtgggggcaggggaaggcagagcccagactcttggagcagttgttccccctggggactctgtcagaggctccatggaggcctctcaagtccaagtgcctctggaagcctctccaattacattcccaccaccctgtgccccagaaaggccccccatcagcccagtcccaggcgcccgtcaagcaggcctctgagagtgctacccttctcttgtaaccttgcagccaacacccctgcccggcccctgagctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtgttctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaaaaaaaaa

SEQ ID NO: 111 - DNA, 2872 bp, Homo sapiens; identical to SEQ ID NO: 110

SEQ ID NO: 112 - DNA, 55 bp, Homo sapiens
tgggagaaaaccaggccagggcagttgatttgtgaattactcaaatccaaatgca

SEQ ID NO: 113 - DNA, 55 bp, Homo sapiens
tcaggagagaatctgctgggctggggatggccaggtagcagttgtcaggcctcct

SEQ ID NO: 114 - DNA, 55 bp, Homo sapiens
caaatgctattggtaatcgatgggttctattgttactgcttttacgtcttggaaa

SEQ ID NO: 115 - DNA, 55 bp, Homo sapiens
catgaccttaactctgctctctcagtctgcccctcccctcttctctctctagcca

SEQ ID NO: 116 - DNA, 55 bp, Homo sapiens
acagcaaccacgtggccagtggcgccccagagcattgatgggaacagaccccctt

SEQ ID NO: 117 - DNA, 55 bp, Homo sapiens
agtgaggtcttcctgcccaacagcaaccacgtggctgccagcagtggtgaggctg

SEQ ID NO: 118 - DNA, 55 bp, Homo sapiens
acagcaagaaattatggaaacccatttgtgttggagtgaagagcagacggcggtg

SEQ ID NO: 119 - DNA, 55 bp, Homo sapiens
gattgcagaaggttccagacactagggtcgtgtgggcatcaactgtcccattgct

SEQ ID NO: 120 - DNA, 55 bp, Homo sapiens
cttctatgaacactgccaggctggtagtcagcccctggtgttcccatgcttcctt

SEQ ID NO: 121 - DNA, 55 bp, Homo sapiens
ccggacatagtattgaggccagacagacagggaggcaggtagggaggtgggtagg

SEQ ID NO: 122 - DNA, 55 bp, Homo sapiens
gctggggagtcccagttttcggggaggcaggtagggaggtgggtagggcagtggc

SEQ ID NO: 123 - DNA, 55 bp, Homo sapiens
gtttcctctgcccccagcttatgtcccttggtcacacactgagcactgactgata

SEQ ID NO: 124 - DNA, 55 bp, Homo sapiens
cccctccccccaccagtctggattgtctaactaaatgaagcgtctttaactctag

SEQ ID NO: 125 - DNA, 55 bp, Homo sapiens
gtccctcattctgactgagccctagcctttccctccctcctcttcaagcgttaac

SEQ ID NO: 126 - DNA, 80 bp, Homo sapiens
aggcagttcataatgcatctccccttccccgtttcagaggcttctccctgtattcagggtatctccccctatctcaggga

SEQ ID NO: 127 - DNA, 55 bp, Homo sapiens
gtaaggggggagggtggtagcacatagtagttgggcaggagttgaggttaaccca

SEQ ID NO: 128 - DNA, 296 bp, Homo sapiens
gatgtctccaatacaacgacagcccagaagaggaagtgcagccagacccagtgccccaggaaggtcatcaagatggagtctgaggaggggaaggaggcaaggttggctctccccgccccgggtccgtactccaccccgctccggactccgctttggaatggctcaaaccactccattgagacccagagcagcagttctgaagagatagtgcccagccctccctcgccaccccctctaccccgcatctacaagccttgctttgtctgtcaggacaagtcctcaggctaccactatgg

SEQ ID NO: 129 - DNA, 210 bp, Homo sapiens
acaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcacagccattgagacccagagcagc

SEQ ID NO: 130 - DNA, 120 bp, Homo sapiens
acaacgacagcccagaagaggaagtgcagccagacccagtgccccaggaaggtcatcaagatggagtctgaggaggggaaggaggcaaggttggctccagccattgagacccagagcagc

SEQ ID NO: 131 - DNA, 4508 bp, Homo sapiens
cagatccagttgttctcatgcagccgtgtgcggtacgtgccgtagagatgctggcccaggcggaagctgggcacctccttactgtacttccataccctatggaagtacagtaaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcgttcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaaggtaatggactcacaaggggaagaaacatgctgagaatggaaagtctaccggccctttctttgtgaacgtcacattggccgagccgtgttcagttcccaggtggcagactcgtttttggtagtttgttttaacttccaaggtggttttacttctgatagccggtgattttccctcctagcagacatgccacaccgggtaagagctctgagtcttagtggttaagcattcctttctcttcagtgcccagcagcacccagtgttggtctgtgtccatcagtgaccaccaacattctgtgttcacatgtgtgggtccaacacttactacctggtgtatgaaattggacctgaactgttggatttttctagttgccgccaaacaaggcaaaaaaatttaaacatgaagcacacacacaaaaaaggcagtaggaaaaatgctggccctgatgacctgtccttattcagaatgagagactgcggggggggcctgggggtagtgtcaatgcccctccagggctggaggggaagaggggccccgaggatgggcctgggctcagcattcgagatcttgagaatgatttttttttaatcatgcaacctttccttaggaagacatttggttttcatcatgattaagatgattcctagatttagcacaatggagagattccatgccatctttactatgtggatggtggtatcagggaagagggctcacaagacacatttgtcccccgggcccaccacatcatcctcacgtgttcggtactgagcagccactacccctgatgagaacagtatgaagaaagggggctgttggagtcccagaattgctgacagcagaggctttgctgctgtgaatcccacctgccaccagcctgcagcacaccccacagccaagtagaggcgaaagcagtggctcatcctacctgttaggagcaggtagggcttgtactcactttaatttgaatcttatcaacttactcataaagggacaggctagctagctgtgttagaagtagcaatgacaatgaccaaggactgctacacctctgattacaattctgatgtgaaaaagatggtgtttggctcttatagagcctgtgtgaaaggcccatggatcagctcttcctgtgtttgtaatttaatgctgctacaaggtgtttctgtttcttagattctgaccatgactcataagcttcttgtcattcttcattgc

SEQ ID NO: 132 - DNA, 2210 bp, Homo sapiens
ccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcgtacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtactgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaaaaaaaaaaaaaaa

SEQ ID NO: 133 - DNA, 3486 bp, Homo sapiens
ccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccagtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcgtacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtactgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaaaaaaaaaaaaaaaaa

SEQ ID NO: 134 - DNA, 5629 bp, Homo sapiens
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaaggtaatggactcacaaggggaagaaacatgctgagaatggaaagtctaccggccctttctttgtgaacgtcacattggccgagccgtgttcagttcccaggtggcagactcgtttttggtagtttgttttaacttccaaggtggttttacttctgatagccggtgattttccctcctagcagacatgccacaccgggtaagagctctgagtcttagtggttaagcattcctttctcttcagtgcccagcagcacccagtgttggtctgtgtccatcagtgaccaccaacattctgtgttcacatgtgtgggtccaacacttactacctggtgtatgaaattggacctgaactgttggatttttctagttgccgccaaacaaggcaaaaaaatttaaacatgaagcacacacacaaaaaaggcagtaggaaaaatgctggccctgatgacctgtccttattcagaatgagagactgcggggggggcctgggggtagtgtcaatgcccctccagggctggaggggaagaggggccccgaggatgggcctgggctcagcattcgagatcttgagaatgatttttttttaatcatgcaacctttccttaggaagacatttggttttcatcatgattaagatgattcctagatttagcacaatggagagattccatgccatctttactatgtggatggtggtatcagggaagagggctcacaagacacatttgtcccccgggcccaccacatcatcctcacgtgttcggtactgagcagccactacccctgatgagaacagtatgaagaaagggggctgttggagtcccagaattgctgacagcagaggctttgctgctgtgaatcccacctgccaccagcctgcagcacaccccacagccaagtagaggcgaaagcagtggctcatcctacctgttaggagcaggtagggcttgtactcactttaatttgaatcttatcaacttactcataaagggacaggctagctagctgtgttagaagtagcaatgacaatgaccaaggactgctacacctctgattacaattctgatgtgaaaaagatggtgtttggctcttatagagcctgtgtgaaaggcccatggatcagctcttcctgtgtttgtaatttaatgctgctacaagatgtttctgtttcttagattctgaccatgactcataagcttcttgtcattcttcattgcttgtttgtggtcacagatgcacaacactcctccagtcttgtgggggcagcttttgggaagtctcagcagctcttctggctgtgttgtcagcactgtaacttcgcagaaaagagtcggattaccaaaacactgcctgctcttcagacttaaagcactgataggacttaaaatagtctcattcaaatactgtattttatataggcatttcacaaaaacagcaaaattgtggcattttgtgaggccaaggcttggatgcgtgtgtaatagagccttgtggtgtgtgcgcacacacccagagggagagtttgaaaaatgcttattggacacgtaacctggctctaatttgggctgtttttcagatacactgtgataagttcttttacaaatatctatagacatggtaaacttttggttttcagatatgcttaatgatagtcttactaaatgcagaaataagaataaactttctcaaattattaaaaatgcctacacagtaagtgtgaattgctgcaacaggtttgttctcaggagggtaagaactccaggtctaaacagctgacccagtgatggggaatttatccttgaccaatttatccttgaccaataacctaattgtctattcctgagttataaaagtccccatccttattagctctactggaattttcatacacgtaaatgcagaagttactaagtattaagtattactgagtattaagtagtaatctgtcagttattaaaatttgtaaaatctatttatgaaaggtcattaaaccagatcatgttcctttttttgtaatcaaggtgactaagaaaatcagttgtgtaaataaaatcatgtatcataaaaaaaaaaaaaaaa

SEQ ID NO: 135 - DNA, 4174 bp, Homo sapiens
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaccaaagtctgctctgaacctttttatttgtaaatgtctgactttgcatccagtttacatttaggcattattgcaactatgtttttctaaaaggaagtgaaaataagtgtaattaccacattgcccagcaacttaggatggtagaggaaaaaacagatcagggcggaactctcaggggagaccaagaacaggttgaataaggcgcttctggggtgggaatcaagtcatagtacttctactttaactaagtggataaatatacaaatctggggaggtattcagttgagaaaggagccaccagcaccactcagcctgcactgggagcacagccaggttcccccagacccctcctgggcaggcaggtgcctctcagaggccacccggcactggcgagcagccactggccaagcctcagccccagtcccagccacatgtcctccatcaggggtagcgaggttgcaggagctggctggccctgggaggacgcacccccactgctgttttcacatcctttcccttacccaccttcaggacggttgtcacttatgaagtcagtgctaaagctggagcagttgctttttgaaagaacatggtctgtggtgctgtggtcttacaatggacagtaaatatggttcttgccaaaactccttcttttgtctttgattaaatactagaaatttaaaaaaaaaaaaaaa

SEQ ID NO: 136 - DNA, 226 bp, Homo sapiens
ctctctgtctgaacttgggcaaggcgatgcaggtccatcctgacctggtatggtcatggaaggggcttccaggagcgatcgtttgcaacctgctctgtgctgcatttcagagaacgcctccccgagtgagcttcgagacctgctgtcagagttcaacgtcctgaagcaggtcaaccacccacatgtcatcaaattgtatggggcctgcagccaggatggtaaggcc

SEQ ID NO: 137 - DNA, 204 bp, Homo sapiens
tcgggacttggcagccagaaacatcctggtagctgaggggcggaagatgaagatttcggatttcggcttgtcccgagatgtttatgaagaggattcgtacgtgaagaggagccaggtgcccagtcccggggatgaggcggggctcccagggatcccaggtgcaccatggggcaggcagtgcccttgggaagcctaggaaagata

SEQ ID NO: 138 - DNA, 266 bp, Homo sapiens
agtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgca

SEQ ID NO: 139 - DNA, 30430 bp, Homo sapiens
  modified_base (221)..(221): a, c, t, g, unknown or other
caagagcaaaactccatctcaaaaataaataaataaataaataaataaataaataaataaataaataaaaattacatagataagatgcacggaccttagctgcacaatactgacaaacgaatatgcccacgtgatctgcacccttctcaggatatgggaccttcacattccccagaaagtgccctgtggcccttcccaatctgtccacctcccccacaccnccgcctgccacaggcagccagtctgatccattgcctaacatccgtccagaatcatgccatgtgcgctcttctctgcctggcttcctcaccttggcatggtttgtgtgtgtgacccatccaggtgtttgcgtgtatccatagttcatccttttccattgctgggcaggattctgtggcatggctggaccacggtgtgcctgtccatccacctgctgaagggcctctaggctcttgacttctggaatcatccttccatttccaactatggcatttgatagacgtatgttttcacttttcttggatgaatggagtggaattgcacggtcatatggtaggcgtatgtttagataaagatttatgactttcctgtaaagtgctttcttcattttttggcaactgttgcctgccccacatgggtggcagtgtctcctgcccttgaacactctggctctgggtgacctgttgagaagtctgtatggttcaggtgcccttccggcttggatgattgagaataggcttgtcactgaggatactgcttcccctgtttccttttctttttctgttttaaagcagttcttttctagcccgtgtgtaactatacaaataattgagtgttcattttctcaggaacagagatgaaaaactcctgctaagatcggaattttaaattaatgatttttttttttttgtccttgaagaagccttattctcaccatccctcactcacttccctacttcccacagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacncggctgcatgagaacaactggatctgcatccaggaggacaccggcctcctctaccttaaccggagcctggaccatagctcctgggagaagctcagtgtccgcataagggagccgtcccaacacccaccccgtgccccaccccaccccttcctcaagccgcccttatcacagccgctgacactgaagcttggcatggcttcccccccaccgctggtgtggaaggcgtcaggggttaagtgaggctggcctgcctctgtgtccagcctggagagaagccaggacaagcttcagggctgcgtgaggctgtaggagtttggagacacacgggaatcatccgggtacactcttctgccagacccgaatccctcttccagtggggctaaaaaggattaggaggtttcgagagagggagtaagtggggctggaaaaatcccaaggttttgatttagcaaatgacctgcaactctctggagaagcggcagttcccaaggaaagcctgacttcttatcagcagctggctgcccttgtacctggcagggacttgggcctgtcccangtgtccagggaggaaagaggagcagtcagagccagagtcacggccacatgtgacattggtaatgggaggggtagggggggaagcagagcccctgtgacaggaaacggtgatgctgcacctggtagctcccatccctagcacccctccagggccacagcttgcacagccctcacaaccaccctgagccctgtctgctggtgagaggcaagcctcaggctatgaatgcagagcaggcatccctgccctacccaggcctatctgccagggacagccagggtgcaggggagggaggagacttgggtgggcaactccctccggcgcagcccaggcagttcctggaggaggcagcatctgaggttggtcgnggttctacgcacttagttgctgccctcttcatcctgagcctccagggtggagcttgaggaatgggagtccctgggtgggggccagtgtggaatttgccctgggcctgcatttcgtaggaggcctaagccctccaaggtggggagaggcttcatgcacagccagttcctgtggaggaagaaaccacagggtaatttaacagggagagtttgtgtaaagaattaattacaacaggggattggagcaatgagggacaggctagcaaaaagcaaagagaatgccagagaatatgggaagagcagctctgaggagcagccgcccacctagggctgagatggaggctgggaaagccctcgcagcccagctgaaatggctatggcgcttcggtgatggaacttgccggaaatgccccctctaggcattggggaagtatgtctgcggccactccaaacctcctgaggagggtgtggcagggggggtgctggtcaccccttactggagaagttgccaccatcagagttggggagccttagacccagtgctcaacacacctccctgaggaaggggcctctaagccaagaactgaatgatgagagctaggcctgtggggcattggtgctggcagcaggactgtgcagcagaggtaaggaggtggcaggcagagctggaacaccagtctgagccttgtggaccttggtggggaccagggtttacaccagccctggagctcctgcctcctccccattcccgactgcctggcagatgtggccgatgcccccacagacctgacttctctctgcagaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagtgccgaccttgtggggccgccccacagtgcctgctactgctggtcttgctctgcgagcccttgacacaagccatctggtttattcttcaccttcatgccatcagttcattcaatattccagaagtacctcttgcatgcctgccaggggccaggtactgttctaggagctaaggatataacaatgaacacaacgggttggaatcttgcctgcctgcagctttcattcttgtggggcagacagagcgtctgagcatgccaaggaaatcaggtggcttctggaaggagctagctggggaggaaactgggaagagggaaacgctgaagaggaggggaacagacgaggagacctccattcctctatcgtaaattgggtggtcagggtgggggctagcagagcaaaggtgggagggtgccgggtaggcaggcctagccccttggagggcaccacagtgtgaccctcgtgtgcctggagtgcactgagcgacagggaagagctgaggctgatggggcagaatagttcctggggcanggggcaggtctgggagtggcttgcagggctttctccggaggaacatggggagctatggaagactttggagggagggagtgctggcaggacctgacttagttttaaaaggattagtcagactactgtgttaggaatggtttggggtgggaaggttcaggggcgaagctgggagacaatcngaaggctacacggagaaacctctgaacaggaggatgtggggtgtgatgccgggatacgggtggaagccgaggcatggcgagaagcaggcagctgcagccagtcagagggtgggatgagctggatcccagtggagaggggagaaggcagctgactaggtgcagggaggccccatttgaccatcagaaagtcaggaagtggggctcaaccaggtcacagaggctgcaccgctggcaagtgtgtcctaaattcacactaactttatcacacaaacatggcaagaatattaaaatctcagtaaaggaagacaagccatcacttatgcttccccagattcctttcattctctggggcagggttgatggagcacttggttttttgcatgctgccctttttcactaaaaattatgtgattaaatttagtttagtggggggttttttgtaatcacactcaacagaggaaagcattatatgataagcatttcccacatacgtaattcatgcgcaaggcatggttctgcctgggaggcttgtggagttgcccccgcctccttggggtgggttgtggggtcttcagcggtgaccttccgccttcagcatgcccccttttctggattatttctggaggacaaatccgaggtcaggctcgttgaagactgtgttgtggttcctggccctgcttcaggggcgatgtagtctagcgcgagctgccatgcagggcacggagcattcgcttttgtctcgacatccggccttcagaggatgaccagcctggcgacgcactggacaaggaggtgtagagtggggatgcacactaggcggtcggaaatcattatgcgttaatgtcacatcataccccagatgtccactgtgtttgttttcaagtggtagaatcgtggctttctcacgttctctaaaatgaacacatattgcttccataattgaaaaaaaaaaaaacctaacagtttttaaaaagcagccaaggtcaccaatatggtgaaaggccgtccctactaaaaatacaaaaattagccgggcatggtggcgggcgcctgtagtcccagctactcgggaggctgaggcaggagaatcgcctgaacccgggagttggaggttgcagtgagccaagatcgcgccactgcactccagcctgggcaacacagcgagactccatcaaaaacaaaaaaaaagcagccaaggccagtagctggctctctagggctgcagtgcagcttgggctgaggcaaagccacccttccccacacaaggccactcctgatcaaggccaggtgggcggaggggtgtcccagtccctactggttgatcagggtgcctggtcctggctcttcccaaccagcacgagtgaggacgcagctgcagcaggacgtaagcacagtcatcgctgcaaactgcaaactcgtaagcacagtcatcgctgcaaactgcaaactcgtgctccgagcgctgccctcccctgtggagcggaggaggggaggcctggggccgcggcggtgtgcgccccgctctgaccgcagagcccccttcccgaggaaagcggctggcccggtcccggctggtgatcacgcggggcccctgtctgcttggtgcgcaggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggtgcttgtccgcgcgtgctgtggtctacccagtgtctgtctccggccacattcgtttctcggtcggtttagtgtccgtgtagccacccaaccgtgtggccgaccattcgcgctttcatttgtccttcgcctccgtctgcgccgtctgtcctagggggaggggaagggggagtcctgccagcacccagctgggccttgcctcgggaggcaaggaccaggacgtggcccgagggctcgcgtctggggcatacttgtgccgctgcaggcgggcgcggcgcgctgcccgggcggggagcatctgccgggagggcactccctcccaccagcagttagcccccaacgggagggcccttgagtgaccacgagcagagccggggattggagaaggacgggaaggcggatcacctccggcgccgcccgccccgcccttctccggctcgcgctggtggagcgcgaccgccacctgctgggcctcggccttcctgcagccggcccacccagcaggggccgtgggagagtgggcgtggggactgaggtaggtagtacgttgccttgttccgcttctctgggcacaccacgtgcaggtttggcagggaaagggggtctggtcgaaagcacaatttgtgagtaaaggctatgtgggcctccctggggtgacagcagaggcctggtgggcagagccatcccaggtgcccagcggctttcctcncnaccccctccagcctggggtaggaggatgcttggaacagagcgctctgatgtacacctggcctcggagctcggctctgccggctgttcctgtgtgaccttgacctgaccttcaccctgtggggcccagcttcctcaggggagagtggaggtgctcatcctgctctctggggacctggatgggacctgccagcagggtgcctgggcatcggctgtaattctccttataagaggtctgcattgtacctgttacacaggcgaggctcagtggctcagggaaggtgacggccaggctggccagtgaccccgtgggaacttgaacccaggtcagactgtccccagacctggctctgacaacacacatctggtccacctatgggctgtgtgggacgtgcagcatcctaaggtctctggttttggggggtctgaggggcccatctcgcctgcactgaccaacgccctctgcatcctgcaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactatagtaagaggggctggtggcacggcctgctaggcccccaggaaatgaggtgctcgctcttcatgggcaagcagcaccctacacacatgcacacctggcatggccctctgtggcccaagccacttcccctccaccctctgcccagcacttcctgtgtctctgccaggcctgccctccagccaccagcagggctttcaccatgggacctctctccctgagctgatccatgccgaccctggcagggccccaccacacccccctgctgacctcaccagggctcaccaccgactgcaaacacacatgtcacctgaggctttcctccagcccctggcactccatcgttgcccctaaggcgtcccaagtgcttacggattattgctccagcctcagcagctctctgctaaacaggctggtgtacagaagcagccccgagccaggtcagggcttctgagcccattatcctgcctgactgctgacatgccaccatgctcacctgcttgcagggcacctttcagcagcggccttgcttcctgcctccctccgccacctcctgcagatttctcctgggatcgccttccaaataaaccacctgcatttggggtctgtgtctggtggaaccccaggccatgagtccttcacccctttctgcctggcctggtcctgttcattcctcagtcctccccacctcaccaagcctatggaggccccacttgtcagaggggagacagcctccttgccctggcactgaacaccctgtgtgccagagtgcccctcccatcattgtttccaccacatgagatccacagtggcagcacgcggcaggtacaccgatggcatgacttccagggcttggcctgcggggtcattgggtctgctggaggttcctgcagnggagggtagttctgccatgctctgggggagctgtctggagggctctagtgtgtccttcccagggccttggtaatgtagacctttaaccccccatgcctnacagacacacacacacacacacacacacacacacacacaccctgttacccaaaaaacgaaactctgtaaaacattttaaagaggtttattctgagccaggatgagtgaccacagcctggagaaaacacaaacccaagaagccttgagtaagtggtccccaggcccaaggtggtggagttacagtttgcttttatacattttagggagacaggagttacaagcaaggacataaatcaacacacagaaggtatacattggtttggccccaaaatgcagggtatcttaaaacaggggcttacaggttagaggtagatgcagagattctttaatttacagttggttgaaagagttaagctttgcctaaagacttcaggtcagtacaaaggaatgttaaggaggcctgctatgtgtcgcctgatgctatacagggtcaggagggaaagtaaaccacgttatacctgggtaacttaaaaaaaaaaggtttttaacaagattttatggaccaggcatagtggctcacgcctgtaatcccagcactttagggagaccgaggcgggtggattgtttgagtccaggagttccagaccagcctaggcaacatggtgaaaccctgtctctacaaaaaaaaaaaaaatacaaaaaattagccaggcgtggtggcacatgcctggattccaggaacctgggaggctgaggtgggaggatggctggagcctgggaggtcgaggctgcaatgagatgcaacagagcaagactctgtctcaaaaaagaaaaccaatgttatggtttgtagggtgtttcttaacccttgcctggcatggccttaggtcctgtttataatttggtatcttactgccacaaagagtccgatctgtcagtcttatgatctctgttttaatgttaatgccggtcagttgtgtccaaactccagcaggaagagggcctaataaggcaagtccaccctgccttcctgtcatggcccagaattctgtttttaaggtttttctggtgtcccttggccaagaggggatctattcagttggtccggggactcaagattttagtttcagtttacaactctatacacaaacaccccatacacacaggcaccaataccctatgcacagacaccacacacatgcatactacacacttacacataccacacacacgcataccatgcaagcatatcatacacacagacaccccatacaatgcgtacacatgcacacacacacaggctgcctcaaattgagaagggttccttggactttcagttcagtaaatcccaacgtttgaacattggtgctaacttaggaccagccccaggcctgttgcatggcactgtatgtgtgaaagtgcgtgtttgcaccagtgtgtgtgcagggctgtgtctgggaagaggtgtgctacacatgaggaagcagccagagcagcttggtggtcattgttgtgcccctacctgcagggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccaggtgagcccatacctattgcctgtctggggaagattgaaaggccaagggacatgggggcacagggaggcaggtgacactgcctcttggcccaaccagcacagagtagactgggtggagtcctgagcccagggccaggaggtacagctgtgtgcacagaagaggcctgggagagctcacagtgggcagggctgggggctccttgggcctctctttttttcccctttccattcttggtatctttaaaatgtattttcaaaaatgcaagagcaatgctgggtaaatctgcatatggtgactcggaagaatcttctggtggattggtgagagtggcttgcagagatgttggtcccacgtgactccctttgtgcaaaagcagatcttccctgacagggatctgcaagtgtgcagagatcacagtgctgtccacagtggtgccctcagggggaggcaggaaacgcttccactttttactttttgtaactaggtttgctagaaagcgtatatttatatttgtctttggaaaataatgattagaaatagaaggcataaaagtaataaatattcattaaagaagatgaggcatgaggcatgaggcgtgatcccttctacccataggcgccagtgttgcacatcgtggngttctttctgggatgtttctcgtaagcacttattaagttgacttgaaactgcagcacgcatgttctcgcatgccactctcccttgcagagcagcccgtggcgcctcctcttcaggggccatctgccagttctttcgggtggttctgtgcccgcatcagctcctggcagccacccgtgtccagcccacgagggagcccacccttggacatatctaagctccccgaccacctccttggcctttaatgggagtgggatcagagacagaggggcgacaagacagagggccccggggggtctcatggggggcggggcaagagcaggcattgctgaagttgggtcacctgccctctgtcaggagcaacgggggcctgaatcccagtccagcctcagcctcactgtggtctcacctttctccccagtttgtaaagctgtgaattagggacaggaaccctgaacaattttgagagtcacatgagcatttggaagtgggctttgtaggggaaagtgaggaggcccaggaggctggtttttgctgtggtcacgtgtgggcctgggagagcccagccctgctctggcccgcccccgtctcgccatctgtggaacttttggtttcaagcagctgcaagagttgccaagaggttgaaagaattcagctgcctgactccaggtcctggcatccccaggagaggcccctctttcccaggggctctggtcaaagtcccagagcctggcatccccaggagaagaggctcctctttcccagaggctctgctcaaagtcccagggaggccccctggtcctgcatgggctggggcaaccatgctcccgagtcttgtggctggggttcagtgctctgaccaggggacaccagggcagggtggacctctaaaccctgtgcactgagggaggagtgaggggtccccagagcactgctggggcagctgggagcagccagggaggagcctgggagacattccggcggctttgttgtctgagtgagggaggaaaggggagtaaagggttgagtcagggcctgcctggggcttttcctgccaccaaactgaacctcgaggccctgggttgccttgacctccagctcccaggaacaggggcagctggtaacatgtggcntgagtggaacggggcagggcagggctggggcaggtgggtgggtatgaaggctntgaggggtggaggacagggtgctcgggggggctgctgtctccaggcccagttgggggctgttccaggacttaggctgtgtgggaatctctaccctcaggccattacaggccggtccagctgcctggntaaggtgttcccctgtgcccccctagatcgggaaagtctgtgtggaaaactgccaggcgttcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatgtgagtgcctgctccagggagggagggtcggggtcctgggggcttctggagcctgggcctcctgccctttgagaaaagcagtacagctgcaaggcttagctggggagtggggaaggcatggaccagcttcaccctgagtgacccagcagtaaatggttgctccttccagataacatacaggaccttgggtaaatttgaattttgggtaaacaacaagcagttttttggtataggtgtgttccatgcaacttttgcagcttctcgaaagacacacctctaggtccatccatgccctcttaggaacatgctgacacagctgccattcatgccattcattgtgtatctgaaatgtaggtcccacagggcgtcgttggctgaatctggctgcactcacatccttcctcctgtacttaccccagcccaggtgacccctgctttgtgaccatgatgtcctgtaccctgccctgcgccctgtgctcctggcactgtctttgctgccctgggtctgtcactccggtccccttgggctccatccgtgggcagctcagctggtgctgttccctgtccttgggcactagctggacgctgggcccaggccagccccctgtgaccctgcttgtctgccacctgcagatgtggccgaggaggcgggctgccccctgtcctgtgcagtcagcaagagacggctggagtgtgaggagtgtggcggcctgggctccccaacaggcaggtgtgagtggaggcaaggagatggcaaaggtaagccctggaaacgcccaagggaggcctgcaggggcgatggcaccggtggaaacggggtcctggggccctgccagcctgggatggtctccctgctccaggtctgcttctggcacctcatcccccatgtggctctcgatgccagcatagcgggcagcagtgcagggcttgggagagggcttgtgagtacagtgaatggtccccaggccggattcactctggacttgggaaggtctgaaccaaagttgggatgtgctggggacagaatggcgtttttctgggaggtcctcggccaggggatgtggtgtgggcaggggactcattgtctgcatcagccagaggccagcctgggtgtgcccaccaccatgaggggccctcaccatgcagccctgagagggtcccggcctctttgctgtaagggccacctgtgtgaggaaccccccatacctcctctcccataagccatggctccccaggatgcttccgctggcaaggctctgtatatggtgtttccctactcaggcctccagttgctcctccctagaggggcaggatctgcctaggaggtggtgggggcgtgtggcggggctcccacatgggtgacagcctgctgtgtgtcctgtgcagggatcaccaggaacttctccacctgctctcccagcaccaagacctgccccgacggccactgcgatgttgtggagacccaagacatcaacatttgccctcaggactgcctccgtaagcagggtttaatcagggcatgggaacaggtaggagatagtaggggaaacctggatcccacaggcacttcagccagagttgccagggctgtcagttctatgcatcaagctgagcctcctgtgcatttcagcatcaccctagccatggggagggagcaggcagggtcagggacagggggaaggcagggacccacagactgtccaggcccctgctgaggtcccagaagtcggcacacacagatttcagaagcagagaatggtcagtagggacactgacagggaaagtgcggccctgagccagacgagggacactgcaatgtgcgggtcaggccaccaggctccagttttggaggcagagtcctttgttcagatgaaatgttaggagggggcctggcttcacccatggcttcagaaaggcactgtgaccaagccctgcccggctaagccaagctgctgggcccctggcctcactgggcctatgcttgcgacaccagttggggagggggctccttgggacactgccctggaaatatgggcgcctggggtggtcaggcgccccaggaggctgagtgggctacgtctgccctcaggggggcagcattgttgggggacacgagcctggggagccccgggggattaaagctggctatggcacctgcaactgcttccctgaggaggagaagtgcttctgcgagcccgaagacatccagggtgagtgggtggcggccgggaccaccaccacctcccagccccacagaggtctcaacagcacatctgaggtcccaacaagggaggaaattgctgggaggcgagtgggccccatgaaacttccctccctccctctgggcctctgttactccacccaggagaggggccagggcccctgtaaagtgtcttctggccataagttctatgatggacaggccagaaaagcagttcttccaccaaacaacttgtcagcctgacaagtcactgtccctgtgaccatgcagctgggacccacccaggaacacatttcaaggtcagcaggtatggtgggttgcacagccacactgactacactcaggggtgctgttctgcctgagcatagggacacgattgagttttctggtattatatagccctacgtccctagccacttagcattttcataaagaaaatgccaaagacatttggaacagaggaaaattttgacctcccctgccagccctccagtgccagctggtgtaatgagcacagcctctgctgtgtgaccttggcaggctgctcagcctctctgagcctctgtctccatctgtaagagggcaatagtggtctaggagggggcagtaaatggcagtacccatgctcgatggggtgttctcaggccttcccacacctccatggccacttcccagctggcgcggacacggcaggctggagagccatgaggcagagcatacgcagcctgtacccagtggtgccgagcctctggcggtgccaagcctcacaccacccccacccacagatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggtgagggtccctgcggggcagggaagatcccctgccctccccagctgccttccagggagggaggccagctggggagacagaggccatcctgtgaggggctgccaacgctgggcagacgaggcctgtgttctgcccccatttccatagggcgctgtgtggggacagtctgtggggtgggactatgatgaggtgccgttcccatctaggtgagaggcagtggtcagggtcacagcatcgggcaggggagcagcagtgtggatggaggggcaccgaagtcagaagggggtgcctttctggggagcctggcctgcaggtctgcatgtgctactcagagcctccaggctgtgccgagtatcctggagcctccttgtcctggccaggcaggcctctgccctctcctggtggtggcctgccccttcagtgttcctactagcactgtccagggcgctggaagccaagcccagttctggaagtaacagaggctcanagccaagggtgtgagtgaacggtgagccacgcagcttatggtggcgtgaatagctcctcggcaggagcctccagggaggaagctgagcacccagtggccacagggccctggcagttcccatctcaggctgggaggtggcctgggattcctgggaggggccatatcccacagtgcagctcagcctgaggcctcggccctggagcctccgttcaggcaacacccagccctcggtaagggtgtgagccaaggaggccttcccagatgtggccctgccgcttccccaccagctttcctaattggtggtccccatcctggcctggctgcagcttagcctcatggcagggctctaggatgagccaccagagtccttcataaacccagtgggtttgtgtgaggctgcccaggaaggccgcactggtctgggctgctgctggcagagaccaccaccctaaccccagtcagctccagagtcacactcatcagcaccaggtcttggacccatgactcaacctcagtatttgagaggatcaggttgatgtcgccctcatgtgcttattgcagtctctagagtgtggtaaacaggtttccagtgccagctgtggaggtgacagcggcagggaagccatggcagtgtcgacactgaccttgactgtgggttcccagggaatgtggggccagaccaggacagcccaggagcaggagacctggggtgacggatgcccagagctggcacatcaagggagggttcctggatcatggcaggctttggcctccctggtcagagttcaagtactgggggccagggtgggggtctgggaaggcatccggagcagtcccaagtgggcccaatgtgtggatagaactttggtgggagggcagggtggtagtgccagcaggcagggtgagcgggtgcgtgagggccagtggcagcccttgaggagcagtgcttccacactctgaggcggaacatggtggcgcctttctttgcaggggtggctatgtagagaagttgtcctggacacttccactgtagtcggaggtcctgggctgggcctggtgctcatttagtcctggggcaggggtcaggggagacagtagaccaggaaccagagagggtcgaagtactgagtccaagccatgctgtgaccacacctgtcatgtagcagctttcaggggcctggctgtggggtcccgcccagggcagagacaggcagcgttgccgctggntcagatgacagccggttctctgcacattggaacttgtccatggggcctcctttaagggtcttgccttcttcctcccctgtcatcctcacacttttcccccctcttctcccccttccctcatttccaacataggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaaggtacctgccaggcacaggcacagtgcccctgggggagtctccggggtggggggcgggtgaggcccctcctgcccagcatgggaccctgaagagcccccaatgctgttctagagcggctgcagttgggggacccctaccatgggccacttgggcctagagaagcagagcgaggactttgctctccctggaaggctctagaggtgttgctgtgccagtgccactgaagatctaagctttaaacatttaaaaagccctttgattttgagctgacagctgtcttcactccagtctgccccattcattcctctcagcccagtgtctcctggggtcttggtgaagagaccaccagtaaggactggccctcaatctcaagagacccccatgggcctgtctggctgcatgcccagtcatgccacaagtttggctcttgatgggcacatgcacctacccacacaggcatgcgactgcactgagtagccccctcttctgtggccaggccccagccctgtgaacagccaggctgaatggcggtgagggagagtggttggtgtacataacaattatctgcagtagtcgggggctgcacagcacagggtgcttccagatgcctacccagccccagactgggttcacctccaagccagtaaaaccccggactctccaggggcacagggagacccttggatttccattgcagccatgcacagccccaggctgcccacacctggtccaaggggtcctggggccccaactcccaactgtcctctcccttccagacaacctttcaggacagaggaggtggcacggcacagctgggcagtgggacagctgacctgggagggatggcttagccatgtgttctgactcttggaataccttcaggcccaggcacctgctgggcattccagcacagctctggccaccatcgggacgcatgtgctgggcaggtgatccctgtggtgcctcgtcctgtttccatcatgcctctacagttaggactctctggggaatcatgctattcattaagaataagccagaatgtcaggtgtgcagtgcactggtaacactgctttatctcaggtgctaatgtgggtcttcttggtaactaacggagtgtgagctgctgacgtgtgtgtgacggatacatcagcagcacaggagatgcctgggctccaggctggccatctcagacaggagcgggaaatggggagcctggtcgcggtgtgtggacctcctttatggctctccaccttctccagggcctctcccgacaagtgggtgtgtgggtacccctcacctttccagaatgattaatgcggggaatttctgtggacgactgtcttctaaagacaatgactacaggaacataatgccacatacacaggtggcccagccctgggacactctggggaaagatccggcatgtgtggttgctggctcctcagggtgcttcttcctcagggtggatgaggcccctgtccactgatcccaaaggctgggagaagcctcaagcagcatcgtctttgcaggcctctctgtctgaacttgggcaaggcgatgcaggtccatcctgacctggtatggtcatggaaggggcttccaggagcgatcgtttgcaacctgctctgtgctgcatttcagagaacgcctccccgagtgagcttcgagacctgctgtcagagttcaacgtcctgaagcaggtcaaccacccacatgtcatcaaattgtatggggcctgcagccaggatgtaaggccagctgcagggtgaggtgggcagccactgcacccaggctgggggctccatacagccctgttctccctctttctccctttccctactgctcctgccctgtttcctgttctccctctttctggaagcctggctcaggccccagcctggagcttgtgtctagctgagtccacgggctgagtggtcactttccatcagaggggccccgcgctagcggcactccctgggcccacagggctactcagaggtctctggtgtgacactgccatgtgtcctcacccagttcggggctgggcccatggggcagggagctctaggaatggacagtgcatcctgggtactagggtaccctgggtaccacagggcaccaggtgtgctgtgacctcaggtgaccccagccccgccntgcatggcaggaacattgtcaccatttctcagataaagacccaggagaccagcctggtttgttggttttccaaaccaccatgctgctcaggggcttcccagcatgngtgtgtgggtgtgtgcgagagggatagggaacccaaacaccgggagtgcgcagcaggcactgtggtcggcaccagtagagttggaggctgggcctgggacaggacaggtgggaacagggagcaggctgtgtgcagagcccagcgccaggcaggaacccttgcatctgcgcaggcctctgcctccatcagcatccccagcctgcactggggcaggacccaggaggcaaaaaggcttggcgtcctagcatcagggaggggtgcctcccagggcagtgtcgcctgcctcaggcacctcgagaaggaggcacccacacgagcagcaggaggcagagagcaagtggttcaagagaaagctgaggcttcaaggtctgcgctctccacagagctgcagcagtgctgcctgaggcagggctgtgtccacccccttactcattgggtggccgggcctggggaccctgcggcctcccacccctggctcctggaagacccaagctgcctgacccgcacgcccagggccccctctctccgcccccaggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaaggtgcgtgcatatggctctgcacccagccagccccggccaggccacaccctgacccaccacgcccctgccacccacaccctggcctgccactcccccaccatgccacactctagcccaccatgcccctgccatggcatgccatgctatggctcaccacgcccctgccatgtcacaccctgactccaccacgcccctgccatgccacacccccggcccaggtctcaccaggccgctacccgggccacacaccacccctctgctggtcacaccaggctgagccagtgaccgctgctgctgccatggcctgacgactcgtgctatttttcctacagctcgttcatcgggacttggcagccagaaacatcctggtagctgaggggcggaagatgaagatttcggatttcggcttgtcccgagatgtttatgaagaggattcctacgtgaagaggagccaggtgcccagtcccggggatgaggcggggctcccagggatcccaggtgcaccatggggcaggcagtgcccttgggaagcctaggaaagataccgaagattagtggagctctaagctttttatagccctcaccccaaatctttctgaccctgggtccccaaggacccaattagaactccgctcagcctctgccatgtccttctcctccagggcctccagggcacccctccctggcagcatactgacccgaggcccttgccgcacttttcagaggccacctcatgctgcggaactaacagtcctcttctgcagaataaaggtcaccgttctgatatgaccttagctcttttctcaaagaagggtgggatgaaattagcaggatcgtcattctttgcaaaaaggaatgaactgctttacaagtgaggcttctccgcacaggggccttggacactgggctgggtgagtttagaggcataggaaccccctggacaggatccagaaacgcagcccagctttgtccaagtctatgaaacaggcagtgggcacccttggggacacctggttccctaccatggtcctagtcccgggacaactgccttggccctcttagtttttctgcccatgtcagcccctctcctgcagcctcccagtggcagcccaggcagggcaagttcagggggctgctcaaaggccatgccgcccagacgttcagagcagaccctgctcaaaaaagaagaagaaaaaaaaaaaccacaaaaggcgacttcctatgattcagctttgctctgaggggtctggagaagccatttgtgttgccaggggttgtgagctgttccgtttcttctggggtttcccttgtgacagcagatctcagaactgctgtggggggaagtggtaggggctggcccctgccctgtggagggtgtggacagagccttcctgttgatgcactggtcctccagggagagaagctgaaggcattcttatacaagacgtgcaggctacactgtcacacactgctgccaagacagccacactgcactaaggacataaatccctcatgtgtttaagtaaaagaagtgacagtaagattgtgttacaacccaaggcacacattttaaaaatggacttgcaccttcaggcttgtctgtggagaagctgtttctcaagtcaccctggaaaggatttgttaggtcagaccccagcactgatgagggatgtagggggctgggggcactcattcagagatgctaaaagcaccctgcaatacaataagagagaaaaaacagcctcccccaaggcacacagacaaatcccttccctcaaggctccttcagtgtggtagctgctggcaaagtgcccaacatggaaatgctagatgttggtttcccaaacacattggccctcagaaccccaggggagatcttgcagggggagttccaggcccatgcgtagggaagcactgctctgcactaccagcaggcctgtggcatgtgacaagctggccctgtgtgcctgtgggtgggcagctgactcccgccagcatctcagcaatccacaggaggttcaggctggagctccagccccttcaaagatgtgtgtggccagttctgtgcccaggagtgtctacagcactcctctggttactgaaagctcagggatagggcctggccttctcctttacccctccttcctagagagttagagtaacttcaatgtctttattccatcttctctttagggtcggattccagttaaatggatggcaattgaatccctttttgatcatatctacaccacgcaaagtgatgtgtaagtgtgggtgttgctctcttggggtggaggttacagaaacacccttatacatgtagtggggccacgacgcccgtctgtgcagcttggccagggaattgcactggccctgagcacctgtctgcagtgctagccctctgcaatgaccccctcactgaggggccttcatgatgtgttcgtgaggcaaatggctgggccaggccgggctgcagttctgaaagctccggtagagcatgagagccgagcaggtggccagtaacccagcctcccccacccttgctagtcccatgcctccctctgggtcccttggtgactcaggcccagcccagcagttaccctcctgtggtcctgagtagggctttgggctcaggcagagcacccagtgacttgcccccacccacaagtggagcttggggactccacctgaagatggccttgtgtgagtttatagattggtgtgccccaaactgtcaaaggtggaagccaaaatctcattcttccaggtccaatgacaagagctgatcacttttaaaacacaggattcttgcctctaaagtgttctacagtcagcagaggtgtgatgtgcctctctttttttgtggggggaggggatagggtctcactctgtcacccaggttggagtgcagtggcgcaatcctggctcactgcaacctccacctcctgggttcaagcgattcctgtgcctcagcctcctgagtagctgggattacaggcacgtgctacttcgcctggctcatttttttgtattttttggtagggacagggtttcaccatgttggtcacactggtcttgaatcctgacctcaaatgatccacccacctcagcctcccaaagtgctgggattacaggcgagagccaccacgcctggccaacatgtgcatcttaaatactgtgggctgtgaataagcaattggacaacagctttccgattatataagctgactgtaggctagttggaaaaggcagagaaaagcataaagaagagactgaatgtcactggaccacaccgcccagtgacctctggctgcccagtgacctctggctgccctctggtgtgctccgtggtgtgcacatgtatgctttttattgccctaaatgggctcatgcgatgctgttttgtgatatgattttgttcccattgcaaagcgagttttccttcacagttaaaatgtttttctatagctttttttccttctaagattttatgaaattttccaagcacatagaaatggcaaaagaactatacaaggcactctgagtctggtggcctgcattctcaccgctttaagtgtgagaatcttaagaattattaagattaattcatgatattaattaatgtatcattaattcattaatcatgaattcatcttaagcctcatgtgtcccttgtgcgtgtggctgctctgatgggagtggcttgggcaaggacttcagcttcatggagtaggggccaggcaggcaggcagcccttcccaccttcatgctacatccatctgagcagccagacccaggctgacatctgtgagcatctgtgcttgaagccgacagggtcagcaggtgcttggtggtgggggtggatatctgggccccccggagggctctgtgagggccaggtggagccactcactggtcctttcactctctgcagatggtcttttggtgtcctgctgtgggagatcgtgaccctagggggaaacccctatcctgggattcctcctgagcggctcttcaaccttctgaagaccggccaccggatggagaggccagacaactgcagcgaggagatgtgagcggggactggctttggcccagcctcacttgggaagggaggggacatctgtgtgcattccctccccatccagagcagccccaggagaaaccaggagaagtggggggtggggagtgggcagggcagaggttagagagccatccttgggtgtggcagtagatgctgggggtcctaccccactgagggctggtgggcttcctagggggtcttataaaaatgagtgagagcagagactggtgcaatgcaagctcaagggctgagagctgtcccagcaggacacttagggaggagaaggtaattccagatggcccagggggcacgtgttaaggggtgcctgagtggaggtgcagagccaggcaggggcagcctgggctgcaacctgagaggttgaaacctttacactctcaggggagatggtcttagaaaccaggctgagaacagatgagaaagggctgcccagaggcagagatgcatggtacatgcattccacagagtgtgcacactgctggggcgtggagggggcatgctgggccgagaagccaggtaccacctccctgggcccccagctggtggccagctggagcgctgtggtctgcctgggcacccaggcaagcaaggacggtgcaggacagacgtgtgattgccaaaggttcttgtttgtctgtctccacacctaaaagccctcaatcaattcattcttcttgagagttttaaggactggcacttctcacttagctgccagagcaagcatagtatccagtgagctgtttaccagtgtctttgcaagggaagtaaaaatgaatctcccttggagggaagggaagcttgtaggattctggaccacagctctgagagacctgagcacagtggcccacgggctgggatcctgcaacaggccatgccccagtcccacgaggctcagagatgtcagcgatgcagaaatagctttggagttggagacagagcacactgggcccagggtacagggcagggtgcgatggctgtggtgggctgtccttctgagacctggccctgcttggatcatattggcctgtctgctcttcccaccaggtaccgcctgatgctgcaatgctggaagcaggagccggacaaaaggccggtgtttgcggacatcagcaaagacctggagaagatgatggttaagaggagagtgagtgcctgggtccaattcccacaagctgaaagtggcttggggagactccagcctcaccccagggcagtagttttagccctcagagttcccagtgtggggccacagtgggattgtgcaaagagagagagtcatgctctcccctgcatgcagacagcagattgaacccttctcaggccaggccgcttgggcttcttttcaagtcatgaagaaaccaagattcttgacatttaggaaaagctctctacagacaggcatgggaggaaaatattccaaagccaaaaagaaatagttttccataatggaaaatgagaaagttgagttaaagcaagtgcccaaaattcagatgcccagaggggggtccggctggaggagggcagtgagtaaggagcagtggagactgtggcaaacagccagccccctccaaagggttccttgctggagtacctgtggcctgcagaaaaaggagccaggaatgggctgggtcttctgaattttatgaggtttatgttgtctcccagtttttattgttaacaattcatttaaatttcccagagccgaacgcaggccaaacaaagcatatcggggggtggccagagtcagcctgtgtgcctggaagtacagtttgtgaggtctacacattttcaaccaatattcatcaggcagctgctaggacccatactatgtctcagaggctgggaatgcagataaacaagaccagcacagccctgccctcacggggtttacacacaggccttgtcttaacattggcacatttactgagttgtcacttcttccgacttggatagcgggatgcaggacctaggaccccataggaaaacttcacatcactgtctcgcttggatgaaaacttcaagtcctgagaatgcagggttgttcaaaacagaagcgatcactggtgctggccttcctagaacagtgctgagaaacagccgggaaaacgctgggcccctctgtgcacattcaagctgggcattgaaaggagcagacgccagtggcccctcgtgctggggagcccgtgcctgtagcagggtcctgtgcaccctgacagcgctgtcagatttcagaactcagccctgacgtgacgttctagatggcaggggtccttggggaaagctggaaaacatcctgcaggctggggctgcgtcctggcatgtggctgcagtgggctcccccagagcccaagggtttgtcagcgggctggttccgtgctgccatctccgggttgggccaggggcagctagatgaggcgtcccccagggctgccctccaggccctggagataagacgctgagggagcacatttctttccccagggaggtgactttccaggggaagagacagaatagacaacacccaggggagtgatggggacctcagagcaggaccaggccagccaggagtcctgggagctctatgcagcctggccctggtctcttggagaggtcaggagattggtgcgaggtggagacacagccagagcctctgccctgaggatggcttgttgtatactgagttgtatctagttgtggcacatggcttggagtgaccggccatctctgtcttccaggactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaccaaagtctgctctgaacctttttatttgtaaatgtctgactttgcatccagtttacatttaggcattattgcaactatgtttttctaaaaggaagtgaaaataagtgtaattaccacattgcccagcaacttaggatggtagaggaaaaaacagatcagggcggaactctcaggggagaccaagaacaggttgaataaggcgcttctggggtgggaatcaagtcatagtacttctactttaactaagtggataaatatacaaatctggggaggtattcagttgagaaaggagccaccagcaccactcagcctgcactgggagcacagccaggttcccccagacccctcctgggcaggcaggtgcctctcagaggccacccggcactggcgagcagccctggccaagcctcagccccagtcccagcccatgtcctccatcaggggtagcgaggttgcaggagctggctggccctgggaggacgcacccccactgctgttttcacatcctttcccttacccaccttcaggacggttgtcacttatgaagtcagtgctaaagctggagcagttgctttttgaaagaacatggtctgnggtgctgtggtcttacaatggacagtaaatatggttcttgccaaaactccttcttttgtcttcgattaaatactagaaattttttctgtttcctaacttcatcattgattgtttgaaatcttggagtttcaagcatttttttcaagctgagacggttccttttgcatgcctcctgactcactgactcctcactggctttgtgtctagtgccacactccatttgtgcngacgatgctatgaggctggcccgtgtgcaccctcgatttggaaggtcctcttctccgttgtaggctcagcaacagacgagtgcctcctttacgttagagaatgttttatagaacgtttccgctgcagtaagccattgagtgcagcagtgtgctgttgccctacactgccttagaaaaagagttaagcagttcgacaaatatacatcagattagagagtctcaagttcataaaagtcaaaattggtagtgtctgttaagttacatttaccaagagagcacatgatatttcccctaactataacagtgtttttggaaacctggaacacaaaaccattaatataattggcacagaaaccacgagtttggtttgaacatcaaagggagttttgccaaggccttactgtctgcacttgaagttttggttcttcagtgcagaacaaatgatctgttttcatttttaggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaaggtaatggactcacaaggggaagaaacatgctgagaatggaaagtctaccggccctttctttgtgaacgtcacattggccgagccgtgttcagttcccaggtggcagactcgtttttggtagtttgttttaacttccaaggtggttttacttctgatagccggtgattttccctcctagcagacatgccacaccgggtaagagctctgagtcttagtggttaagcattcctttctcttcagtgcccagcagcacccagtgttggtctgtgtccatcagtgaccaccaacattctgtgttcacatgtgtgggtccaacacttactacctggtgtatgaaattggacctgaactgttggatttttctagttgccgccaaacaaggcanaaaaatttaaacatgaagcacacacacaaaaaaggcagtaggaaaaatgctggccctgatgacctgtccttattcagaatgagagactgcggggggggcctgggggtagtgtcaatgcccctccagggctggaggggaagaggggccccgaggatgggcctgggctcagcattcgagatcttgagaatgatttttttttaatcatgcaacctttccttaggaagacatttggttttcatcatgattaagatgattcctagatttagcacaatggagagattccatgccatctttactatgtggatggtggtatcagggaagagggctcacaagacacatttgtcccccgggcccaccacatcatcctcacgtgttcggtactgagcagccactacccctgatgagaacagtatgaagaaagggggctgttggagtcccagaattgctgacagcagaggctttgctgctgtgaatcccacctgccaccagcctgcagcacaccccacagccaagtagaggcgaaagcagtggctcatcctacctgttaggagcaggtagggcttgtactcactttaatttgaatcttatcaacttactcataaagggacaggctagctagctgtgttagaagtagcaatgacaatgaccaaggactgctacacctctgattacaattctgatgtgaaaaagatggtgtttggctcttatagagcctgtgtgaaaggcccatggatcagctcttcctgtgttngtaatttaatgctgctacaagatgtttctgtttcttagattctgaccatgactcataagcttcttgtcattcttcattgcttgtttgnggtcacagatgcacaacactcctccagtcttgtgggggcagcttttgggaagtctcagcagctcttctggctgtgttgtcagcactgtaacttcgcagaaaagagtcggattaccaaaacactgcctgctcttcagacttaaagcactgataggacttaaaatagtctcattcaaatactgtattttatataggcatttcacaaaaacagcaaaattgtggcattttgtgaggccaaggcttggatgcgtgtgtaatagagccttatggtgtgtgcgcacacacccagagggagagtttgaaaaatgcttattggacacgtaacctgctctaatttgggctgtttttcagatacactgtgataagttcttttacaaatatctatagacatggtaaacttttggtttcagatatgcttaatgata

SEQ ID NO: 140 - DNA, 1843 bp, Homo sapiens
agaagcaggggggaatcctgaatcgagctgagagggcttccccggttctcctgggaaccccatcggccccctgccagcacacacctgagcagcatcacaggacatggccccctcagccacctagctggggcccatctaggagtggcatcttttttggtgccctgaaggccagctctggaccttcccaggaaaagtgccagctcacagaactgcttgaccaaaggaccggctcttgagacatcccccaacccacctggcccccagctagggtgggggctccaggagactgagattagcctgccctctttggacagcagctccaggacagggcgggtgggctgaccacccaaaccccatctgggcccaggccccatgccccgaggaggggtggtctgaagcccaccagagccccctgccagactgtctgcctcccttctgactgtggccgcttggcatggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtga

SEQ ID NO: 141 - DNA, 2432 bp, Homo sapiens
cagaagcaggggggaatcctgaatcgagctgagagggcttccccggttctcctgggaaccccatcggccccctgccagcacacacctgagcagcatcacaggacatggccccctcagccacctagctggggcccatctaggagtggcatcttttttggtgccctgaaggccagctctggaccttcccaggaaaagtgccagctcacagaactgcttgaccaaaggaccggctcttgagacatcccccaacccacctggcccccagctagggtgggggctccaggagactgagattagcctgccctctttggacagcagctccaggacagggcgggtgggctgaccacccaaaccccatctgggcccaggccccatgccccgaggaggggtggtctgaagcccaccagagccccctgccagactgtctgcctcccttctgactgtggccgcttggcatggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtgaccgcccacgccacatggacacagccctcgccctccgccccggcttttctctgcctttctaccgaccatgtgaccccgcaccagccctgcccccacctgccctcccgggcagtactggggaccttccctgggggacggggagggaggaggcagcgactccttggacagaggcctgggccctcagtggactgcctgctcccacagcctgggctgacgtcagaggccgaggccaggaactgagtgaggcccctggtcctgggtctcaggatgggtcctgggggcctcgtgttcatcaagacacccctctgcccagctcaccacatcttcatcaccagcaaacgccaggacttggctcccccatcctcagaactcacaagccattgctccccagctggggaacctcaacctcccccctgcctcggttggtgacagagggggtgggacaggggcggggggttccccctgtacataccctgccataccaaccccaggtattaattctcgctggttttgtttttattttaattttttttgttttgatttttttaataagaattttcattttaagcacaaaaaaaaaaaaaaaaaa

SEQ ID NO: 142 - DNA, 1450 bp, Homo sapiens
  modified_base (1378)..(1378): a, c, t, g, unknown or other
ctgcctgggaacagggcgagaggggagtattctaagcaattctgcttccccctaagcccgccagtggactgctagcgatggggacaggatcctcccaaacagagctgcaggctgggaggggggtcatgactgtattggggactgggggtctctcagatggagggtgattcagatcctgtccagctggaacatgaaaaagagctttcctggcttctccagacttgttcgggagagagaagcttgagtgtgagtctttgagcacggaggggtgctcctgggcgcccctactggaagatccaaaagtcttgccctttgaggagaagagtactttaagggggctgggagggttgggttgagccagggttcctaggacccactggagctgcagaggtgtttggggaaagaagagatatacttgcgggagatctactcctggactctttttttttttttctttttcttgcagctgagataattaagatccagggaagggaagtgacttggtcaaggtcacacagctctcagttccagctggtccctagaagaggattataattataggattcaggggcttgacagctagggccaggagtcaccgccatcacttccatattacgccgccgcctcacttctcagatttaggtgtgggtgtgtgtgtggttggggggaaaggagtgtaggataccacacgctgcggtcttctccaccgagcgctattttcattctttccgcagaacctcaccccgttcttgctctgaatcttcggttctgggtctgagggagggattctcccggattcccacggtccagtcttcaactaggagtggctcctttaagactcgcccttcccgaggtctattaaggagaggcgggggcgggcgtgagcctgtagatccgcccctgactggtgattggtcggtgggcgggcaggggcgggcctgagggacagggcctccccctacctctgctccgtaccctccgccccttcagtctggggctccgggtaaagtttcagcctccgcacgtgactcgctatggccgctgccatcgccccgcgcccctgagccgcggccccctggacggctcctctcccgggaccccgcaccctgatgccgagcagcaccagggcgccgggttagggcagacgctgtgctcgctggcaccccgaacgggttgcttcccccgctgcgaggtaattcctcccctggggattttggggagcccctggttcccgcaggcgtcggggccccatgtcctgccccagattggtgctgcgggaggggactgggtaccgggaggcttccgacgggaacccggggtttcctgggcttcccaactcgctgccgccgtgtccagggtgggggaacccgtctgtgantctgcctgccgccgtgtcttccgcgcagccttgangcgtctggggccgtctgctgcgcgtgtcccccgcgct

SEQ ID NO: 143 - DNA, 3122 bp, Homo sapiens
ctgctccgtaccctccgccccttcagtctggggctccgggtaaagtttcagcctccgcacgtgactcgctatggccgctgccatcgccccgcgcccctgagccgcggccccctggacggctcctctcccgggaccccgcaccctgatgccgagcagcaccagggcgccgggttagggcagacgctgtgctcgctggcaccccgaacgggttgcttcccccgctgcgagcatcacaggacatggccccctcagccacctagctggggcccatctaggagtggcatcttttttggtgccctgaaggccagctctggaccttcccaggaaaagtgccagctcacagaactgcttgaccaaaggaccggctcttgagacatcccccaacccacctggcccccagctagggtgggggctccaggagactgagattagcctgccctctttggacagcagctccaggacagggcgggtgggctgaccacccaaaccccatctgggcccaggccccatgccccgaggaggggtggtctgaagcccaccagagccccctgccagactgtctgcctcccttctgactgtggccgcttggcatggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccactgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtgaccgcccacgccacatggacacagccctcgccctccgccccggcttttctctgcctttctaccgaccatgtgaccccgcaccagccctgcccccacctgccctcccgggcagtactggggaccttccctgggggacggggagggaggaggcagcgactccttggacagaggcctgggccctcagtggactgcctgctcccacagcctgggctgacgtcagaggccgaggccaggaactgagtgaggcccctggtcctgggtctcaggatgggtcctgggggcctcgtgttcatcaagacacccctctgcccagctcaccacatcttcatcaccagcaaacgccaggacttggctcccccatcctcagaactcacaagccattgctccccagctggggaacctcaacctcccccctgcctcggttggtgacagagggggtgggacaggggcggggggttccccctgtacataccctgccataccaaccccaggtattaattctcgctggttttgtttttattttaatttttttgttttgatttttttaataagaattttcattttaagcacatttatactgaaggaatttgtgctgtgtattggggggagctggatccagagctggagggggtgggtccgggggagggagtggctcggaaggggcccccactctcctttcatgtccctgtgccccccagttctcctcctcagccttttcctcctcagttttctctttaaaactgtgaagtactaactttccaaggcctgccttcccctccctcccactggagaagccgccagcccctttctccctctgcctgaccactgggtgtggacggtgtggggcagccctgaaaggacaggctcctggccttggcacttgcctgcacccaccatgaggcatggagcagggcagagcaagggccccgggacagagttttcccagacctggctcctcggcagagctgcctcccgtcagggcccacatcatctaggctccccagcccccactgtgaaggggctggccaggggcccgagctgcccccacccccggcctcagccaccagcacccccatagggcccccagacaccacacacatgcgcgtgcgcacacacacaaacacacacacactggacagtagatgggccgacacacacttggcccgagttcctccatttccctggcctgccccccacccccaacctgtcccacccccgtgccccctccttaccccgcaggacgggcctacaggggggtctcccctcacccctgcacccccagctgggggagctggctctgccccgacctccttcaccaggggttggggccccttcccctggagcccgtgggtgcacctgttactgttgggctttccactgagatctactggataaagaataaagttctatttattctaaaaaaaaaaaaaaaaaa

SEQ ID NO: 144 - DNA, 3413 bp, Homo sapiens
ctgctccgtaccctccgccccttcagtctggggctccgggtaaagtttcagcctccgcacgtgactcgctatggccgctgccatcgccccgcgcccctgagccgcggccccctggacggctcctctcccgggaccccgcaccctgatgccgagcagcaccagggcgccgggttagggcagacgctgtgctcgctggcaccccgaacgggttgcttcccccgctgcgagcatcacaggacatggccccctcagccacctagctggggcccatctaggagtggcatcttttttggtgccctgaaggccagctctggaccttcccaggaaaagtgccagctcacagaactgcttgaccaaaggaccggctcttgagacatcccccaacccacctggcccccagctagggtgggggctccaggagactgagattagcctgccctctttggacagcagctccaggacagggcgggtgggctgaccacccaaaccccatctgggcccaggccccatgccccgaggaggggtggtctgaagcccaccagagccccctgccagactgtctgcctcccttctgactgtggccgcttggcatggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtgaccgcccacgccacatggacacagccctcgccctccgccccggcttttctctgcctttctaccgaccatgtgaccccgcaccagccctgcccccacctgccctcccgggcagtactggggaccttccctgggggacggggagggaggaggcagcgactccttggacagaggcctgggccctcagtggactgcctgctcccacagcctgggctgacgtcagaggccgaggccaggaactgagtgaggcccctggtcctgggtctcaggatgggtcctgggggcctcgtgttcatcaagacacccctctgcccagctcaccacatcttcatcaccagcaaacgccaggacttggctcccccatcctcagaactcacaagccattgctccccagctggggaacctcaacctcccccctgcctcggttggtgacagagggggtgggacaggggcggggggttccccctgtacataccctgccataccaaccccaggtattaattctcgctggttttgtttttattttaatttttttgttttgatttttttaataagaattttcattttaagcacatttatactgaaggaatttgtgctgtgtattggggggagctggatccagagctggagggggtgggtccgggggagggagtggctcggaaggggcccccactctcctttcatgtccctgtgccccccagttctcctcctcagccttttcctcctcagttttctctttaaaactgtgaagtactaactttccaaggcctgccttcccctccctcccactggagaagccgccagcccctttctccctctgcctgaccactgggtgtggacggtgtggggcagccctgaaaggacaggctcctggccttggcacttgcctgcacccaccatgaggcatggagcagggcagagcaagggccccgggacagagttttcccagacctggctcctcggcagagctgcctcccgtcagggcccacatcatctaggctccccagcccccactgtgaaggggctggccaggggcccgagctgcccccacccccggcctcagccaccagcacccccatagggcccccagacaccacacacatgcgcgtgcgcacacacacaaacacacacacactggacagtagatgggccgacacacacttggcccgagttcctccatttccctggcctgccccccacccccaacctgtcccacccccgtgccccctccttaccccgcaggacgggcctacaggggggtctcccctcacccctgcacccccagctgggggagctggctctgccccgacctccttcaccaggggttggggccccttcccctggagcccgtgggtgcacctgttactgttgggctttccactgagatctactggataaagaataaagttctatttattctaaaaaaaaaaaaaaaaaa

SEQ ID NO: 145 - DNA, 3301 bp, Homo sapiens
gtgcctcttgcagcagcctaacccagaagcaggggggaatcctgaatcgagctgagagggcttccccggttctcctgggaaccccatcggccccctgccagcacacacctgagcagcatcacaggacatggccccctcagccacctagctggggcccatctaggagtggcatcttttttggtgccctgaaggccagctctggaccttcccaggaaaagtgccagctcacagaactgcttgaccaaaggaccggctcttgagacatcccccaacccacctggcccccagctagggtgggggctccaggagactgagattagcctgccctctttggacagcagctccaggacagggcgggtgggctgaccacccaaaccccatctgggcccaggccccatgccccgaggaggggtggtctgaagcccaccagagccccctgccagactgtctgcctcccttctgactgtggccgcttggcatggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtgaccgcccacgccacatggacacagccctcgccctccgccccggcttttctctgcctttctaccgaccatgtgaccccgcaccagccctgcccccacctgccctcccgggcagtactggggaccttccctgggggacggggagggaggaggcagcgactccttggacagaggcctgggccctcagtggactgcctgctcccacagcctgggctgacgtcagaggccgaggccaggaactgagtgaggcccctggtcctgggtctcaggatgggtcctgggggcctcgtgttcatcaagacacccctctgcccagctcaccacatcttcatcaccagcaaacgccaggacttggctcccccatcctcagaactcacaagccattgctccccagctggggaacctcaacctcccccctgcctcggttggtgacagagggggtgggacaggggcggggggttccccctgtacataccctgccataccaaccccaggtattaattctcgctggttttgtttttattttaatttttttgttttgatttttttaataagaattttcattttaagcacatttatactgaaggaatttgtgctgtgtattggggggagctggatccagagctggagggggtgggtccgggggagggagtggctcggaaggggcccccactctcctttcatgtccctgtgccccccagttctcctcctcagccttttcctcctcagttttctctttaaaactgtgaagtactaactttccaaggcctgccttcccctccctcccactggagaagccgccagcccctttctccctctgcctgaccactgggtgtggacggtgtggggcagccctgaaaggacaggctcctggccttggcacttgcctgcacccaccatgaggcatggagcagggcagagcaagggccccgggacagagttttcccagacctggctcctcggcagagctgcctcccgtcagggcccacatcatctaggctccccagcccccactgtgaaggggctggccaggggcccgagctgcccccacccccggcctcagccaccagcacccccatagggcccccagacaccacacacatgcgcgtgcgcacacacacaaacacacacacactggacagtagatgggccgacacacacttggcccgagttcctccatttccctggcctgccccccacccccaacctgtcccacccccgtgccccctccttaccccgcaggacgggcctacaggggggtctcccctcacccctgcacccccagctgggggagctggctctgccccgacctccttcaccaggggttggggccccttcccctggagcccgtgggtgcacctgttactgttgggctttccactgagatctactggataaagaataaagttctatttattctaaaaaaaaaaaaaaaaaa

SEQ ID NO: 146 - DNA, 3494 bp, Homo sapiens
ctcggttccctgcgtttctcccgctgcagccggacgcgccgggaatgggttaagccaggggcggtgcctggacggggcggggcggtggaaagggggtggtgcccggaggggagggggcgcgcagagctggggtgggggggccgtggcgcgtaccaccagagaccgagcgagtcgccagctgcccctggcctggcgggggcggaaccgcgcgggatccccacccccacccggaatcctcgccacggagaatccctggagaagccccggatccccggctgggaggaggaagtgctcgttgacccccagccccgcgctgatcccgcccccggcctgcggacttggggagccgctgtactctgcctcggacgccacgagactctagacgggagtcccctcgaggtgaagccgctgagttcccgggccccgccaggcttccctgggagagccgacggaccccccctcccagcacacacaacttccctgcttttcaccgggactggcggagcggccggcggacttagacgcggggacttcagggcagggggcgccccctgcccgggtcaccagtcggggcgaggggacgtctcctctcccccagctgctctgctcggatggcgccgccggctgagtgacgggggcggcgcgcaggacttcccagctcggacctcttgccttcgaggggaaagatgtacgagagtgtagaagtggggggtcccacccctaatcccttcctagtggtggatttttataaccagaaccgggcctgtttgctcccagagaaggggctccccgccccgggtccgtactccaccccgctccggactccgctttggaatggctcaaaccactccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtgaccgcccacgccacatggacacagccctcgccctccgccccggcttttctctgcctttctaccgaccatgtgaccccgcaccagccctgcccccacctgccctcccgggcagtactggggaccttccctgggggacggggagggaggaggcagcgactccttggacagaggcctgggccctcagtggactgcctgctcccacagcctgggctgacgtcagaggccgaggccaggaactgagtgaggcccctggtcctgggtctcaggatgggtcctgggggcctcgtgttcatcaagacacccctctgcccagctcaccacatcttcatcaccagcaaacgccaggacttggctcccccatcctcagaactcacaagccattgctccccagctggggaacctcaacctcccccctgcctcggttggtgacagagggggtgggacaggggcggggggttccccctgtacataccctgccataccaaccccaggtattaattctcgctggttttgtttttattttaatttttttgttttgatttttttaataagaattttcattttaagcacatttatactgaaggaatttgtgctgtgtattggggggagctggatccagagctggagggggtgggtccgggggagggagtggctcggaaggggcccccactctcctttcatgtccctgtgccccccagttctcctcctcagccttttcctcctcagttttctctttaaaactgtgaagtactaactttccaaggcctgccttcccctccctcccactggagaagccgccagcccctttctccctctgcctgaccactgggtgtggacggtgtggggcagccctgaaaggacaggctcctggccttggcacttgcctgcacccaccatgaggcatggagcagggcagagcaagggccccgggacagagttttcccagacctggctcctcggcagagctgcctcccgtcagggcccacatcatctaggctccccagcccccactgtgaaggggctggccaggggcccgagctgcccccacccccggcctcagccaccagcacccccatagggcccccagacaccacacacatgcgcgtgcgcacacacacaaacacacacacactggacagtagatgggccgacacacacttggcccgagttcctccatttccctggcctgccccccacccccaacctgtcccacccccgtgccccctccttaccccgcaggacgggcctacaggggggtctcccctcacccctgcacccccagctgggggagctggctctgccccgacctccttcaccaggggttggggccccttcccctggagcccgtgggtgcacctgttactgttgggctttccactgagatctactggataaagaataaagttctatttattctaaaaaaaaaaaaaaaaaa

SEQ ID NO: 147 - DNA, 1450 bp, Homo sapiens; identical to SEQ ID NO: 142
  modified_base (1378)..(1378): a, c, t, g, unknown or other

SEQ ID NO: 148 - DNA, 1081 bp, Homo sapiens
  modified_base (94)..(94): a, c, t, g, unknown or other
gcacaggggaaggaagggcttggcgtctagcccaggccggcagtctggccctggagccggagttcgggaccactttgccccattgccaccagcntctggacctgggggcttaagagagctggctcgtgtcaaagaactgaatcccaagaaagatgctaatatcagcagtattgatcttcccacctcgagccaggcttgctggggctgggggtgggagggctggcccagcgtgctgacctctgccccctcctttcctgcaggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtgaccgcccacgccacatggacacagccctcgccctccgccccggcttttctctgcctttctaccgaccatgtgaccccgcaccagccctgcccccacctgccctcccgggcagtactggggaccttccctgggggacggggagggaggaggcagcgactccttggacagaggcctgggccctcagtggactgcctgctcccacagcctgggctgacgtcagaggccgaggccaggaactgagtgaggcccctggtcctgggtctcaggatgggtcctgggggcctcgtgttcatcaagacacccctctgcccagctcaccacatcttcatcaccagcaaacgccaggacttggctcccccatcctcagaactcacaagccattgctccccagctggggaacctcaacctcccccctgcctcggttggtgacagagggggtgggacaggggcggggggttccccctgtacataccctgccataccaaccccaggtattaattctcgctggttttgtttttattttaatttttttgttttgatttttttaataagaattttcattttaagcacatttatactgaaggaatttgtgctgtgtattggg

SEQ ID NO: 149 - DNA, 732 bp, Homo sapiens
  modified_base (579)..(579): a, c, t, g, unknown or other
gaggacctggacctgcactatccagtacagtagctgctcaccacatgtgactctttaaatttaaattaattaaaattaaactcaattcagttcctcagttgcattagccacatttcaagtactcagtagacgcatgtggctggtggctgaggtatggatggtgcagacgtagaacctttccatcattgtagaaaattctatcagacagcattgctccggccacctgccaggtggtcctccgggagtgctggtgcggagtgctggtgccgagtgctcagagtgggttcgggttcagtccctgaacccaagcatcctctgcacccagatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggaggtgggcagggggcctgttgtctgggggctgggctgggacgggggtgcanccctgtnagtctcttccaggganctctttcaggccacctctgttaggtatctctagagggcagggtctggtctgcaactacacagcaagggggccatgtggggcctggactcctgttcccgatttctgggcaacaccccttctaggaggttaa

SEQ ID NO: 150 - DNA, 689 bp, Homo sapiens
  modified_base (19)..(19): a, c, t, g, unknown or other
ccccaagcacagtcacggnacacatacaaatgtgatggtttatcattgtatctttgtggttttgaaggtgggggtcctaggagtccagaggagtgatggggtgctggaggcttcattggcagcctcctgccctgagtctggctggggagtcccagttttcttaagacttgaatcctgccagcagtggtgaggctgggagaggntcttaggagggacggtgaggcagggtggagcttggtactaaggatggcgacctaggtctctaactgcccctcccctcttctctctctagccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaaggtgagttgaaggggtcattgggaaagacagcttgatgaggtcaatgggatgtccccacttctgtgtcctgggagtgtgcagttggggggtgtccctgaagttgctgctcttctttctctgtggaagttggcagcaagcagggacacctaccacagtttccccacaggtcctcccccataaatgtgcagggctccctcaaaccagaggtcccctcctgcctcagctcctttccctgtctctatcctcca

SEQ ID NO: 151 - DNA, 1021 bp, Homo sapiens
  modified_base (704)..(704): a, c, t, g, unknown or other
gggtgccacgggaaaatctccggcagcccaggttggggagctccacggggaatgcctgtgtgcctgttcttcagtgcccactcacctgtctttcttttttctgcagcatcacaggacatggccccctcagccacctagctggggcccatctaggagtggcatcttttttggtgccctgaaggccagctctggaccttcccaggaaaagtgccagctcacagaactgcttgaccaaaggaccggctcttgagacatcccccaacccacctggcccccagctagggtgggggctccaggagactgagattagcctgccctctttggacagcagctccaggacagggcgggtgggctgaccacccaaaccccatctgggcccaggccccatgccccgaggaggggtggtctgaagcccaccagagccccctgccagactgtctgcctcccttctgactgtggccgcttggcatggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccagtaagtctgggtgtgggggctggggtgggaagggactgtggagggtggcaggcctcanagcttgggctctgggcacgtctgttctgctgtgagtggaaggcacggtgagcgacaaggtcttctccagttggggtgaccatcatttgaggtcttaaaaatgaaagccctatggcctataggctgatcatggtggttgtgtttgaagttgggggtggcagaggggganatgaaaccatgctgttctggcctgcagtggccctcgcacccctcttctgtgctctgctgttggggccttataaatagctccaaggactggacacacaggttggaggtgggtaaacaaacctcttgacatttgatggaaatggcagctct

SEQ ID NO: 152 - DNA, 629 bp, Homo sapiens
  modified_base (102)..(102): a, c, t, g, unknown or other
ctgaggaggatccttttaggtgaatcattctccccagcttttctggcctgctcaggtaggcgatgggcaaacgcttgggggcagcagctggcctggccctcntccctagactgagaccgtagccaggcactgctcccactgtgggtgtggacaacctgactccctcccctccatacccagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtgtgagtgccatagggcaggggccgagtcccgcctcagttggggtctcagatgctcctaaagaccaagggagcagggctctgtggatgtttgtgcacatgcatgaacacgcatgccgtggtgtgcgggctcacggttgaagatggtttgtgtgtanctgcaaggacctgtttgcgagtctggctggctgtgtgtccacgggcaggtctgtgctccgggaccgtgtatgtgtaaccattcctgtttctgcacgtctggctgtgtgtgcttgcgtatgtgtgtgtgtgtgcatgctccaagatggctttc

SEQ ID NO: 153 - DNA, 615 bp, Homo sapiens
  modified_base (61)..(61): a, c, t, g, unknown or other
accctgcggcagcagagaccccatgccctgccctgtgtggggaggcgcctgcgagctgccntcctccatggcctgggcaggcacgccccccggtggccgaggctgggggtgcagctgtgttcccagctgctcagggggtggttctgcttcctcagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggtgaggctcacagacctggaggggtaccggcccccgacacctggcccaggcccccacatccaagccagcaccccatgtctttgtgccaggacaatacgacacctgtccccatctgtgtctaggctgaggtcccctagtgactccactttgccgaggtggcccgcctgtgtcacctttgtgtggtagttcagatcgtggctctggaaccagacacgtgggtgtgtgtccttgtgtgggtcactcaacagctcctanctacagtttcccttccgagggcggggataacattcgtgtttacaga

SEQ ID NO: 154 - DNA, 1233 bp, Homo sapiens
agtcccaagggacccccagggagactgcagctgggagggctgggtgagtggaggcgggagaaggaccttcctggggaaagaggaggcagagcacctaggagggcaccgtcgcctggagtgtgagctggagtagacgcgtgggggatagcatgcggctggctatggggtggggtggggggtgtgtgcagggccacagctgtgctgatggggcttctggggcagaacttgatgtgtgggttgggtgggcatggagggctggagtgcgtggcaatgccttgcctgcccgtgaacgcgtgctgtgtgcgcgtgcttacaagcctgggtgacctcctcagcagctggcagctctctgtcaggctgggggtggacgaggccctgagcagcctgcagctgccctcttaaccccctctgccctccacagctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacggtatggctttcccccggcctgcagggtgggatttgcccagggccacagggccaggatgggcccctctcaggcaccccttcttgtgccaggcaagatctctgcgtccttcccttcccctctcttctccctcctcctgctgcctcttcccaaggagctcccaggaagtgaaggctgggtagagggcaggcctgtgggggctggagccaggctgagaaggggtgccatggagaagaaggccctcactctccctcctcccccagaacaacagctcagaacaacgtgtctctctggacattgacctctgggacaagttcagtgaactctccaccaagtgcatcattaagactgtggacttcgccaagcagctgcccggcttcaccaccctcaccatcgccgaccagatcaccctcctcaaggctgcctgcctggacatcctggtgagggtctgcaccctggcccccaggcactgcccctgtgtcctgggtagatgtccttccagccagacagccaccctcctaaatgtctgtctgcaatcaacctgtccaaatgcccaccgcccaaatgtctgcccttcctctccccatatgtccacctgtccactcgtctccctgtccactcagccacctagcagccagatgtgcaggagctcac

SEQ ID NO: 155 - DNA, 3431 bp, Homo sapiens
gggggaggagggcggtcgtccggggttaggttgagggggggcgtcggtccgttctgggcgggggatgactcacagcccatcccatctccccgacgccgcccgcccgcgcagtgctagctccatggcttagcggaggaggcggcagtggcgagctggggggaggggggactcttattttgttagggggaccgggccgaggcccgaccggcctggcagggctcgcccggggccgggcgtcatgtctcatgcggccgaaccagctcgggatggcgtagaggccagcgcggagggccctcgagccgtgttcgtgctgttggaggagcgcaggccggccgactcggctcagctgctcagcctgaactctttgcttccggaatccgggattgttgctgacatagaattagaaaacgtccttgatcctgacagcttctacgagctcaaaagccaacccttaccccttcgctcaagcctcccaatatcactgcaggccacaccagccaccccagctacactctctgcatcgtcttctgcagggggctccaggacccctgccatgtcgtcatcttcttcatcgagggtcttgctgcggcagcagctaatgcgggcccaggcgcaggagcaggagaggcgtgagcgtcgggaacaggccgccgcggctcccttccccagtcctgcacctgcctctcctgccatctctgtggttggcgtctctgctgggggccacacattgagccgtccaccccctgctcaggtgcccagggaggtgctcaaggtgcagacccatctggagaacccaacgcgctaccacctgcagcaggcgcgccggcagcaggtgaaacagtacctgtccaccacactcgggcccaagctggcttcccaggccctcaccccaccgccggggcccgcaagtgcccagccactgcctgcccctgaggctgcccacactaccggccccacaggcagtgcgcccaacagccccatggcgctgctcaccatcgggtccagctcagagaaggagattgatgatgtcattgatgagatcatcagcctggagtccagttacaatgatgaaatgctcagctatctgcccggaggcaccacaggactgcagctccccagcacgctgcctgtgtcagggaatctgcttgatgtgtacagtagtcaaggcgtggccacaccagccatcactgtcagcaactcctgcccagctgagctgcccaacatcaaacgggagatctctgagaccgaggcaaaggcccttttgaaggaacggcagaagaaagacaatcacaacctaattgagcgtcgcaggcgattcaacattaacgacaggatcaaggaactgggcactctcatccctaagtccagtgacccggagatgcgctggaacaagggcaccatcctgaaggcctctgtggattatatccgcaagctgcagaaggagcagcagcgctccaaagacctggagagccggcagcgatccctggagcaggccaaccgcagcctgcagctccgaattcaggaactagaactgcaggcccagatccatggcctgccagtgcctcccactccagggctgctttccttggccacgacttcggcttctgacagcctcaagccagagcagctggacattgaggaggagggcaggccaggcgcagcaacgttccatgtaggggggggacctgcccagaatgctccccatcagcagccccctgcaccgccctcagatgcccttctggacctgcactttcccagcgaccacctgggggacctgggagaccccttccacctggggctggaggacattctgatggaggaggaggagggggtggtgggaggactgtcggggggtgccctgtccccactgcgggctgcctccgatcccctgctctcttcagtgtcccctgctgtctccaaggccagcagccgccgcagcagcttcagcatggaagaggagtcctgatcaggcctcacccctcccctgggactttcccacccaggaaaggaggaccagtcaggatgaggccccgccttttcccccaccctcccatgagactgccctgcccaggtatcctgggggaagaggagatgtgatcaggccccacccctgtaatcaggcaaggaggaggagtcagatgaggccctgcaccttccccaaaggaaccgcccagtgcaggtatttcagaaggagaaggctggagaaggacatgagatcagggcctgccccctggggatcacagcctcacccctgcccctgtgggactcatccttgcccaggtgagggaaggagacaggatgaggtctcgaccctgtcccctagggactgtcctagccaggtctcctgggaaagggagatgtcaggatgttgctccatcctttgtcttggaaccaccagtctagtccgtcctggcacagaagaggagtcaagtaatggaggtcccagccctgggggtttaagctctgccccttccccatgaaccctgccctgctctgcccaggcaaggaacagaagtgaggatgagacccagccccttcccctgggaactctcctggccttctaggaatggaggagccaggccccaccccttccctataggaacagcccagcacaggtatttcaggtgtgaaagaatcagtaggaccaggccaccgctagtgcttgtggagatcacagccccacccttgtccctcagcaacatcccatctaagcattccacactgcagggaggagtggtacttaagctcccctgccttaacctgggaccaacctgacctaacctaggagggctctgagccaaccttgctcttggggaaggggacagattatgaaatttcatggatgaattttccagacctatatctggagtgagaggcccccacccttgggcagagtcctgccttcttccttgaggggcagtttgggaaggtgatgggtattagtgggggactgagttcaggttaccagaaccagtacctcagtattctttttcaacatgtagggcaagaggatgaaggaaggggctatcctgggacctccccagcccaggaaaaactggaagccttcccccagcaaggcagaagcttggaggagggttgtaaaagcatattgtaccccctcatttgtttatctgatttttttattgctccgcatactgagaatctaggccaccccaacctctgttccccacccagttcttcatttggaggaatcaccccatttcagagttatcaagagacactcccccctccattcccacccctcatacctacacccaaggttgtcagctttggattgctggggccaggccccatggagggtatactgaggggtctataggtttgtgattaaaataataaaagctaggcgtgtttgatgcgcttttaactttgaaaaaaaaaaaaaaaaaaaaaaaaaaaaaaaaaaaaaa

SEQ ID NO: 156 - DNA, 1449 bp, Homo sapiens
agaaaggagtggggttgaaaagcgcttgcgcaggacggctacggtacgggggtgggagggcttcggagcacgcgcgcggaggcgggacttgggaagcgctcgcgagatcttcagggtctatatataagcgcggggagcctgcgtcctttccctggtgtgattccgtcctgcgcggttgttctctggagcagcgttcttttatctccgtccgccttctctcctacctaagtgcgtgccgccacccgatggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtggggagacaaaaatatacatgtgaaataaaactcagtattttaataaagta

SEQ ID NO: 157 - DNA, 11711 bp, Homo sapiens
cacagtggtcctccgccggctacggcgctgcgtcactggtttgcaggcgctttcctcttggaagtggcgactgctgcgggcctgagcgctggtctcacgcgcctcgggagccaggttggcggcgcgatgaggcgcagcaaggctgacgtggagcggtacatcgcctcggtgcagggctccaccccgtcgcctcgacagaagtcaatgaaaggattctattttgcaaagctgtattatgaagctaaagaatatgatcttgctaaaaaatacatatgtacttacattaatgtgcaagagagggatcccaaagctcacagatttctgggtcttctttatgaattggaagaaaacacagacaaagccgttgaatgttacaggcgttcagtggaattaaacccaacacaaaaagatcttgtgttgaagattgcagaattgctttgtaaaaatgatgttactgatggaagagcaaaatactggcttgaaagagcagccaaacttttcccaggaagtcctgcaatttataaactaaaggaacagcttctagattgtgaaggtgaagatggatggaataaactttttgacttgattcagtcagaactttatgtaagacctgatgacgtccatgtgaacatccggctagtggaggtgtatcgctcaactaaaagattgaaggatgctgtggcccactgccatgaggcagagaggaacatagctttgcgttcaagtttagaatggaattcgtgtgttgtacagacccttaaggaatatctggagtctttacagtgtttggagtctgataaaagtgactggcgagcaaccaatacagacttactgctggcctatgctaatcttatgcttcttacgctttccactagagatgtgcaggaaagtagagaattactgcaaagttttgatagtgctcttcagtctgtgaaatctttgggtggaaatgatgaactgtcagctactttcttagaaatgaaaggacatttctacatgcatgctggttctctgcttttgaagatgggtcagcatagtagtaatgttcaatggcgagctctttctgagctggctgcattgtgctatctcatagcatttcaggttccaagaccaaagattaaattaataaaaggtgaagctggacaaaatctgctggaaatgatggcctgtgaccgactgagccaatcagggcacatgttgctaaacttaagtcgtggcaagcaagattttttaaaagagattgttgaaacttttgccaacaaaagcgggcagtctgcattatatgatgctctgttttctagtcagtcacctaaggatacatcttttcttggtagcgatgatattggaaacattgatgtacgagaaccagagcttgaagatttgactagatacgatgttggtgctattcgagcacataatggtagtcttcagcaccttacttggcttggcttacagtggaattcattgcctgctttacctggaatccgaaaatggctaaaacagcttttccatcatttgccccatgaaacctcaaggcttgaaacaaatgcacctgaatcaatatgtattttagatcttgaagtatttctccttggagtagtatataccagccacttacaattaaaggagaaatgtaattctcaccacagctcctatcagccgttatgcctgccccttcctgtgtgtaaacagctttgtacagaaagacaaaaatcttggtgggatgcggtttgtactctgattcacagaaaagcagtacctggaaacgtagcaaaattgagacttctagttcagcatgaaataaacactctaagagcccaggaaaaacatggccttcaacctgctctgcttgtacattgggcagaatgccttcagaaaacgggcagcggtcttaattctttttatgatcaacgagaatacatagggagaagtgttcattattggaagaaagttttgccattgttgaagataataaaaaagaagaacagtattcctgaacctattgatcctctgtttaaacattttcatagtgtagacattcaggcatcagaaattgttgaatatgaagaagacgcacacataacttttgctatattggatgcagtaaatggaaatatagaagatgctgtgactgcttttgaatctataaaaagtgttgtttcttattggaatcttgcactgatttttcacaggaaggcagaagacattgaaaatgatgccctttctcctgaagaacaagaagaatgcaaaaattatctgagaaagaccagggactacctaataaagattatagatgacagtgattcaaatctttcagtggtcaagaaattgcctgtgcccctggagtctgtaaaagagatgcttaattcagtcatgcaggaactcgaagactatagtgaaggaggtcctctctataaaaatggttctttgcgaaatgcagattcagaaataaaacattctacaccgtctcctaccagatattcactatcaccaagtaaaagttacaagtattctcccaaaacaccacctcgatgggcagaagatcagaattctttactgaaaatgatttgccaacaagtagaggccattaagaaagaaatgcaggagttgaaactaaatagcagtaactcagcatcccctcatcgttggcccacagagaattatggaccagactcagtgcctgatggatatcaggggtcacagacatttcatggggctccactaacagttgcaactactggcccttcagtatattatagtcagtcaccagcatataattcccagtatcttctcagaccagcagctaatgttactcccacaaagggcccagtctatggcatgaataggcttccaccccaacagcatatttatgcctatccgcaacagatgcacacaccgccagtgcaaagctcatctgcttgtatgttctctcaggagatgtatggtcctcctgcattgcgttttgagtctcctgcaacgggaattctatcgcccaggggtgatgattactttaattacaatgttcaacagacaagcacaaatccacctttgccagaaccaggatatttcacaaaacctccgattgcagctcatgcttcaagatctgcagaatctaagactatagaatttgggaaaactaattttgttcagcccatgccgggtgaaggattaaggccatctttgccaacacaagcacacacaacacagccaactccttttaaatttaactcaaatttcaaatcaaatgatggtgacttcacgttttcctcaccacaggttgtgacacagccccctcctgcagcttacagtaacagtgaaagccttttaggtctcctgacttcagataaacccttgcaaggagatggctatagtggagccaaaccaattcctggtggtcaaaccattgggcctcgaaatacattcaattttggaagcaaaaatgtgtctggaatttcatttacagaaaacatggggtcgagtcagcaaaagaattctggttttcggcgaagtgatgatatgtttactttccatggtccagggaaatcagtatttggaacacccactttagagacagcaaacaagaatcatgagacagatggaggaagtgcccatggggatgatgatgatgacggtcctcactttgagcctgtagtacctcttcctgataagattgaagtaaaaactggtgaggaagatgaagaagaattcttttgcaaccgcgcgaaattgtttcgtttcgatgtagaatccaaagaatggaaagaacgtgggattggcaatgtaaaaatactgaggcataaaacatctggtaaaattcgccttctaatgagacgagagcaagtattgaaaatctgtgcaaatcattacatcagtccagatatgaaattgacaccaaatgctggatcagacagatcttttgtatggcatgcccttgattatgcagatgagttgccaaaaccagaacaacttgctattaggttcaaaactcctgaggaagcagcactttttaaatgcaagtttgaagaagcccagagcattttaaaagccccaggaacaaatgtagccatggcgtcaaatcaggctgtcagaattgtaaaagaacccacaagtcatgataacaaggatatttgcaaatctgatgctggaaacctgaattttgaatttcaggttgcaaagaaagaagggtcttggtggcattgtaacagctgctcattaaagaatgcttcaactgctaagaaatgtgtatcatgccaaaatctaaacccaagcaataaagagctcgttggcccaccattagctgaaactgtttttactcctaaaaccagcccagagaatgttcaagatcgatttgcattggtgactccaaagaaagaaggtcactgggattgtagtatttgtttagtaagaaatgaacctactgtatctaggtgcattgcgtgtcagaatacaaaatctgctaacaaaagtggatcttcatttgttcatcaagcttcatttaaatttggccagggagatcttcctaaacctattaacagtgatttcagatctgttttttctacaaaggaaggacagtgggattgcagtgcatgtttggtacaaaatgaggggagctctacaaaatgtgctgcttgtcagaatccgagaaaacagagtctacctgctacttctattccaacacctgcctcttttaagtttggtacttcagagacaagtaaaactctaaaaagtggatttgaagacatgtttgctaagaaggaaggacagtgggattgcagttcatgcttagtgcgaaatgaagcaaatgctacaagatgtgttgcttgtcagaatccggataaaccaagtccatctacttctgttccagctcctgcctcttttaagtttggtacttcagagacaagcaaggctccaaagagcggatttgagggaatgttcactaagaaggagggacagtgggattgcagtgtgtgcttagtaagaaatgaagccagtgctaccaaatgtattgcttgtcagaatccaggtaaacaaaatcaaactacttctgcagtttcaacacctgcctcttcagagacaagcaaggctccaaagagcggatttgagggaatgttcactaagaaggagggacagtgggattgcagtgtgtgcttagtaagaaatgaagccagtgctaccaaatgtattgcttgtcagaatccaggtaaacaaaatcaaactacttctgcagtttcaacacctgcctcttcagagacaagcaaggctccaaagagcggatttgagggaatgttcactaagaaggaaggacagtgggattgcagtgtgtgcttagtaagaaatgaagccagtgctaccaaatgtattgcttgtcagtgtccaagtaaacaaaatcaaacaactgcaatttcaacacctgcctcttcggagataagcaaggctccaaagagtggatttgaaggaatgttcatcaggaaaggacagtgggattgtagtgtttgctgtgtacaaaatgagagttcttccttaaaatgtgtggcttgtgatgcctctaaaccaactcataaacctattgcagaagctccttcagctttcacactgggctcagaaatgaagttgcatgactcttctggaagtcaggtgggaacaggatttaaaagtaatttctcagaaaaagcttctaagtttggcaatacagagcaaggattcaaatttgggcatgtggatcaagaaaattcaccttcatttatgtttcagggttcttctaatacagaatttaagtcaaccaaagaaggattttccatccctgtgtctgctgatggatttaaatttggcatttcggaaccaggaaatcaagaaaagaaaagtgaaaagcctcttgaaaatggtactggcttccaggctcaggatattagtggccagaagaatggccgtggtgtgatttttggccaaacaagtagcacttttacatttgcagatcttgcaaaatcaacttcaggagaaggatttcagtttggcaaaaaagaccccaatttcaagggattttcaggtgctggagaaaaattattctcatcacaatacggtaaaatggccaataaagcaaacacttccggtgactttgagaaagatgatgatgcctataagactgaggacagcgatgacatccattttgaaccagtagttcaaatgcccgaaaaagtagaacttgtaacaggagaagaagatgaaaaagttctgtattcacagcgggtaaaactatttagatttgatgctgaggtaagtcagtggaaagaaaggggcttggggaacttaaaaattctcaaaaacgaggtcaatggcaaactaagaatgctgatgcgaagagaacaagtactaaaagtgtgtgctaatcattggataacgactacgatgaacctgaagcctctctctggatcagatagagcatggatgtggttagccagtgatttctctgatggtgatgccaaactagagcagttggcagcaaaatttaaaacaccagagctggctgaagaattcaagcagaaatttgaggaatgccagcggcttctgttagacataccacttcaaactccccataaacttgtagatactggcagagctgccaagttaatacagagagctgaagaaatgaagagtggactgaaagatttcaaaacatttttgacaaatgatcaaacaaaagtcactgaggaagaaaataagggttcaggtacaggtgcggccggtgcctcagacacaacaataaaacccaatcctgaaaacactgggcccacattagaatgggataactatgatttaagggaagatgctttggatgatagtgtcagtagtagctcagtacatgcttctccattggcaagtagccctgtgagaaaaaatcttttccgttttggtgagtcaacaacaggatttaacttcagttttaaatctgctttgagtccatctaagtctcctgccaagttgaatcagagtgggacttcagttggcactgatgaagaatctgatgttactcaagaagaagagagagatggacagtactttgaacctgttgttcctttacctgatctagttgaagtatccagtggtgaggaaaatgaacaagttgtttttagtcacagggcaaaactctacagatatgataaagatgttggtcaatggaaagaaaggggcattggtgatataaagattttacagaattatgataataagcaagttcgtatagtgatgagaagggaccaagtattaaaactttgtgccaatcacagaataactccagacatgactttgcaaaatatgaaagggacagaaagagtatggttgtggactgcatgtgattttgcagatggagaaagaaaagtagagcatttagctgttcgttttaaactacaggatgttgcagactcgtttaagaaaatttttgatgaagcaaaaacagcccaggaaaaagattctttgataacacctcatgtttctcggtcaagcactcccagagagtcaccatgtggcaaaattgctgtagctgtattagaagaaaccacaagagagaggacagatgttattcagggtgatgatgtagcagatgcaacttcagaagttgaagtgtctagcacatctgaaacaacaccaaaagcagtggtttctcctccaaagtttgtatttggttcagagtctgttaaaagcatttttagtagtgaaaaatcaaaaccatttgcattcggcaacagttcagccactgggtctttgtttggatttagttttaatgcacctttgaaaagtaacaatagtgaaactagttcagtagcccagagtggatctgaaagcaaagtggaacctaaaaaatgtgaactgtcaaagaactctgatatcgaacagtcttcagatagcaaagtcaaaaatctctttgcttcctttccaacggaagaatcttcaatcaactacacatttaaaacaccagaaaaggcaaaagagaagaaaaaacctgaagattctccctcagatgatgatgttctcattgtatatgaactaactccaaccgctgagcagaaagcccttgcaaccaaacttaaacttcctccaactttcttctgctacaagaatagaccagattatgttagtgaagaagaggaggatgatgaagatttcgaaacagctgtcaagaaacttaatggaaaactatatttggatggctcagaaaaatgtagacccttggaagaaaatacagcagataatgagaaagaatgtattattgtttgggaaaagaaaccaacagttgaagagaaggcaaaagcagatacgttaaaacttccacctacatttttttgtggagtctgtagtgatactgatgaagacaatggaaatggggaagactttcaatcagagcttcaaaaagttcaggaagctcaaaaatctcagacagaagaaataactagcacaactgacagtgtatatacaggtgggactgaagtgatggtaccttctttctgtaaatctgaagaacctgattctattaccaaatccattagttcaccatctgtttcctctgaaactatggacaaacctgtagatttgtcaactagaaaggaaattgatacagattctacaagccaaggggaaagcaagatagtttcatttggatttggaagtagcacagggctctcatttgcagacttggcttccagtaattctggagattttgcttttggttctaaagataaaaatttccaatgggcaaatactggagcagctgtgtttggaacacagtcagtcggaacccagtcagccggtaaagttggtgaagatgaagatggtagtgatgaagaagtagttcataatgaagatatccattttgaaccaatagtgtcactaccagaggtagaagtaaaatctggagaagaagatgaagaaattttgtttaaagagagagccaaactttatagatgggatcgggatgtcagtcagtggaaggagcgcggtgttggagatataaagattctttggcatacaatgaagaattattaccggatcctaatgagaagagaccaggtttttaaagtgtgtgcaaaccacgttattactaaaacaatggaattaaagcccttaaatgtttcaaataatgctttagtttggactgcctcagattatgctgatggagaagcaaaagtagaacagcttgcagtgagatttaaaactaaagaagtagctgattgtttcaagaaaacatttgaagaatgtcagcagaatttaatgaaactccagaaaggacatgtatcactggcagcagaattatcaaaggagaccaatcctgtggtgttttttgatgtttgtgcggacggtgaacctctagggcggataactatggaattattttcaaacattgttcctcggactgctgagaacttcagagcactatgcactggagagaaaggctttggtttcaagaattccatttttcacagagtaattccagattttgtttgccaaggaggagatatcaccaaacatgatggaacaggcggacagtccatttatggagacaaatttgaagatgaaaattttgatgtgaaacatactggtcctggtttactatccatggccaatcaaggccagaataccaataattctcaatttgttataacactgaagaaagcagaacatttggactttaagcatgtagtatttgggtttgttaaggatggcatggatactgtgaaaaagattgaatcatttggttctcccaaagggtctgtttgtcgaagaataactatcacagaatgtggacagatataaaatcattgttgttcatagaaaatttcatctgtataagcagttggattgaagcttagctattacaatttgatagttatgttcagcttttgaaaatggacgtttccgatttacaaatgtaaaattgcagcttatagctgttgtcactttttaatgtgttataattgaccttgcatggtgtgaaataaaagtttaaacactggtgtatttcaggtgtacttgtgtttatgtactcctgacgtattaaaatggaataatactaatcttgttaaaagcaatagacctcaaactattgaaggaatatgatatatgcaatttaattttaattccttttaagatatttggacttcctgcatggatatacttaccatttgaataaagggaccacaacttggataatttaattttaggtttgaaatatatttggtaatcttaactattggtgtactcatttatgcatagagactcgtttatgaatgggtagagccacagaacgtatagagttaaccaaagtgctcttctctagaatctttacacctcctgtgtggttacaagttaactttgtaagtagcgtaccttccttccttaaaatatctagcttcctgtgccctttcatagatattcgattaatttttacattttaaacaagttgactatttcctttaggggttttgtttcaaacttttctgtcatctgtctctactacctcagaaactgcagcttggttctgatgatagaaattgaatttttccttgtagttattgtgataaagtatgaatatttttagaaagtctataccatgttctttcgttaaagatttgctttatacaagattgttgcagtacctttttctggtaaattttgtagcagaaataaaatgacaattcctaagagccactgacatccaaaaaattcattacttacgcttcgggttcattctaaagtaaggaagacaatttaaaggcagtaaattcaaactgctgcataatttccagagctcctagtttctcaagtttgatacacaccaaaaacgtatttggaaatggcttgtatcaaatgttaggcaaattgctaaagaaaagaggatgttcttattggcctactcaatatggaactacaaagaatccaggtagaacacaaaattttgtatattgcaattatgaatattgactgtcttccacccatctgtgttctttcgggtgaaattacctcattttatttagtgaggaaagacaggtttattccctgttacatggggatttggaaattgggtatcctaaagcaagtaactgttcaaccaccagtcaaaagaggggagggatgttgtgcgagtaatgagtgatggtataccatcaccattccactcggccacaaagccagatacttgaaataaacctactccaaatgtatcagttcagtcttgaaccatggattacatatgtttacactaaatatttcaaattggcttatttggaaatctatgtaatataaactgatgtaaagtgtgttgtaacttttcagctgagacagttgatgccttcgtcatgattttagaataaattcttaagttaatgcaagtgctttttaagagactttttacagatttgtatgctttcctaaagcactaaagttacaaattaaaaagctttaaaaactttgaccaaaaatttgacaaaatgacatgtaaactgacttttcccgtattagtattccaaagatgcttaaaagtggcttgtggcatttatgagaaagtctttgtgtcacatttcaggaaaggactttgatttctctttgttatttaatcactgatgtggtctaaacccacgataatatgtatctttccttttaaactggattttatgttgtctcattaaaatctgcttaaagatagaataaaattcattatttgtaca

SEQ ID NO: 158 - DNA, 3212 bp, Homo sapiens
gtcacatccgggcgggttggtgagttccggtatttcagggcgtagcaggcggaagtaagggtgagaggaggctgcaacgccgagcggaggaggcaggaaccggagcgcgagcagtagctgggtgggcaccatggctgggatcaccaccatcgaggcggtgaagcgcaagatccaggttctgcagcagcaggcagatgatgcagaggagcgagctgagcgcctccagcgagaagttgagggagaaaggcgggcccgggaacaggctgaggctgaggtggcctccttgaaccgtaggatccagctggttgaagaagagctggaccgtgctcaggagcgcctggccactgccctgcaaaagctggaagaagctgaaaaagctgctgatgagagtgagagaggtatgaaggttattgaaaaccgggccttaaaagatgaagaaaagatggaactccaggaaatccaactcaaagaagctaagcacattgcagaagaggcagataggaagtatgaagaggtggctcgtaagttggtgatcattgaaggagacttggaacgcacagaggaacgagctgagctggcagagtcccgttgccgagagatggatgagcagattagactgatggaccagaacctgaagtgtctgagtgctgctgaagaaaagtactctcaaaaagaagataaatatgaggaagaaatcaagattcttactgataaactcaaggaggcagagacccgtgctgagtttgctgagagatcggtagccaagctggaaaagacaattgatgacctggaagataaactgaaatgcaccaaagaggagcacctctgtacacaaaggatgctggaccagaccctgcttgacctgaatgagatgtagaacgccccagtcccaccctgctgctgctcctccctctgacccagactccgcctgaggccagcctgcgggaagctgacctttaactgagggctgatctttaactggaaggctgctttctcctttcaccaccccctccttccctgtgtctttttcgccaaactgtctctgcctcttcccggagaatccagctgggctagaggctgagcacctttggaaacaacatttaagggaatgtgagcacaatgcataatgtctttaaaaagcatgttgtgatgtacacattttgtaattaccttttttgttgttttgtagcaaccatttgtaaaacattccaaataattccacagtcctgaagcagcaatcgaatccctttctcacttttggaaggtgacttttcaccttaatgcatattcccctctccatagaggagaggaaaaggtgtaggcctgccttaccgagagccaaacagagcccagggagactccgctgtgggaaacctcattgttctgtacaaagtactagctaaaccagaaaggtgattccaggaggagttagccaaacaacaacaaaaacaaaaaatgtgctgttcaagttttcagctttaagatatctttggataatgttatttctattttttatttttttcattagaagttaccaaattaagatggtaagacctctgagaccaaaattttgtcccatctctaccccctcacaactgcttacagaatggatcatgtcccccttatgttgaggtgaccacttaattgctttcctgcctccttgaaagaaagaaagaaagaagactgtgtttttgccactgatttagccatgtgaaactcatctcattacccttttctgggtttgaagctgctgtctctagaagtgccatctcaattgtgctttgtatcagtcagtgctggagaaatcttgaatagcttatgtacaaaactttttaaattttatattattttgaaactttgctttgggtttgtggcaccctggccaccccatctggctgtgacagcctctgcagtccgtgggctggcagtttgttgatcttttaagtttccttccctacccagtccccattttctggtaaggtttctaggaggtctgttaggtgtacatcctgcagcttattggcttaaaatgtactctccttttatgtggtctctttggggccgattgggagaaagagaaatcaatagtgcaactgttttgatactgaatattgacaagtgtctttttgaaataaagaaccagtccctccaaccctcagacctatttgacttttatttattaaaactaaatgtgctttctccacagaagctatgaggtttgggttaaaaatagcatctttgtgggtggtagcaacaggatttattctttattattattatttttgagatgaagtttcattcttgttgcctgggctggagcgtaatggctcgatctcggctcactgcaacctccgcctcctggttcaagagattctcctgcctcagcctcccgagtagctgggattacaggcacctgccaccatgcccgggtaattttttatattttaagtagagacagggcttcaccatgttggccaggctggtctcgaactcctgaccttcaggtgatccacctgcctcagcttcccaaaatgctgggattacgggcgtgagccaccgcacccagctggagcaacaggatttaatatagagcaaatgtttagttttatcatctgtaaaatggagataagtattgtcagagtaaacatgaagattagaaagaacacttaatgtgctgggccttttataggttaacactgacatctcaggctgaactatatacattttccttcacaaccatatcaatccttataaactatggatttatgctccttaaaacaatatataatgctgatcactactataaatgcgtggttttaaccaactgtactgaaacagctttgagtttatattctgtttggatatttggagaaaacaacaagtgctctcaagagtatttgcttagaggccggctgtgtgagtggataactttgaaagctgcttttgagacgccagtgtctggcatttcctgcattctggcctggaggccggacgtgaatctgacttctagtaaaaatacacggttcccttgacaaagtcgagctgtttatcccagagactgcacaattttccgttgataggcatggaccaatgctaactggaaatcattgcaaaaagtttttttgtcgggcggagggtgtggtgttaagataaacagtgtgcaacagaagaaattaaaactggaagaaattaaagggttttttttagacttt

SEQ ID NO: 159 - DNA, 1316 bp, Homo sapiens
tcctggagcaatgacattgcagaatattttctcctcctccagccacactttgtcaccaactgctgccaactcgccaccactgctgccgacctcgcaaccactgctttgtctctgaagtgagactgctcctggtgccatgaacggagacgacacctttgcaaagagacccagggatgatgctaaagcatcagagaagagaagcaaggcctttgatgatattgccacatacttctctaagaaagagtggaaaaagatgaaatactcggagaaaatcagctatgtgtatatgaagagaaactataaggccatgactaaactaggtttcaaagtcaccctcccacctttcatgtgtaataaacaggccacagacttccaggggaatgattttgataatgaccataaccgcaggattcaggttgaacatcctcagatgactttcggcaggctccacagaatcatcccgaagatcatgcccaagaagccagcagaggacgaaaatgattcgaagggagtgtcagaagcatctggcccacaaaacgatgggaaacaactgcaccccccaggaaaagcaaatatttctgagaagattaataagagatctggacccaaaagggggaaacatgcctggacccacagactgcgtgagagaaagcagctggtgatttatgaagagatcagtgaccctgaggaagatgacgagtaactcccctgggggatacgacacatgcccttgatgagaagcagaacgtggtgacctttcacgaacatgggcatggctgcggctccctcgtcatcaggtgcatagcaagtgaaagcaagtgttcacaacggtgaaacttgagcgtcatttttcttagtgtgccaagagttcgatgttagtgtttccattgtattttcttacagtgtgccattctgttagatactatccttataattgatgagcaagacatactgaatgcatatttcggtttgtgtatccatgcacctacgtcagaaaacaagtattgtcaggtattctctccatagaacagcactatcctcatctctccccagatgtgactactgagggcagttctgagtgtttaatttcagactttttcctctgcatttacacacacacacacacacacacgcacacacacacaccaagtaccagtataagcatctcccatctgcttttcccattgccatgcgtcctggtcaagcccccctcactctgtttcctgttcagcatgtactcccctcatccgattcccctgtatcagtcactgacagttaataaacctttgcaaacgttcaaaaaaaaaaaaaaaaaa

SEQ ID NO: 160 - DNA, 1494 bp, Homo sapiens
gggattggctactttaagttcagagtacgcatgctctgactttctctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggaaatagggaggcccaagaaaaggaagagagacgcggaacagctcatcggtggagcagtcagaacacacacaacattggtcgattcagtttgtcaacttctatgggtgcagttcatggtacccccaaaacaattacacacaacagggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagattttttcctctgcatttacacacacacgcacacaaaccacaccacacacacacacacacacacacacacacacacacacacacaccaagtaccagtataagcatctgccatctgcttttcccattgccatgcgtcctggtcaagctcccctcactctgtttcctggtcagcatgtactcccctcatccgattcccctgtagcagtcactgacagttaataaacctttgcaaacgttcaaaaaaaaaaaaaaaaaa

SEQ ID NO: 161 - DNA, 3393 bp, Homo sapiens
cctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagctcgcagcccgcagcccgcagaggacgcccagagcggcgagcgggcgggcagacggaccgacggactcgcgccgcgtccacctgtcggccgggcccagccgagcgcgcagcgggcacgccgcgcgcgcggagcagccgtgcccgccgcccgggccccgcgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctcactcgggctcgggatccgcccaggtagcggcctcggacccaggtcctgcgcccaggtcctcccctgccccccagcgacggagccggggccgggggcggcggcgcccgggggccatgcgggtgagccgcggctgcagaggcctgagcgcctgatcgccgcggacccgagccgagcccacccccctccccagccccccaccctggccgcgggggcggcgcgctcgatctacgcgtccggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggtccttggagtgataatattgtttccctcgtccgtctgtctcgatgcctgattcggacggccaatggtgcttcccccacccctccacgtgtccgtccacccttccatcagcgggtctcctcccagcggcctccggcgtcttgcccagcagctcaagaagaaaaagaaggactgaactccatcgccatcttcttcccttaactccaagaacttgggataagagtgtgagagagactgatggggtcgctctttgggggaaacgggctccttcccctgcacctggcctgggccacacctgagcgctgtggactgtcctgaggagccctgaggacctctcagcatagcctgcctgatccctgaacccctggccagctctgaggggaggcacctccaggcaggccaggctgcctcggactccatggctaagaccacagacgggcacacagactggagaaaacccctcccacggtgcccaaacaccagtcacctcgtctccctggtgcctctgtgcacagtggcttcttttcgttttcgttttgaagacgtggactcctcttggtgggtgtggccagcacaccaagtggctgggtgccctctcaggtgggttagagatggagtttgctgttgaggtggctgtagatggtgacctgggtatcccctgcctcctgccaccccttcctccccacactccactctgattcacctcttcctctggttcctttcatctctctacctccaccctgcattttcctcttgtcctggcccttcagtctgctccaccaaggggctcttgaaccccttattaaggccccagatgatcccagtcactcctctctagggcagaagactagaggccagggcagcaagggacctgctcatcatattccaacccagccacgactgccatgtaaggttgtgcagggtgtgtactgcacaaggacattgtatgcagggagcactgttcacatcatagataaagctgatttgtatatttattatgacaatttctggcagatgtaggtaaagaggaaaaggatccttttcctaattcacacaaagactccttgtggactggctgtgcccctgatgcagcctgtggcttggagtggccaaataggagggagactgtggtaggggcagggaggcaacactgctgtccacatgacctccatttcccaaagtcctctgctccagcaactgcccttccaggtgggtgtgggacacctgggagaaggtctccaagggagggtgcagccctcttgcccgcacccctccctgcttgcacacttccccatctttgatccttctgagctccacctctggtggctcctcctaggaaaccagctcgtgggctgggaatgggggagagaagggaaaagatccccaagaccccctggggtgggatctgagctcccacctcccttcccacctactgcactttcccccttcccgccttccaaaacctgcttccttcagtttgtaaagtcggtgattatatttttgggggctttccttttattttttaaatgtaaaatttatttatattccgtatttaaagttgtaaaaaaaaataaccacaaaacaaaaccaaatgaaaaaaaaaaaaaaaaaa

SEQ ID NO: 162 - DNA, 7471 bp, Homo sapiens
gctgggtcctggagcagagccgaggagccctggggtccctcaaagtttgtgtctggagccgtagcggcaagtgggcttgcggctaagggattttcctgggatgagagcgggtcttctgccttcattttggatgcacatcccgctttagccccggcagcctttggtccggctcgtgtccctggggattctcggatctccgaggacaccggacgggagcgcttggccatcctctctccggcagaggagcagacgtttgctttccaagtgcaaaactacagacacgcgcgcgcacacacgcaagcacacgcggagagagaggaaccttgccggtccgaggcagctctgcgcgtcccctcctgcgcttagcatcctcggcccagcgcggcccgcaccgccatggaggtgctggagagcggggagcagggcgtgctgcagtgggaccgcaagctgagcgagctgtcagagcccggggacggcgaggccctcatgtaccacacgcacttctcagaacttctggatgagttttcccagaacgtcttgggtcagctcctgaatgatcctttcctctcagagaagagtgtgtcaatggaggtggaaccttccccgacgtccccggcgcctctcatccaggctgagcacagctactccctgtgcgaggagcctcgggcccagtcgcccttcacccacattaccaccagtgacagcttcaatgacgatgaggtggaaagtgagaaatggtacctgtctacagacttcccttcaacatccatcaagacagagccagttacagacgaaccacccccaggactcgttccgtctgtcactctgaccatcacagccatctccaccccgttggaaaaggaggaacctcctctggaaatgaacactggggttgattcctcgtgccagaccattattcctaaaattaagctggagcctcatgaagtggatcagtttctaaacttctctcctaaagaagccccagtggaccacctgcatttgccgcccacccctccgagcagtcacggcagtgactcagagggcagcctgagtcccaacccacgcctgcaccccttcagcctgcctcagacccacagcccctccagagctgcaccccgggccccctccgccctctccagctcccctctcctcacggctcctcataaactgcagggatcaggccctctggtcctgacagaggaggagaagaggaccctgatcgctgagggctatcccatccccaccaaattgcccctgtcaaaatcagaggagaaggccctgaagaaaattcggaggaagatcaagaataagatttctgctcaggaaagtaggagaaagaagaaagaatacatggacagcctggagaaaaaagtggagtcttgttcaactgagaacttggagcttcggaagaaggtagaggttctagagaacactaataggactctccttcagcaactccagaagcttcagactttggtgatgggcaaggtttctcgaacctgcaagttagctggcacgcagactggcacctgcctcatggttgtggtgctgtgctttgccgttgcattcggcagcttctttcaaggctacgggccctatccttctgccaccaagatggctctgcccagccagcattccctgcaggagccctacacagcctccgtggtgagatccagaaacctgctgatctacgaggaacattctcccccagaggagtcatccagcccgggctcggctggggagctggggggctgggatagaggttcctccctgctcagggtgtcagggctggagtccaggccggatgtggatcttccccatttcattatctcgaatgagaccagcctggagaagtcagtgcttttggagctgcagcagcacctggtcagcgccaaactggaggggaatgaaacactaaaagttgtagaactcgacagaagagtgaacaccactttctaaagaggctgcctgcaccccctccctttcccttaactctacttttacatccccaaaccacctttgtcatcagcttttcctctttgccactggatcttcatggagacatgggcaagcattagtggcttcagattggagaccagcctgggacttccctgcagtgagagagcatctccccctggtccatgcccctcctgtgcagaagggagcctgcatccctcccttcctttctcttactgccataggaaattattttaggggttggaggtgggacaagcaggcttgtttccaccaatagtgccaaaaagatattgcctaatgtgcacctgtgaggtgtaaccccccgctttggagacgagatggctcttgttcagtcaagaccccagactctggccacaaaaatgccataatgcctgttggtatttggcaaagcactgacccgtgtcctccgttgctcgcactggggtctctggtgtgaacacccccgacagcagccctccgcccactctgccccctgggagccctcgctggatcgtctcgtctcctgcagcagcactggcaggcgagggctctcgttcatattctcaggccgcaagtgcaatgcctgaggggatcaggcttttctactccaggcaaacctgccccatcttgtcgcttttaggacctcccacaacctggttccccacacatccatagttctgcctccccagcttctcctccccagttgtaaatagtatttattagcttgccgaggcttcctgctagcaaccacactgaagagatcgatgcctcctttcaagctagccaagttttctgcgagccttcagagctaggagggcaccctaggctctgggatcccgtgtctttccagacaatgttttgtttcctttcctttgttttttcttttaactggaataattaccattgaaaaagaagttcctttgagcatgtatgtgtctgcctctaggatgagctcagagcgagagatgacacaatgcctcactcaggccccgggctccctggccacaagctttttctatcctgttttcatgacagagaaggggaagccctgttctgacaacagacatttcagacaaccttgctggctttccacacctgcctggccccctcctccctccacacttccactttgtcctcctcgtcccctacctcaacaaagcagggtggggtaggtgacatttgtgtatccacattcttacctttggtagtcaggtttggctactttgcagctcgcccaaagagatacaacctaatccccaacctacttttagtttttttgttttttttttatggttaaaagtaacttttgtagtttaaaaaaatctttcctctttcatataaataagaagtggaaattgcctttttattgtgtaatgtagaaaaccctcaagtgttttttccgagcttgggaaagattttgtgtaggaaatgtgcatagagtttgtattttatttttattagcagctgaaatgcctttggttttggcttctctctctccctctctctctctgtctctccttctctctctccccccaccacccacccccacacacgtcatctgcattgttattggagcctgtacttagagggattaagcccacaccctggcttccattccatatcaggtacaggatttgatgttattaacatttgtcgtcatacctcataagtcggtccctgccttgtctgtctaggcccatttggggctccctgtgagtgattcccctctctctgctatgctggagacggttccagcctggaaagcggccaagttcatcttctcactgtgagtggaagctggatcgggcccccgtagtcctggcagccctgttgtctggagggttcttgttgtccctcccattagccagggcggagactgtctgagctgtgcaggaggagggttgctagtaggttctgcttctgcttctctctgctccactgtctgcagcccagatcctgttgggcctggctggtgtctggtaaccatgggcctccactgacccatccctctcttttaaactgtcaggtcattatcaggcataggcagcctatagggcccaaagaaggcaaaaagataagatttactcaagtagcatttgggcaatgaggaaggaaaggtttcaaatttaggggcagaagtgagagaatgagccaacccatgtacctgctgcaactgaaccagactgggttttcaaggctcccagacgtagagtaggaaacgtgctcttctaaatgaggagggagaagataaaggaaacttctagcccctgtccttagtgctttgaggattttattttctcccttactacgcttgcttgacgtcactctctctcgacctccaaacagcaggactctttctctgggaaaccatccttccaaaacggaatctatgtagacaatgggacgttaggcagagagctcagatggcccttttaagggggctccaagaaccaacatcactgctcttttagataaacctctgccctccactccttgcttgagtgggttaaaggaactaacagttgtccctttaggaggacaaaatggggtcaagaggacacagaagagttgtatagcaccagattggttccaaatagttaatggatgtgtgcacattttctgttcagggattaagaccagaatatcagtggatttgttttccccaccaagtggcctcttagactagtcattaacttatgattagctctaaagatttcaaatagtggcagacagtgtcttctgaatgtaagttttgagaaatacgagtctgtcagagcggccataagccataaagagtcaatctcttaattatatttttcatcatgtaaacaagtttcccatttccctttcttagattgcaccagtgaaggagatgttttgcaaagattcagagaactaatttttcactggataagacctgagtaacccagaccccccaccgtggttcttttcacagccctcgactttgcacttaaaaagggatattgtaaatgaaaggctgcagtgccagttttaagaaagaatttctgtgaagtgtgaggactctggagtctagctcacataaagagagtgttatataaaaatccgacagctgaactaggttgctcttttttggcagggagtggggatgagatttgacaccaatatgggcaaaattagataaccttttggttaatataaatgattttgatttggaggcctaatttgtagattgtgaaagcagcttttagtttaacttattcacagaccccttataattaccatgtttttttttttcttcctaaatctcttggttcagcttgtgaatcttacgtgcccgtaaagttgggatgttgaattggctcttctttgttctggcagtgagtcaagtgtccagcattttttcataagtgttttttaaaattgttctccagcattttatggctcctccctcccatgtcctcagacccagcaaaagcgtagaggcagaattagaggcctctccaggccagctcctctgcccacatgtcatacaaggtgtgaatttgagcacagtccagaaatggagacatcccacccccagttgaataatggcccattcatgccaaccttgccaacacggagagggcagagatgcactagaagaccttcatcctccccttcctctgccccaagtcactacagttggttctattgaagccagtctttaagaaacctgggttaaagacaccagcacttctgcttgctgggctggctggacctgtgaagccatgggcaggtagtgccctcttgagagtcattttatttggccaccttcaggtgagactatccatagacacatgctaggataggccccgctgggagggcagttacaggagagagtaggtggtggtgacgtgagggctgtgaaggatccagagacaagacttagatgtttcgttcattcactcactcattcagttactcctaagacttttcagtttcataaggaagagtgttgcctgaggccctagggaatattggggaatagaagggattgaggaaacattaataatagttattcaaaagacccaaatgcttatacttctctctcccttcttctctctctgacacacacacacacacacacacacacacacacacacacgtgcacattcctcccttacatgctcatttgtgccttaaatgtgccttataggtaaatccaggatgactgaggaatccctcgtcactgggagattttgtatatattcttttattattagattgagttgggtgtggggaaaaatttttttctgaaggctcaaaagtggtttcctaaaagtgagccactatcagatttgcacatcaggagaaaagaaatagggttacgtccattaggaaaatcccagtttgcaggagtgcaatcacatcaaaaaaacaaccagccaggattaaaggtattataaatcctcatagcggaacatttctcagggcaaaggaacctggctcatttgaagattaatgttccatgcctttgtggtcaaagggtcagcacttaacacaggaaaaaactaggtgttgttttgttttgttattttggacaacataaaattcaggaatgttttatttagccttggtttctagaaggaagggaaataatatttcttgagcatttactagggtgttgcgtgctgtgctaagtaaattttaagtctttcagttttatagatacggaaaacaagggtgactctttaccacaggatgaataaagaactaagtaatatgggaaatgcagcaatttctggactagctgagccgattccttcctgtgagcacactgtaagctttcaagttctctgggcaggaattacagcacctgtcccctgcaatggccctgctgtgtgatgctcatcgcttcccttcgtgctggagcagtcccccaggtgtccatctcctatctttttgttccaatcttctgtgagttccagctagcaggctttacatctggggaaaggaaaaccaggggttttagctctgttctctgctcccatccttcgctcaccagctgagtgagaacatgaactttttgcaccatgtacccatggcttacactacttagaaaatcaccttttcagataaaacagtttatgagttcatagagaacaccagcactctttgacaaaactgtgagtgaccctttttaaacaatgctgagcaggccctgagctataatcaacggtgagctttaatgtctatgctgacagttaggttttgctctcttttgtaacaggttacgtagaccagcagtgtttaaatctaaatacgttgtgagtctgttatctgtcctatcgcgttttttaaatgactttttattctttatcatagctaagtaaataccaaaaaaaaaaaaaagctttgtaggacacttgtacttagtttgggaaaaaaaaataaattgaaattgttatgcttttgtatttccatttcttgcaaataaatattttttcttaaatagtaaaaaaaaaaaaaaaaa

SEQ ID NO: 163 - DNA, 924 bp, Homo sapiens
gaggtcagagacttaagtctaaggcactgagcgtatcatgttaaagatgagcgggtggcagcgacagagccaaaatcagagctggaacctgaggagagagtgttcaagaaggaagtgtatcttcatacatcaccacacctgaaagcagatgtgcttttccagactgatccaactgcagagatggcagctgagtcattgcctttctccttcgggacactgtccagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagaaccaggaaacggaaacagagtggtcattccccagcccgggctggaaagcagcgcatgaaggagaaagaacaggagaatgaaaggaaagtggcacagctagctgaagagaatgaacggctcaagcaggaaatcgagcgcctgaccagggaagtagaggcgactcgccgagctctgattgaccgaatggtgaatctgcaccaagcatgaacaattgggagcatcagtcccccacttgggccacactacccacctttcccagaagtggctactgactaccctctcactagtgccaatgatgtgaccctcaatcccacatacgcagggggaaggcttggagtagacaaaaggaaaggtctcagcttgtatatagagattgtacatttatttattactgtccctatctattaaagtgactttctatgagccaaaaaaaaaaaaaaa

SEQ ID NO: 164 - DNA, 5042 bp, Homo sapiens
gttttcacttggtcggaatggggagagtgtgcaagagatcgctgcgggacaggttcctagagatcgctccgggacggtcgtgacggcccccgagggacatgagagaagaggagcggcgctcaggttattccaggatctttggagacccgaggaaagccgtgttgaccaaaagcaagacaaatgactcacagagaaaaaagatggcagaaccaagggcaactaaagccgtcaggttctgaacagctggtagatgggctggcttactgaaggacatgattcagactgtcccggacccagcagctcatatcaaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagatttaccatatgagccccccaggagatcagcctggaccggtcacggccaccccacgccccagtcgaaagctgctcaaccatctccttccacagtgcccaaaactgaagaccagcgtcctcagttagatccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactctatcggagaacatgaatcaaaagtgcctcaagaggaatgaaaaaagctttactggggctggggaaggaagccggggaagagatccaaagactcttgggagggagttactgaagtcttactacagaaatgaggaggatgctaaaaatgtcacgaatatggacatatcatctgtggactgaccttgtaaaagacagtgtatgtagaagcatgaagtcttaaggacaaagtgccaaagaaagtggtcttaagaaatgtataaactttagagtagagtttggaatcccactaatgcaaactgggatgaaactaaagcaatagaaacaacacagttttgacctaacataccgtttataatgccattttaaggaaaactacctgtatttaaaaatagaaacatatcaaaaacaagagaaaagacacgagagagactgtggcccatcaacagacgttgatatgcaactgcatggcatgtgctgttttggttgaaatcaaatacattccgtttgatggacagctgtcagctttctcaaactgtgaagatgacccaaagtttccaactcctttacagtattaccgggactatgaactaaaaggtgggactgaggatgtgtatagagtgagcgtgtgattgtagacagaggggtgaagaaggaggaggaagaggcagagaaggaggagaccagggctgggaaagaaacttctcaagcaatgaagactggactcaggacatttggggactgtgtacaatgagttatggagactcgagggttcatgcagtcagtgttataccaaacccagtgttaggagaaaggacacagcgtaatggagaaaggggaagtagtagaattcagaaacaaaaatgcgcatctctttctttgtttgtcaaatgaaaattttaactggaattgtctgatatttaagagaaacattcaggacctcatcattatgtgggggctttgttctccacagggtcaggtaagagatggccttcttggctgccacaatcagaaatcacgcaggcattttgggtaggcggcctccagttttcctttgagtcgcgaacgctgtgcgtttgtcagaatgaagtatacaagtcaatgtttttccccctttttatataataattatataacttatgcatttatacactacgagttgatctcggccagccaaagacacacgacaaaagagacaatcgatataatgtggccttgaattttaactctgtatgcttaatgtttacaatatgaagttattagttcttagaatgcagaatgtatgtaataaaataagcttggcctagcatggcaaatcagatttatacaggagtctgcatttgcactttttttagtgactaaagttgcttaatgaaaacatgtgctgaatgttgtggattttgtgttataatttactttgtccaggaacttgtgcaagggagagccaaggaaataggatgtttggcacccaaatggcgtcagcctctccaggtccttcttgcctcccctcctgtcttttatttctagccccttttggaacagaaggaccccgggtttcacattggagcctccatatttatgcctggaatggaaagaggcctatgaagctggggttgtcattgagaaattctagttcagcacctggtcacaaatcacccttaattcctgctatgattaaaatacatttgttgaacagtgaacaagctaccactcgtaaggcaaactgtattattactggcaaataaagcgtcatggatagctgcaatttctcactttacagaaacaagggataacgtctagatttgctgcggggtttctctttcaggagctctcactaggtagacagctttagtcctgctacatcagagttacctgggcactgtggcttgggattcactagccctgagcctgatgttgctggctatcccttgaagacaatgtttatttccataatctagagtcagtttccctgggcatcttttctttgaatcacaaatgctgccaaccttggtccaggtgaaggcaactcaaaaggtgaaaatacaaggtgaccgtgcgaaggcgctagccgaaacatcttagctgaataggtttctgaactggcccttttcatagctgtttcagggcctgtttttttcacgttgcagtccttttgctatgattatgtgaagttgccaaacctctgtgctgtggatgttttggcagtgggctttgaagtcggcaggacacgattaccaatgctcctgacaccccgtgtcatttggattagacggagcccaaccatccatcattttgcagcagcctgggaaggcccacaaagtgcccgtatctccttagggaaaataaataaatacaatcatgaaagctggcagttaggctgacccaaactgtgctaatggaaaagatcagtcatttttattttggaatgcaaagtcaagacacacctacattcttcatagaaatacacatttacttggataatcactcagttctctcttcaagactgtctcatgagcaagatcataaaaacaagacatgattatcatattcaattttaacagatgttttccattagatccctcaaccctccacccccagtccaggttattagcaagtcttatgagcaactgggataattttggataacatgataatactgagttccttcaaatacataattcttaaattgtttcaaaatggcattaactctctgttactgttgtaatctaattccaaagccccctccaggtcatattcataattgcatgaaccttttctctctgtttgtccctgtctcttggcttgccctgatgtatactcagactcctgtacaatcttactcctgctggcaagagatttgtcttcttttcttgtcttcaattggctttcgggccttgtatgtggtaaaatcaccaaatcacagtcaagactgtgtttttgttcctagtttgatgcccttatgtcccggaggggttcacaaagtgctttgtcaggactgctgcagttagaaggctcactgcttctcctaagccttctgcacagatgtggcacctgcaacccaggagcaggagccggaggagctgccctctgacagcaggtgcagcagagatggctacagctcaggagctgggaaggtgatggggcacagggaaagcacagatgttctgcagcgccccaaagtgacccattgcctggagaaagagaagaaaatattttttaaaaagctagtttatttagcttctcattaattcattcaaataaagtcgtgaggtgactaattagagaataaaaattactttggactactcaaaaatacaccaaaaaaaa

SEQ ID NO: 165 - DNA, 2644 bp, Homo sapiens
attccaaacagcctagtctcgtgctgagagcctctccggtttcacgctgagacccgctcacccccgctctggccccttagatgctattttggcccgagtgtcacgtcgggcgctctttagagaggactgggacaagagttgcggacgcgaagaacgagtaagcggtggttcatccctcctgaccccacccccgtggcctggcccgatggtcgcgcccggggttgcgagatttgcgcctgcgcagtgcggcgcctagagggaaagcgagagggagacggacgttgagagaacgaggaggaaggagagaaaatggcgtccacggattacagtacctatagccaagctgcagcgcagcagggctacagtgcttacaccgcccagcccactcaaggatatgcacagaccacccaggcatatgggcaacaaagctatggaacctatggacagcccactgatgtcagctatacccaggctcagaccactgcaacctatgggcagaccgcctatgcaacttcttatggacagcctcccactggttatactactccaactgccccccaggcatacagccagcctgtccaggggtatggcactggtgcttatgataccaccactgctacagtcaccaccacccaggcctcctatgcagctcagtctgcatatggcactcagcctgcttatccagcctatgggcagcagccagcagccactgcacctacaagaccgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagagttcattccgacaggaccaccccagtagcatgggtgtttatgggcaggagtctggaggattttccggaccaggagagaaccggagcatgagtggccctgataaccggggcaggggaagagggggatttgatcgtggaggcatgagcagaggtgggcggggaggaggacgcggtggaatgggcagcgctggagagcgaggtggcttcaataagcctggtggacccatggatgaaggaccagatcttgatctaggcccacctgtagatccagatgaagactctgacaacagtgcaatttatgtacaaggattaaatgacagtgtgactctagatgatctggcagacttctttaagcagtgtggggttgttaagatgaacaagagaactgggcaacccatgatccacatctacctggacaaggaaacaggaaagcccaaaggcgatgccacagtgtcctatgaagacccacccactgccaaggctgccgtggaatggtttgatgggaaagattttcaagggagcaaacttaaagtctcccttgctcggaagaagcctccaatgaacagtatgcggggtggtctgccaccccgtgagggcagaggcatgccaccaccactccgtggaggtccaggaggcccaggaggtcctgggggacccatgggtcgcatgggaggccgtggaggagatagaggaggcttccctccaagaggaccccggggttcccgagggaacccctctggaggaggaaacgtccagcaccgagctggagactggcagtgtcccaatccgggttgtggaaaccagaacttcgcctggagaacagagtgcaaccagtgtaaggccccaaagcctgaaggcttcctcccgccaccctttccgcccccgggtggtgatcgtggcagaggtggccctggtggcatgcggggaggaagaggtggcctcatggatcgtggtggtcccggtggaatgttcagaggtggccgtggtggagacagaggtggcttccgtggtggccggggcatggaccgaggtggctttggtggaggaagacgaggtggccctggggggccccctggacctttgatggaacagatgggaggaagaagaggaggacgtggaggacctggaaaaatggataaaggcgagcaccgtcaggagcgcagagatcggccctactagatgcagagaccccgcagagctgcattgactaccagatttattttttaaaccagaaaatgttttaaatttataattccatatttataatgttggccacaacattatgattattccttgtctgtactttagtatttttcaccatttgtgaagaaacattaaaacaagttaaatggtagtgtgcggagtttttttttcttccttcttttaaaaatggttgtttaagactttaacaatgggaaccccttgtgagcatgctcagtatcattgtggagaaccaagagggcctcttaactgtaacaatgttcatggttgtgatgttttttttttttttttaaataaaattccaaatgtttataaagagtca

SEQ ID NO: 166 - DNA, 2957 bp, Homo sapiens
gaattcccaaacgtgcacaggggagtgagggcagggcgctcgcagggggcacgcagggagggcccagggcgccagggaggccgcgccgggctaatccgaaggggctgcgaggtcaggctgtaaccgggtcaatgtgtggaatattggggggctcggctgcagacttggccaaatggacgggactattaaggaggctctgtcggtggtgagcgacgaccagtccctctttgactcagcgtacggagcggcagcccatctccccaaggccgacatgactgcctcggggagtcctgactacgggcagccccacaagatcaaccccctcccaccacagcaggagtggatcaatcagccagtgagggtcaacgtcaagcgggagtatgaccacatgaatggatccagggagtctccggtggactgcagcgttagcaaatgcagcaagctggtgggcggaggcgagtccaaccccatgaactacaacagctatatggacgagaagaatggcccccctcctcccaacatgaccaccaacgagaggagagtcatcgtccccgcagaccccacactgtggacacaggagcatgtgaggcaatggctggagtgggccataaaggagtacagcttgatggagatcgacacatcctttttccagaacatggatggcaaggaactgtgtaaaatgaacaaggaggacttcctccgcgccaccaccctctacaacacggaagtgctgttgtcacacctcagttacctcagggaaagttcactgctggcctataatacaacctcccacaccgaccaatcctcacgattgagtgtcaaagaagacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactagaagcttcttctagctgaagcccatcctgcacacttactggatgctttggactcaacaggacatatgtggccttgaagggaagacaaaactggatgttctttcttgttggatagaacctttgtatttgttctttaaaaacattttttttaatgttggtaacttttgcttcctctacctgaacaaagagatgaataattccatgggccagtatgccagtttgaattctcagtctcctagcatcttgtgagttgcatattaagattactggaatggttaagtcatggttctgagaaagaagctgtacgttttctttatgtttttatgaccaaagcagtttcttgtcaatacacggggttcagtatgacacagaatcatggacttaacccgtcatgttctggtttgagatttagtgacaaatagaggtgggaagcttataatctaattttaggaggaccaaattcagcggatggcaactggaacattgattgtaaggccagtgaagttttcacccaactggaatttgatggaaagaaggtttgtgtgtttaagacgccaagggcattgcagaatccctctcagtggacagtatgcactcagctgaccactctctctagaaatagtcaagatatgaactaagaaattttaatgcaaatacatacattcctgaaagacggggaattaaattactaattttttttttttaaatgatgacagtggtcccagaacttggaaaagttgtagggatttctaaactcaagcagattcgcaagtgctgtgcgcttgtcagaccatcagaccagggccaaccaatcagaaggcaacttactgtataaattatgcagagttattttcctatatctcacagtattaaaaaataaataattaaaaattaagaataaataaacgagttgacctcggtcacaaaagcagttttactatcgaatcaatcgctgttatttttttttaatgtaatttgtacatcttttttcaatctgtacatttgggctgtcttgtatgtttttatgctcctttttaaaaagcataatatgcctatagctgaaaaggaaacagggctgtttaagtcactgacttatgagaaagcaaagcactggtacagttatttaacaggcatacacaagcagggaaaagataatccatttagatctttaatgctttggaaatgcgtgtaacagtactgcaataatcacagctctgggaaaaacaacgaaactttcccttgtggagaggagggattttcctgctctatataagcaacatatttttagacattaaaatatatataattttgcaggtaattgttgacttttttaactatattaagtgttaagctgacaactgtcaaagaagaccatgttgtaaaataatttgactaaataaatggttccttctctcaaaaaaaaaaaaa

SEQ ID NO: 167 - DNA, 3029 bp, Homo sapiens
ccaggcagctggggtaaggagttcaaggcagcgcccacacccgggggctctccgcaacccgaccgcctgtccgctcccccacttcccgccctccctcccacctactcattcacccacccacccacccagagccgggacggcagcccaggcgcccgggccccgccgtctcctcgccgcgatcctggacttcctcttgctgcaggacccggcttccacgtgtgtcccggagccggcgtctcagcacacgctccgctccgggcctgggtgcctacagcagccagagcagcagggagtccgggacccgggcggcatctgggccaagttaggcgccgccgaggccagcgctgaacgtctccagggccggaggagccgcggggcgtccgggtctgagccgcagcaaatgggctccgacgtgcgggacctgaacgcgctgctgcccgccgtcccctccctgggtggcggcggcggctgtgccctgcctgtgagcggcgcggcgcagtgggcgccggtgctggactttgcgcccccgggcgcttcggcttacgggtcgttgggcggccccgcgccgccaccggctccgccgccacccccgccgccgccgcctcactccttcatcaaacaggagccgagctggggcggcgcggagccgcacgaggagcagtgcctgagcgccttcactgtccacttttccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggagttgctgctgggagctccagctcagtgaaatggacagaagggcagagcaaccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttctcgttcagaccagctcaaaagacaccaaaggagacatacaggtgtgaaaccattccagtgtaaaacttgtcagcgaaagttctcccggtccgaccacctgaagacccacaccaggactcatacaggtaaaacaagtgaaaagcccttcagctgtcggtggccaagttgtcagaaaaagtttgcccggtcagatgaattagtccgccatcacaacatgcatcagagaaacatgaccaaactccagctggcgctttgaggggtctccctcggggaccgttcagtgtcccaggcagcacagtgtgtgaactgctttcaagtctgactctccactcctcctcactaaaaaggaaacttcagttgatcttcttcatccaacttccaagacaagataccggtgcttctggaaactaccaggtgtgcctggaagagttggtctctgccctgcctacttttagttgactcacaggccctggagaagcagctaacaatgtctggttagttaaaagcccattgccatttggtgtggattttctactgtaagaagagccatagctgatcatgtccccctgacccttcccttctttttttatgctcgttttcgctggggatggaattattgtaccattttctatcatggaatatttataggccagggcatgtgtatgtgtctgctaatgtaaactttgtcatggtttccatttactaacagcaacagcaagaaataaatcagagagcaaggcatcgggggtgaatcttgtctaacattcccgaggtcagccaggctgctaacctggaaagcaggatgtagttctgccaggcaacttttaaagctcatgcatttcaagcagctgaagaaaaaatcagaactaaccagtacctctgtatagaaatctaaaagaattttaccattcagttaattcaatgtgaacactggcacactgctcttaagaaactatgaagatctgagatttttttgtgtatgtttttgactcttttgagtggtaatcatatgtgtctttatagatgtacatacctccttgcacaaatggaggggaattcattttcatcactgggagtgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtcgtgtgtgtgtgtgtgtgtgtgtgtgtgtgtgtgttgtgttgtgttttgttttttaagggagggaatttattatttaccgttgcttgaaattactgtgtaaatatatgtctgataatgatttgctctttgacaactaaaattaggactgtataagtactagatgcatcactgggtgttgatcttacaagatattgatgataacacttaaaattgtaacctgcatttttcactttgctctcaattaaagtctattcaaaag

SEQ ID NO: 168 - DNA, 1901 bp, Homo sapiens
gcggcgagtggagcgggagccgactggaagaagggctctagggagggggctgtggctgctggggtccgaggtggggccgggtacaccagccccatcactgtttgcagagagtcagggaggcggaaaagacacgcgctctaggctcccatcagggcacatggcccgggcccatcccccgcgcgtctccccggctgcggggcgcggggggctgccgggtgcgcttggctgtggcgcggcgcgttggagactttattgcgatgggacgataagaggggcgggggcggggtcctgggggccgaggcggcagcgctttaattaaaacggaaattgcggccccgggccgcgcgggggccggagggttccaagcggccccttagctggaagcgtttctccaggacccccccgcaacccccgccacgcccgggctgccccctcccgccaggccctgccggacccggcgccgtcttctcctccttgtcacccgcggtcgcttcgggcggggatcggtgccaccgagcgcaaagcctgcctcgccccccttccccgtcccccccatctcccaccgcccagtccccggcggcgatgagacagagcggcgcctcccagcccctgctgatcaacatgtacctgccagatcccgtcggagacggtctcttcaaggacgggaagaacccgagctgggggccgctgagccccgcggttcagaaaggcagcggacagatccagctgtggcagtttctgctggagctgctggctgaccgcgcgaacgccggctgcatcgcgtgggagggcggtcacggcgagttcaagctcacggacccggacgaggtggcgcggcggtggggcgagcgcaagagcaagcccaacatgaactacgacaagctgagccgcgccctgcgctactactacgacaagaacatcatgagcaaggtgcatggcaagcgctacgcctaccgcttcgacttccagggcctggcgcaggcctgccagccgccgcccgcgcacgctcatgccgccgccgcagctgctgccgccgccgcggccgcccaggacggcgcgctctacaagctgcccgccggcctcgccccgctgcccttccccggcctctccaaactcaacctcatggccgcctcggccggggtcgcgcccgccggcttctcctactggccgggcccgggccccgccgccaccgctgccgccgccaccgccgcgctctaccccagtcccagcttgcagcccccgcccgggcccttcggggccgtggccgcagcctcgcacttggggggccattaccactagacggggcggtcgggtgcctgcggcctcgcccgcacgcctagagtctcgcccgatcccatcggcatcccggggagggcccgggagcctccgtcaaccgtcctctaatccagagtttactccacctgccgcacttagcagggggacgggaccgaagctccctcaatccttgtctggtactagatttgctcctgtcccaccccgcagtcccctgaggagggcgatgtgcgccctctttcactttttttcttctaggtctccaggtcccggaggggatttgtggacctctcttgtctccccaccactccagtgcatttccgcctggctcctagaagccccattcaatatcactactctttaacgagtgccaaatcttttcccacttttgctcttccccaaggaactgctcccacctcagcacgtggaggcctctcacggtcctccttcctgggacctgagcaggtttggtgaaagccaccgtcctccgtgacacacggcccccttcctcctgtccccacactcccaggagaaactcccggtgtgtttctgaccctttcagccccattaaagctcctgagctctcaaaaaaaaaaaaaaaaaaaaaa

SEQ ID NO: 169 - DNA, 5635 bp, Homo sapiens
ataaatgacgtgccgagagagcgagcgaacgcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctgcagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggca

SEQ ID NO: 170 - DNA, 2949 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 171 - DNA, 3212 bp, Homo sapiens
gagtaggcgcgagctaagcaggaggcggaggcggaggcggagggcgaggggcggggagcgccgcctggagcgcggcaggtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaaaaaaaaaa

(19) United States
(12) Patent Application Publication
(10) Pub. No.: US 2014/0272956 A1
(43) Pub. Date: Sep. 18, 2014

(54) METHOD FOR AMPLIFICATION AND ASSAY OF RNA FUSION GENE VARIANTS, METHOD OF DISTINGUISHING SAME AND RELATED PRIMERS, PROBES, AND KITS

(71) Applicant: ABBOTT MOLECULAR INC., Des Plaines, IL (US)

(72) Inventors: Shihai X. Huang, Lincolnshire, IL (US); Jeffrey D. Wuitschick, Wauwatosa, WI (US)

(73) Assignee: ABBOTT MOLECULAR INC., Des Plaines, IL (US)

(21) Appl. No.: 14/203,591

(22) Filed: Mar. 11, 2014

Related U.S. Application Data
(60) Provisional application No. 61/800,593, filed on Mar. 15, 2013.

(51) Int. Cl. C12Q 1/68

(52) U.S. Cl. CPC C12Q 1/6844
USPC 435/6.11

(57) ABSTRACT

Method for amplification, alone or in further combination with detection or detection and quantitation, of RNA from fusion gene variants, method of distinguishing same, and oligonucleotide primers and probes and kits for use in the methods.

### CROSS REFERENCE TO RELATED APPLICATIONS

This application claims priority to U.S. provisional patent application No. 61/800,593, which was filed on Mar. 15, 2013, and which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 7, 2014, is named 11438USO1_SEQ.txt and is 399,606 bytes in size.

### TECHNICAL FIELD

The present disclosure relates to fusion gene variants, a method of amplifying, alone or in further combination with detecting or detecting and quantitating, polymerase chain reaction (PCR), such as real-time PCR, a method of distinguishing fusion gene variants, oligonucleotide primers and probes, and kits.

### BACKGROUND

Certain types of human leukemia are commonly associated with the presence of a fusion gene, specifically a fusion of breakpoint cluster region (BCR) gene and Abelson murine leukemia (ABL) proto-oncogene, which results from a chromosomal translocation that fuses the 5′ portion of the BCR gene on chromosome 22 with the 3′ portion of the ABL gene on chromosome 9. Due to heterogeneity in the translocation breakpoints within the BCR gene, patient populations have been observed to contain different BCR-ABL fusion gene variants including e1a2, b2a2, b3a2, and e19a2, which are produced by translocations that juxtapose BCR exons e1, b2, b3, or e19, respectively, with ABL exon a2.

The current recommended method for monitoring disease status during treatment is periodic assessment of the level of BCR-ABL mRNA by comparison of the level of BCR-ABL transcripts with the level of mRNA of an endogenous housekeeping gene. Jones et al., for example, describes monitoring p210 and p190 bcr-abl RNA fusion transcripts (p210 and p190 are designations used to refer to protein variants) as a method of monitoring disease load in patients with chronic myelogenous leukemia (Jones et al., Am J. Clin. Pathol. 120: 42-48 (2003)). Lee et al., for example, describes screening newly diagnosed patients with acute lymphoblastic leukemia for p210 and p190 bcr-abl RNA fusion transcripts and monitoring the course of disease during therapy (Lee et al., Genome Res. 4: 283-287 (1995)). Detection and quantitation of BCR-ABL mRNA may also have diagnostic and/or prognostic utility.

Methods currently available in the art lack the ability to detect and differentiate RNA from BCR-ABL variants, such as e1a2, b2a2, b3a2, and e19a2. In some cases, the methods are limited to detecting either a single BCR-ABL variant (e.g., e1a2) or only a subset of variants (e.g., b2a2 and b3a2, but not e1a2 or e19a2) in a single reaction. Those methods that detect more than one variant, such as b2a2 and b3a2, are not able to distinguish between the detected variants without the use of reflex testing, such as capillary gel electrophoresis. Reflex testing can disrupt routine laboratory workflow and cause contamination.

A variety of cancer tumors, including non-small cell lung cancer, breast cancer, and colorectal cancer, are associated with a fusion of the Echinoderm Microtubule-Associated Protein-like 4 (EML4) gene and the Anaplastic Lymphoma Kinase (ALK) gene, which results from the an inversion on chromosome 2 that fuses a 5′ portion of the EML4 gene with a 3′ portion of the ALK gene. Due to heterogeneity in the inversion sites, patient populations have been observed to contain different EML4-ALK fusion variants including E13A20, E6aA20, E6bA20, E14A20, and E20A20, which are produced by inversions that juxtapose EML4 exon 13 (E13), exon 6a (E6a), exon 6b (E6b), exon 14 (E14) or exon 20 (E20), respectively, with ALK exon 20 (A20).

Identification of tumors expressing the EML4-ALK fusion gene is medically relevant due to their potential responsiveness to ALK tyrosine kinase inhibitor therapy and due to their general non-responsiveness to EGFR antagonist therapies. Like BCR-ABL, methods currently available in the art lack the ability to detect and differentiate RNA from EML4-ALK variants.

A portion of lung cancer tumors are associated with a fusion of the Kinesin Family Member 5B (KIF5B) gene and the Ret Proto-oncogene (RET), which results from an inversion on chromosome 10 that fuses a 5′ portion of the KIF5B gene with a 3′ portion of the RET gene. Due to heterogeneity in the inversion sites, patient populations have been observed to contain different KIF5B-RET fusion variants including K15R12, K16R12, K22R12, and K23R12, which are produced by inversions that juxtapose KIF5B exon 15 (K15), exon 16 (K16), exon 22 (K22), or exon 23 (K23), respectively, with RET exon 12 (R12).

Identification of tumors expressing the KIF5B-RET fusion gene is medically relevant due to their potential responsiveness to tyrosine kinase inhibitor therapy. Like BCR-ABL, methods currently available in the art lack the ability to detect and differentiate RNA from KIF5B-RET variants.

Acute promyelocytic leukemia (APL) patients frequently harbor a fusion of the Promyelocytic Leukemia gene (PML) with the Retinoic Acid Receptor Alpha gene (RARα), which results from a chromosomal translocation that fuses a 5′ portion of the PML gene on chromosome 15 with a 3′ portion of the RARα gene on chromosome 17. Due to heterogeneity in the translocation breakpoints, patient populations have been observed to contain different PML-RARα fusion gene variants termed PML-RARα S form, PML-RARα V form, and PML-RARα L form, which are produced by translocations that juxtapose PML exon 3 (P3), the 5′ region of PML exon 6 (P6a), or the 3′ region of PML exon 6 (P6b), respectively, with RARα exon 3/4 (R3/4; the exon designation R3 will be used herein but is intended to encompass the alternative designation R4).

Identification of leukemias expressing the PML-RARα fusion gene is medically relevant due to their high rate of response to all-trans-retinoic acid (ATRA) therapy. Detection and differentiation of PML-RARα fusion gene variants (forms S, V or L) may also be of prognostic value since the distinct translocation forms may carry different prognoses. For example, PML-RARα form S may be associated with a worse prognosis than PML-RARα forms V and L.

In view of the foregoing, the present disclosure seeks to provide a method for amplifying, detecting and/or quantitating mRNA from gene fusion variants, such as BCR-ABL, EML4-ALK, KIF5B-RET, and PML-RARα gene fusion variants, a method of distinguishing same, as well as materials and kits for use in such methods. These and other objects and advantages, as well as inventive features, will become apparent from the detailed description provided herein.

### SUMMARY

A method of amplifying mRNA from variants of a fusion between a first gene and a second gene in a sample of mRNA is provided. The method comprises (a) (i) obtaining cDNA, which has been reverse-transcribed from the sample of mRNA, or reverse-transcribing the sample of mRNA to cDNA and (ii) amplifying the reverse-transcribed cDNA using primers comprising a primer for an exon of the first gene, which becomes contiguous with a variant exon of the second gene, and a primer for each of two or more variant exons of the second gene. One or more primers can be detectably labeled. The method can further comprise (b) detecting the amplified cDNA or detecting and quantitating the amplified cDNA. Detecting the amplified cDNA can comprise contacting the amplified cDNA with at least one probe under hybridizing conditions and detecting hybridization of the at least one probe to the amplified cDNA. The first gene and the second gene can be the breakpoint cluster region (BCR) gene and the Abelson murine leukemia (ABL) proto-oncogene, the echinoderm microtubule-associated protein-like 4 (EML4) gene and the anaplastic lymphoma kinase (ALK) gene, the kinesin family member 5B (KIF5B) gene and the Ret (RET) proto-oncogene, or the promyelocytic leukemia (PML) gene and the retinoic acid receptor alpha (RARα) gene. The at least one probe can be a probe that hybridizes to a nucleotide sequence at or near a junction of a variant of a fusion between the first gene and the second gene comprising the 3′ end of an exon of the first gene and the 5′ end of an exon of the second gene.

In an embodiment of the method of amplifying mRNA from variants of a fusion between a first gene and a second gene in a sample of mRNA, the primers can comprise primers for a group of BCR-ABL gene fusion variants comprising at least two gene fusion variants selected from the group consisting of an e1a2 gene fusion, a b2a2 gene fusion, a b3a2 gene fusion, and an e19a2 gene fusion. The primers can comprise (i) a primer that hybridizes to exon a2 of ABL and (ii) two or more of a primer that hybridizes to exon e1 of BCR, a primer that hybridizes to exon b2 of BCR, and a primer that hybridizes to exon e19 of BCR. The primer that hybridizes to exon b2 of BCR can amplify reverse-transcribed cDNA from a BCR-ABL gene fusion variant comprising a b2a2 gene fusion and reverse-transcribed cDNA from a BCR-ABL gene fusion variant comprising a b3a2 gene fusion. The at least one probe can be a probe that hybridizes to a nucleotide sequence at or near a junction of a BCR-ABL gene fusion comprising the 3′ end of exon e1 of BCR and the 5′ end of exon a2 of ABL, a nucleotide sequence at or near a junction of a BCR-ABL gene fusion comprising the 3′ end of exon b2 of BCR and the 5′ end of exon a2 of ABL, a nucleotide sequence at or near a junction of a BCR-ABL gene fusion comprising the 3′ end of exon b3 of BCR and the 5′ end of exon a2 of ABL, or a nucleotide sequence at or near a junction of a BCR-ABL gene fusion comprising the 3′ end of exon e19 of BCR and the 5′ end of exon a2 of ABL. The primer that hybridizes to exon a2 of ABL can comprise a nucleotide sequence selected from the group consisting of SEQ ID NOs: 4, 25, 26, and 27. The primer that hybridizes to exon e1 of BCR can comprise a nucleotide sequence selected from the group consisting of SEQ ID NOs: 1, 18, 19, 20, 21, and 22, the primer that hybridizes to exon b2 of BCR can comprise a nucleotide sequence selected from the group consisting of SEQ ID NOs: 2 and 23, and/or the primer that hybridizes to exon e19 of BCR can comprise a nucleotide sequence selected from the group consisting of SEQ ID NOs: 3 and 24. The probe that hybridizes to a nucleotide sequence at or near a junction of a BCR-ABL gene fusion comprising the 3′ end of exon e1 of BCR and the 5′ end of exon a2 of ABL can comprise a nucleotide sequence selected from the group consisting of SEQ ID NOs: 5, 28, 29, 30, and a sequence complementary thereto, the probe that hybridizes to a nucleotide sequence at or near a junction of a BCR-ABL gene fusion comprising the 3′ end of exon b2 of BCR and the 5′ end of exon a2 of ABL can comprise a nucleotide sequence selected from the group consisting of SEQ ID NOs: 6, 31, 32, and a sequence complementary thereto, the probe that hybridizes to a nucleotide sequence at or near a junction of a BCR-ABL gene fusion comprising the 3′ end of exon b3 of BCR and the 5′ end of exon a2 of ABL can comprise a nucleotide sequence selected from the group consisting of SEQ ID NOs: 7, 33, 34, 35, and a sequence complementary thereto, and/or the probe that hybridizes to a nucleotide sequence at or near a junction of a BCR-ABL gene fusion comprising the 3′ end of exon e19 of BCR and the 5′ end of exon a2 of ABL can comprise a nucleotide sequence selected from the group consisting of SEQ ID NOs: 8, 36, and a sequence complementary thereto.

In another embodiment of the method of amplifying mRNA from variants of a fusion between a first gene and a second gene in a sample of mRNA, the primers can comprise primers for a group of EML4-ALK gene fusion variants comprising at least two gene fusion variants selected from the group consisting of an E13A20 gene fusion, an E6aA20 gene fusion, an E6bA20 gene fusion, an E14A20 gene fusion, and an E20A20 gene fusion. The primers can comprise (i) a primer that hybridizes to exon A20 of ALK and (ii) two or more of a primer that hybridizes to exon E13 of EML4, a primer that hybridizes to exon E6a of EML4, and a primer that hybridizes to exon E20 of EML4. The primer that hybridizes to exon E6a of EML4 can amplify reverse-transcribed cDNA from an EML4-ALK gene fusion variant comprising an E6a gene fusion and reverse-transcribed cDNA from an EML4-ALK gene fusion variant comprising an E6b gene fusion and/or the primer that hybridizes to exon E13 of EML4 can amplify reverse-transcribed cDNA from an EML4-ALK gene fusion variant comprising an E13 gene fusion and reverse-transcribed cDNA from an EML4-ALK gene fusion variant comprising an E14 gene fusion. The at least one probe can be a probe that hybridizes to a nucleotide sequence at or near a junction of an EML4-ALK gene fusion comprising the 3′ end of exon E13 of EML4 and the 5′ end of exon A20 of ALK, a nucleotide sequence at or near a junction of an EML4-ALK gene fusion comprising the 3′ end of exon E6a of EML4 and the 5′ end of exon A20 of ALK, a nucleotide sequence at or near a junction of an EML4-ALK gene fusion comprising the 3′ end of exon E6b of EML4 and the 5′ end of exon A20 of ALK, a nucleotide sequence at or near a junction of a EML4-ALK gene fusion comprising the 3′ end of exon E14 of EML4 and the 5′ end of exon A20 of ALK, or a nucleotide sequence at or near a junction of an EML4-ALK gene fusion comprising the 3′ end of exon E20 of EML4 and the 5′ end of exon A20 of ALK. In the method (a)(ii) can further comprise amplifying the reverse-transcribed cDNA using primers for at least one further EML4-ALK gene fusion variant selected from the group consisting of an E18A20 gene fusion, an E15A20 gene fusion, an E2A20 gene fusion, and an E17A20 gene fusion.

In yet another embodiment of the method of amplifying mRNA from variants of a fusion between a first gene and a second gene in a sample of mRNA, the primers comprise primers for a group of KIF5B-RET gene fusion variants comprising at least two gene fusion variants selected from the group consisting of a K15R12 gene fusion, a K16R12 gene fusion, a K22R12 gene fusion, and a K23R12 gene fusion. The primers can comprise (i) a primer that hybridizes to exon R12 of RET and (ii) a primer that hybridizes to exon K15 of KIF5B and/or a primer that hybridizes to exon K22 of KIF5B. The primer that hybridizes to exon K15 of KIF5B can amplify reverse-transcribed cDNA from a KIF5B-RET gene fusion variant comprising a K15R12 gene fusion and reverse-transcribed cDNA from a KIF5B-RET gene fusion variant comprising a K16R12 gene fusion and/or the primer that hybridizes to exon K22 of KIF5B can amplify reverse-transcribed cDNA from a KIF5B-RET gene fusion variant comprising a K22R12 gene fusion and reverse-transcribed cDNA from a KIF5B-RET gene fusion variant comprising a K23R12 gene fusion. The at least one probe can be a probe that hybridizes to a nucleotide sequence at or near a junction of a KIF5B-RET gene fusion comprising the 3′ end of exon K15 of KIF5B and the 5′ end of exon R12 of RET, a nucleotide sequence at or near a junction of a KIF5B-RET gene fusion comprising the 3′ end of exon K16 of KIF5B and the 5′ end of exon R12 of RET, a nucleotide sequence at or near a junction of a KIF5B-RET gene fusion comprising the 3′ end of exon K22 of KIF5B and the 5′ end of exon R12 of RET, or a nucleotide sequence at or near a junction of a KIF5B-RET gene fusion comprising the 3′ end of exon K23 of KIF5B and the 5′ end of exon R12 of RET.

In still yet another embodiment of the method of amplifying mRNA from variants of a fusion between a first gene and a second gene in a sample of mRNA, the primers can comprise primers for a group of PML-RARα gene fusion variants comprising at least two gene fusion variants selected from the group consisting of a P3R3 gene fusion, a P6aR3 gene fusion, and a P6bR3 gene fusion. The primers can comprise (i) a primer that hybridizes to exon R3 of RARα and (ii) a primer that hybridizes to exon P3 of PML and/or a primer that hybridizes to exon 6a of PML. The primer that hybridizes to exon 6a of PML can amplify reverse-transcribed cDNA from a PML-RARα gene fusion variant comprising a P6aR3 gene fusion and reverse-transcribed cDNA from a PML-RARα gene fusion variant comprising a P6bR3 gene fusion. The at least one probe can be a probe that hybridizes to a nucleotide sequence at or near a junction of a PML-RARα gene fusion comprising the 3′ end of exon P3 of PML and the 5′ end of exon R3 of RARα, a nucleotide sequence at or near a junction of a PML-RARα gene fusion comprising the 3′ end of exon P6a of PML and the 5′ end of exon R3 of RARα, or a nucleotide sequence at or near a junction of a PML-RARα gene fusion comprising the 3′ end of exon P6b of PML and the 5′ end of exon R3 of RARα.

Further provided is a set of primers. The set of primers comprises at least two primers selected from the group consisting of a primer that hybridizes to exon e1 of BCR, a primer that hybridizes to exon b2 of BCR, and a primer that hybridizes to exon e19 of BCR, wherein the primer can be detectably labeled and/or wherein the set of primers is combined with at least one detectably labeled probe selected from the group consisting of a probe that hybridizes to a nucleotide sequence at or near a junction of a BCR-ABL gene fusion comprising the 3′ end of exon e1 of BCR and the 5′ end of exon a2 of ABL, a probe that hybridizes to a nucleotide sequence at or near a junction of a BCR-ABL gene fusion comprising the 3′ end of exon b2 of BCR and the 5′ end of exon a2 of ABL, a probe that hybridizes to a nucleotide sequence at or near a junction of a BCR-ABL gene fusion comprising the 3′ end of exon b3 of BCR and the 5′ end of exon a2 of ABL, and a probe that hybridizes to a nucleotide sequence at or near a junction of a BCR-ABL gene fusion comprising the 3′ end of exon e19 of BCR and the 5′ end of exon a2 of ABL. The primer that hybridizes to exon e1 of BCR can comprise a nucleotide sequence selected from the group consisting of SEQ ID NOs: 1, 18, 19, 20, 21, and 22, the primer that hybridizes to exon b2 of BCR comprises a nucleotide sequence selected from the group consisting of SEQ ID NOs: 2 and 23, and/or the primer that hybridizes to exon e19 of BCR comprises a nucleotide sequence selected from the group consisting of SEQ ID NOs: 3 and 24. The set of primers can further comprise a primer that hybridizes to exon a2 of ABL. The primer that hybridizes to exon a2 of ABL can comprise a nucleotide sequence selected from the group consisting of SEQ ID NOs: 4, 25, 26, and 27.

Still further provided is a set of probes. The set of probes comprises at least two probes selected from the group consisting of a probe that hybridizes to a nucleotide sequence at or near a junction of a BCR-ABL gene fusion comprising the 3′ end of exon e1 of BCR and the 5′ end of exon a2 of ABL, a probe that hybridizes to a nucleotide sequence at or near a junction of a BCR-ABL gene fusion comprising the 3′ end of exon b2 of BCR and the 5′ end of exon a2 of ABL, a probe that hybridizes to a nucleotide sequence at or near a junction of a BCR-ABL gene fusion comprising the 3′ end of exon b3 of BCR and the 5′ end of exon a2 of ABL, and a probe that hybridizes to a nucleotide sequence at or near a junction of a BCR-ABL gene fusion comprising the 3′ end of exon e19 of BCR and the 5′ end of exon a2 of ABL. The probe that hybridizes to a nucleotide sequence at or near a junction of a BCR-ABL gene fusion comprising the 3′ end of exon e1 of BCR and the 5′ end of exon a2 of ABL can comprise a nucleotide sequence selected from the group consisting of SEQ ID NOs: 5, 28, 29, 30, and a sequence complementary thereto, the probe that hybridizes to a nucleotide sequence at or near a junction of a BCR-ABL gene fusion comprising the 3′ end of exon b2 of BCR and the 5′ end of exon a2 of ABL can comprise a nucleotide sequence selected from the group consisting of SEQ ID NOs: 6, 31, 32, and a sequence complementary thereto, the probe that hybridizes to a nucleotide sequence at or near a junction of a BCR-ABL gene fusion comprising the 3′ end of exon b3 of BCR and the 5′ end of exon a2 of ABL can comprise a nucleotide sequence selected from the group consisting of SEQ ID NOs: 7, 33, 34, 35, and a sequence complementary thereto, and/or the probe that hybridizes to a nucleotide sequence at or near a junction of a BCR-ABL gene fusion comprising the 3′ end of exon e19 of BCR and the 5′ end of exon a2 of ABL can comprise a nucleotide sequence selected from the group consisting of SEQ ID NOs: 8, 36, and a sequence complementary thereto.

Even still further provided is a kit. The kit comprises (i) a set of primers comprising a primer that hybridizes to an exon of a first gene, which becomes contiguous with a variant exon of a second gene, and a primer for each of two or more variant exons of the second gene; and (ii) instructions for a method of detecting mRNA from fusions of the first gene and the second gene in a sample of mRNA. The method comprises (a) (i′) obtaining cDNA, which has been reverse-transcribed from the sample of mRNA, or reverse-transcribing the sample of mRNA to cDNA and (ii′) amplifying the reverse-transcribed cDNA using primers comprising a primer for an exon of the first gene, which becomes contiguous with a variant exon of the second gene, and a primer for each of two or more variant exons of the second gene, wherein one or more of the primers can be detectably labeled, and (b) detecting the amplified cDNA or detecting and quantitating the amplified cDNA. The kit can further comprise a probe for each gene fusion variant that is not detected using a detectably labeled primer, wherein the probe hybridizes to a nucleotide sequence at or near a junction of a variant of a fusion between the first gene and the second gene comprising the 3′ end of an exon of the first gene and the 5′ end of an exon of the second gene. The first gene and the second gene can be the BCR gene and the ABL proto-oncogene, the EML4 gene and the ALK gene, the KIF5B gene and the RET proto-oncogene, or the PML gene and the RARα gene.

In one embodiment, the set of primers comprises (a) at least two primers selected from the group consisting of a primer that hybridizes to exon e1 of BCR, a primer that hybridizes to exon b2 of BCR, and a primer that hybridizes to exon e19 of BCR or (b) a primer comprising a nucleotide sequence selected from the group consisting of SEQ ID NOs: 1, 2, 3, 18, 19, 20, 21, 22, 23, and 24, and the instructions are for a method of detecting mRNA from fusions of the BCR gene and the ABL proto-oncogene in a sample of mRNA from a human. The method comprises (a) (i) obtaining cDNA, which has been reverse-transcribed from the sample of mRNA, or reverse-transcribing the sample of mRNA to cDNA and (ii) amplifying the reverse-transcribed cDNA using primers for a group of BCR-ABL gene fusion variants comprising at least two gene fusion variants selected from the group consisting of an e1a2 gene fusion, a b2a2 gene fusion, a b3a2 gene fusion, and an e19a2 gene fusion, wherein one or more of the primers can be detectably labeled, and (b) detecting the amplified cDNA or detecting and quantitating the amplified cDNA. The kit can comprise a primer that hybridizes to exon a2 of ABL. The kit can further comprise a probe for each gene fusion variant that is not detected using a detectably labeled primer, wherein (a) the probe hybridizes to a nucleotide sequence at or near a junction of a BCR-ABL gene fusion comprising the 3′ end of exon e1 of BCR and the 5′ end of exon a2 of ABL, a nucleotide sequence at or near a junction of a BCR-ABL gene fusion comprising the 3′ end of exon b2 of BCR and the 5′ end of exon a2 of ABL, a nucleotide sequence at or near a junction of a BCR-ABL gene fusion comprising the 3′ end of exon b3 of BCR and the 5′ end of exon a2 of ABL, or a nucleotide sequence at or near a junction of a BCR-ABL gene fusion comprising the 3′ end of exon e19 of BCR and the 5′ end of exon a2 of ABL or (b) the probe comprises a nucleotide sequence selected from the group consisting of SEQ ID NOs: 5, 6, 7, 8, 28, 29, 30, 31, 32, 33, 34, 35, 36, and a sequence complementary thereto. The kit can further comprise a primer comprising a nucleotide sequence selected from the group consisting of SEQ ID NOs: 4, 25, 26, and 27.

### DETAILED DESCRIPTION

The present disclosure is predicated, at least in part, on oligonucleotide primers and detectable oligonucleotide probes for the real-time amplification, detection and/or quantitation of mRNA of variants of a gene fusion, such as BCR-ABL gene fusion variants, EML4-ALK gene fusion variants, KIF5B-RET gene fusion variants, and PML-RARα gene fusion variants. With regard to BCR-ABL, the primers direct reverse transcription and amplification of mRNA from at least two BCR-ABL gene fusion variants, such as two, three, or four gene fusion variants, including, but not limited to, e1a2, b2a2, b3a2, and e19a2. The probes detect multiple (such as two or more, e.g., two, three, or four) BCR-ABL fusion gene mRNAs in a variant-specific manner. Oligonucleotide primers and detectable oligonucleotide probes for endogenous housekeeping genes, such as, but not limited to, β-glucuronidase (GUSB), ABL, and glucose-6-phosphate dehydrogenase (G6PD), enable comparison of the levels of BCR-ABL fusion gene mRNAs with the levels of mRNAs of housekeeping genes. Use of the BCR-ABL primers and probes enables highly specific and sensitive differentiation and quantitation of multiple BCR-ABL fusion gene mRNAs in a single real-time polymerase chain reaction (RT-PCR). Use of the housekeeping primers and probes provides a control for cell adequacy, sample extraction and amplification efficiency, and standardization of quantitation of BCR-ABL mRNA. The present disclosure enables the detection, or detection and quantitation, of two or more BCR-ABL variants, such as three variants, four variants, or more variants, in a single reaction while differentiating between such variants without reliance on reflux testing. This is achieved by combining the use of multiplexed PCR to amplify sequences transcribed from two or more BCR-ABL variants, such as three, four, or more variants, with the use of variant-specific hybridization probes to detect sequences that are unique to the exon translocation junction of each targeted BCR-ABL variant. BCR-ABL variants are detected/quantified with sensitivity, specificity, and dynamic ranges that are equivalent to, or better than, those realized with existing methods.

### Terms

The following terms are relevant to the present disclosure:

(a) The Abelson murine leukemia (ABL) proto-oncogene is located on chromosome 1. The Entrez Gene, Ensembl, and HGNC cytogenetic bands are 1q25.2. Aliases include v-abl Abelson murine leukemia viral oncogene homolog 2, ABLL, ARG, Abelson-related gene protein, tyrosine-protein kinase ARG, EC 2.7.10.2, v-abl Abelson murine leukemia viral oncogene homolog 2, Abelson tyrosine-protein kinase, Abelson murine leukemia viral oncogene homolog 2, and EC 2.7.10. Reference DNA sequences include, but are not limited to, NC_{—}000001.10 and NT_{—}004487.19. The sequence of H. sapiens ABL mRNA containing alternative first exons is available from NCBI as Accession Nos. M14754.1 [SEQ ID NO:37] and M14753.1 [SEQ ID NO:38]. The complete CDS sequence is available from NCBI as Accession No. U07563.1.

(b) “About” refers to approximately a +/−10% variation from the stated value. It is to be understood that such a variation is always included in any given value provided herein, whether or not specific reference is made to it.

(c) The anaplastic lymphoma kinase (ALK) gene is located on chromosome 2. The Entrez Gene and HGNC cytogenetic bands are 2p23, whereas the Ensembl cytogenetic band is 2p23.1. Aliases include anaplastic lymphoma receptor tyrosine kinase, CD246, CD246 antigen, EC 2.7.10.1, NBLST3, Ki-1, ALK tyrosine kinase receptor, and mutant anaplastic lymphoma kinase. Reference DNA sequences include, but are not limited to, NC_{—}000002.11 and NT_{—}022184.15. The mRNA sequence is available from NCBI as Accession No. NM_{—}004304.4 [SEQ ID NO:39; exons: 1-1619, 1620-1739, 1740-1904, 1905-2106, 2107-2234, 2235-2366, 2367-2498, 2499-2599, 2600-2769, 2770-2864, 2865-2993, 2994-3156, 3157-3307, 3308-3439, 3440-3584, 3585-3767, 3768-3866, 3867-4019, 4020-4124, 4125-4311, 4312-4402, 4403-4467, 4468-4597, 4598-4695, 4696-4788, 4789-4890, 4891-5025, 5026-5116, and 5117-6265].

(d) The breakpoint cluster region (BCR) gene is located on chromosome 22. The Entrez Gene and Ensembl cytogenetic bands are 22q11.23, whereas the HGNC cytogenetic band is 22q11. Aliases include BCR1, D22S11, ALL, CML, PHL, D22S662, renal carcinoma antigen NY-REN-26, EC 2.7.11.1, BCR/FGFR1 chimera protein, breakpoint cluster region protein, and FGFR1/BCR chimera protein. Reference DNA sequences include, but are not limited to, NC_{—}000022.10, NT_{—}011520.12, AH001427.1 (BCR DNA exon b3; [SEQ ID NO:40]; exon 1-75), M25948.1 (BCR DNA exon a1; [SEQ ID NO:41]; exon 16-189), M25946.1 (BCR DNA partial CDS; [SEQ ID NO:42]), and M25947.1 (exon b3 BCR DNA; [SEQ ID NO:43]; exon 1-75). The sequence of H. sapiens BCR transcript variant 1 is available from NCBI (National Center for Biotechnology Information; www.ncbi.nlm.nih.gov) as Accession No. NM_{—}004327.3 [SEQ ID NO:44; exons: 1-1875, 1876-2057, 2058-2162, 2163-2348, 2349-2456, 2457-2517, 2518-2570, 2571-2711, 2712-2833, 2834-3002, 3003-3122, 3123-3198, 3199-3303, 3304-3378, 3379-3476, 3477-3608, 3609-3668, 3669-3778, 3779-3918, 3919-4053, 4054-4159, 4160-4322, and 4323-6927; 1872-1877 breakpoint for translocation to form BCR-ABL], whereas the sequence of H. sapiens BCR transcript variant 2 is available from NCBI as Accession No. NM_{—}021574.2 [SEQ ID NO:45; exons: 1-1875, 1876-2057, 2058-2162, 2163-2348, 2349-2456, 2457-2517, 2518-2570, 2571-2711, 2712-2833, 2834-3002, 3003-3122, 3123-3198, 3199-3303, 3304-3378, 3379-3476, 3477-3536, 3537-3646, 3647-3786, 3787-3921, 3922-4027, 4028-4190, and 4191-6795]. The complete coding domain sequence (CDS) for H. sapiens BCR is available from NCBI as Accession No. U07000.1. Other sequences, which are relevant to BCR-ABL gene fusions, include NCBI Accession Nos. M17542.1 (exons 1 and 2 BCR-ABL mRNA; [SEQ ID NO:46]; exons: 1-31 and 32-63), M17541.1 (exons 1 and 2 BCR-ABL mRNA; [SEQ ID NO:47]; exons: 1-31 and 32-63), M19730.1 (BCR-ABL mRNA encoding P-185-ALL-ABL; [SEQ ID NO:48]), AY789120.1 (BCR-ABL fusion mRNA; [SEQ ID NO:49]), EU394717.1 (BCR-ABL e8a2 fusion mRNA, exons 7, 8 and a2; [SEQ ID NO:50]; exons: 1-52 (BCR exon 7), 53-166 (BCR exon 8), and 183-238 (ABL exon a2); intron: 167-182 (ABL intron 1a)), EU394718.1 (BCR-ABL e14a2 fusion mRNA, exons 12-14, a2, a3, a2; [SEQ ID NO:51]; exons: 1-64 (BCR exon 12), 65-169 (BCR exon 13), 170-243 (BCR exon 14), 244-254 (ABL exon a2 complement), 255-361 (ABL exon a2), and 362-486 (ABL exon a3)), EU394716.1 (BCR-ABL e18-intlb-a2 fusion mRNA, exon 2; [SEQ ID NO:52]; exons: 1-87 (BCR e18) and 128-163 (ABL a2); intron: 88-127 (ABL intron 1b)), EU236680.1 (BCR-ABL b3a3 fusion mRNA; [SEQ ID NO:53]; gene 1-250 (BCR) and gene 251-297 (ABL)), X06418.1 (BCR-ABL mRNA; [SEQ ID NO:54]; 1322-1323 bcr-abl recombination site), M13096.1 (BCR-ABL fusion mRNA, exons 2-5; [SEQ ID NO:55]), EU216062.1 (BCR-ABL fusion protein isoform X5 mRNA; [SEQ ID NO:56]), EU216060.1 (BCR-ABL fusion protein isoform X3 mRNA; [SEQ ID NO:57]), EU216058.1 (BCR-ABL fusion protein isoform X1; [SEQ ID NO:58]), AM886138.1 (t(9;22)(q34;q11) translocation breakpoint for BCR-ABL fusion protein DNA; [SEQ ID NO:59]; exons: 1-31 (BCR e13) and 791-853 (ABL1 a3); introns: 32-280 (BCR intron 13) and 281-790 (ABL1 intron 2)), DQ912590.1 (BCR-ABL fusion protein e14a5 mRNA; [SEQ ID NO:60]; 1-185 (BCR including exons 12-14) and 186-253 (ABL including exon 5)), DQ912588.1 (BCR-ABL fusion protein e1a5; [SEQ ID NO:61]; 1-166 (BCR including exon 1) and 167-234 (ABL including exon 5)), DQ898315.1 (BCR-ABL fusion protein e14a4 mRNA; [SEQ ID NO:62]; exons: 1-70 (BCR exon 13), 71-145 (BCR exon 14), and 146-216 (ABL exon 4); 145-146 BCR-ABL breakpoint), DQ898313.1 (BCR-ABL fusion protein e1a4 mRNA; [SEQ ID NO:63]; exons: 1-166 (BCR exon 1) and 167-237 (ABL exon 4); 166-167 BCR-ABL breakpoint), DQ912589.1 (BCR-ABL fusion protein e13a5 mRNA; [SEQ ID NO:64]; 1-110 (BCR including exons 12 and 13) and 111-178 (ABL including exon 5)), DQ898314.1 (BCR-ABL fusion protein e13a14 mRNA; [SEQ ID NO:65]; exons: 1-70 (BCR exon 13) and 71-141 (ABL exon 4); 70-71 BCR-ABL breakpoint), EF158045.1 (BCR-ABL p210 fusion protein mRNA; [SEQ ID NO:66]), EU154998.1 (BCR-ABL fusion protein e8a2 mRNA; [SEQ ID NO:67]; 194-221 breakpoint junction of BCR exons 7-8, LOC653203 intron and ABL exon a2; 195-220 LOC653203 intron), EU216071.1 (BCR-ABL fusion protein isoform Y5 mRNA; [SEQ ID NO:68]), EU216069.1 (BCR-ABL fusion protein isoform Y3 mRNA; [SEQ ID NO:69]), EU216067.1 (BCR-ABL fusion protein isoform Y1 mRNA; [SEQ ID NO:70]), EU216065.1 (BCR-ABL fusion protein isoform X8 mRNA; [SEQ ID NO:71]), EU216063.1 (BCR-ABL fusion protein isoform X6 mRNA; [SEQ ID NO:72]), EU216061.1 (BCR-ABL fusion protein isoform X4 mRNA; [SEQ ID NO:73]), EU216059.1 (BCR-ABL fusion protein isoform X2 mRNA; [SEQ ID NO:74]), EU216072.1 (BCR-ABL fusion protein isoform Y6 mRNA; [SEQ ID NO:75]), EU216070.1 (BCR-ABL fusion protein isoform Y4 mRNA; [SEQ ID NO:76]), EU216068.1 (BCR-ABL fusion protein isoform Y2 mRNA; [SEQ ID NO:77]), EU216066.1 (BCR-ABL fusion protein isoform X9 mRNA; [SEQ ID NO:78]), and EU216064.1 (BCR-ABL fusion protein isoform X7 mRNA; [SEQ ID NO:79]), AJ131466.1 (BCR-ABL e14a2 fusion protein partial mRNA; [SEQ ID NO:80]; exons: 1-117 (BCR exon 11), 118-193 (BCR exon 12), 194-298 (BCR exon 13), 299-373 (BCR exon 14), 374-547 (ABL exon 2), 548-843 (ABL exon 3), and 844-997 (ABL exon 4)), AJ131467.1 (BCR-ABL e13a2 fusion protein partial mRNA; [SEQ ID NO:81]; exons: 1-117 (BCR exon 1), 118-193 (BCR exon 12), 194-298 (BCR exon 13), 299-472 (ABL exon 2), 473-768 (ABL exon 3), and 769-922 (ABL exon 4)), AF113911.1 (BCR-ABL1 e1a2 fusion protein mRNA; [SEQ ID NO:82]; 456-457 fusion junction of Philadelphia translocation found in approximately 25% of cases of adult ALL), AM491363.1 (BCR-ABL1 e19a2 fusion protein mRNA; [SEQ ID NO:83]), AM491361.1 (BCR-ABL1 e1a3 fusion protein mRNA; [SEQ ID NO:84]), AM491359.1 (BCR-ABL1 e13a3 fusion protein mRNA; [SEQ ID NO:85]), AM491362.1 (BCR-ABL1 e6a2 fusion protein mRNA; [SEQ ID NO:86]), and AM491360.1 (BCR-ABL1 e14a3 fusion protein mRNA; [SEQ ID NO:87]).

(e) The echinoderm microtubule-associated protein-like 4 (EML4) gene is located on chromosome 2. The Entrez Gene, Ensembl, and HGNC cytogenetic bands are 2p21. Aliases include C2orf2, ROPP120, ELP120, restrictedly overexpressed proliferation-associated protein, Ropp120, EMAP-4, and EMAPL4. Reference DNA sequences include, but are not limited to, NC_{—}000002.11 and NT_{—}022184.15. The sequence of H. sapiens EML4 mRNA is available from NCBI as Accession No. BC008685.1 [SEQ ID NO:88], whereas the sequence of the H. sapiens EML4 transcript variant 1 is available from NCBI as Accession No. NM_{—}019063.3 [SEQ ID NO:89; exons: 1-287 (EML4), 288-470, 471-600, 601-774, 775-903, 904-929, 930-1053, 1054-1203, 1204-1273, 1274-1384, 1385-1480, 1481-1615, 1616-1751, 1752-1903, 1904-2029, 2030-2161, 2162-2229, 2230-2318, 2319-2416, 2417-2504, 2505-2603, 2604-2734, and 2735-5549] and the sequence of H. sapiens EML4 mRNA transcript variant 2 is available from NCBI as Accession No. NM_{—}001145076.1 [SEQ ID NO:90; exon: 1-287, 288-470, 471-600, 601-729, 730-755, 756-879, 880-1029, 1030-1099, 1100-1210, 1211-1306, 1307-1441, 1442-1577, 1578-1729, 1730-1855, 1856-1987, 1988-2055, 2056-2144, 2145-2242, 2243-2330, 2331-2429, 2430-2560, and 2561-5375]. Complete CDSs for H. sapiens EML4-ALK fusions are available from NCBI as Accession Nos. AB663645.1 [SEQ ID NO:91; exons: 1-1655 and 1714-3421; introns: 1656-1657 (intron 14 EML4) and 1658-1713 (intron 19 ALK)], JQ828841.1 (variant 3+20; [SEQ ID NO:92]; 338-339 EML4-ALK breakpoint junction), AB374364.1 (variant 5 splicing isoform a; [SEQ ID NO:93]; exons: 1-275 (exons 1-2 EML4) and 276-2014 (exons 20-ALK)), AB374365.1 (variant 5 splicing isoform b; [SEQ ID NO:94]; exons: 1-275 (exons 1-2 EML4) and 393-2131 (exons 20-ALK); intron: 276-392 (intron 19 ALK)), AB374363.1 (variant 4; [SEQ ID NO:95]; exons: 1-1708 (exons 1-14 EML4) and 1720-3409 (exons 20-ALK)), AB374362.1 (variant 3 splicing isoform b; [SEQ ID NO:96]; exons: 1-767 (exons 1-6b EML4), 735-767 (exon 6b EML4), and 768-2506 (exons 20-ALK)), AB374361.1 (variant 3 splicing isoform a; [SEQ ID NO:97]; exons: 1-734 (exons 1-6a EML4) and 735-2473 (exons 20-ALK)), AB274722.1 (variant 1; [SEQ ID NO:98]; exons: 1-1759 (exons 1-13 EML4) and 1760-3926 (exons 20-ALK)), and AB275889.1 (variant 2; [SEQ ID NO:99]; exons: 1-2512 (exons 1-20 EML4) and 2513-4679 (exons 20-ALK)).

(f) “Hybridization” refers to the formation of a duplex structure by complementary base pairing between two single-stranded nucleic acids. Hybridization can occur between exactly complementary nucleic acid strands or between complementary nucleic acid strands that contain a low number of mismatches.

(g) “Isothermal amplification” refers to methods of making copies of a DNA sequence at a constant temperature, i.e., without thermal cycling. Examples include helicase-dependent amplification, PAN-AC, nicking enzyme amplification reaction (NEAR), and recombinase polymerase amplification (RPA).

(h) The kinesin family member 5B gene (KIF5B) is located on chromosome 10. The Entrez Gene, Ensembl, and HGNC cytogenetic bands are 10p11.22. Aliases include KNS1, KNS, UKHC, conventional kinesin heavy chain, ubiquitous kinesin heavy chain, KINH, kinesin 1, kinesin heavy chain, and kinesin-1 heavy chain. Reference DNA sequences include, but are not limited to, NC_{—}000010.10 and NT_{—}008705.16. The mRNA sequence is available from NCBI as Accession No. NM_{—}004521.2 [SEQ ID NO:100] (exons: 1-596, 597-684, 685-758, 759-863, 864-912, 913-968, 969-1056, 1057-1181, 1182-1286, 1287-1432, 1433-1581, 1582-1775, 1776-1844, 1845-2051, 2052-2195, 2196-2384, 2385-2502, 2503-2564, 2565-2674, 2675-2776, 2777-2837, 2838-2909, 2910-3014, 3015-3231, 3232-3382, and 3383-5889).

(i) “Nucleic acid,” “polynucleotide,” and “oligonucleotide” refer to primers, detectable oligonucleotides, and oligomers, irrespective of length, and include polydeoxyribonucleotides, polyribonucleotides, and any other N-glycoside of a modified/unmodified, purine/pyrimidine base. Examples include single-stranded DNA (ssDNA), double-stranded DNA (dsDNA), single-stranded RNA (ssRNA), and double-stranded RNA (dsRNA). Such molecules can comprise phosphodiester linkages or modified linkages including, but not limited to, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations thereof. Such molecules can comprise adenine, guanine, thymine, cytosine and/or uracil, as well as other modified, non-standard, or derivatized bases. Alternatively or additionally, such molecules can comprise one or more modified sugar moieties.

(j) “Polymerase chain reaction (PCR)” is a method of making copies of a DNA sequence. The method employs thermal cycling (i.e., cycles of heating and cooling for denaturation (or melting) and replication of the DNA, respectively). Primers, which are short DNA fragments containing sequences complementary to the DNA sequence to be copied, and a heat-stable DNA polymerase, such as the one from Therms aquaticus, which is referred to as Taq polymerase, are used to select the DNA sequence and copy it (see, e.g., U.S. Pat. Nos. 4,683,195; 4,800,195, and 4,965,188, all of which are incorporated by reference herein for their teachings regarding same). With repeated cycling the copies, which are made, are used as templates for generating further copies (i.e., a chain reaction). PCR techniques include, but are not limited to, standard PCR, allele-specific PCR, assembly PCR, asymmetric PCR, digital PCR, Hot-start PCR, intersequence-specific PCR, inverse PCR, ligation-mediated PCR, methylation-specific PCR, mini-primer PCR, multiplex ligation-dependent detectable oligonucleotide amplification, nested PCR, overlap-extension PCR, real-time PCR, reverse transcription-PCR, solid phase PCR, thermal asymmetric interlaced PCR, and Touchdown PCR.

(k) “Primer” as used herein refers to an oligonucleotide that initiates template-dependent nucleic acid synthesis. In the presence of a nucleic acid template, nucleoside triphosphate precursors, a polymerase, and cofactors, under suitable conditions of temperature and pH, the primer can be extended at its 3′ terminus by the addition of nucleotides by the polymerase to yield a primer extension product. The primer may vary in length depending on the particular conditions employed and the purpose of the amplification. For example, a primer for amplification for a diagnostic purpose is typically from about 15 to about 35 nucleotides in length. The primer must be of sufficient complementarity to the desired template to prime the synthesis of the desired extension product. In other words, the primer must be able to anneal with the desired template strand in a manner sufficient to provide the 3′ hydroxyl moiety of the primer in appropriate juxtaposition for use in the initiation of synthesis by a polymerase. It is not necessary for the primer to be an exact complement of the desired template. For example, a non-complementary nucleotide sequence can be present at the 5′ end of an otherwise complementary primer. Alternatively, non-complementary bases can be interspersed within the oligonucleotide primer, provided that the primer sequence has sufficient complementarity with the sequence of the desired template strand to provide a template-primer complex for the synthesis of the extension product. An oligonucleotide, which has a sequence complementary to a primer, can be used as a probe, for example.

(l) “Probe” refers to an oligonucleotide that selectively hybridizes to a target nucleic acid under suitable conditions and can be detected.

(m) The promyelocytic leukemia gene (PML) is located on chromosome 15. The Ensembl and HGNC cytogenetic bands are 15q24.1, whereas the Entrez Gene cytogenetic band is 15q22. Aliases include MYL, RNF71, TRIM19, PP8675, promyelocytic leukemia protein, tripartite motif-containing protein 19, RING finger protein 71, probable transcription factor PML, protein PML, inducer of promyelocytic leukemia, and tripartite motif protein TRIM 19. Reference DNA sequences include, but are not limited to, NC_{—}000015.9 and NT_{—}010194.17. The sequence of PML mRNA transcript variant 5 is available from NCBI as Accession No. NM_{—}033244.3 [SEQ ID NO:101; exons: 1-269, 270-742, 743-1323, 1324-1394, 1395-1538, 1539-1805, 1806-1858, and 1859-3081], whereas the sequences of PML mRNA transcript variants 11, 9, 6 and 10 are available as Accession Nos. NM_{—}033250.2 [SEQ ID NO:102; exons: 1-269, 270-742, 743-1323, 1324-1394, 1395-1653, 1654-1706, and 1707-2929], NM_{—}033239.2 [SEQ ID NO:103; exons: 1-269, 270-742, 743-1323, 1324-1394, 1395-1538, 1539-1797, 1798-1850, and 1851-3073; 1320-1325 (breakpoint for translocation to form PML-RARA); 1794-1799 (breakpoint for translocation to form PML-RARA oncogene in type B APL)], NM_{—}002675.3 [SEQ ID NO:104; exons: 1-269, 270-742, 743-1323, 1324-1394, 1395-1538, 1539-1797, 1798-1850, and 1851-2238; 1320-1325 (breakpoint for translocation to form PML-RARA oncogene in type A APL)], and NM_{—}033249.2 [SEQ ID NO:105; exons: 1-269, 270-742, 743-1323, 1324-1394, 1395-1653, 1654-1706, and 1707-2094], respectively. The sequences of PML mRNA transcript variants 2, 8, 7 and 1 are available as Accession Nos. NM_{—}033240.2 [SEQ ID NO:106; exons: 1-269, 270-742, 743-1323, 1324-1394, 1395-1538, 1539-1797, and 1798-3714; 1320-1325 (breakpoint for translocation to form PML-RARA oncogene in type A APL); 1794-1799 (breakpoint for translocation to form PML-RARA in type B APL)], NM_{—}033247.2 [SEQ ID NO:107; exons: 1-269, 270-742, 743-1323, 1324-1394, and 1395-1782; 1320-1325 (breakpoint for translocation to form PML-RARA oncogene in type A APL)], NM_{—}033246.2 [SEQ ID NO:108; exons: 1-269, 270-742, 743-1323, 1324-1394, 1395-1447, and 1448-1835; 1320-1325 (breakpoint for translocation to form PML-RARA oncogene in type A APL)], and NM_{—}033238.2 [SEQ ID NO:109; exons: 1-269, 270-742, 743-1323, 1324-1394, 1395-1538, 1539-1797, 1798-1850, 1851-2001, and 2002-5600; 1320-1325 (breakpoint for translocation to form PML-RARA oncogene in type A APL); 1794-1799 (breakpoint for translocation to form PML-RARA oncogene in type B APL)], respectively. Complete CDS sequences are available from NCBI as Accession Nos. BC000080.2 [SEQ ID NO:110] and BCO20994.2 [SEQ ID NO:111]. Various PML-RARα gene fusion mRNA sequences are available from NCBI as Accession Nos. 576405.1 [SEQ ID NO:112], S76399.1 [SEQ ID NO:113], 576389.1 [SEQ ID NO:114], 576373.1 [SEQ ID NO:115], 576371.1 [SEQ ID NO:116], 576369.1 [SEQ ID NO:117], 576402.1 [SEQ ID NO:118], 576397.1 [SEQ ID NO:119], 576375.1 [SEQ ID NO:120], 576372.1 [SEQ ID NO:121], 576370.1 [SEQ ID NO:122], 576387.1 [SEQ ID NO:123], S76395.1 [SEQ ID NO:124], 576382.1 [SEQ ID NO:125], 557796.1 [SEQ ID NO:126], 576379.1 [SEQ ID NO:127], AJ417079.1 [SEQ ID NO:128; exons: 1-109 (PML exon 6) and 173-296 (RARA exon 3); intron: 110-172 (RARA intron 2)], AF388194.1 [SEQ ID NO:129], and AF388193.1 [SEQ ID NO:130].

(n) The ret proto-oncogene (RET) is located on chromosome 10. The Entrez Gene and HGNC cytogenetic bands are 10q11.2, whereas the Ensembl cytogenetic band is 10q11.21. Aliases include CDHF12, CDHR16, PTC, RET51, HSCR1, MEN2A, MEN2B, MTC1, EC 2.7.10.1, EC 2.7.10, cadherin family member 12, proto-oncogene c-Ret, Hirschsprung disease 1, multiple endocrine neoplasia and medullary thyroid carcinoma 1, RET-ELE1, cadherin-related family member 16, hydroxyaryl-protein kinase, proto-oncogene tyrosine-protein kinase receptor Ret, receptor tyroskine kinase, and RET transforming sequence. Reference DNA sequences include, but are not limited to, NC_{—}000010.10 and NT_{—}033985.7. The sequence for H. sapiens mRNA is available from NCBI as Accession No. X12949.1 [SEQ ID NO:131]. Complete CDS sequences for H. sapiens RET are available from NCBI as Accession Nos. BC003072.2 [SEQ ID NO:132] and BC004257.1 [SEQ ID NO:133]. The sequence for RET transcript variant 2 mRNA is available from NCBI as Accession No. NM_{—}020975.4 [SEQ ID NO:134; exons: 1-263, 264-527, 528-815, 816-1057, 1058-1253, 1254-1453, 1454-1712, 1713-1838, 1839-1949, 1950-2069, 2070-2326, 2327-2474, 2475-2582, 2583-2797, 2798-2920, 2921-2991, 2992-3129, 3130-3229, 3230-3377, and 3378-5617; 1949-1954 (breakpoint for translocation to form TRIM27-RET oncogene); 2324-2329 (breakpoint for translocation to form PCM1-RET, RET-CCDC6, RET-GOLGA5, RET-TRIM24, and RET-TRIM33 oncogenes)], whereas the sequence for RET transcript variant 4 mRNA is available as Accession No. NM_{—}020630.4 [SEQ ID NO:135; exons 1-263, 264-527, 528-815, 1058-1253, 1254-1453, 1454-1712, 1713-1838, 1839-1949, 1950-2069, 2070-2326, 2327-2474, 2475-2582, 2583-2797, 2798-2920, 2921-2991, 2992-3129, 3130-3229, and 3230-4159; 1949-1954 (breakpoint for translocation to form TRIM27-RET oncogene]. The sequences for H. sapiens RET exons 13, 15, and 2 are available from NCBI as Accession Nos. AF520983.1 [SEQ ID NO:136; exon 111-218], AF520979.1 [SEQ ID NO:137; exon 1-115], and AF520975.1 [SEQ ID NO:138; exon 1-266], respectively. The DNA sequence for exons 2-20 of H. sapiens RET is available from NCBI as Accession No. AJ243297.1 [SEQ ID NO:139; intron 1-964, exon 965-1229, intron 1230-2847, exon 2848-3140, intron 3141-5463, exon 5464-5700, intron 5701-6882, exon 6883-7079, intron 7080-9534, exon 9535-9733, intron 9734-11708, exon 11709-11967, intron 11968-12600, exon 12601-12727, intron 12728-13354, exon 13355-13464, intron 13465-14057, exon 14058-14177, intron 14178-14973, exon 14974-15230, intron 15231-17076, exon 17077-17224, intron 17225-18865, exon 18866-18973, intron 18974-20022, exon 20023-20237, intron 20238-20569, exon 20570-20695, intron 20696-22430, exon 22431-22501, intron 22502-24171, exon 24172-24310, intron 24311-25384, exon 25385-25483, intron 25484-27074, exon 27075-27221, intron 27222-28607, and exon 28608-28765].

(o) The retinoic acid receptor alpha gene (RARα) is located on chromosome 17. The Entrez Gene cytogenetic band is 17q21, whereas the Ensembl cytogenetic band is 17q21.2 and the HGNC cytogenetic band is 17q21.1. Aliases include NR1B1, RAR, nuclear receptor subfamily 1 group B member 1, nucleophosmin-retinoic acid receptor alpha fusion protein NPM-RAR long form, retinoic acid nuclear receptor alpha variant 1, retinoic acid nuclear receptor alpha variant 2, and retinoic acid receptor alpha polypeptide. Reference DNA sequences include, but are not limited to, NC_{—}000017.10 and NT_{—}010783.15. cDNA sequence is available from NCBI as Accession No. AK312564.1 [SEQ ID NO:140]. Complete CDS sequences of RARα are available from NCBI as Accession Nos. BC008727.2 [SEQ ID NO:141] and AH007261.5 [SEQ ID NO:142; exon 989-1192]. The sequences of RARα variant transcripts 4 and 3 are available from NCBI as Accession Nos. NM_{—}001145302.2 [SEQ ID NO:143; exons: 1-228, 229-768, 769-929, 930-1106, 1107-1311, 1312-1470, and 1471-3105] and NM_{—}001145301.2 [SEQ ID NO:144; exons: 1-228, 229-768, 769-917, 918-1059, 1060-1220, 1221-1397, 1398-1602, 1603-1761, and 1762-3396; 768-773 (breakpoint for translocation to form PLZF-RARA, RARA-PLZF, and PML-RARA oncogenes)], respectively, whereas the sequences of RARα variant transcripts 1 and 2 are available as Accession Nos. NM_{—}000964.3 [SEQ ID NO:145; exons: 1-116, 117-656, 657-805, 806-947, 948-1108, 1109-1285, 1286-1490, 1491-1649, and 1650-3284; 656-661 (breakpoint for translocation to form PLZF-RARA, RARA1-PLZF, and PML-RARA oncogenes)] and NM_{—}001024809.3 [SEQ ID NO:146; exons: 1-849, 850-998, 999-1140, 1141-1301, 1302-1478, 1479-1683, 1684-1842, and 1843-3477], respectively. Sequences of exons 1, 9, 7, and 3 are available as Accession Nos. AF088888.2 [SEQ ID NO:147; exon 989-1192], AF088895.2 [SEQ ID NO:148; exon 261-1081], AF088893.1 [SEQ ID NO:149; exon 326-530], and AF088890.1 [SEQ ID NO:150; exon 293-441], respectively. Sequences of exons 2, 4, 8, and 5-6 are available as Accession Nos. AF088889.2 [SEQ ID NO:151; exon 107-646], AF088891.2 [SEQ ID NO:152; exon 181-322], AF088894.1 [SEQ ID NO:153; exon 156-314], and AF088892.1 [SEQ ID NO:154; exons 422-582 and 843-1019], respectively.

(p) “Specifically hybridize(s),” as used herein, refers to the ability of a given nucleic acid, such as a primer or detectable oligonucleotide, to bind specifically to another nucleic acid.

(q) “Stringent” or “sequence-specific” hybridization conditions refers to conditions under which only exactly complementary nucleic acid strand will hybridize. Stringent hybridization conditions are well-known in the art. Stringent conditions are sequence-dependent and will be different under different circumstances. Generally, stringent conditions are selected to be about 5° C. lower than the thermal melting point (Tₘ) for the specific sequence under defined conditions of pH and ionic strength at which 50% of the base pairs are dissociated.

(r) “Substantially complementary” refers to sequences that are complementary except for minor regions of mismatches. Typically, the total number of mismatches in a nucleic acid that is about 15 nucleotides in length is about 3 nucleotides or less.

(s) “Target sequence” and “target region” refer to a region of a nucleic acid that it to be detected, or detected and analyzed, and comprises the fusion site of interest, i.e., e1a2, b2a2, b3a2, and e19a2 in the context of the present disclosure.

(t) “Variant-specific” refers to an oligonucleotide, such as an oligonucleotide primer or an oligonucleotide probe, which specifically amplifies or specifically hybridizes to, respectively, a nucleic acid sequence of a given variant of a gene fusion but not other variants of the same gene fusion or other gene fusion variants.

The terminology used herein is for the purpose of describing particular embodiments only and is not otherwise intended to be limiting.

### Method of Amplification, Detection, and Quantitation

A method of amplifying, alone or in further combination with detecting or detecting and quantitating, mRNA from variants of a fusion between a first gene and a second gene in a sample of mRNA is provided. The fusion can result from a chromosomal rearrangement, such as a translocation or an inversion. When a gene fusion is described in terms of a part of one gene (e.g., an exon) becoming contiguous with a part of another gene (e.g., an exon), for example, it is to be understood that the fusion is not limited to a particular type of chromosomal rearrangement and can be the result of a translocation or an inversion, for example.

A method of amplifying mRNA from variants of a fusion between a first gene and a second gene in a sample of mRNA is provided. The first gene and the second gene can be any known genes that generate variants. For example, the first gene and the second gene can be the breakpoint cluster region (BCR) gene and the Abelson murine leukemia (ABL) proto-oncogene, the echinoderm microtubule-associated protein-like 4 (EML4) gene and the anaplastic lymphoma kinase (ALK) gene, the kinesin family member 5B (KIF5B) gene and the Ret (RET) proto-oncogene, or the promyelocytic leukemia (PML) gene and the retinoic acid receptor alpha (RARα) gene. The method comprises (a) (i) obtaining cDNA, which has been reverse-transcribed from the sample of mRNA, or reverse-transcribing the sample of mRNA to cDNA and (ii) amplifying the reverse-transcribed cDNA, such as by polymerase chain reaction, using primers comprising a primer for an exon of the first gene, which becomes contiguous with a variant exon of the second gene, and a primer for each of two or more variant exons of the second gene. Use of “first” and “second” does not require that the first gene be 5′ to the second gene; rather, the first gene is the common fusion partner, whereas the second gene is the variant fusion partner. The method can further comprise (b) detecting the amplified cDNA or detecting and quantitating the amplified cDNA. The reverse-transcribed cDNA can be amplified, by polymerase chain reaction, for example, in the presence of a mixture of deoxyribonucleotide triphosphates (dNTP), a buffer, a polymerase, and a divalent ion, while cycling between a temperature of about 58° C. to about 62° C. for about 30 seconds to about 40 seconds and a temperature of about 92° C. for about 30 seconds. One or more primers can be detectably labeled. When more than one primer is detectably labeled, preferably the detection of the one or more detectably labeled primers can be distinguished. Detecting the amplified cDNA can comprise contacting the amplified cDNA with at least one probe under hybridizing conditions and detecting hybridization of at least one probe to the amplified cDNA. The probe can hybridize to a nucleotide sequence at or near a junction of a variant of a fusion between the first gene and the second gene comprising the 3′ end of an exon of the first gene and the 5′ end of an exon of the second gene. Detecting the amplified cDNA can comprise contacting the amplified cDNA with at least two probes, wherein hybridization of each probe to the amplified cDNA is distinguishable. Step (a) of the method can further comprise using primers for at least one internal control (IC) gene, wherein the primers can hybridize to the same or different exons of the at least one IC gene, and step (b) of the method can further comprise detecting the amplified IC cDNA. One or more of the primers for the at least one IC gene can be detectably labeled. When more than one primer for the at least one IC gene is detectably labeled, preferably the detection of the one or more detectably labeled primers can be distinguished. Detecting the amplified IC cDNA can comprise contacting the amplified IC cDNA with at least one IC probe and/or at least one IC primer under hybridizing conditions and detecting hybridization of at least one IC probe/primer to the amplified IC cDNA. Detecting the amplified IC cDNA can comprise contacting the amplified IC cDNA with at least two IC probes/primers, wherein each IC probe/primer is specific for a different IC cDNA and hybridization of each IC probe/primer to the amplified IC cDNA is distinguishable.

A method of amplifying mRNA from fusion variants of the breakpoint cluster region (BCR) gene and the Abelson murine leukemia (ABL) proto-oncogene in a sample of mRNA from a human is also provided. The method comprises (a) (i) obtaining cDNA, which has been reverse-transcribed from the sample of mRNA, or reverse-transcribing the sample of mRNA to cDNA and (ii) amplifying the reverse-transcribed cDNA using primers comprising primers (such as two, three, or four primers) for a group of BCR-ABL gene fusion variants comprising at least two gene fusion variants selected from the group consisting of an e1a2 gene fusion, a b2a2 gene fusion, a b3a2 gene fusion, and an e19a2 gene fusion. The method can further comprise (b) detecting the amplified cDNA or detecting and quantitating the amplified cDNA. The reverse-transcribed cDNA can be amplified by polymerase chain reaction in the presence of a mixture of dNTP, a buffer, a polymerase, and a divalent ion, while cycling between a temperature of about 58° C. to about 62° C. for about 30 seconds to about 40 seconds and a temperature of about 92° C. for about 30 seconds. The primers can comprise a primer that hybridizes to exon a2 of ABL. The primers can comprise a primer that hybridizes to exon e1 of BCR, a primer that hybridizes to exon b2 of BCR, and a primer that hybridizes to exon e19 of BCR. The primer that hybridizes to exon b2 of BCR can amplify reverse-transcribed cDNA from a BCR-ABL gene fusion variant comprising a b2a2 gene fusion and reverse-transcribed cDNA from a BCR-ABL gene fusion variant comprising a b3a2 gene fusion. One or more primers can be detectably labeled. If more than one primer is detectably labeled, preferably detection of the one or more detectably labeled primers can be distinguished. Detecting the amplified cDNA can comprise contacting the amplified cDNA with at least one probe under hybridizing conditions and detecting hybridization of at least one probe to the amplified cDNA. At least one probe can be a probe that hybridizes to a nucleotide sequence at or near a junction of a BCR-ABL gene fusion comprising the 3′ end of exon e1 of BCR and the 5′ end of exon a2 of ABL, a nucleotide sequence at or near a junction of a BCR-ABL gene fusion comprising the 3′ end of exon b2 of BCR and the 5′ end of exon a2 of ABL, a nucleotide sequence at or near a junction of a BCR-ABL gene fusion comprising the 3′ end of exon b3 of BCR and the 5′ end of exon a2 of ABL, or a nucleotide sequence at or near a junction of a BCR-ABL gene fusion comprising the 3′ end of exon e19 of BCR and the 5′ end of exon a2 of ABL. Detecting the amplified cDNA can comprise contacting the amplified cDNA with at least two probes, wherein hybridization of each probe to the amplified cDNA is distinguishable. Step (a) of the method can further comprise using primers for at least one IC gene, wherein the IC primers can hybridize to the same or different exons of the at least one IC gene, and step (b) of the method can further comprise detecting the amplified IC cDNA. One or more primers can be detectably labeled. If more than one primer is detectably labeled, preferably the detection of the one or more detectably labeled primers can be distinguished. Detecting the amplified IC cDNA can comprise contacting the amplified IC cDNA with at least one IC probe and/or at least one IC primer under hybridizing conditions and detecting hybridization of at least one IC probe/primer to the amplified IC cDNA. Detecting the amplified IC cDNA can comprise contacting the amplified IC cDNA with at least two IC probes/primers, wherein each IC probe/primer is specific for a different IC cDNA and hybridization of each IC probe/primer to the amplified IC cDNA is distinguishable. The primer that hybridizes to exon a2 of ABL can comprise the nucleotide sequence of SEQ ID NO: 4, 25, 26, or 27, such as SEQ ID NO: 4. The primer that hybridizes to exon e1 of BCR can comprise the nucleotide sequence of SEQ ID NO: 1, 18, 19, 20, 21, or 22, such as SEQ ID NO: 1. The primer that hybridizes to exon b2 of BCR can comprise the nucleotide sequence of SEQ ID NO: 2 or 23, such as SEQ ID NO: 2. The primer that hybridizes to exon e19 of BCR can comprise the nucleotide sequence of SEQ ID NO: 3 or 24, such as SEQ ID NO: 3. The probe that hybridizes to a nucleotide sequence at or near a junction of a BCR-ABL gene fusion comprising the 3′ end of exon e1 of BCR and the 5′ end of exon a2 of ABL can comprise SEQ ID NO: 5, 28, 29, 30, or a sequence complementary thereto, such as SEQ ID NO: 5. The probe that hybridizes to a nucleotide sequence at or near a junction of a BCR-ABL gene fusion comprising the 3′ end of exon b2 of BCR and the 5′ end of exon a2 of ABL can comprise SEQ ID NO: 6, 31, 32, or a sequence complementary thereto, such as SEQ ID NO: 6. The probe that hybridizes to a nucleotide sequence at or near a junction of a BCR-ABL gene fusion comprising the 3′ end of exon b3 of BCR and the 5′ end of exon a2 of ABL can comprise SEQ ID NO: 7, 33, 34, 35, or a sequence complementary thereto, such as SEQ ID NO: 7. The probe that hybridizes to a nucleotide sequence at or near a junction of a BCR-ABL gene fusion comprising the 3′ end of exon e19 of BCR and the 5′ end of exon a2 of ABL can comprise SEQ ID NO: 8, 36, or a sequence complementary thereto, such as SEQ ID NO: 8.

A method of amplifying mRNA from fusion variants of the echinoderm microtubule-associated protein-like 4 (EML4) gene and the anaplastic lymphoma kinase (ALK) gene in a sample of mRNA from a human is also provided. The method comprises (a) (i) obtaining cDNA, which has been reverse-transcribed from the sample of mRNA, or reverse-transcribing the sample of mRNA to cDNA and (ii) amplifying the reverse-transcribed cDNA using primers comprising primers (such as two, three, four, or five primers) for a group of EML4-ALK gene fusion variants comprising at least two gene fusion variants selected from the group consisting of an E13A20 gene fusion, an E6aA20 gene fusion, an E6bA20 gene fusion, an E14A20 gene fusion, and an E20A20 gene fusion. The method can further comprise (b) detecting the amplified cDNA or detecting and quantitating the amplified cDNA. The reverse-transcribed cDNA can be amplified by polymerase chain reaction in the presence of a mixture of dNTP, a buffer, a polymerase, and a divalent ion, while cycling between a temperature of about 58° C. to about 62° C. for about 30 seconds to about 40 seconds and a temperature of about 92° C. for about 30 seconds. The primers can comprise a primer that hybridizes to exon A20 of ALK. The primers can comprise two or more of a primer that hybridizes to exon E13 of EML4, a primer that hybridizes to exon E6a of EML4, and a primer that hybridizes to exon E20 of EML4. The primer that hybridizes to exon E6a of EML4 can amplify reverse-transcribed cDNA from an EML4-ALK gene fusion variant comprising an E6a gene fusion and reverse-transcribed cDNA from an EML4-ALK gene fusion variant comprising an E6b gene fusion. The primer that hybridizes to exon E13 of EML4 can amplify reverse-transcribed cDNA from an EML4-ALK gene fusion variant comprising an E13 gene fusion and reverse-transcribed cDNA from an EML4-ALK gene fusion variant comprising an E14 gene fusion. One or more primers can be detectably labeled. If more than one primer is detectably labeled, preferably detection of the one or more detectably labeled primers can be distinguished. Detecting the amplified cDNA can comprise contacting the amplified cDNA with at least one probe under hybridizing conditions and detecting hybridization of the at least one probe to the amplified cDNA. The at least one probe can be a probe that hybridizes to a nucleotide sequence at or near a junction of an EML4-ALK gene fusion comprising the 3′ end of exon E13 of EML4 and the 5′ end of exon A20 of ALK, a nucleotide sequence at or near a junction of an EML4-ALK gene fusion comprising the 3′ end of exon E6a of EML4 and the 5′ end of exon A20 of ALK, a nucleotide sequence at or near a junction of an EML4-ALK gene fusion comprising the 3′ end of exon E6b of EML4 and the 5′ end of exon A20 of ALK, a nucleotide sequence at or near a junction of a EML4-ALK gene fusion comprising the 3′ end of exon E14 of EML4 and the 5′ end of exon A20 of ALK, or a nucleotide sequence at or near a junction of an EML4-ALK gene fusion comprising the 3′ end of exon E20 of EML4 and the 5′ end of exon A20 of ALK. Detecting the amplified cDNA can comprise contacting the amplified cDNA with at least two probes, wherein hybridization of each probe to the amplified cDNA is distinguishable. Step (a) of the method can further comprise using primers for at least one IC gene, wherein the primers hybridize to the same or different exons of the at least one IC gene, and step (b) of the method can further comprise detecting the amplified IC cDNA. One or more of the IC primers can be detectably labeled. Preferably, detection of the one or more detectably labeled IC primers can be distinguished. Detecting the amplified IC cDNA can comprise contacting the amplified IC cDNA with at least one IC probe and/or at least one IC primer under hybridizing conditions and detecting hybridization of at least one IC probe/primer to the amplified IC cDNA. Detecting the amplified IC cDNA can comprise contacting the amplified IC cDNA with at least two IC probes/primers, wherein each IC probe/primer is specific for a different IC cDNA and hybridization of each IC probe/primer to the amplified IC cDNA is distinguishable. Step (a)(ii) of the method can further comprise amplifying the reverse-transcribed cDNA using primers for at least one further EML4-ALK gene fusion variant selected from the group consisting of an E18A20 gene fusion, an E15A20 gene fusion, an E2A20 gene fusion, and an E17A20 gene fusion.

A method of amplifying mRNA from fusion variants of the kinesin family member 5B (KIF5B) gene and the Ret (RET) proto-oncogene in a sample of mRNA from a human is also provided. The method comprises (a) (i) obtaining cDNA, which has been reverse-transcribed from the sample of mRNA, or reverse-transcribing the sample of mRNA to cDNA and (ii) amplifying the reverse-transcribed cDNA using primers comprising primers (such as two, three or four primers) for a group of KIF5B-RET gene fusion variants comprising at least two gene fusion variants selected from the group consisting of a K15R12 gene fusion, a K16R12 gene fusion, a K22R12 gene fusion, and a K23R12 gene fusion. The method can further comprise (b) detecting the amplified cDNA or detecting and quantitating the amplified cDNA. The reverse-transcribed cDNA can be amplified by polymerase chain reaction in the presence of a mixture of dNTP, a buffer, a polymerase, and a divalent ion, while cycling between a temperature of about 58° C. to about 62° C. for about 30 seconds to about 40 seconds and a temperature of about 92° C. for about 30 seconds. The primers can comprise a primer that hybridizes to exon R12 of RET. The primers can comprise a primer that hybridizes to exon K15 of KIF5B and/or a primer that hybridizes to exon K22 of KIF5B. The primer that hybridizes to exon K15 of KIF5B can amplify reverse-transcribed cDNA from a KIF5B-RET gene fusion variant comprising a K15R12 gene fusion and reverse-transcribed cDNA from a KIF5B-RET gene fusion variant comprising a K16R12 gene fusion. The primer that hybridizes to exon K22 of KIF5B can amplify reverse-transcribed cDNA from a KIF5B-RET gene fusion variant comprising a K22R12 gene fusion and reverse-transcribed cDNA from a KIF5B-RET gene fusion variant comprising a K23R12 gene fusion. One or more of the primers can be detectably labeled. Preferably, detection of the one or more detectably labeled primers can be distinguished. Detecting the amplified cDNA can comprise contacting the amplified cDNA with at least one probe under hybridizing conditions and detecting hybridization of at least one probe to the amplified cDNA. At least one probe can be a probe that hybridizes to a nucleotide sequence at or near a junction of a KIF5B-RET gene fusion comprising the 3′ end of exon K15 of KIF5B and the 5′ end of exon R12 of RET, a nucleotide sequence at or near a junction of a KIF5B-RET gene fusion comprising the 3′ end of exon K16 of KIF5B and the 5′ end of exon R12 of RET, a nucleotide sequence at or near a junction of a KIF5B-RET gene fusion comprising the 3′ end of exon K22 of KIF5B and the 5′ end of exon R12 of RET, or a nucleotide sequence at or near a junction of a KIF5B-RET gene fusion comprising the 3′ end of exon K23 of KIF5B and the 5′ end of exon R12 of RET. Detecting the amplified cDNA can comprise contacting the amplified cDNA with at least two probes, wherein hybridization of each probe to the amplified cDNA is distinguishable. Step (a) of the method can further comprise using primers for at least one IC gene, wherein the primers hybridize to the same or different exons of the at least one IC gene, and (b) further comprises detecting the amplified IC cDNA. One or more of the IC primers can be detectably labeled. Preferably, the detection of the one or more detectably labeled IC primers can be distinguished. Detecting the amplified IC cDNA can comprise contacting the amplified IC cDNA with at least one IC probe and/or at least one IC primer under hybridizing conditions and detecting hybridization of the at least one IC probe/primer to the amplified IC cDNA. Detecting the amplified IC cDNA can comprise contacting the amplified IC cDNA with at least two IC probes/primers, wherein each IC probe/primer is specific for a different IC cDNA and hybridization of each IC probe/primer to the amplified IC cDNA is distinguishable.

A method of amplifying mRNA from fusion variants of the promyelocytic leukemia (PML) gene and the retinoic acid receptor alpha (RARα) gene in a sample of mRNA from a human is also provided. The method comprises (a) (i) obtaining cDNA, which has been reverse-transcribed from the sample of mRNA, or reverse-transcribing the sample of mRNA to cDNA and (ii) amplifying the reverse-transcribed cDNA using primers comprising primers (such as two or three primers) for a group of PML-RARα gene fusion variants comprising at least two gene fusion variants selected from the group consisting of a P3R3 gene fusion, a P6aR3 gene fusion, and a P6bR3 gene fusion. The method can further comprise (b) detecting the amplified cDNA or detecting and quantitating the amplified cDNA. The reverse-transcribed cDNA can be amplified by polymerase chain reaction in the presence of a mixture of dNTP, a buffer, a polymerase, and a divalent ion, while cycling between a temperature of about 58° C. to about 62° C. for about 30 seconds to about 40 seconds and a temperature of about 92° C. for about 30 seconds. The primers can comprise a primer that hybridizes to exon R3 of RARα. The primers can comprise a primer that hybridizes to exon P3 of PML and/or a primer that hybridizes to exon 6a of PML. The primer that hybridizes to exon 6a of PML can amplify reverse-transcribed cDNA from a PML-RARα gene fusion variant comprising a P6aR3 gene fusion and reverse-transcribed cDNA from a PML-RARα gene fusion variant comprising a P6bR3 gene fusion. One or more primers can be detectably labeled. Preferably, detection of the one or more detectably labeled primers can be distinguished. Detecting the amplified cDNA can comprise contacting the amplified cDNA with at least one probe under hybridizing conditions and detecting hybridization of the at least one probe to the amplified cDNA. At least one probe can be a probe that hybridizes to a nucleotide sequence at or near a junction of a PML-RARα gene fusion comprising the 3′ end of exon P3 of PML and the 5′ end of exon R3 of RARα, a nucleotide sequence at or near a junction of a PML-RARα gene fusion comprising the 3′ end of exon P6a of PML and the 5′ end of exon R3 of RARα, or a nucleotide sequence at or near a junction of a PML-RARα gene fusion comprising the 3′ end of exon P6b of PML and the 5′ end of exon R3 of RARα. Detecting the amplified cDNA can comprise contacting the amplified cDNA with at least two probes, wherein hybridization of each probe to the amplified cDNA is distinguishable. Step (a) of the method can further comprise using primers for at least one IC gene, wherein the IC primers hybridize to the same or different exons of the at least one IC gene, and (b) further comprises detecting the amplified IC cDNA. One or more of the IC primers can be detectably labeled. Preferably, detection of the one or more detectably labeled IC primers can be distinguished. Detecting the amplified IC cDNA can comprise contacting the amplified IC cDNA with at least one IC probe and/or at least one IC primer under hybridizing conditions and detecting hybridization of at least one IC probe/primer to the amplified IC cDNA. Detecting the amplified IC cDNA can comprise contacting the amplified IC cDNA with at least two IC probes/primers, wherein each IC probe/primer is specific for a different IC cDNA and hybridization of each IC probe/primer to the amplified IC cDNA is distinguishable.

A method of detecting mRNA from variants of a fusion between a first gene and a second gene in a sample of mRNA is also provided. The method comprises (a) (i) obtaining cDNA, which has been reverse-transcribed from the sample of mRNA, or reverse-transcribing the sample of mRNA to cDNA and (ii) amplifying the reverse-transcribed cDNA using a primer for an exon of the first gene, which becomes contiguous with a variant exon of the second gene, and a primer for each of two or more variant exons of the second gene, and (b) detecting the amplified cDNA, and optionally, simultaneously or sequentially quantitating the amplified cDNA. Use of “first” and “second” does not require that the first gene be 5′ to the second gene; rather, the first gene is the common fusion partner, whereas the second gene is the variant fusion partner. One or more primers can be detectably labeled, in which case the detection of the one or more detectably labeled primers preferably can be distinguished. Detecting the amplified cDNA can comprise contacting the amplified cDNA with at least one probe under hybridizing conditions and detecting hybridization of at least one probe to the amplified cDNA. At least one probe can be a probe that hybridizes to a nucleotide sequence at or near a junction of a variant of a fusion between the first gene and the second gene comprising the 3′ end of an exon of the first gene and the 5′ end of an exon of the second gene. Detecting the amplified cDNA can comprise contacting the amplified cDNA with at least two probes, wherein hybridization of each probe to the amplified cDNA is distinguishable. Step (a) of the method can further comprise using primers for at least one IC gene, wherein the IC primers can hybridize to the same or different exons of the at least one IC gene, and step (b) of the method can further comprise detecting the amplified IC cDNA. One or more of the IC primers can be detectably labeled, in which case the detection of the one or more detectably labeled IC primers preferably can be distinguished. Detecting the amplified IC cDNA can comprise contacting the amplified IC cDNA with at least one IC probe and/or at least one IC primer under hybridizing conditions and detecting hybridization of at least one IC probe/primer to the amplified IC cDNA. Detecting the amplified IC cDNA can comprise contacting the amplified IC cDNA with at least two IC probes/primers, wherein each IC probe/primer is specific for a different IC cDNA and hybridization of each IC probe/primer to the amplified IC cDNA is distinguishable.

A method of detecting mRNA from fusion variants of the BCR gene and the ABL proto-oncogene in a sample of mRNA from a human is also provided. The method comprises (a) (i) obtaining cDNA, which has been reverse-transcribed from the sample of mRNA, or reverse-transcribing the sample of mRNA to cDNA and (ii) amplifying the reverse-transcribed cDNA using primers, i.e., forward and reverse primers, that effect reverse-transcription and amplification of a group of BCR-ABL gene fusion variants comprising at least two gene fusion variants, such as two, three or four gene fusion variants, selected from the group consisting of an e1a2 gene fusion, a b2a2 gene fusion, a b3a2 gene fusion, and an e19a2 gene fusion, whereupon, if mRNA from an e1a2, b2a2, b3a2, or e19a2 BCR-ABL gene fusion variant is present in the sample of mRNA, the mRNA is reverse-transcribed to cDNA (see (a)(i)) and the reverse-transcribed cDNA is amplified (see (a) (ii)), and (b) detecting the amplified cDNA or detecting and quantitating the amplified cDNA. The primers can comprise a primer, such as a reverse primer, that hybridizes to exon a2 of ABL. The primer, which hybridizes to exon a2 of ABL can reverse-transcribe cDNA from mRNA of two or more BCR-ABL gene fusion variants. The primers can comprise primers, such as forward primers, that hybridize to exon e1 of BCR, exon b2 of BCR, and exon e19 of BCR. The primer, which hybridizes to exon b2 of BCR, such as a forward primer, can amplify reverse-transcribed cDNA from a BCR-ABL gene fusion variant comprising a b2a2 gene fusion and reverse-transcribed cDNA from a BCR-ABL gene fusion variant comprising a b3a2 gene fusion. One or more of the primers can be detectably labeled, such as distinctly detectably labeled, in which case preferably, and even desirably, detection of the one or more detectably labeled primers can be distinguished.

Alternatively or additionally to detectably labeling one or more of the primers, step (b) can comprise contacting the amplified cDNA with at least one probe under hybridizing conditions and detecting hybridization of at least one probe to the amplified cDNA. At least one probe can be a probe that hybridizes to a nucleotide sequence at or near a junction of a BCR-ABL gene fusion comprising the 3′ end of exon e1 of BCR and the 5′ end of exon a2 of ABL, a nucleotide sequence at or near a junction of a BCR-ABL gene fusion comprising the 3′ end of exon b2 of BCR and the 5′ end of exon a2 of ABL, a nucleotide sequence at or near a junction of a BCR-ABL gene fusion comprising the 3′ end of exon b3 of BCR and the 5′ end of exon a2 of ABL, or a nucleotide sequence at or near a junction of a BCR-ABL gene fusion comprising the 3′ end of exon e19 of BCR and the 5′ end of exon a2 of ABL. Step (b) can comprise contacting the amplified cDNA with at least two probes, wherein each probe hybridizes to a different BCR-ABL gene fusion variant cDNA and hybridization of each probe to the amplified cDNA is preferably, even desirably, distinguishable.

Step (a) can further comprise using primers, i.e., forward and reverse primers, that effect reverse-transcription of mRNA from at least one IC gene to cDNA (see (a)(i)) and amplification of the reverse-transcribed IC cDNA (see (a)(ii)), in which case step (b) can further comprise detecting the amplified IC cDNA. The IC primers can hybridize to the same or different exons of the at least one IC gene. One or more of the IC primers can be detectably labeled, such as distinctly detectably labeled for each IC cDNA, in which case preferably, even desirably, detection of the one or more detectably labeled IC primers can be distinguished. Thus, detecting amplified IC cDNA can comprise detecting a labeled IC primer. Alternatively or additionally to detecting amplified IC cDNA by detecting a labeled IC primer, detecting amplified IC cDNA can comprise contacting the amplified IC cDNA with at least one IC probe, such as a probe that hybridizes to a nucleotide sequence at or near a junction of two exons of an IC gene comprising the 3′ end of an exon and the 5′ end of an adjacent exon, and/or at least one IC primer, under hybridizing conditions and detecting hybridization of at least one IC probe and/or at least one IC primer to the amplified IC cDNA. Detecting the amplified IC cDNA can comprise contacting the amplified IC cDNA with at least two IC probes and/or IC primers, wherein each IC probe/primer is specific for a different IC cDNA and hybridization of each IC probe/primer to the amplified IC cDNA is preferably, even desirably, distinguishable. Signals from two or more IC genes, such as two IC genes, three IC genes, four IC genes, or more than four IC genes, can be averaged, and the average signal can be used to quantitate one or more gene fusions amplified and detected in accordance with the method described herein.

The primer, such as a reverse primer, that hybridizes to exon a2 of ABL can comprise the nucleotide sequence of SEQ ID NO: 4, 25, 26, or 27. A preferred nucleotide sequence for a primer, in particular a reverse primer, is SEQ ID NO: 4.

The primer, such as a forward primer, that hybridizes to exon e1 of BCR can comprise the nucleotide sequence of SEQ ID NO: 1, 18, 19, 20, 21, or 22. A preferred nucleotide sequence is SEQ ID NO: 1.

The primer, such as a forward primer, that hybridizes to exon b2 of BCR can comprise the nucleotide sequence of SEQ ID NO: 2 or 23. A preferred nucleotide sequence is SEQ ID NO: 2.

The primer, such as a forward primer, that hybridizes to exon e19 of BCR can comprise the nucleotide sequence of SEQ ID NO: 3 or 24. A preferred nucleotide sequence is SEQ ID NO: 3.

The probe that hybridizes to a nucleotide sequence at or near a junction of a BCR-ABL gene fusion comprising the 3′ end of exon e1 of BCR and the 5′ end of exon a2 of ABL can comprise SEQ ID NO: 5, 28, 29, 30 or a sequence complementary thereto. SEQ ID NO: 5 or a sequence complementary thereto is preferred.

The probe that hybridizes to a nucleotide sequence at or near a junction of a BCR-ABL gene fusion comprising the 3′ end of exon b2 of BCR and the 5′ end of exon a2 of ABL can comprise SEQ ID NO: 6, 31, 32 or a sequence complementary thereto. SEQ ID NO: 6 or a sequence complementary thereto is preferred.

The probe that hybridizes to a nucleotide sequence at or near a junction of a BCR-ABL gene fusion comprising the 3′ end of exon b3 of BCR and the 5′ end of exon a2 of ABL can comprise SEQ ID NO: 7, 33, 34, 35, or a sequence complementary thereto. SEQ ID NO: 7 or a sequence complementary thereto is preferred.

The probe that hybridizes to a nucleotide sequence at or near a junction of a BCR-ABL gene fusion comprising the 3′ end of exon e19 of BCR and the 5′ end of exon a2 of ABL can comprise SEQ ID NO: 8, 36, or a sequence complementary thereto. SEQ ID NO: 8 or a sequence complementary thereto is preferred.

A method of detecting mRNA from a fusion variant of the BCR gene and the ABL proto-oncogene in a sample of mRNA from a human is also provided. The method comprises (a) (i) obtaining cDNA, which has been reverse-transcribed from the sample of mRNA, or reverse-transcribing the sample of mRNA to cDNA and (ii) amplifying the reverse-transcribed cDNA using primers, i.e., forward and reverse primers, that effect reverse-transcription and amplification of at least one BCR-ABL gene fusion variant comprising an e1a2 gene fusion, a b2a2 gene fusion, a b3a2 gene fusion, or an e19a2 gene fusion, wherein the primers comprise a primer, such as a forward primer, comprising the nucleotide sequence of SEQ ID NO: 1, 2, 3, 18, 19, 20, 21, 22, 23, or 24, whereupon, if mRNA from the at least one BCR-ABL gene fusion variant is present in the sample of mRNA and the forward and reverse primers that effect reverse-transcription and amplification of the at least one BCR-ABL gene fusion variant are used, the mRNA is reverse-transcribed to cDNA (see (a)(i)) and the reverse-transcribed cDNA is amplified (see (a)(ii)), and (b) detecting the amplified cDNA or detecting and quantitating the amplified cDNA. One or more of the primers can be detectably labeled, such as distinctly detectably labeled, in which case preferably, even desirably, detection of the one or more labeled primers can be distinguished. Alternatively or additionally to detectably labeling one or more of the primers, step (b) can comprise contacting the amplified cDNA with at least one probe and detecting hybridization of the probe to the amplified cDNA. Detecting the amplified cDNA can comprise contacting the amplified cDNA with at least two probes, wherein each probe hybridizes to a different BCR-ABL gene fusion variant cDNA and hybridization of each probe to the amplified BCR-ABL gene fusion variant cDNA is preferably, even desirably, distinguishable.

Step (a) can further comprise using primers, i.e., forward and reverse primers, that effect reverse-transcription of mRNA from at least one IC gene to cDNA and amplification of the reverse-transcribed IC cDNA, in which case step (b) further comprises detecting the amplified IC cDNA. The IC primers optionally hybridize to different exons of the at least one IC gene. One or more of the IC primers can be detectably labeled, such as distinctly detectably labeled for each IC cDNA, in which case detection of the one or more detectably labeled IC primers can be preferably, even desirably, distinguished. Thus, detecting amplified IC cDNA can comprise detecting a labeled IC primer. The conditions of reverse-transcription and amplification of BCR-ABL gene fusion variants are desirably suitable for reverse-transcription and amplification of at least one IC gene. Alternatively or additionally to detecting amplified IC cDNA by detecting a labeled IC primer, detecting the amplified IC cDNA can comprise contacting the amplified IC cDNA with at least one probe and/or at least one primer, wherein each probe/primer is specific for each amplified IC cDNA and hybridization of each probe/primer to the amplified IC cDNA is preferably, even desirably, distinguishable.

The primers can comprise a primer, such as a forward primer, comprising the nucleotide sequence of SEQ ID NO: 1, 18, 19, 20, 21, or 22. A preferred nucleotide sequence is SEQ ID NO: 1 or a sequence complementary thereto.

The primers can comprise a primer, such as a forward primer, comprising the nucleotide sequence of SEQ ID NO: 2 or 23. A preferred nucleotide sequence is SEQ ID NO: 2.

The primers can comprise a primer, such as a forward primer, comprising the nucleotide sequence of SEQ ID NO: 3 or 24. A preferred nucleotide sequence is SEQ ID NO: 3.

The primer, such as a reverse primer, can comprise a nucleotide sequence selected from the group consisting of SEQ ID NOS: 4, 25, 26, or 27. SEQ ID NO: 4 is preferred, particularly for a reverse primer.

The at least one probe can comprise a probe comprising the nucleotide sequence of SEQ ID NO: 5, 6, 7, 8, 28, 29, 30, 31, 32, 33, 34, 35, 36, or a sequence complementary thereto. Preferably, the at least one probe comprises a probe comprising the nucleotide sequence of SEQ ID NO: 5, 28, 29, 30, or a sequence complementary thereto, a probe comprising the nucleotide sequence of SEQ ID NO: 6, 31, 32, or a sequence complementary thereto, a probe comprising the nucleotide sequence of SEQ ID NO: 7, 33, 34, 35, or a sequence complementary thereto, and/or a probe comprising the nucleotide sequence of SEQ ID NO: 8, 36, or a sequence complementary thereto. More preferably, the at least one probe comprises a probe comprising the nucleotide sequence of SEQ ID NO: 5 or a sequence complementary thereto, a probe comprising the nucleotide sequence of SEQ ID NO: 6 or a sequence complementary thereto, a probe comprising the nucleotide sequence of SEQ ID NO: 7 or a sequence complementary thereto, and/or a probe comprising the nucleotide sequence of SEQ ID NO: 8 or a sequence complementary thereto.

When the method comprises detecting mRNA from two or more BCR-ABL gene fusion variants, the method can comprise (i) obtaining cDNA reverse-transcribed from the sample of mRNA or reverse-transcribing the sample of mRNA to cDNA and (ii) amplifying the reverse-transcribed cDNA for the BCR-ABL gene fusion variants together or separately. The presence of a BCR-ABL fusion variant can be detected, or detected and quantitated, using any suitable method. When BCR-ABL gene fusion variants are, in the case of obtained cDNA, amplified together, and, in the case of mRNA, reverse-transcribed and amplified together, the use of probes, which can be distinguished from each other, for example, enables the presence/absence/quantity of each BCR-ABL gene fusion variant to be determined.

A method of detecting mRNA from fusion variants of the echinoderm microtubule-associated protein-like 4 (EML4) gene and the anaplastic lymphoma kinase (ALK) gene in a sample of mRNA from a human is also provided. The method comprises (a) (i) obtaining cDNA, which has been reverse-transcribed from the sample of mRNA, or reverse-transcribing the sample of mRNA to cDNA and (ii) amplifying the reverse-transcribed cDNA using primers for a group of EML4-ALK gene fusion variants comprising at least two gene fusion variants selected from the group consisting of an E13A20 gene fusion, an E6aA20 gene fusion, an E6bA20 gene fusion, an E14A20 gene fusion, and an E20A20 gene fusion, and (b) detecting the amplified cDNA or detecting and quantitating the amplified cDNA. The primers comprise a primer that hybridizes to exon A20 of ALK. The primers can comprise a primer that hybridizes to exon E13 of EML4, a primer that hybridizes to exon E6a of EML4, and a primer that hybridizes to exon E20 of EML4. The primer that hybridizes to exon E6a of EML4 can amplify reverse-transcribed cDNA from an EML4-ALK gene fusion variant comprising an E6a gene fusion and reverse-transcribed cDNA from an EML4-ALK gene fusion variant comprising an Ebb gene fusion and/or the primer that hybridizes to exon E13 of EML4 can amplify reverse-transcribed cDNA from an EML4-ALK gene fusion variant comprising an E13 gene fusion and reverse-transcribed cDNA from an EML4-ALK gene fusion variant comprising an E14 gene fusion. One or more primers is/are detectably labeled. The detection of the one or more detectably labeled primers can be distinguished. Detecting the amplified cDNA can comprise contacting the amplified cDNA with at least one probe under hybridizing conditions and detecting hybridization of at least one probe to the amplified cDNA. At least one probe is a probe that hybridizes to a nucleotide sequence at or near a junction of an EML4-ALK gene fusion comprising the 3′ end of exon E13 of EML4 and the 5′ end of exon A20 of ALK, a nucleotide sequence at or near a junction of an EML4-ALK gene fusion comprising the 3′ end of exon E6a of EML4 and the 5′ end of exon A20 of ALK, a nucleotide sequence at or near a junction of an EML4-ALK gene fusion comprising the 3′ end of exon Ebb of EML4 and the 5′ end of exon A20 of ALK, a nucleotide sequence at or near a junction of a BCR-ABL gene fusion comprising the 3′ end of exon E14 of EML4 and the 5′ end of exon A20 of ALK, or a nucleotide sequence at or near a junction of an EML4-ALK gene fusion comprising the 3′ end of exon E20 of EML4 and the 5′ end of exon A20 of ALK. Detecting the amplified cDNA can comprise contacting the amplified cDNA with at least two probes, wherein hybridization of each probe to the amplified cDNA is distinguishable. In the method (a) can further comprise using primers for at least one IC gene, wherein the primers optionally hybridize to different exons of the at least one IC gene, and (b) further comprises detecting the amplified IC cDNA. One or more IC primers can be detectably labeled. Detection of the one or more detectably labeled IC primers can be distinguished. Detecting the amplified IC cDNA can comprise contacting the amplified IC cDNA with at least one IC probe and/or at least one IC primer under hybridizing conditions and detecting hybridization of at least one IC probe/primer to the amplified IC cDNA. Detecting the amplified IC cDNA can comprise contacting the amplified IC cDNA with at least two IC probes/primers, wherein each IC probe/primer is specific for a different IC cDNA and hybridization of each IC probe/primer to the amplified IC cDNA is distinguishable. In the method (a)(ii) can further comprise amplifying the reverse-transcribed cDNA using primers for at least one further EML4-ALK gene fusion variant selected from the group consisting of an E18A20 gene fusion, an E15A20 gene fusion, an E2A20 gene fusion, and an E17A20 gene fusion.

A method of detecting mRNA from fusion variants of the kinesin family member 5B (KIF5B) gene and the Ret (RET) proto-oncogene in a sample of mRNA from a human is also provided. The method comprises (a) (i) obtaining cDNA, which has been reverse-transcribed from the sample of mRNA, or reverse-transcribing the sample of mRNA to cDNA and (ii) amplifying the reverse-transcribed cDNA using primers for a group of KIF5B-RET gene fusion variants comprising at least two gene fusion variants selected from the group consisting of a K15R12 gene fusion, a K16R12 gene fusion, a K22R12 gene fusion, and a K23R12 gene fusion, and (b) detecting the amplified cDNA or detecting and quantitating the amplified cDNA. The primers comprise a primer that hybridizes to exon R12 of RET. The primers can comprise a primer that hybridizes to exon K15 of KIF5B and a primer that hybridizes to exon K22 of KIF5B. The primer that hybridizes to exon K15 of KIF5B can amplify reverse-transcribed cDNA from a KIF5B-RET gene fusion variant comprising a K15R12 gene fusion and reverse-transcribed cDNA from a KIF5B-RET gene fusion variant comprising a K16R12 gene fusion and/or the primer that hybridizes to exon K22 of KIF5B can amplify reverse-transcribed cDNA from a KIF5B-RET gene fusion variant comprising a K22R12 gene fusion and reverse-transcribed cDNA from a KIF5B-RET gene fusion variant comprising a K23R12 gene fusion. One or more primers is/are detectably labeled. Detection of the one or more detectably labeled primers can be distinguished. Detecting the amplified cDNA comprises contacting the amplified cDNA with at least one probe under hybridizing conditions and detecting hybridization of at least one probe to the amplified cDNA. At least one probe can be a probe that hybridizes to a nucleotide sequence at or near a junction of a KIF5B-RET gene fusion comprising the 3′ end of exon K15 of KIF5B and the 5′ end of exon R12 of RET, a nucleotide sequence at or near a junction of a KIF5B-RET gene fusion comprising the 3′ end of exon K16 of KIF5B and the 5′ end of exon R12 of RET, a nucleotide sequence at or near a junction of a KIF5B-RET gene fusion comprising the 3′ end of exon K22 of KIF5B and the 5′ end of exon R12 of RET, or a nucleotide sequence at or near a junction of a KIF5B-RET gene fusion comprising the 3′ end of exon K23 of KIF5B and the 5′ end of exon R12 of RET. Detecting the amplified cDNA can comprise contacting the amplified cDNA with at least two probes, wherein hybridization of each probe to the amplified cDNA is distinguishable. In the method (a) can further comprise using primers for at least one IC gene, wherein the primers optionally hybridize to different exons of the at least one IC gene, and (b) further comprises detecting the amplified IC cDNA. One or more IC primers can be detectably labeled. Detection of the one or more detectably labeled IC primers can be distinguished. Detecting the amplified IC cDNA can comprise contacting the amplified IC cDNA with at least one IC probe and/or at least one IC primer under hybridizing conditions and detecting hybridization of at least one IC probe/primer to the amplified IC cDNA. Detecting the amplified IC cDNA comprises contacting the amplified IC cDNA with at least two IC probes/primers, wherein each IC probe/primer is specific for a different IC cDNA and hybridization of each IC probe/primer to the amplified IC cDNA is distinguishable.

A method of detecting mRNA from fusion variants of the promyelocytic leukemia (PML) gene and the retinoic acid receptor alpha (RARα) gene in a sample of mRNA from a human is also provided. The method comprises (a) (i) obtaining cDNA, which has been reverse-transcribed from the sample of mRNA, or reverse-transcribing the sample of mRNA to cDNA and (ii) amplifying the reverse-transcribed cDNA using primers for a group of PML-RARα gene fusion variants comprising at least two of a P3R3 gene fusion, a P6aR3 gene fusion, and a P6bR3 gene fusion, and (b) detecting the amplified cDNA or detecting and quantitating the amplified cDNA. The primers comprise a primer that hybridizes to exon R3 of RARα. The primers can comprise a primer that hybridizes to exon P3 of PML and a primer that hybridizes to exon 6a of PML. The primer that hybridizes to exon 6a of PML can amplify reverse-transcribed cDNA from a PML-RARα gene fusion variant comprising a P6aR3 gene fusion and reverse-transcribed cDNA from a PML-RARα gene fusion variant comprising a P6bR3 gene fusion. One or more primers is/are detectably labeled. Detection of the one or more detectably labeled primers can be distinguished. Detecting the amplified cDNA can comprise contacting the amplified cDNA with at least one probe under hybridizing conditions and detecting hybridization of at least one probe to the amplified cDNA. At least one probe is a probe that hybridizes to a nucleotide sequence at or near a junction of a PML-RARα gene fusion comprising the 3′ end of exon P3 of PML and the 5′ end of exon R3 of RARα, a nucleotide sequence at or near a junction of a PML-RARα gene fusion comprising the 3′ end of exon P6a of PML and the 5′ end of exon R3 of RARα, or a nucleotide sequence at or near a junction of a PML-RARα gene fusion comprising the 3′ end of exon P6b of PML and the 5′ end of exon R3 of RARα. Detecting the amplified cDNA can comprise contacting the amplified cDNA with at least two probes, wherein hybridization of each probe to the amplified cDNA is distinguishable. In the method (a) can further comprise using primers for at least one IC gene, wherein the primers optionally hybridize to different exons of the at least one IC gene, and (b) can further comprise detecting the amplified IC cDNA. One or more IC primers can be detectably labeled. Detection of the one or more detectably labeled IC primers can be distinguished. Detecting the amplified IC cDNA can comprise contacting the amplified IC cDNA with at least one IC probe and/or at least one IC primer under hybridizing conditions and detecting hybridization of at least one IC probe/primer to the amplified IC cDNA. Detecting the amplified IC cDNA can comprise contacting the amplified IC cDNA with at least two IC probes/primers, wherein each IC probe/primer is specific for a different IC cDNA and hybridization of each IC probe/primer to the amplified IC cDNA is distinguishable.

In view of the above and the “EXAMPLES” herein, the above methods can be used to analyze other gene fusion variants. See, for example, the almost 9,000 fusions reported in COSMIC (Catalogue of Somatic Mutations in Cancer; cancer.sanger.ac.uk/cosmic/fusion), for which breakpoints have been observed or inferred (portions of mRNA present are set forth in HGVS (Human Genome Variation Society) format on COSMIC).

For example, APSCR1 (alveolar soft part sarcoma chromosome region, candidate 1; gene ID ENSG00000169696; transcript ID ENST00000306739) reportedly forms gene fusions with TFE3 (transcription factor binding to IGHM enhancer 3; NCBI (National Center for Biotechnology Information) Acc. No. NM_{—}006521.3 (cDNA); [SEQ ID NO:155]; exons: 1-354, 355-468, 469-772, 773-1018, 1019-1123, 1124-1241, 1242-1298, 1299-1374, 1375-1522, and 1523-3393). Breakpoints have been inferred as follows: 1_{—}1030 ASPSCR1 and 1031_{—}1841 TFE3, 1_{—}1030 ASPSCR1 and 1019_{—}3431 TFE3, and 1_{—}1030 ASPSCR1 and 1124_{—}3431 TFE3 (NM_{—}006521.3; [SEQ ID NO:155]).

ALK (anaplastic lymphoma receptor tyrosine kinase; NCBI Acc. No. NM_{—}004304.4; [SEQ ID NO:39]) reportedly forms gene fusions with C2orf44 (chromosome 2 open reading frame 44; transcript ID ENST00000295148), CARS (cysteinyl-tRNA synthetase; transcript ID ENST00000278224), NPM1 (nucleophosmin (nucleolar phosphoprotein B23, numatrin); NCBI Acc. No. NM_{—}002520.6; [SEQ ID NO:156]; exons: 1-303, 304-383, 384-503, 504-597, 598-704, 705-769, 770-827, 828-914, 915-1016, 1017-1091, and 1092-1449), RANBP2 (RAN binding protein 2; NCBI Acc. No. NM_{—}006267.4; [SEQ ID NO:157]; exons: 1-198, 199-266, 267-378, 379-531, 532-762, 763-908, 909-1101, 1102-1189, 1190-1399, 1400-1581, 1582-1757, 1758-1881, 1882-2043, 2044-2181, 2182-2328, 2329-2508, 2509-2592, 2593-2728, 2729-2823, 2824-7975, 7976-8146, 8147-8239, 8240-8418, 8419-8623, 8624-8725, 8726-8886, 8887-9160, 9161-9495, and 9496-11711), and TPM3 (tropomyosin 3; NCBI Acc. No. NM_{—}153649.3; [SEQ ID NO:158]; exons: 1-262, 263-396, 397-514, 515-585, 586-661, 662-724, 725-794, and 795-3212). When ALK fuses with C2orf44, the inferred breakpoint is 1_{—}3817+654 C2orf44 and 4080-117_{—}6222 ALK, whereas when ALK fuses with CARS, the inferred breakpoint is 1_{—}1893+1117 CARS and 4080-229_{—}6222 ALK, when ALK fuses with NPM1, the inferred breakpoint is 1_{—}448 NPM1 and 4080_{—}6222 ALK, when ALK fuses with RANBP2, the inferred breakpoint is 1_{—}2728 RANBP2 and 4080_{—}6222 ALK, and when ALK fuses with TPM3, the inferred breakpoint is 1_{—}794 TPM3 and 4080_{—}6222 ALK. In this regard, we point out that TPM3 reportedly also forms a gene fusion with NTRK1 (neurotrophic tyrosine kinase, receptor, type 1; transcript ID ENST00000392302), for which the inferred breakpoint is 1_{—}794 TPM3 and 1269_{—}2609 NTRK1.

In addition to TPM3, NTRK1 (neurotrophic tyrosine kinase, receptor, type 1; transcript ID ENST00000392302) also reportedly forms a gene fusion with TPR (transcript ID ENST00000367578). The inferred breakpoint is 1_{—}3073 TPR and 1262_{—}2609 NTRK1.

SS18 (synovial sarcoma translocation, chromosome 18; transcript ID ENST00000269138) reportedly forms gene fusions with SSX1 (synovial sarcoma, X breakpoint 1; NCBI Acc. No. NM_{—}005635.3; [SEQ ID NO:159]; exons: 1-116, 117-205, 206-320, 321-416, 417-466, 467-602, 603-707, and 708-1298; breakpoints for translocation to form the SSXT-SSX1 fusion protein: 320-325 and 464-469) and SSX2 (synovial sarcoma, X breakpoint 2; NCBI Acc. No. NM_{—}003147.5; [SEQ ID NO:160]; exons: 1-116, 117-205, 206-320, 321-416, 417-466, 467-602, 603-748, 749-853, and 854-1476). When SS18 fuses with SSX1, the inferred breakpoints are 1_{—}1308 SS18 and 422_{—}1271 SSX1 and 1_{—}1308 SS18 and 422-1104_{—}1271 SSX1. When SS18 fuses with SSX2, the inferred breakpoint is 1_{—}1308 SS18 and 439_{—}1466 SSX2.

COL1A1 (collagen, type 1, alpha 1; transcript ID ENST00000225964) reportedly forms gene fusions with PDGFB (platelet-derived growth factor beta polypeptide; NCBI Acc. No. NM_{—}002608.2; [SEQ ID NO:161]; exons: 1-1052, 1053-1149, 1150-1239, 1240-1445, 1446-1590, 1591-1743, and 1744-3377). Breakpoints have been inferred as follows: 1_{—}768 COL1A1 and 1086_{—}3373 PDGFB, 1_{—}1182 COL1A1 and 1086_{—}3373 PDGFB, 1_{—}1740 COL1A1 and 1086_{—}3373 PDGFB, 1_{—}1893 COL1A1 and 1086_{—}3373 PDGFB, 1_{—}2739 COL1A1 and 1086_{—}3373 PDGFB, 1_{—}2955 COL1A1 and 1086_{—}3373 PDGFB, 1_{—}3171 COL1A1 and 1086_{—}3373 PDGFB, 1_{—}3333 COL1A1 and 1086_{—}3373 PDGFB, and 1_{—}3549 COL1A1 and 1086_{—}3373 PDGFB.

FUS (fused in sarcoma; NCBI Acc. No. NM_{—}004960.2) reportedly forms gene fusions with CREB3L2 (cAMP responsive element binding protein 3-like 2; NCBI Acc. No. NM_{—}194071.3; [SEQ ID NO:162]; exons: 1-498, 499-715, 716-891, 892-979, 980-1164, 1165-1311, 1312-1370, 1371-1439, 1440-1539, 1540-1666, 1667-1883, and 1884-7456), DDIT3 (DNA-damage-inducible transcript 3; NCBI Acc. No. NM_{—}004083.5; [SEQ ID NO:163]; exons: 1-100, 101-148, and 149-318), and ERG (v-ets erythroblastosis virus #26 oncogene homolog (avian); NCBI Acc. No. NM_{—}004449.4; [SEQ ID NO:164]; exons: 1-123, 124-225, 226-311, 312-529, 530-681, 682-885, 886-996, 967-1035, 1036-1092, 1093-1140, and 1141-5037). Breakpoints have been inferred as shown in Tables Ia and Ib.

TABLE Ia
Inferred Breakpoints for FUS Gene Fusion
Variants by Common 5′ Gene Fragment

| 5′ Gene | 3′ Gene |
|---|---|
| FUS 1_606 | CREB3L2 1015_7455 |
| | DDIT3 101_927 |
| FUS 1_704 | CREB3L2 1060_7455 |
| CREB3L2 1_1063 | FUS 710_2012 |
| FUS 1_882 | DDIT3 101_927 |
| | ERG 1055_3097 |

TABLE 1b
Inferred Breakpoints for FUS Gene Fusion
Variants by Common 3′ Gene Fragment

| 5′ Gene | 3′ Gene |
|---|---|
| FUS 1_606 | CREB3L2 1015_7455 |
| FUS1_606 | DDIT3 101_927 |
| FUS 1_882 | |
| FUS 1_704 | CREB3L2 1060_7455 |
| CREB3L2 1_1063 | FUS710_2012 |
| FUS 1_882 | ERG 1055_3097 |

EWSR1 (Ewing sarcoma breakpoint region 1; NCBI Acc. No. NM_{—}005243.2; [SEQ ID NO:165]; exons: 1-322, 323-359, 360-411, 412-535, 536-722, 723-890, 891-1102, 1103-1283, 1284-1321, 1322-1354, 1355-1473, 1474-1603, 1604-1726, 1727-1889, 1890-1987, and 1988-2240) reportedly forms gene fusions with CREB1 (cAMP responsive element binding protein 1; transcript ID ENST00000236996), FLI1 (Friend leukemia virus integration 1; NCBI Acc. No. NM_{—}002017.2; [SEQ ID NO:166]; exons: 8-190, 191-402, 403-557, 558-761, 762-827, 828-893, 894-953, 954-1001, and 1002-2945), ERG (v-ets erythroblastosis virus E26 oncogene homolog (avian); NCBI Acc. No. NM_{—}004449.4; [SEQ ID NO:164]), WT1 (Wilms tumor 1; NCBI Acc. No. NM_{—}024426.3; [SEQ ID NO:167]; exons: 1-842, 843-965, 966-1068, 1069-1146, 1147-1197, 1198-1294, 1295-1445, 1446-1535, and 1536-1628), FEV (ETS oncogene family; NCBI Acc. No. NM_{—}017521.2; [SEQ ID NO:168]; exons: 1-634, 635-709, and 710-1879), and NR4A3 (nuclear receptor subfamily 4, group A member 3; NCBI Acc. No. NM_{—}006981.2; [SEQ ID NO:169]; exons: 1-553, 554-727, 728-1680, 1681-1810, 1811-1983, 1984-2183, 2184-2362, and 2363-5634). In this regard, we note that NR4A3 reportedly also forms a gene fusion with TAF15 (RNA polymerase II, TATA box binding protein (TBP)-associated factor; Transcript No. ENST00000311979). Breakpoints have been inferred as shown in Tables Ic (which includes TAF15:NR4A3 for each of reference) and Id.

TABLE Ic
Inferred Breakpoints for EWSR1 Gene Fusion
Variants by Common 5′ Gene Fragment

| 5′ Gene | 3′ Gene |
|---|---|
| EWSR1 1_836 | CREB1 729_2986 |
| | FLI1 828_2957 |
| | FLI1 762_2957 |
| | ERG 950_3097 |
| | WT1 1436_3030 |
| EWSR1 1_1088 | FLI1 762_2957 |
| | FLI1 828_2957 |
| | FEV 635_1901 |
| | WT1 1436_3030 |
| EWSR1 1_1017 | FLI1 828_2957 |
| EWSR1 1_1337 | NR4A3 728_5635 |
| TAF15 1_570 | |

TABLE Id
Inferred Breakpoints for EWSR1 Gene Fusion
Variants by Common 3′ Gene Fragment

| 5′ Gene | 3′ Gene |
|---|---|
| EWSR1 1_836 | CREB1 729_2986 |
| EWSR1 1_836 | FLI1 828_2957 |
| EWSR1 1_1088 | |
| EWSR1 1_1017 | |
| EWSR1 1_836 | FLI1 762_2957 |
| EWSR1 1_1088 | |
| EWSR1 1_836 | ERG 950_3097 |
| EWSR1 1_836 | WT1 1436_3030 |
| EWSR1 1_1088 | |
| EWSR1 1_1088 | FEV 635_1901 |
| EWSR1 1_1377 | NR4A3 728_5635 |

KIAA1549 (transcript ID ENST00000242365) reportedly forms gene fusions with BRAF (v-raf murine sarcoma viral oncogene homolog B1; NCBI Acc. No. NM_{—}004333.4; [SEQ ID NO:170]; exons: 1-199, 200-301, 302-565, 566-669, 670-772, 773-921, 922-1041, 1042-1201, 1202-1238, 1239-1375, 1376-1493, 1494-1578, 1579-1755, 1756-1802, 1803-1921, 1922-2053, 2054-2188, and 2189-2947). The inferred breakpoints are shown in Table Ie.

TABLE Ie
Inferred Breakpoints for KIAA1549 Gene Fusion
Variants by Common 5′ Gene Fragment

| 5′ Gene | 3′ Gene |
|---|---|
| KIAA1549 1_5446 | BRAF 1202_2513 |
| | BRAF 1376_2513 |
| KIAA 1_5128 | BRAF 1202_2513 |

TMPRSS2 (transmembrane protease, serine 2; NCBI Acc. No. NM_{—}005656.3; [SEQ ID NO:171]; exons: 1-78, 79-149, 150-372, 373-459, 460-579, 580-706, 707-817, 818-861, 862-1033, 1034-1209, 1210-1305, 1306-1448, 1449-1601, and 1602-3204) reportedly forms gene fusions with ERG (v-ets erythroblastosis virus E26 oncogene homolog (avian); NCBI Acc. No. NM_{—}004449.4; [SEQ ID NO:164]). The inferred breakpoints are shown in Table If.

TABLE If
Inferred Breakpoints for TMPRSS2 Gene Fusion
Variants by Common 5′ Gene Fragment

| 5′ Gene | 3′ Gene |
|---|---|
| TMPRSS2 1_71 | ERG 38_3097 |
| | ERG 226_3097 |
| TMPRSS2 1_142 | ERG 226_3097 |
| | ERG 444_3097 |

Any suitable sample of a tissue or a body fluid can be used as the source of the sample of nucleic acid, i.e., mRNA. Since BCR-ABL gene fusion variants typically are found in people with chronic myelogenous leukemia (CML) and in some people with acute lymphoblastic leukemia (ALL), typically, the source is blood (or cellular components thereof, such as white blood cells). Since PML-RARα gene fusion variants are typically found in people with acute promyelocytic leukemia (APL), typically the source is also blood (or cellular components thereof, such as white blood cells). Bone marrow also can be used as a source of the sample nucleic acid, i.e., mRNA, for BCR-ABL and PML-RARα testing but, given that sampling bone marrow is a very invasive procedure, blood and components thereof are generally preferred. In the event that BCR-ABL and/or PML-RARα gene fusions are determined to be diagnostic/prognostic of other diseases, disorders or conditions, it is possible that other samples can be used as the source of the sample of nucleic acid, i.e., mRNA. Examples of such samples include, but are not limited to, tumor biopsies, touch preparations, and fine-needle aspirates. The biological sample can be preserved, such as by the addition of a chelating agent, e.g., ethylene diamine tetraacetic acid (EDTA) or a salt thereof, such as a disodium salt or a calcium disodium salt. A proteinase, such as proteinase K, can be added to the sample to digest unwanted proteins.

Since EML4-ALK and KIF5B-RET fusion variants are typically found in people with solid tumors (e.g., lung cancer, breast cancer, and colorectal cancer for EML4-ALK and lung cancer for KIF5B-RET), typically the source of nucleic acid, i.e., mRNA, is resected cancer tumor, biopsied tumor, and/or fine needle aspirates. The biological sample can be preserved, such as by formalin-fixed, paraffin-embedding (FFPE).

The sample may be prepared for assay using any suitable method as is known in the art. Desirably, the method extracts and concentrates nucleic acids, in particular mRNA. The method also desirably makes the nucleic acid, i.e., mRNA, accessible for reverse transcription and amplification, and removes potential inhibitors of reverse transcription and amplification from the extract.

RNA can be isolated from peripheral blood using, for example, an RNeasy RNA isolation kit from Qiagen Inc. (Valencia, Calif.). RNA can be isolated from FFPE speciments, for example using an RNeasy FFPE isolation kit from Qiagen (Valencia, Calif.). Any other RNA extraction and purification technique also can be used, including liquid-liquid and solid-phase techniques ranging from chemical extraction to automated magnetic bead nucleic acid capture systems. RNA can be isolated and reverse-transcribed and the resulting cDNA can be amplified (e.g., reverse-transcription polymerase chain reaction (RT-PCR) as described in U.S. Pat. Nos. 5,310,652; 5,322,770; 5,561,058; 5,641,864; and 5,693,517, for example).

Once nucleic acid has been obtained and, if necessary, reverse-transcribed (e.g., mRNA to cDNA), it can be contacted with primers that result in specific amplification of a specific gene fusion variant, if the specific gene fusion variant is present in the sample. “Specific amplification” means that the primers amplify specific gene fusion variant(s) of interest and not other gene fusion variants. See, e.g., PCR Technology: Principles and Applications for DNA Amplification (Erlich, Editor, Freeman Press, NY (1992)); PCR Protocols: A Guide to Methods and Applications (Innis et al., Editors, Academic Press, San Diego, Calif. (1990)); Current Protocols in Molecular Biology (Ausubel, 1994-1999, including supplemental updates through April 2004); and Molecular Cloning: A Laboratory Manual (Sambrook & Russell, 3rd ed., 2001). Various other amplification-based methods or extension-based methods are described in Intl Pat. App. Pub. No. WO 93/22456 and U.S. Pat. Nos. 4,851,331; 5,137,806; 5,595,890; and 5,639,611, all of which are specifically incorporated herein by reference for their teachings regarding same. While methods such as ligase chain reaction, strand displacement assay, and various transcription-based amplification methods can be used (see, e.g., review by Abramson and Myers, Current Opinion in Biotechnology 4:41-47 (1993)), PCR is preferred.

Primers for two or more gene fusion variants can be employed simultaneously in a single amplification reaction. Amplification products can be distinguished by different labels or size (e.g., using gel electrophoresis).

A primer can be detectably labeled with a label that can be detected by spectroscopic, photochemical, biochemical, immunochemical or chemical means, for example (see, e.g., Sambrook et al.). Useful labels include a dye, such as a fluorescent dye, a radioactive label, such as ³²P, an electron-dense reagent, an enzyme, such as peroxidase or alkaline phosphatase, biotin, or haptens and proteins for which antisera or monoclonal antibodies are available.

A probe can be similarly labeled, such as with fluorescein. In this regard, if the primer is labeled with a dye and the detectable oligonucleotide is labeled with fluorescein and is designed to bind to the nascent strand opposite from the dye, fluorescence resonance energy transfer (FRET) across the DNA helix can occur.

Any suitable sequence can be used as the IC. Examples of IC genes are set forth in the “EXAMPLES” herein, namely, β-glucuronidase (GUSB), ABL, and glucose-6-phosphate dehydrogenase (G6PD).

Nucleic acid amplification reagents, such as for RT-PCR in the same well, include an enzyme having polymerase activity (e.g., rTth), one or more enzyme co-factors (e.g., MnCl₂), and deoxyribonucleotide triphosphates (dNTPs; e.g., dATP, dGTP, dCTP, and dTTP). Preferred concentrations of nucleic acid amplification reagents are exemplified herein.

Conditions that promote amplification are those that promote annealing of primers and extension of nucleic acid sequences. Annealing is dependent on various parameters, such as temperature, ionic strength, length of sequences being amplified, complementarity, and G:C content of the sequences being amplified. For example, lowering the temperature promotes annealing of complementary nucleic acid sequences. High G:C content and longer length stabilize duplex formation. Generally, primers and detectable oligonucleotides of about 30 bp or less and having a high G:C content work well. Preferred amplification conditions, primers and detectable oligonucleotides are exemplified herein.

Amplification can be repeated any suitable number of times by thermal cycling the reaction mixture between about 10 and about 100 times, such as between about 20 and about 75 times, such as between about 25 and about 50 times.

Once the amplification reactions are completed, the presence of an amplified product can be detected using any suitable method. Such methods include, without limitation, those known in the art, such as gel electrophoresis with or without a fluorescent dye (depending on whether the product was amplified with a dye-labeled primer), a melting profile with an intercalating dye (see, e.g., PCR Technology, Principles, and Applications for DNA Amplification, Erlich, Ed., W. H. Freeman and Co., New York, 1992, Chapter 7), and hybridization with an internal probe. Other examples of methods include enzyme-linked immunosorbent assay (ELISA), electro-chemiluminescence, reverse dot blots, high pressure liquid chromatography (HPLC) (see, e.g., Lazar, Genome Res. 4: S1-S14 (1994)), and single-strand conformation polymorphism analysis of single-stranded PCR products also can be used (see, e.g., Orita et al., PNAS USA 86: 2766-2770 (1989)).

Amplified nucleic acid can be detected by monitoring an increase in the total amount of double-stranded DNA (dsDNA) in the reaction mixture (see, e.g., U.S. Pat. No. 5,994,056 and European Pat. Pub. Nos. 487,218 and 512,334). A DNA-binding dye, such as SYBR Green, is used. The dye fluoresces when bound to dsDNA, and the increase in fluorescence is used to determine the increase in dsDNA.

Dideoxy sequencing-based methods and Pyrosequencing of oligonucleotide-length products also can be used to detect amplified nucleic acid. Another sequencing method is described by Kobayashi et al., Mol. Cell. Probes 9: 175-182 (1995)).

When PCR is used, conditions, such as those exemplified in the EXAMPLES herein, can be used. When standard PCR is used, detection can occur after amplification is complete, such as after using a labeled primer during amplification, by using a labeled primer as a probe after amplification, or by using a probe, which differs in sequence from the primers, after amplification to hybridize to the amplified target sequence. Labeled amplification products then can be separated and detected by other means.

Alternatively, the amplification and detection can be combined in a real-time PCR assay. When real-time PCR is used, the mixture can further comprise nucleic acid detection reagents, such as a non-specific fluorescent dye that intercalates with any double-stranded DNA, for example, or a sequence-specific DNA probe, which permits detection only after the probe hybridizes with its complementary DNA target, thereby enabling simultaneous amplification and detection. When a probe is present in the mixture during amplification, the probe should be stable under the conditions that promote amplification, should not interfere with amplification, should bind to its target sequence under amplification conditions, and emit a signal only upon binding its target sequence. Examples of probes that are particularly well-suited in this regard include molecular beacon probes, TAQMAN® probes, and linear probes, such as those described by Abravaya et al. (U.S. Pat. App. Pub. No. 2005/0227257). The probes can form the loop region, alone or in further combination with part of the stem region, of a molecular beacon. The probes also can be used as linear probes with a fluorophore (e.g., FAM) at one end and a high-efficiency quencher, such as the Black Hole Quencher (BHQ®; BioSearch Technologies, Inc., Novato, Calif.), at the other end.

The detection of an amplified product indicates that cells containing a specific gene fusion variant or variants (depending on whether or not two or more gene fusion variants are simultaneously detected) were present in the sample, while the lack of detection of an amplified product indicates that cells containing a specific gene fusion variant were not present in the sample. In this regard, if two or more specific gene fusion variants are amplified at the same time (or one or more specific gene fusion variants and an internal control (IC) gene), a primer for each specific gene fusion variant can be labeled with a distinct detectable label, thereby enabling the detection of two or more specific gene fusion variants (or one or more specific gene fusion variants and an IC gene) to be distinguished. The relative levels of gene fusion variant and IC products can indicate the fraction of cells in the sample that contain a gene fusion variant.

If desired, the method can further comprise an initial universal amplification step. For example, the sample can be contacted with degenerate primers and amplified prior to specific amplification of one or more gene fusion variants, alone or in further combination with an IC sequence.

If desired, the nucleic acid sample or the probe can be immobilized on a solid support. Examples of assay formats utilizing solid supports include dot-blot formats and reverse dot-blot formats (see, e.g., U.S. Pat. Nos. 5,310,893; 5,451,512; 5,468,613; and 5,604,099, all of which are specifically incorporated herein by reference for their teachings regarding same).

Following amplification, it may be desirable to separate the amplification product from the template and the excess primer to determine whether specific amplification occurred. Separation can be effected by agarose, agarose-acrylamide or polyacrylamide gel electrophoresis using standard methodology (see, e.g., Sambrook et al., Molecular Cloning, Fritsch and Maniatis, eds., Cold Spring Harbor Lab. Press, Cold Spring Harbor, N.Y. (1989)). Alternatively, chromatography can be used to effect separation. Examples of type of chromatography include adsorption, partition, ion-exchange and molecular sieve, and examples of types of chromatographic techniques include column, paper, thin-layer and gas chromatography (see, e.g., Freifelder, Physical Biochemistry Applications to Biochemistry and Molecular Biology, 2nd ed., Wm. Freeman & Co., New York, N.Y. (1982)).

Amplification is confirmed by visualization. For example, a gel stained with ethidium bromide can be visualized with UV light. Amplification products labeled with a radioisotope can be visualized by exposing and developing an x-ray film, whereas amplification products labeled with a fluorometric label can be visualized by subjecting the amplification products to stimulating spectra. A preferred method of visualization of amplification is the use of a labeled probe that hybridizes to the amplified products.

A manual column, such as one available from Qiagen, can be used. The use of an automated sample preparation system, such as an automated sample preparation system designed to use magnetic microparticle processes for the purification of nucleic acids, can be preferred. An example of an automated sample preparation system is m2000sp, which is available from Abbott Laboratories, Abbott Park, Ill. Alternatively, samples can be prepared using the m24sp automated sample preparation system (Abbott) or prepared manually, e.g., by using a column-based RNA extraction kit, such as Paxgene, which is available from PreAnalytix/Qiagen.

An unrelated nucleic acid sequence can be used as an internal control (IC) to demonstrate that the process has proceeded correctly for each sample. The IC is detected along with the gene fusion sequence. The IC also can be used to standardize quantitation of a given gene fusion sequence, for example, by comparing the IC signal to the gene fusion signal.

Amplification/detection can be carried out as known in the art, such as by use of the m2000rt instrument (Abbott Molecular Inc., Des Plaines, Ill.). The target nucleic acid is amplified by DNA polymerase in the presence of deoxyribonucleotide triphosphates (dNTPs) and magnesium. The amplification reagent contains specific sets of amplification primers for one or more gene fusion variants and, preferably, an IC gene. During PCR amplification, high temperature is used to separate the strands of double-stranded DNA. When the reaction is cooled to a temperature where DNA annealing can occur, the analyte-specific, single-stranded DNA oligonucleotide primers bind to the analyte DNA. The primers are extended by DNA polymerase, thereby making an exact copy of a short target stretch of the analyte DNA. During each round of thermal cycling, amplification products dissociate to single strands at high temperature, allowing primer annealing and extension as the temperature is lowered. Exponential amplification of the target is achieved through repeated cycling between high and low temperatures. Amplification of the gene fusion variants and, if targeted, the IC gene takes place simultaneously in the same reaction.

The method can be used to determine the gene fusion variant status for the purpose of evaluating treatment options. For example, imatinib, which is marketed by Novartis as Gleevec in the U.S. and Glivec in other countries, reportedly has been shown to improve survival in people with CML (BCR-ABL reciprocal translocation or “Philadelphia chromosome”). Imatinib is also used to treat people with gastrointestinal stromal tumors (GISTs) and other malignancies. Another drug that has been used to treat people with CML is nilotinib. Other kinase inhibitors include bosutinib and dastinib. HHT also has been used for treatment.

The method also can be used to predict outcome for a patient diagnosed with cancer, such as leukemia, to assess risk of metastasis, such as in patients with early stages of disease (stage I/II), and to monitor patients with advanced, metastatic cancer (stage III/IV). Since metastatic spread of cancer often occurs hematogenously, the method also can be used to assay peripheral blood to assess recurrence.

### Primers and Probes

A set of primers is also provided. The set of primers comprises at least two primers selected from the group consisting of a primer, such as a forward primer, that hybridizes to exon e1 of BCR, a primer, such as a forward primer, that hybridizes to exon b2 of BCR, and a primer, such as a forward primer, that hybridizes to exon e19 of BCR. Each primer can be detectably labeled and/or the set of primers can be combined with at least one detectably labeled probe selected from the group consisting of a probe that hybridizes to a nucleotide sequence at or near a junction of a BCR-ABL gene fusion comprising the 3′ end of exon e1 of BCR and the 5′ end of exon a2 of ABL, a probe that hybridizes to a nucleotide sequence at or near a junction of a BCR-ABL gene fusion comprising the 3′ end of exon b2 of BCR and the 5′ end of exon a2 of ABL, a probe that hybridizes to a nucleotide sequence at or near a junction of a BCR-ABL gene fusion comprising the 3′ end of exon b3 of BCR and the 5′ end of exon a2 of ABL, and a probe that hybridizes to a nucleotide sequence at or near a junction of a BCR-ABL gene fusion comprising the 3′ end of exon e19 of BCR and the 5′ end of exon a2 of ABL. The primer, which hybridizes to exon e1 of BCR, can comprise a nucleotide sequence selected from the group consisting of SEQ ID NOS: 1, 18, 19, 20, 21, and 22. The primer, which hybridizes to exon b2 of BCR, can comprise a nucleotide sequence selected from the group consisting of SEQ ID NOS: 2 and 23. The primer, which hybridizes to exon e19 of BCR, can comprise a nucleotide sequence selected from the group consisting of SEQ ID NOS: 3 and 24. The set of primers can further comprise a primer, such as a reverse primer, that hybridizes to exon a2 of ABL. The primer, which hybridizes to exon a2 of ABL, can comprise a nucleotide sequence selected from the group consisting of SEQ ID NOS: 4, 25, 26, and 27. A preferred nucleotide sequence for a primer, in particular a reverse primer, is SEQ ID NO: 4.

Another set of primers is also provided. The set of primers comprises at least two primers selected from the group consisting of a primer that hybridizes to exon E13 of EML4, a primer that hybridizes to exon E6a of EML4, a primer that hybridizes to exon Ebb of EML4, a primer that hybridizes to exon E14 of EML4, and a primer that hybridizes to exon E20 of EML4, wherein the primer can be detectably labeled and/or wherein the set of primers can be combined with at least one detectably labeled probe selected from the group consisting of a probe that hybridizes to a nucleotide sequence at or near a junction of a EML4-ALK gene fusion comprising the 3′ end of exon E13 of EML4 and the 5′ end of exon A20 of ALK, a probe that hybridizes to a nucleotide sequence at or near a junction of a EML4-ALK gene fusion comprising the 3′ end of exon E6a of EML4 and the 5′ end of exon A20 of ALK, a probe that hybridizes to a nucleotide sequence at or near a junction of a EML4-ALK gene fusion comprising the 3′ end of exon Ebb of EML4 and the 5′ end of exon A20 of ALK, a probe that hybridizes to a nucleotide sequence at or near a junction of a EML4-ALK gene fusion comprising the 3′ end of exon E14 of EML4 and the 5′ end of exon A20 of ALK, and a probe that hybridizes to a nucleotide sequence at or near a junction of a EML4-AKL gene fusion comprising the 3′ end of exon E20 of EML4 and the 5′ end of exon A20 of ALK. The set of primers can further comprise a primer that hybridizes to exon A20 of ALK.

Yet another set of primers is provided. The set of primers comprises at least two primers selected from the group consisting of a primer that hybridizes to exon K15 of KIF5B, a primer that hybridizes to exon K16 of KIF5B, a primer that hybridizes to exon K22 of KIF5B, and a primer that hybridizes to exon K23 of KIF5B, wherein the primer can be detectably labeled and/or wherein the set of primers can be combined with at least one detectably labeled probe selected from the group consisting of a probe that hybridizes to a nucleotide sequence at or near a junction of a KIF5B-RET gene fusion comprising the 3′ end of exon K15 of KIF5B and the 5′ end of exon R12 of RET, a probe that hybridizes to a nucleotide sequence at or near a junction of a KIF5B-RET gene fusion comprising the 3′ end of exon K16 of KIF5B and the 5′ end of exon R12 of RET, a probe that hybridizes to a nucleotide sequence at or near a junction of a KIF5B-RET gene fusion comprising the 3′ end of exon K22 of KIF5B and the 5′ end of exon R12 of RET, and a probe that hybridizes to a nucleotide sequence at or near a junction of a KIF5B-RET gene fusion comprising the 3′ end of exon K23 of KIF5B and the 5′ end of exon R12 of RET. The set of primer can further comprise a primer that hybridizes to exon R12 of RET.

Still yet another set of primers is provided. The set of primers comprises at least two primers selected from the group consisting of a primer that hybridizes to exon P3 of PML, a primer that hybridizes to exon 6a of PML, and a primer that hybridizes to exon 6b of PML, wherein the primer can be detectably labeled and/or wherein the set of primers can be combined with at least one detectably labeled probe selected from the group consisting of a probe that hybridizes to a nucleotide sequence at or near a junction of a PML-RARα gene fusion comprising the 3′ end of exon P3 of PML and the 5′ end of exon R3 of RARα, a probe that hybridizes to a nucleotide sequence at or near a junction of a PML-RARα gene fusion comprising the 3′ end of exon P6a of PML and the 5′ end of exon R3 of RARα, and a probe that hybridizes to a nucleotide sequence at or near a junction of a PML-RARα gene fusion comprising the 3′ end of exon P6b of PML and the 5′ end of exon R3 of RARα. The set of primers can further comprise a primer that hybridizes to exon R3 of RARα.

Further provided is a set of probes. The set of probes comprises a probe that hybridizes to a nucleotide sequence at or near a junction of a BCR-ABL gene fusion comprising the 3′ end of exon e1 of BCR and the 5′ end of exon a2 of ABL, a probe that hybridizes to a nucleotide sequence at or near a junction of a BCR-ABL gene fusion comprising the 3′ end of exon b2 of BCR and the 5′ end of exon a2 of ABL, a probe that hybridizes to a nucleotide sequence at or near a junction of a BCR-ABL gene fusion comprising the 3′ end of exon b3 of BCR and the 5′ end of exon a2 of ABL, and a probe that hybridizes to a nucleotide sequence at or near a junction of a BCR-ABL gene fusion comprising the 3′ end of exon e19 of BCR and the 5′ end of exon a2 of ABL. The probe, which hybridizes to a nucleotide sequence at or near a junction of a BCR-ABL gene fusion comprising the 3′ end of exon e1 of BCR and the 5′ end of exon a2 of ABL, can comprise a nucleotide sequence selected from the group consisting of SEQ ID NOS: 5, 28, 29, 30, and a sequence complementary thereto. The probe, which hybridizes to a nucleotide sequence at or near a junction of a BCR-ABL gene fusion comprising the 3′ end of exon b2 of BCR and the 5′ end of exon a2 of ABL, can comprise a nucleotide sequence selected from the group consisting of SEQ ID NOS: 6, 31, 32, and a sequence complementary thereto. The probe, which hybridizes to a nucleotide sequence at or near a junction of a BCR-ABL gene fusion comprising the 3′ end of exon b3 of BCR and the 5′ end of exon a2 of ABL, can comprise a nucleotide sequence selected from the group consisting of SEQ ID NOS: 7, 33, 34, 35, and a sequence complementary thereto. The probe, which hybridizes to a nucleotide sequence at or near a junction of a BCR-ABL gene fusion comprising the 3′ end of exon e19 of BCR and the 5′ end of exon a2 of ABL, can comprise a nucleotide sequence selected from the group consisting of SEQ ID NOS: 8, 36, and a sequence complementary thereto.

Another set of probes is provided. The set of probes comprises at least two probes selected from the group consisting of a probe that hybridizes to a nucleotide sequence at or near a junction of a EML4-ALK gene fusion comprising the 3′ end of exon E13 of EML4 and the 5′ end of exon A20 of ALK, a nucleotide sequence at or near a junction of a EML4-ALK gene fusion comprising the 3′ end of exon E6a of EML4 and the 5′ end of exon A20 of ALK, a nucleotide sequence at or near a junction of a EML4-ALK gene fusion comprising the 3′ end of exon Ebb of EML4 and the 5′ end of exon A20 of ALK, a nucleotide sequence at or near a junction of a EML4-ALK gene fusion comprising the 3′ end of exon E14 of EML4 and the 5′ end of exon A20 of ALK, and a nucleotide sequence at or near a junction of a EML4-AKL gene fusion comprising the 3′ end of exon E20 of EML4 and the 5′ end of exon A20 of ALK.

Yet another set of probes is provided. The set of probes comprises at least two probes selected from the group consisting of a probe that hybridizes to a nucleotide sequence at or near a junction of a KIF5B-RET gene fusion comprising the 3′ end of exon K15 of KIF5B and the 5′ end of exon R12 of RET, a nucleotide sequence at or near a junction of a KIF5B-RET gene fusion comprising the 3′ end of exon K16 of KIF5B and the 5′ end of exon R12 of RET, a nucleotide sequence at or near a junction of a KIF5B-RET gene fusion comprising the 3′ end of exon K22 of KIF5B and the 5′ end of exon R12 of RET, and a nucleotide sequence at or near a junction of a KIF5B-RET gene fusion comprising the 3′ end of exon K23 of KIF5B and the 5′ end of exon R12 of RET.

Still yet another set of probes is provided. The set of probes comprises at least two probes selected from the group consisting of a probe that hybridizes to a nucleotide sequence at or near a junction of a PML-RARα gene fusion comprising the 3′ end of exon P3 of PML and the 5′ end of exon R3 of RARα, a nucleotide sequence at or near a junction of a PML-RARα gene fusion comprising the 3′ end of exon P6a of PML and the 5′ end of exon R3 of RARα, and a nucleotide sequence at or near a junction of a PML-RARα gene fusion comprising the 3′ end of exon P6b of PML and the 5′ end of exon R3 of RARα.

Oligonucleotides can be prepared by any suitable method, usually chemical synthesis (e.g., solid-phase synthesis) employing commercially available reagents and instruments (see, e.g., Applied Biosystems, Inc. (Foster City, Calif.), DuPont (Wilmington, Del.), and Milligen (Bedford, Mass.)). Alternatively, they can be purchased through commercial sources. Methods of synthesizing oligonucleotides are well-known in the art (see, e.g., Narang et al., Meth. Enzymol. 68: 90-99 (1979); Brown et al., Meth. Enzymol. 68: 109-151 (1979); Beaucage et al., Tetrahedron Lett. 22: 1859-1862 (1981); and U.S. Pat. No. 4,458,066).

The ability to carry out the method in a closed-tube, homogeneous format minimizes the risk of contamination (see, e.g., Kreuzer et al., Ann. Hematol. 82: 284-289 (2003)). The sample can be contacted with primers by any means routinely applied for contacting a sample with a pair of PCR primers. For example, the sample and the primers can be contacted in a microwell plate or in a microvial adapted for the mixture of small volumes.

Also provided are diagnostic real-time PCR (rtPCR) methods that use the aforementioned primers and probes to amplify and detect BCR-ABL gene fusion variants in separate reactions or a pooled reaction.

Primers and probes that are at least about 80% identical with the primers and probes described herein also can be used. With regard to primers, preferably, even desirably, the last ten bases are at least about 75% identical with the primers described herein. If desired, one or both primers (i.e., forward and reverse primers) can be tagged or labeled. Use of labeled primers results in labeled amplification products. Fluorescently labeled amplification products can be detected using any suitable equipment designed to detect fluorescence, such as the ABI 310 Genetic Analyzer and Genescan 3.1.2 software (Applied Biosystems), for example.

If desired, the primers described above can be modified so that they no longer act as primers for DNA synthesis and can be labeled and used as detectable oligonucleotides. The probes can be used in different assay formats. For example, the probes can be used in a 5′-nuclease assay (see, e.g., U.S. Pat. Nos. 5,210,015; 5,487,972; and 5,804,375; and Holland et al., PNAS USA 88: 7276-7280 (1988), all of which are specifically incorporated by reference for their teachings regarding same).

While the primers and probes have been described herein in the context of their use in nucleic acid-based amplification methods, such as PCR, in particular real-time PCR, such primers and probes can be useful as probes in other nucleic acid-based methods, such as hybridization techniques (e.g., membrane-based hybridization techniques (Southern blots and Northern blots), modified nucleic acid hybridization techniques (see, e.g., Pandian et al., U.S. Pat. No. 5,627,030), and enzyme-linked immunoadsorbent assay (ELISA)-like techniques), which are used to detect identical, similar and complementary polynucleotide sequences.

The probes, which are single-stranded, linear DNA oligonucleotides, are detectably labeled in accordance with methods known in the art. Alternatively, primers can be similarly labeled, if desired. Any suitable label, such as a fluorophore, a luminophore, a chemiluminophore, a photoluminophore, or a radioisotope, can be used. For example, a fluorescent moiety can be covalently linked to one end of the probe and a quenching moiety can be covalently linked to the other end. Examples of suitable fluorophores include, but are not limited to, FAM (e.g., 6′-FAM), fluorescein and derivatives thereof, rhodamine, coumarin and derivatives thereof, TET, HEX, JOE, TAMA, TAMRA, NTB, ROX, VIC, NED, 4,7-dichloro-fluorescein, 4,7-dichloro-rhodamine, DABCYL, DABSYL, malachite green, LC-Red 610, LC-Red 640, LC-Red 670, LC-Red 705, Lucifer yellow, TEXAS RED®, tetramethylrhodamine, tetrachloro-6-carboxyfluoroscein, 5-carboxyrhodamine, and cyanine dyes (e.g., Cy3 and Cy5) and derivatives thereof. FAM is a preferred label. Examples of quenchers include DABCYL, DABSYL, DABMI, tetramethylrhodamine, TAMRA, MGB, BHQ®, and BHQplus. MGB can be a preferred quencher. As indicated above, during each round of real-time PCR amplification, the detectably labeled probes anneal to the amplified BCR-ABL gene fusion variant DNA (i.e., target sequence), if present. In the absence of a target sequence, each of the probes adopts a conformation that brings the quencher close enough to the excited fluorophore to absorb its energy before it can be fluorescently emitted. In the presence of a target sequence, each probe binds to its complementary sequence in the target and the fluorophore and the quencher are held apart, allowing fluorescent emission and detection. Preferably, and even desirably, the BCR-ABL gene fusion variant probes and the IC-specific probes are labeled differently so that target DNA and IC DNA can be distinguished.

### Kits

Kits are also provided. One kit comprises:

(i) a set of primers comprising a primer that hybridizes to an exon of a first gene, which becomes contiguous with a variant exon of the second gene, and a primer for each of two or more variant exons of the second gene; and

(ii) instructions for a method of detecting mRNA from fusions of the first gene and the second gene in a sample of mRNA, which method comprises:
(a) (i′) obtaining cDNA, which has been reverse-transcribed from the sample of mRNA, or reverse-transcribing the sample of mRNA to cDNA and (ii′) amplifying the reverse-transcribed cDNA using primers comprising a primer for an exon of the first gene, which becomes contiguous with a variant exon of the second gene, and a primer for each of two or more variant exons of the second gene, wherein one or more of the primers can be detectably labeled, and
(b) detecting the amplified cDNA, and optionally, simultaneously or sequentially quantitating the amplified cDNA. The first gene and the second gene can be any genes that generate fusions. For example, the first gene and the second gene can be the BCR gene and the ABL proto-oncogene, the EML4 gene and the ALK gene, the KIF5B gene and the RET proto-oncogene, or the PML gene and the RARα gene. The kit can further comprise a probe for each gene fusion variant that is not detected using a detectably labeled primer, wherein the probe hybridizes to a nucleotide sequence at or near a junction of a variant of a fusion between the first gene and the second gene comprising the 3′ end of an exon of the first gene and the 5′ end of an exon of the second gene. Step (ii) (a) of the method can further comprise using primers for at least one IC gene, wherein the IC primers hybridize to the same or different exons of the at least one IC gene, and step (ii) (b) can further comprise detecting the amplified IC cDNA.

Another kit comprises:

(i) a set of primers comprising at least two primers, such as forward primers, selected from the group consisting of a primer that hybridizes to exon e1 of BCR, a primer that hybridizes to exon b2 of BCR, and a primer that hybridizes to exon e19 of BCR; and

(ii) instructions for a method of detecting mRNA from fusions of the BCR gene and the ABL proto-oncogene in a sample of mRNA from a human, which method comprises:
(a) (i) obtaining cDNA, which has been reverse-transcribed from the sample of mRNA, or reverse-transcribing the sample of mRNA to cDNA and (ii) amplifying the reverse-transcribed cDNA using primers, i.e., forward and reverse primers, for a group of BCR-ABL gene fusion variants comprising at least two gene fusion variants selected from the group consisting of an e1a2 gene fusion, a b2a2 gene fusion, a b3a2 gene fusion, and an e19a2 gene fusion, wherein one or more of the primers can be detectably labeled, and
(b) detecting the amplified cDNA or detecting and quantitating the amplified cDNA. The kit can comprise a primer, such as a reverse primer, that hybridizes to exon a2 of ABL. The primers effect reverse transcription and amplification of BCR-ABL gene fusion variant mRNA or amplification of BCR-ABL gene fusion variant cDNA. The kit can further comprise a probe for each gene fusion variant that is not detected using a detectably labeled primer, wherein the probe hybridizes to a nucleotide sequence at or near a junction of a BCR-ABL gene fusion comprising the 3′ end of exon e1 of BCR and the 5′ end of exon a2 of ABL, a nucleotide sequence at or near a junction of a BCR-ABL gene fusion comprising the 3′ end of exon b2 of BCR and the 5′ end of exon a2 of ABL, a nucleotide sequence at or near a junction of a BCR-ABL gene fusion comprising the 3′ end of exon b3 of BCR and the 5′ end of exon a2 of ABL, or a nucleotide sequence at or near a junction of a BCR-ABL gene fusion comprising the 3′ end of exon e19 of BCR and the 5′ end of exon a2 of ABL. Step (ii) (a) can further comprise using primers, i.e., forward and reverse primers, for at least one IC gene, wherein the IC primers hybridize to the same or different exons of the at least one IC gene, and step (ii) (b) can further comprise detecting the amplified IC cDNA. The primers effect reverse transcription and amplification of IC mRNA or amplification of IC cDNA.

Yet another kit comprises:

(i) a set of primers comprising a primer, such as a forward primer, comprising a nucleotide sequence selected from the group consisting of SEQ ID NOS: 1, 2, 3, 18, 19, 20, 21, 22, 23, and 24; and

(ii) instructions for a method detecting mRNA from fusions of the BCR gene and the ABL proto-oncogene in a sample of mRNA from a human, which method comprises:
(a) (i) obtaining cDNA, which has been reverse-transcribed from the sample of mRNA, or reverse-transcribing the sample of mRNA to cDNA and (ii) amplifying the reverse-transcribed cDNA using primers, i.e., forward and reverse primers, for at least one BCR-ABL gene fusion variant selected from the group consisting of an e1a2 gene fusion, a b2a2 gene fusion, a b3a2 gene fusion, and an e19a2 gene fusion, wherein one or more of the primers can be detectably labeled, and
(b) detecting the amplified cDNA and optionally, simultaneously or sequentially, quantitating the amplified cDNA. The kit can further comprise a primer, such as a reverse primer, comprising a nucleotide sequence selected from the group consisting of SEQ ID NOS: 4, 25, 26, and 27. SEQ ID NO: 4 is preferred for a primer, in particular a reverse primer. The primers effect reverse transcription and amplification of BCR-ABL gene fusion variant mRNA or amplification of BCR-ABL gene fusion variant cDNA. The kit can further comprise a probe for each gene fusion variant that is not detected using a detectably labeled primer, wherein the probe is selected from the group consisting of SEQ ID NOS: 5, 6, 7, 8, 28, 29, 30, 31, 32, 33, 34, 35, 36, and a sequence complementary thereto. Step (ii) (a) can further comprise using primers, i.e., forward and reverse primers, for at least one IC gene, wherein the IC primers hybridize to the same or different exons of the at least one IC gene, and step (b) can further comprise detecting the amplified IC cDNA. The primers effect reverse transcription and amplification of IC mRNA or amplification of IC cDNA.

Still yet another kit is also provided. The kit comprises:

(i) a set of primers comprising at least two primers selected from the group consisting of a primer that hybridizes to exon E13 of EML4, a primer that hybridizes to exon E6a of EML4, a primer that hybridizes to exon Ebb of EML4, a primer that hybridizes to exon E14 of EML4, and a primer that hybridizes to exon E20 of EML4; and

(ii) instructions for a method of detecting mRNA from fusions of the EML4 gene and the ALK gene in a sample of mRNA from a human, which method comprises:
(a) (i) obtaining cDNA, which has been reverse-transcribed from the sample of mRNA, or reverse-transcribing the sample of mRNA to cDNA and (ii) amplifying the reverse-transcribed cDNA using primers for a group of EML4-ALK gene fusion variants comprising at least two gene fusion variants selected from the group consisting of an E13A20 gene fusion, an E6aA20 gene fusion, an E6bA20 gene fusion, an E14A20 gene fusion, and an E20A20 gene fusion, wherein one or more of the primers can be detectably labeled, and
(b) detecting the amplified cDNA and optionally, simultaneously or sequentially, quantitating the amplified cDNA. The kit can comprise a primer that hybridizes to exon A20 of ALK. The kit can further comprise a probe for each gene fusion variant that is not detected using a detectably labeled primer, wherein the probe hybridizes to a nucleotide sequence at or near a junction of a EML4-ALK gene fusion comprising the 3′ end of exon E13 of EML4 and the 5′ end of exon A20 of ALK, a nucleotide sequence at or near a junction of a EML4-ALK gene fusion comprising the 3′ end of exon E6a of EML4 and the 5′ end of exon A20 of ALK, a nucleotide sequence at or near a junction of a EML4-ALK gene fusion comprising the 3′ end of exon Ebb of EML4 and the 5′ end of exon A20 of ALK, a nucleotide sequence at or near a junction of a EML4-ALK gene fusion comprising the 3′ end of exon E14 of EML4 and the 5′ end of exon A20 of ALK, or a nucleotide sequence at or near a junction of a EML4-AKL gene fusion comprising the 3′ end of exon E20 of EML4 and the 5′ end of exon A20 of ALK. In the kit (ii)(a) can further comprise using primers for at least one IC gene, wherein the primers optionally hybridize to different exons of the at least one IC gene, and (b) can further comprise detecting the amplified IC cDNA.

A further kit is provided. The kit comprises:

(i) a set of primers comprising at least two primers selected from the group consisting of a primer that hybridizes to exon K15 of KIF5B, a primer that hybridizes to exon K16 of KIF5B, a primer that hybridizes to exon K22 of KIF5B, and a primer that hybridizes to exon K23 of KIF5B; and

(ii) instructions for a method of detecting mRNA from fusions of the KIF5B gene and the RET gene in a sample of mRNA from a human, which method comprises:
(a) (i) obtaining cDNA, which has been reverse-transcribed from the sample of mRNA, or reverse-transcribing the sample of mRNA to cDNA and (ii) amplifying the reverse-transcribed cDNA using primers for a group of KIF5B-RET gene fusion variants comprising at least two gene fusion variants selected from the group consisting of a K15R12 gene fusion, a K16R12 gene fusion, a K22R12 gene fusion, and a K23R12 gene fusion, wherein one or more of the primers can be detectably labeled, and
(b) detecting the amplified cDNA and optionally, simultaneously or sequentially, quantitating the amplified cDNA. The kit can comprise a primer that hybridizes to exon R12 of RET. The kit can further comprise a probe for each gene fusion variant that is not detected using a detectably labeled primer, wherein the probe hybridizes to a nucleotide sequence at or near a junction of a KIF5B-RET gene fusion comprising the 3′ end of exon K15 of KIF5B and the 5′ end of exon R12 of RET, a nucleotide sequence at or near a junction of a KIF5B-RET gene fusion comprising the 3′ end of exon K16 of KIF5B and the 5′ end of exon R12 of RET, a nucleotide sequence at or near a junction of a KIF5B-RET gene fusion comprising the 3′ end of exon K22 of KIF5B and the 5′ end of exon R12 of RET, or a nucleotide sequence at or near a junction of a KIF5B-RET gene fusion comprising the 3′ end of exon K23 of KIF5B and the 5′ end of exon R12 of RET. In the kit (ii) (a) can further comprise using primers for at least one IC gene, wherein the primers optionally hybridize to different exons of the at least one IC gene, and (b) further comprises detecting the amplified IC cDNA.

A still further kit is provided. The kit comprises:

(i) a set of primers comprising at least two primers selected from the group consisting of a primer that hybridizes to exon P3 of PML, a primer that hybridizes to exon 6a of PML, and a primer that hybridizes to exon 6b of PML; and

(ii) instructions for a method of detecting mRNA from fusions of the PML gene and the RARα gene in a sample of mRNA from a human, which method comprises:
(a) (i) obtaining cDNA, which has been reverse-transcribed from the sample of mRNA, or reverse-transcribing the sample of mRNA to cDNA and (ii) amplifying the reverse-transcribed cDNA using primers for a group of PML-RARα gene fusion variants comprising at least two gene fusion variants selected from the group consisting of a P3R3 gene fusion, a P6aR3 gene fusion, and a P6bR3 gene fusion, wherein one or more of the primers can be detectably labeled, and
(b) detecting the amplified cDNA and optionally, simultaneously or sequentially, quantitating the amplified cDNA. The kit can comprise a primer that hybridizes to exon R3 of RARα. The kit can further comprise a probe for each gene fusion that is not detected using a detectably labeled primer, wherein the probe hybridizes to a nucleotide sequence at or near a junction of a PML-RARα gene fusion comprising the 3′ end of exon P3 of PML and the 5′ end of exon R3 of RARα, a nucleotide sequence at or near a junction of a PML-RARα gene fusion comprising the 3′ end of exon P6a of PML and the 5′ end of exon R3 of RARα, or a nucleotide sequence at or near a junction of a PML-RARα gene fusion comprising the 3′ end of exon P6b of PML and the 5′ end of exon R3 of RARα. In the kit (ii) (a) can further comprise using primers for at least one IC gene, wherein the IC primers can hybridize to the same or different exons of the at least one IC gene, and (b) further comprises detecting the amplified IC cDNA.

A kit can contain a container or a sample vial for storing a sample of a tissue or a body fluid. The primers, such as a pair of primers, specifically a forward primer and a reverse primer, can be in a composition in amounts effective to permit detection of one or more gene fusion variants. Detection of gene fusion variants is accomplished using any of the methods described herein or known in the art for detecting a specific nucleic acid molecule in a sample. A kit can also comprise buffers, nucleotide bases, and other compositions to be used in hybridization and/or amplification reactions.

The kit can further comprise dNTPs. Preferably, the dNTPs are supplied in a buffered solution with a reference dye.

The primers, probes and dNTPs can be packaged in various configurations. Preferably, the primers, probes and dNTPS are in a single container. The container preferably also contains a preservative, such as sodium azide and/or ProClin® 950.

The kit can further comprise an RNA polymerase or a mixture of a reverse transcriptase and a DNA polymerase. Any suitable RNA polymerase can be used. An example of a preferred RNA polymerase is rTth. Any suitable reverse transcriptase also can be used. An example of a preferred reverse transcriptase is SuperScript® III (Life Technologies Corp., Carlsbad, Calif.). Likewise, any suitable DNA polymerase can be used. An example of a preferred DNA polymerase is AmpliTaq Gold® (Life Technologies Corp., Carlsbad, Calif.). The polymerase and/or transcriptase can be supplied in a buffered solution, which optionally contains, and preferably does contain, stabilizers.

The kit can further comprise an activation reagent, such as manganese chloride, in a buffered solution. The buffered solution preferably includes a preservative, such as sodium azide and/or ProClin® 950.

The kit can optionally further comprise an IC. The IC is an unrelated human nucleic acid sequence that demonstrates that the process has proceeded correctly for each sample. The IC also can be used to quantify the relative expression of BCR-ABL gene fusion variants. Any suitable sequence can be used as the IC. Examples of IC genes are set forth in the “EXAMPLES” herein, namely, GUSB, ABL, and G6PD. The BCR-ABL gene fusion variant-specific probes and the IC-specific probes are labeled differently so that BCR-ABL DNA and IC DNA can be distinguished.

The kit optionally further comprises a processing control that is a non-human nucleic acid sequence. The processing control can be added to the process to ensure further that the assay has proceeded correctly.

### EXAMPLES

The following examples serve to illustrate the present disclosure. The examples are not intended to limit the scope of the claimed invention in any way.

### Example 1

This example describes the design of the BCR-ABL amplification primers.

Amplification involves the use of forward and reverse primers located on opposite sides of the translocation breakpoints between fused BCR and ABL gene segments. Thus, each of the resulting BCR-ABL amplicons contains a variant-specific junction sequence. One reverse primer (RP) and three forward primers (FP) were generated. Reverse primer a2RP anneals to a specific sequence within exon a2 of ABL that is common to the variants e1a2, b2a2, b3a2, and e19a2. The reverse primer a2RP directs reverse transcription of RNA from multiple variants. Forward primers e1FP, b2FP, and e19FP anneal to specific sequences within exons e1, b2, and e19, respectively, of BCR. The pair of primers e1FP and a2RP amplify a cDNA target sequence that is reverse-transcribed from variant e1a2 RNA. The pair of primers b2FP and a2RP amplify cDNA target sequences that are reverse-transcribed from variants b2a2 and b3a2 RNA. The pair of primers e19FP and a2RP amplify a cDNA target sequence that is reverse-transcribed from variant e19a2 RNA. A pair of primers can be used in a single reaction to amplify a specific variant (or specific variants, when primers b2FP and a2RP are used). Alternatively, two or more pairs of primers can be used in a single reaction to amplify two or more variants in a multiplexed reaction. The sequences of the primers are shown in Table II.

TABLE II
BCR-ABL Primers

| | | SEQ |
|---|---|---|
| Primer | | ID |
| Designation | Sequence 5′→3′ | NO |
| e1FP | ATCGTGGGCGTCCGCAAGA | 1 |
| b2FP | AGATGCTGACCAACTCGTGTGTGA | 2 |
| e19FP | TGGAGGAGGTGGGCATCTACC | 3 |
| a2RP | TGAGGCTCAAAGTCAGATGCTACTG | 4 |

Alternative primers can be used. Examples of alternative primers are shown in Table III.

TABLE III
Alternative BCR-ABL Primers

| | | SEQ |
|---|---|---|
| Primer | | ID |
| Designation | Sequence 5′→3′ | NO |
| e1FP_alt1 | TGGGCGTCCGCAAGACC | 18 |
| e1FP_alt2 | GTCGTGTCCGAGGCCACCAT | 19 |
| e1FP_alt3 | CGGTTGTCGTGTCCGAGGC | 20 |
| e1FP_alt4 | CACGCCGCAGTGCCATAAG | 21 |
| e1FP_alt5 | ACAGTCCTTCGACAGCAGCAGTC | 22 |
| b2FP_alt1 | CTTCTCCCTGACATCCGTGGAG | 23 |
| e19FP_alt1 | GGAGGAGGTGGGCATCTACCG | 24 |
| a2RP_alt1 | GAGTTCCAACGAGCGGCTTCA | 25 |
| a2RP_alt2 | acccggagcttttcacCTTTAGTT | 26 |
| a2RP_alt3 | agacccggagcttttcacCTTTAG | 27 |

Primers e1FP_alt1, e1FP_alt2, e1FP_alt3, e1FP_alt4, and e1FP_alt5 function similarly to e1FP. Primer b2FP_alt1 functions similarly to b2FP. Primer e19FP_alt1 functions similarly to e19FP. Primers a2RP-alt1, a2RP_alt2, and a2RP_alt3 function similarly to a2RP. Primers a2RP_alt2 and a2RP_alt3 anneal to specific sequences at the junction of ABL exons a2 and a3. Since these sequences are separated by an intron in genomic DNA, a2RP_alt2 and a2RP_alt3 specifically target RNA. Primer a2RP_alt1 is derived from exon a2 of ABL, whereas only the uppercase nucleotides in a2RP_alt2 and a2RP_alt3 are derived from exon a2 of ABL and the lowercase nucleotides in a2RP_alt2 and a2RP_alt3 are derived from exon a3 of ABL.

### Example 2

This example describes the design of the BCR-ABL probes.

Four short oligonucleotide probes were designed to hybridize specifically to the junction-specific sequence from each targeted BCR-ABL variant (i.e., e1a2, b2a2, b3a2, and e19a2) with high affinity. The BCR-ABL exon junction spanned by each probe is separated by intron sequences in genomic DNA. Therefore, the probes will only hybridize to amplicons derived from BCR-ABL RNA/cDNA. The specificity of the probes enables differentiation of variants. The probes can be used individually to detect single BCR-ABL variants. Alternatively, two or more probes can be used together to detect multiple BCR-ABL variants in a single reaction. The probes can be identically labeled for pooled detection of the targeted BCR-ABL variants. Alternatively, the probes can be differently labeled for differential detection of two or more BCR-ABL variants in a single reaction. The sequences of the probes are shown in Table IV.

TABLE IV
BCR-ABL Probes

| | | SEQ |
|---|---|---|
| Probe | | ID |
| Designation | Sequence 5′-3′ | NO |
| e1a2probe | dye ACGCAGAAGCCCT quencher | 5 |
| b2a2probe | dye AAGGAAGAAGCCC quencher | 6 |
| b3a2probe | dye AGTTCAAAAGCCC quencher | 7 |
| e19a2probe | dye ACGTCAAAGCCCTT quencher | 8 |

Probe sequences derived from the BCR side of each translation variant are italicized. Each probe comprises a sequence from the sense strand of its associated amplicon. While Table IV shows the dye at the 5′ end of the probe and the quencher at the 3′ end of the probe, the quencher can be at the 5′ end of the probe and the dye can be at the 3′ end of the probe. The SEQ ID NO set forth in Table IV is in reference to the nucleotide sequence in the absence of the dye and the quencher.

The e1a2 probe consists of the last six nucleotides at the 3′ end of exon e1 of BCR and the first seven nucleotides at the 5′ end of exon a2 of ABL. Therefore, the e1a2 probe recognizes the junction-specific sequence amplified from BCR-ABL variant e1a2 mRNA.

The b2a2 probe consists of the last seven nucleotides at the 3′ end of exon b2 of BCR and the first six nucleotides at the 5′ end of exon a2 of ABL. Therefore, the b2a2 probe recognizes the junction-specific sequence amplified from BCR-ABL variant b2a2 mRNA.

The b3a2 probe consists of the last seven nucleotides at the 3′ end of exon b3 of BCR and the first six nucleotides at the 5′ end of exon a2 of ABL. Therefore, the b3a2 probe recognizes the junction-specific sequence amplified from BCR-ABL variant b3a2 mRNA.

The e19a2 probe consists of the last six nucleotides at the 3′ end of exon e19 of BCR and the first eight nucleotides at the 5′ end of exon a2 of ABL. Therefore, the e19a2 probe recognizes the junction-specific sequence amplified from BCR-ABL variant e19a2a2 RNA.

Alternative probes can be used. Examples of alternative probes are shown in Table V.

TABLE V
Alternative BCR-ABL Probes

| | | SEQ |
|---|---|---|
| Probe | | ID |
| Designation | Sequence 5′-3′ | NO |
| e1a2probe_alt1 | dye AAGGGCTTCTGCGTC quencher | 28 |
| e1a2probe_alt2 | dye GACGCAGAAGCCC quencher | 29 |
| e1a2probe_alt3 | dye GACGCAGAAGCCCT quencher | 30 |
| b2a2probe_alt1 | dye AAGGGCTTCTTCC quencher | 31 |
| b2a2probe_alt2 | dye AGGGCTTCTTCCTT quencher | 32 |
| b3a2probe_alt1 | dye AGAGTTCAAAAGCC quencher | 33 |
| b3a2probe_alt2 | dye AAGGGCTTTTGAACTC quencher | 34 |
| b3a2probe_alt3 | dye AAGGGCTTTTGAACTC quencher | 35 |
| e19a2probe_alt1 | dye AAGGGCTTTGACGTC quencher | 36 |

Probe sequences derived from the BCR side of each translation variant are italicized. Each of the probes e1a2probe_alt2, e1a2probe_alt3, and b3a2probe_alt1 comprises a sequence from the sense strand of its associated amplicon. All other alternative probes comprise a sequence that is complementary to the sense strand of its associated amplicon. While Table V shows the dye at the 5′ end of the probe and the quencher at the 3′ end of the probe, the quencher can be at the 5′ end of the probe and the dye can be at the 3′ end of the probe. The SEQ ID NO set forth in Table V is in reference to the nucleotide sequence in the absence of the dye and the quencher.

For example, the alternate e1a2 probe designated alt2 in Table V consists of the last seven nucleotides at the 3′ end of exon e1 of BCR and the first six nucleotides at the 5′ end of exon a2 of ABL. Therefore, the e1a2 probe recognizes the junction-specific sequence amplified from BCR-ABL variant e1a2 mRNA.

The alternate b2a2 probe designated alt2 in Table V consists of the last seven nucleotides at the 3′ end of exon b2 of BCR and the first seven nucleotides at the 5′ end of exon a2 of ABL. Therefore, the b2a2 probe recognizes the junction-specific sequence amplified from BCR-ABL variant b2a2 mRNA.

The alternate b3a2 probe designated alt1 in Table V consists of the last nine nucleotides at the 3′ end of exon b3 of BCR and the first five nucleotides at the 5′ end of exon a2 of ABL. Therefore, the b3a2 probe recognizes the junction-specific sequence amplified from BCR-ABL variant b3a2 mRNA.

The alternate e19a2 probe designated alt1 in Table V consists of the last seven nucleotides at the 3′ end of exon e19 of BCR and the first eight nucleotides at the 5′ end of exon a2 of ABL. Therefore, the e19a2 probe recognizes the junction-specific sequence amplified from BCR-ABL variant e19a2a2 RNA.

### Example 3

This example describes the design of internal control (IC) amplification primers and probe.

Two oligonucleotide primers, i.e., a forward primer and a reverse primer, were designed for amplification of each of three endogenous genes for use as internal controls. The primers were designed to amplify the well-characterized housekeeping gene β-glucuronidase (GUSB), ABL, and glucose-6-phosphate dehydrogenase (G6PD).

The forward primer for GUSB (GUSBFP) anneals to a sequence located in exon 8 of GUSB. The reverse primer for GUSB (GUSBRP) anneals to a sequence located in exon 10 of GUSB and, in combination with GUSBFP, directs the amplification of the reverse-transcribed GUSB cDNA. The resulting amplicon spans sequences from exons 8, 9 and 10 of GUSB.

An oligonucleotide probe was designed for hybridization and detection of the GUSB amplicon. The probe was designed to hybridize to a sequence in exon 9 of GUSB.

The forward primer for ABL (ABLFP) anneals to a sequence located in exon 3 of ABL. The reverse primer for ABL (ABLRP) anneals to a sequence located in exon 4 of ABL and, in combination with ABLFP, directs the amplification of the reverse-transcribed ABL cDNA. The resulting amplicon spans sequences from exons 3 and 4 of ABL.

An oligonucleotide probe was designed for hybridization and detection of the ABL amplicon. The probe was designed to hybridize to a sequence in exon 3 (3′ to ABLFP) of ABL.

The forward primer for G6PD (G6PDFP) anneals to a sequence located in exon 2 of G6PD. The reverse primer for G6PD (G6PDRP) anneals to a sequence located in exon 4 and, in combination with G6PDFP, directs the amplification of the reverse-transcribed G6PD cDNA. The resulting amplicon spans sequences from exons 2, 3 and 4 of G6PD.

An oligonucleotide probe was designed for hybridization and detection of the G6PD cDNA amplicon. The probe was designed to hybridize to a sequence at the junction of G6PD exons 2 and 3.

The sequences of the IC primers and probes are shown in Table VI. The IC primers and probe can be included in the BCR-ABL primer and probe mixes for simultaneous amplification and detection of BCR-ABL variant RNA and IC RNA. In such a configuration, however, the BCR-ABL probe and the IC probe(s) are differentially labeled.

TABLE VI
IC Primers and Probes

| Primer/ | | SEQ |
|---|---|---|
| Probe | | ID |
| Designation | Sequence 5′→3′ | NO |
| GUSBFP | TCCCACCTAGAATCTGCTGGCTAC | 9 |
| GUSBRP | GCTGTTCAAACAGATCACATCCACA | 10 |
| GUSBprobe | dye ATCGCTCACACCAAATCCTTGGACC | 11 |
| | quencher | |
| ABLFP | GTGCGTGAGAGTGAGAGCAGTCCT | 12 |
| ABLRP | CAGGGTGTTGAAGCGGCTCTC | 13 |
| ABLprobe | dye CCATCTCGCTGAGATACGAAGGGAGG | 14 |
| | quencher | |
| G6PDFP | GCGATGCCTTCCATCAGTCG | 15 |
| G6PDRP | TGAAGGTGTTTTCGGGCAGAAG | 16 |
| G6PDprobe | dye CATGGGTGCATCGGGTGACCTG | 17 |
| | quencher | |

While Table VI shows the dye at the 5′ end of the probe and the quencher at the 3′ end of the probe, the quencher can be at the 5′ end of the probe and the dye can be at the 3′ end of the probe. The SEQ ID NO set forth in Table VI is in reference to the nucleotide sequence in the absence of the dye and the quencher.

### Example 4

This example describes an exemplary PCR reaction.

Primers and probes and other components essential for nucleic acid amplification, such as dNTP mix, buffer, polymerase, and divalent ion, were mixed. Purified RNA potentially containing BCR-ABL transcripts was added to the mixture. The mixture was subjected to specific conditions for reverse transcription of BCR-ABL mRNA and IC mRNA. The reverse-transcribed cDNA was then amplified. The use of a polymerase that can catalyze reverse transcription of RNA and amplification of DNA enables reverse transcription and amplification to be carried out in a single-run, closed-tube format by an instrument that can concurrently carry out thermal cycling and signal detection. Amplified BCR-ABL and IC was detected. Optionally, the level of BCR-ABL mRNA present in the purified RNA sample was determined by comparison to the level(s) of IC mRNA (i.e., relative quantitation). Use of differentially detectable labels enables detection and differentiation of RNA from two or more BCR-ABL variants and, depending on the number of IC used, RNA from one or more IC. An exemplary PCR reagent mixture is shown in Table VII. In an exemplary PCR reaction 25 μl of purified RNA were mixed with 25 μl of PCR reagent mixture. PCR cycling conditions are shown in Table VIII.

TABLE VII
PCR Reagent Mixture

| Component | Reaction Concentration | Unit of Measure |
|---|---|---|
| e1FP | 0.2 | μM |
| b2FP | 0.1 | μM |
| e19FP | 0.1 | μM |
| a2RP | 0.6 | μM |
| GUSBFP | 0.1 | μM |
| GUSBRP | 0.3 | μM |
| e1a2 probe | 0.4 | μM |
| b2a2 probe | 0.4 | μM |
| b3a2probe | 0.4 | μM |
| e19a2 probe | 0.2 | μM |
| GUSB probe | 0.2 | μM |
| PCR buffer | 1.0 | X |
| dNTPs | 0.325 | mM |
| ROX | 0.015 | μM |
| reference dye | | |
| aptamer | 0.2 | μM |
| rTth polymerase | 10 | Units |
| MnCl₂ | 3 | mM |

dTNPs = deoxyribonucleotide triphosphates

TABLE VIII
PCR Cycling Conditions

| Cycles | Parameters | Description |
|---|---|---|
| 1 | 58° C./30 min | reverse transcription |
| 4 | 92° C./30 sec, 60° C./30 sec | DNA amplification |
| | | (low stringency) |
| 56 | 92° C./30 sec, 62° C./30 sec, | DNA amplification |
| | 58° C./40 sec | (DNA amplification and |
| | | fluorescence reads) |

### Example 5

This example describes methodology relevant to the detection of EML4-ALK gene fusion variants.

Amplification, detection, quantification and differentiation of EML4-ALK fusion variants from an RNA sample can be achieved using a method similar to those described above.

The PCR amplification principle of this method utilizes an oligonucleotide mix comprised of a reverse primer and multiple forward primers. The reverse primer (designated A20RP) anneals to a specific sequence region within ALK exon 20 that is common to the targeted EML4-ALK fusion variants. Forward primers, designated E6aFP, E6bFP, E13FP, E14FP and E20FP anneal to specific sequence regions of EML4 exon 6a, exon 6b, exon 13, exon 14, and exon 20, respectively. The utility of each primer in the functionality of the method is summarized as follows:
Reverse primer A20RP directs reverse transcription of RNA from multiple EML4-ALK fusion variants.
The combination of forward primer E6aFP and reverse primer A20RP amplifies cDNA target sequences that are reverse-transcribed from EML4-ALK E6aA20.
The combination of forward primer E6bFP and reverse primer A20RP amplifies cDNA target sequences that are reverse-transcribed from EML4-ALK E6bA20. Note that, due to the proximity of EML exons 6a and 6b, a single EML forward primer (e.g., E6aFP), in combination with ALK reverse primer A20RP, can be used to amplify both E6aA20 and E6bA20 fusion variants, if desired.
The combination of forward primer E13FP and reverse primer A20RP amplifies cDNA target sequences that are reverse-transcribed from EML4-ALK E13A20.
The combination of forward primer E14FP and reverse primer A20RP amplifies cDNA target sequences that are reverse-transcribed from EML4-ALK E14A20. Note that, due to the proximity of EML exons 13 and 14, a single EML forward primer (e.g., E13FP), in combination with ALK reverse primer A20RP, can be used to amplify both E13A20 and E14A20 fusion variants, if desired.
The combination of forward primer E20FP and reverse primer A20RP amplifies cDNA target sequences that are reverse-transcribed from EML4-ALK E20A20.
Note that this method can be modified to incorporate forward primers from other EML4 regions if amplification of additional (rare) EML4-ALK fusion variants (e.g., E18A20, E15A20, E2A20 and/or E17A20) is desired. In this regard, we point out that COSMIC has inferred the following breakpoints for EML4-ALK: 1_{—}1725 EML4 (transcript ID: ENST00000318522) and 4080_{—}6222 ALK (NCBI Acc. No. NM_{—}004304; [SEQ ID NO:39]), 1_{—}903+220 EML4 and 4080_{—}6222 ALK, and 1_{—}2478 EML4 and 4080_{—}6222 ALK.

These primer sets can be used either individually to amplify specific variants in separate PCR reactions or in a multiplex configuration to amplify multiple variants in one PCR reaction.

The amplification principal discussed above utilizes forward and reverse primers located on opposite sides of the inversion breakpoints between fused EML4 and ALK gene segments. Thus, each of the resulting EML4-ALK amplicons will contain a variant-specific junction sequence. The detection principle uses multiple oligonucleotide probes (designated E6aA20probe, E6bA20probe, E13A20probe, E14A20probe, and E20A20probe) designed to hybridize specifically with high affinity to sequences at exon fusion junctions of targeted EML4-ALK variants. The utility of each probe in the functionality of the method is summarized as follows:
E6aA20probe consists of nucleotide sequences at the junction of EML4 exon 6a and ALK exon 20. Therefore, this probe recognizes the sequences amplified from EML4-ALK E6aA20.
E6bA20probe consists of nucleotide sequences at the junction of EML4 exon 6b and ALK exon 20. Therefore, this probe recognizes the sequences amplified from EML4-ALK E6bA20.
E13A20probe consists of nucleotide sequences at junction of EML4 exon 13 and ALK exon 20. Therefore, this probe recognizes the sequences amplified from EML4-ALK E13A20.
E14A20probe consists of nucleotide sequences at the junction of EML4 exon 14 and ALK exon 20. Therefore, this probe recognizes the sequences amplified from EML4-ALK E14A20.
E20A20probe consists of nucleotide sequences at the junction of EML4 exon 20 and ALK exon 20. Therefore, this probe recognizes the sequences amplified from EML4-ALK E20A20.
EML4-ALK exon junctions are separated by intron sequences in genomic DNA. Therefore, the probes spanning exon junctions will only hybridize to amplicons derived from EML4-ALK RNA/cDNA (and not genomic DNA).
Note that this method can be modified to incorporate probes from other EML4-ALK exon fusion junctions to achieve detection of additional, rare EML4-ALK fusion variants (e.g., E18A20, E15A20, E2A20 and/or E17A20), if desired.
Note detection of amplified sequences can also be achieved using a probe (or probes) that do not span EML4-ALK fusion junction(s). For example, a probe targeting sequence within ALK exon 20 (5′ to reverse primer A20RP and 3′ to the exon fusion junction) can be used for common detection of all targeted EML4-ALK fusion variants amplified using the above-mentioned primer sets. Alternatively, probes within the amplified region of targeted EML4 exons (3′ to the applicable EML4 forward primer and 5′ to the exon fusion junction) can also be used for detection of fusion variants.

Probes can either be used individually to detect single EML4-ALK variants or as a mixture to detect multiple variants in a single reaction. In addition, probes can either be labeled with a common dye for pooled detection of the targeted EML4-ALK variants or labeled with different dyes enabling differentiated detection of multiple EML4-ALK variants in a single reaction.

Like the aforementioned BCR-ABL method, this ELM4-ALK method can also utilize an additional primer/probe set for detection of cellular RNA expressed by the endogenous housekeeping gene, such as GUSB, ABL or G6PD. RNA levels from the endogenous housekeeping gene can be used to normalize the ELM4-ALK transcript quantitation process against variations in cell adequacy, sample extraction, and amplification efficiency. RNA levels from this housekeeping gene can also serve as a sample validity control.

### Example 6

This example describes methodology relevant to the detection of KIF5B-RET gene fusion variants.

Amplification, detection, quantification and differentiation of KIF5B-RET fusion variants from an RNA sample can be achieved using a method similar to those described above.

The PCR amplification principle of this method utilizes an oligonucleotide mix comprised of a reverse primer and multiple forward primers. The reverse primer (designated RET12RP) anneals to a specific sequence region within RET exon 12 that is common to the targeted KIF5B-RET fusion variants. The forward primers, designated K15FP, K16FP, K22FP and K23FP, anneal to specific sequence regions of KIF5B exon 15, exon 16, exon 22, and exon 23, respectively. The utility of each primer in the functionality of this method is summarized as follows:
Reverse primer RET12RP directs reverse transcription of RNA from multiple KIF5B-RET fusion variants.
The combination of forward primer K15FP and reverse primer RET12RP amplifies cDNA target sequences that are reverse-transcribed from KIF5B-RET fusion variant K15R12.
The combination of forward primer K16FP and reverse primer RET12RP amplifies cDNA target sequences that are reverse-transcribed from KIF5B-RET fusion variant K16R12. Note that, due to the proximity of KIF5B exons 15 and 16, a single KIF5B forward primer (e.g., K15FP), in combination with RET reverse primer RET12RP, can be used to amplify both K15R12 and K16R12 fusion variants, if desired.
The combination of forward primer K22FP and reverse primer RET12RP amplifies cDNA target sequences that are reverse-transcribed from KIF5B-RET fusion variant K22R12.
The combination of forward primer K23FP and reverse primer RET12RP amplifies cDNA target sequences that are reverse-transcribed from KIF5B-RET K23R12. Note that, due to the proximity of KIF5B exons 22 and 23, a single KIF5B forward primer (e.g., K22FP), in combination with RET reverse primer RET12RP, can be used to amplify both K22R12 and K23R12 fusion variants, if desired.
This method can be modified to incorporate forward primers from other KIF5B regions if amplification of additional KIF5B-RET fusion variants is desired. In this regard, we point out that COSMIC has inferred the following breakpoints for KIF5B-RET: 1_{—}2183 KIF5B (transcript ID: ENST00000302418) and 2327_{—}5629 RET (NCBI Acc. No. NM_{—}020975.4 [SEQ ID NO:134]) and 1_{—}2372+476 KIF5B and 2327-436_{—}5629 RET.

These primer sets can be used either individually to amplify specific variants in separate PCR reactions or in a multiplex configuration to amplify multiple variants in one PCR reaction.

The amplification principle discussed above utilizes forward and reverse primers located on opposite sides of the inversion breakpoints between fused KIF5B and RET gene segments. Thus, each of the resulting KIF5B-RET amplicons will contain a variant-specific junction sequence. The detection principle for this method uses multiple oligonucleotide probes (designated K15R12probe, K16R12probe, K22R12probe, and K23R12probe) designed to hybridize specifically with high affinity to sequences at the exon fusion junctions of targeted KIF5B-RET variants. The utility of each probe in the functionality of this method is summarized as follows:
K15R12probe consists of nucleotide sequences at the junction of KIF5B exon 15 and RET exon 12. Therefore, this probe recognizes the sequences amplified from KIF5B-RET fusion variant K15R12.
K16R12probe consists of nucleotide sequences at the junction of KIF5B exon 16 and RET exon 12. Therefore, this probe recognizes the sequences amplified from KIF5B-RET fusion variant K16R12.
K22R12probe consists of nucleotide sequences at the junction of KIF5B exon 22 and RET exon 12. Therefore, this probe recognizes the sequences amplified from KIF5B-RET fusion variant K22R12.
K23R12probe consists of nucleotide sequences at the junction of KIF5B exon 23 and RET exon 12. Therefore, this probe recognizes the sequences amplified from KIF5B-RET fusion variant K23R12.
KIF5B-RET exon fusion junctions are separated by intron sequences in genomic DNA. Therefore, the probes spanning these junctions will only hybridize to amplicons derived from KIF5B-RET RNA/cDNA (and not from genomic DNA).
Note that the method can be modified to incorporate probes from other KIF5B-RET exon fusion junctions to achieve detection of additional KIF5B-RET fusion variants, if desired.
Note that detection of amplified sequences can also be achieved using a probe (or probes) that do not span KIF5B-RET fusion junction(s). For example, a probe targeting sequence within RET exon 12 (5′ to reverse primer RET12RP and 3′ to the exon fusion junction) can be used for common detection of all targeted KIF5B-RET fusion variants amplified using the above-mentioned primer sets. Alternatively, probes within the amplified region of targeted KIF5B exons (3′ to the applicable KIF5B forward primer and 5′ to the exon fusion junction) can also be used for detection of fusion variants.

Probes can either be used individually to detect single KIF5B-RET variants or be used as a mixture to detect multiple variants in a single reaction. In addition, the probes can either be labeled with a common dye for pooled detection of the targeted KIF5B-RET fusion variants or labeled with different dyes enabling differentiated detection of multiple KIF5B-RET fusion variants in a single reaction.

Like the aforementioned BCR-ABL and ELM4-ALK methods, this KIF5B-RET method can also utilize an additional primer/probe set for detection of cellular RNA expressed by the endogenous housekeeping gene, such as GUSB, ABL or G6PD. RNA levels from the endogenous housekeeping gene can be used to normalize the KIF5B-RET transcript quantitation process against variations in cell adequacy, sample extraction, and amplification efficiency. RNA levels from this housekeeping gene can also serve as a sample validity control.

### Example 7

This example describes methodology relevant to the detection of PML-RARα gene fusion variants.

Amplification, detection, quantification and differentiation of PML-RARα translocation variants from an RNA sample can be achieved using a method similar to those described above.

The PCR amplification principle of this method utilizes an oligonucleotide mix comprised of a reverse primer and multiple forward primers. The reverse primer (designated R3RP) anneals to a specific sequence region within RARα exon 3 that is common between the targeted PML-RARα translocation variants. The forward primers, designated P3FP, P6aFP and P6bFP, anneal to specific sequence regions of PML exon 3, the 5′ region of PML exon 6, or the 3′ region of PML exon 6, respectively. The utility of each primer in the functionality of this method is summarized as follows:
Reverse primer R3RP directs reverse transcription of RNA from multiple PML-RARα translocation variants.
The combination of forward primer P3FP and reverse primer R3RP amplifies cDNA target sequences that are reverse-transcribed from PML-RARα S form (the translocation variant in which PML exon 3 is fused to RARα exon 3).
The combination of forward primer P6aFP and reverse primer R3RP amplifies cDNA target sequences that are reverse-transcribed from PML-RARα V form (the translocation variant in which the 5′ region of PML exon 6 is fused to RARα exon 3).
The combination of forward primer P6bFP and reverse primer R3RP amplifies cDNA target sequences that are reverse-transcribed from PML-RARα L form (the translocation variant in which the 5′ region of PML exon 6 is fused to RARα exon 3). Note that due to the proximity of the exon fusion junctions from PML-RARα V form and PML-RARα L form, a single PML forward primer (e.g., P6aFP), in combination with RARα reverse primer R3RP, can be used to amplify both exon fusion junctions, if desired.
Note that this method can be modified to incorporate forward primers from other PML regions if amplification of additional (rare) PML-RARα fusion variants is desired.

These primer sets can be used individually to amplify specific variants in separate PCR reactions or in a multiplex configuration to amplify multiple variants in one PCR reaction.

The amplification principle discussed above utilizes forward and reverse primers located on opposite sides of the translocation breakpoints between fused PML and RARα gene segments. Thus, each of the resulting PML-RARα amplicons will contain a variant-specific junction sequence. The detection principle for this method uses three oligonucleotide probes (designated Sprobe, Vprobe, and Lprobe) designed to hybridize specifically hybridize with high affinity to sequences at the targeted PML-RARα variants (S form, V form, or L form). The utility of each probe in the functionality of this method is summarized as follows:
Sprobe consists of nucleotide sequences at the junction of PML exon 3 and RARα exon 3. Therefore, this probe recognizes the sequences amplified from PML-RARα form S RNA.
Vprobe consists of nucleotide sequences at the junction of the 5′ region of PML exon 6 and RARα exon 3. Therefore, this probe recognizes the sequences amplified from PML-RARα form V RNA.
Lprobe consists of nucleotide sequences at the junction of the 3′ region of PML exon 6 and RARα exon 3. Therefore, this probe recognizes the sequences amplified from PML-RARα form L RNA.
PML-RARα exon junctions are separated by intron sequences in genomic DNA. Therefore, the probes spanning exon junctions will only hybridize to amplicons derived from PML-RARα RNA/cDNA (and not genomic DNA).
Note that this method can be modified to incorporate probes from other PML-RARα exon fusion junctions to achieve detection of additional, rare EML4-ALK fusion variants, if desired.
Note detection of amplified sequences can also be achieved using a probe (or probes) that does not span PML-RARα fusion junction(s). For example, a probe targeting sequences within RARα exon 3 (5′ to reverse primer R3RP and 3′ to the exon fusion junction) can be used for common detection of all targeted PML-RARα fusion variants amplified using the above-mentioned primer sets. Alternatively, probes within the amplified region of targeted PML exons (3′ to the applicable PML forward primer and 5′ to the exon fusion junction) can also be used for detection of fusion variants.

Probes can either be used individually to detect single PML-RARα variants or be used as a mixture to detect multiple variants in a single reaction. In addition, the probes can either be labeled with a common dye for pooled detection of the targeted PML-RARα variants or labeled with different dyes enabling differentiated detection of multiple PML-RARα variants in a single reaction.

Like the aforementioned BCR-ABL, ELM4-ALK, and KIF5B-RET methods, this PML-RARα method can also utilize an additional primer/probe set for detection of cellular RNA expressed by the endogenous housekeeping gene, such as GUSB, ABL or G6PD. RNA levels from the endogenous housekeeping gene can be used to normalize the PML-RARα transcript quantitation process against variations in cell adequacy, sample extraction, and amplification efficiency. RNA levels from this housekeeping gene can also serve as a sample validity control.

Thus, in view of the above, the present disclosure provides the following:

A. A method of amplifying mRNA from variants of a fusion between a first gene and a second gene in a sample of mRNA, which method comprises:

(a) (i) obtaining cDNA, which has been reverse-transcribed from the sample of mRNA, or reverse-transcribing the sample of mRNA to cDNA and (ii) amplifying the reverse-transcribed cDNA by polymerase chain reaction using primers comprising a primer for an exon of the first gene, which becomes contiguous with a variant exon of the second gene, and a primer for each of two or more variant exons of the second gene,

whereupon mRNA from variants of a fusion between a first gene and a second gene in a sample of mRNA is amplified.

B. The method of A, which further comprises:

(b) detecting the amplified cDNA or detecting and quantitating the amplified cDNA,

whereupon mRNA from variants of a fusion between the first gene and the second in a sample of mRNA is detected.

C. The method of A, wherein the reverse-transcribed cDNA is amplified by polymerase chain reaction in the presence of a mixture of deoxyribonucleotide triphosphates (dNTP), a buffer, a polymerase, and a divalent ion, while cycling between a temperature of about 58° C. to about 62° C. for about 30 seconds to about 40 seconds and a temperature of about 92° C. for about 30 seconds.

D. The method of A or B, wherein one or more primers is/are detectably labeled.

E. The method of D, wherein detection of the one or more detectably labeled primers can be distinguished.

F. The method of B, wherein detecting the amplified cDNA comprises contacting the amplified cDNA with at least one probe under hybridizing conditions and detecting hybridization of the at least one probe to the amplified cDNA.

G. The method of F, wherein at least one probe is a probe that hybridizes to a nucleotide sequence at or near a junction of a variant of a fusion between the first gene and the second gene comprising the 3′ end of an exon of the first gene and the 5′ end of an exon of the second gene.

H. The method of F or G, wherein detecting the amplified cDNA comprises contacting the amplified cDNA with at least two probes, wherein hybridization of each probe to the amplified cDNA is distinguishable.

I. The method of B, wherein (a) further comprises using primers for at least one internal control (IC) gene, wherein the IC primers can hybridize to the same or different exons of the at least one IC gene, and (b) further comprises detecting the amplified IC cDNA.

J. The method of I, wherein one or more IC primers is/are detectably labeled.

K. The method of J, wherein detection of the one or more detectably labeled IC primers can be distinguished.

L. The method of I, wherein detecting the amplified IC cDNA comprises contacting the amplified IC cDNA with at least one IC probe and/or at least one IC primer under hybridizing conditions and detecting hybridization of the at least one IC probe/primer to the amplified IC cDNA.

M. The method of L, wherein detecting the amplified IC cDNA comprises contacting the amplified IC cDNA with at least two IC probes/primers, wherein each IC probe/primer is specific for a different IC cDNA and hybridization of each IC probe/primer to the amplified IC cDNA is distinguishable.

N. A method of amplifying mRNA from fusion variants of the breakpoint cluster region (BCR) gene and the Abelson murine leukemia (ABL) proto-oncogene in a sample of mRNA from a human, which method comprises:

(a) (i) obtaining cDNA, which has been reverse-transcribed from the sample of mRNA, or reverse-transcribing the sample of mRNA to cDNA and (ii) amplifying the reverse-transcribed cDNA using primers comprising primers for a group of BCR-ABL gene fusion variants comprising at least two gene fusion variants selected from the group consisting of an e1a2 gene fusion, a b2a2 gene fusion, a b3a2 gene fusion, and an e19a2 gene fusion, whereupon mRNA from BCR-ABL gene fusion variants in a sample of mRNA from a human is amplified.

O. The method of N, which further comprises:

(b) detecting the amplified cDNA or detecting and quantitating the amplified cDNA,

whereupon mRNA from BCR-ABL gene fusion variants in a sample of mRNA from a human is detected.

P. The method of N, wherein the reverse-transcribed cDNA is amplified by polymerase chain reaction in the presence of a mixture of dNTP, a buffer, a polymerase, and a divalent ion, while cycling between a temperature of about 58° C. to about 62° C. for about 30 seconds to about 40 seconds and a temperature of about 92° C. for about 30 seconds.

Q. The method of N or O, wherein the primers comprise a primer that hybridizes to exon a2 of ABL.

R. The method of N, O or Q, wherein the primers comprise a primer that hybridizes to exon e1 of BCR, a primer that hybridizes to exon b2 of BCR, and a primer that hybridizes to exon e19 of BCR.

S. The method of R, wherein the primer that hybridizes to exon b2 of BCR amplifies reverse-transcribed cDNA from a BCR-ABL gene fusion variant comprising a b2a2 gene fusion and reverse-transcribed cDNA from a BCR-ABL gene fusion variant comprising a b3a2 gene fusion.

T. The method of N or O, wherein one or more primers is/are detectably labeled.

U. The method of T, wherein detection of the one or more detectably labeled primers can be distinguished.

V. The method of O, wherein detecting the amplified cDNA comprises contacting the amplified cDNA with at least one probe under hybridizing conditions and detecting hybridization of at least one probe to the amplified cDNA.

W. The method of V, wherein at least one probe is a probe that hybridizes to a nucleotide sequence at or near a junction of a BCR-ABL gene fusion comprising the 3′ end of exon e1 of BCR and the 5′ end of exon a2 of ABL, a nucleotide sequence at or near a junction of a BCR-ABL gene fusion comprising the 3′ end of exon b2 of BCR and the 5′ end of exon a2 of ABL, a nucleotide sequence at or near a junction of a BCR-ABL gene fusion comprising the 3′ end of exon b3 of BCR and the 5′ end of exon a2 of ABL, or a nucleotide sequence at or near a junction of a BCR-ABL gene fusion comprising the 3′ end of exon e19 of BCR and the 5′ end of exon a2 of ABL.

X. The method of V or W, wherein detecting the amplified cDNA comprises contacting the amplified cDNA with at least two probes, wherein hybridization of each probe to the amplified cDNA is distinguishable.

Y. The method of O, wherein (a) further comprises using primers for at least one IC gene, wherein the IC primers can hybridize to the same or different exons of the at least one IC gene, and (b) further comprises detecting the amplified IC cDNA.

Z. The method of Y, wherein one or more IC primers is/are detectably labeled.

AA. The method of Z, wherein detection of the one or more detectably labeled IC primers can be distinguished.

AB. The method of Y, wherein detecting the amplified IC cDNA comprises contacting the amplified IC cDNA with at least one IC probe and/or at least one IC primer under hybridizing conditions and detecting hybridization of the at least one IC probe/primer to the amplified IC cDNA.

AC. The method of AB, wherein detecting the amplified IC cDNA comprises contacting the amplified IC cDNA with at least two IC probes/primers, wherein each IC probe/primer is specific for a different IC cDNA and hybridization of each IC probe/primer to the amplified IC cDNA is distinguishable.

AD. The method of Q, wherein the primer that hybridizes to exon a2 of ABL comprises the nucleotide sequence of SEQ ID NO: 4, 25, 26, or 27.

AE. The method of Q, wherein the primer that hybridizes to exon a2 of ABL comprises the nucleotide sequence of SEQ ID NO: 4.

AF. The method of R, wherein the primer that hybridizes to exon e1 of BCR comprises the nucleotide sequence of SEQ ID NO: 1, 18, 19, 20, 21, or 22, the primer that hybridizes to exon b2 of BCR comprises the nucleotide sequence of SEQ ID NO: 2 or 23, and/or the primer that hybridizes to exon e19 of BCR comprises the nucleotide sequence of SEQ ID NO: 3 or 24.

AG. The method of R, wherein the primer that hybridizes to exon e1 of BCR comprises the nucleotide sequence of SEQ ID NO: 1, the primer that hybridizes to exon b2 of BCR comprises the nucleotide sequence of SEQ ID NO: 2, and/or the primer that hybridizes to exon e19 of BCR comprises the nucleotide sequence of SEQ ID NO: 3.

AH. The method of V, wherein the probe that hybridizes to a nucleotide sequence at or near a junction of a BCR-ABL gene fusion comprising the 3′ end of exon e1 of BCR and the 5′ end of exon a2 of ABL comprises SEQ ID NO: 5, 28, 29, 30, or a sequence complementary thereto, the probe that hybridizes to a nucleotide sequence at or near a junction of a BCR-ABL gene fusion comprising the 3′ end of exon b2 of BCR and the 5′ end of exon a2 of ABL comprises SEQ ID NO: 6, 31, 32, or a sequence complementary thereto, the probe that hybridizes to a nucleotide sequence at or near a junction of a BCR-ABL gene fusion comprising the 3′ end of exon b3 of BCR and the 5′ end of exon a2 of ABL comprises SEQ ID NO: 7, 33, 34, 35, or a sequence complementary thereto, and/or the probe that hybridizes to a nucleotide sequence at or near a junction of a BCR-ABL gene fusion comprising the 3′ end of exon e19 of BCR and the 5′ end of exon a2 of ABL comprises SEQ ID NO: 8, 36, or a sequence complementary thereto.

AI. The method of V, wherein the probe that hybridizes to a nucleotide sequence at or near a junction of a BCR-ABL gene fusion comprising the 3′ end of exon e1 of BCR and the 5′ end of exon a2 of ABL comprises SEQ ID NO: 5 or a sequence complementary thereto, the probe that hybridizes to a nucleotide sequence at or near a junction of a BCR-ABL gene fusion comprising the 3′ end of exon b2 of BCR and the 5′ end of exon a2 of ABL comprises SEQ ID NO: 6 or a sequence complementary thereto, the probe that hybridizes to a nucleotide sequence at or near a junction of a BCR-ABL gene fusion comprising the 3′ end of exon b3 of BCR and the 5′ end of exon a2 of ABL comprises SEQ ID NO: 7 or a sequence complementary thereto, and/or the probe that hybridizes to a nucleotide sequence at or near a junction of a BCR-ABL gene fusion comprising the 3′ end of exon e19 of BCR and the 5′ end of exon a2 of ABL comprises SEQ ID NO: 8 or a sequence complementary thereto.

AJ. A method of amplifying mRNA from fusion variants of the echinoderm microtubule-associated protein-like 4 (EML4) gene and the anaplastic lymphoma kinase (ALK) gene in a sample of mRNA from a human, which method comprises:

(a) (i) obtaining cDNA, which has been reverse-transcribed from the sample of mRNA, or reverse-transcribing the sample of mRNA to cDNA and (ii) amplifying the reverse-transcribed cDNA using primers comprising primers for a group of EML4-ALK gene fusion variants comprising at least two gene fusion variants selected from the group consisting of an E13A20 gene fusion, an E6aA20 gene fusion, an E6bA20 gene fusion, an E14A20 gene fusion, and an E20A20 gene fusion,

whereupon mRNA from EML4-ALK gene fusion variants in a sample of mRNA from a human is amplified.

AK. The method of AJ, which further comprises:

(b) detecting the amplified cDNA or detecting and quantitating the amplified cDNA,

whereupon mRNA from EML4-ALK gene fusion variants in a sample of mRNA from a human is detected.

AL. The method of AJ, wherein the reverse-transcribed cDNA is amplified by polymerase chain reaction in the presence of a mixture of dNTP, a buffer, a polymerase, and a divalent ion, while cycling between a temperature of about 58° C. to about 62° C. for about 30 seconds to about 40 seconds and a temperature of about 92° C. for about 30 seconds.

AM. The method of AJ or AK, wherein the primers comprise a primer that hybridizes to exon A20 of ALK.

AN. The method of AJ, AK or AM, wherein the primers comprise two or more of a primer that hybridizes to exon E13 of EML4, a primer that hybridizes to exon E6a of EML4, and a primer that hybridizes to exon E20 of EML4.

AO. The method of AN, wherein the primer that hybridizes to exon E6a of EML4 amplifies reverse-transcribed cDNA from an EML4-ALK gene fusion variant comprising an E6a gene fusion and reverse-transcribed cDNA from an EML4-ALK gene fusion variant comprising an Ebb gene fusion and/or the primer that hybridizes to exon E13 of EML4 amplifies reverse-transcribed cDNA from an EML4-ALK gene fusion variant comprising an E13 gene fusion and reverse-transcribed cDNA from an EML4-ALK gene fusion variant comprising an E14 gene fusion.

AP. The method of AJ or AK, wherein one or more primers is/are detectably labeled.

AQ. The method of AP, wherein detection of the one or more detectably labeled primers can be distinguished.

AR. The method of AK, wherein detecting the amplified cDNA comprises contacting the amplified cDNA with at least one probe under hybridizing conditions and detecting hybridization of the at least one probe to the amplified cDNA.

AS. The method of AR, wherein at least one probe is a probe that hybridizes to a nucleotide sequence at or near a junction of an EML4-ALK gene fusion comprising the 3′ end of exon E13 of EML4 and the 5′ end of exon A20 of ALK, a nucleotide sequence at or near a junction of an EML4-ALK gene fusion comprising the 3′ end of exon E6a of EML4 and the 5′ end of exon A20 of ALK, a nucleotide sequence at or near a junction of an EML4-ALK gene fusion comprising the 3′ end of exon Ebb of EML4 and the 5′ end of exon A20 of ALK, a nucleotide sequence at or near a junction of a EML4-ALK gene fusion comprising the 3′ end of exon E14 of EML4 and the 5′ end of exon A20 of ALK, or a nucleotide sequence at or near a junction of an EML4-ALK gene fusion comprising the 3′ end of exon E20 of EML4 and the 5′ end of exon A20 of ALK.

AT. The method of AR or AS, wherein detecting the amplified cDNA comprises contacting the amplified cDNA with at least two probes, wherein hybridization of each probe to the amplified cDNA is distinguishable.

AU. The method of AK, wherein (a) further comprises using primers for at least one IC gene, wherein the IC primers hybridize to the same or different exons of the at least one IC gene, and (b) further comprises detecting the amplified IC cDNA.

AV. The method of AU, wherein one or more IC primers is/are detectably labeled.

AW. The method of AV, wherein detection of the one or more detectably labeled IC primers can be distinguished.

AX. The method of AV, wherein detecting the amplified IC cDNA comprises contacting the amplified IC cDNA with at least one IC probe and/or at least one IC primer under hybridizing conditions and detecting hybridization of at least one IC probe/primer to the amplified IC cDNA.

AY. The method of AX, wherein detecting the amplified IC cDNA comprises contacting the amplified IC cDNA with at least two IC probes/primers, wherein each IC probe/primer is specific for a different IC cDNA and hybridization of each IC probe/primer to the amplified IC cDNA is distinguishable.

AZ. The method of AJ-AY, wherein (a)(ii) further comprises amplifying the reverse-transcribed cDNA using primers for at least one further EML4-ALK gene fusion variant selected from the group consisting of an E18A20 gene fusion, an E15A20 gene fusion, an E2A20 gene fusion, and an E17A20 gene fusion.

BA. A method of amplifying mRNA from fusion variants of the kinesin family member 5B (KIF5B) gene and the Ret (RET) proto-oncogene in a sample of mRNA from a human, which method comprises:

(a) (i) obtaining cDNA, which has been reverse-transcribed from the sample of mRNA, or reverse-transcribing the sample of mRNA to cDNA and (ii) amplifying the reverse-transcribed cDNA using primers comprising primers for a group of KIF5B-RET gene fusion variants comprising at least two gene fusion variants selected from the group consisting of a K15R12 gene fusion, a K16R12 gene fusion, a K22R12 gene fusion, and a K23R12 gene fusion,

whereupon mRNA from KIF5B-RET gene fusion variants in a sample of mRNA from a human is amplified.

BB. The method of BA, which further comprises:

(b) detecting the amplified cDNA or detecting and quantitating the amplified cDNA,

whereupon mRNA from KIF5B-RET gene fusion variants in a sample of mRNA from a human is detected.

BC. The method of BA, wherein the reverse-transcribed cDNA is amplified by polymerase chain reaction in the presence of a mixture of dNTP, a buffer, a polymerase, and a divalent ion, while cycling between a temperature of about 58° C. to about 62° C. for about 30 seconds to about 40 seconds and a temperature of about 92° C. for about 30 seconds.

BD. The method of BA or BB, wherein the primers comprise a primer that hybridizes to exon R12 of RET.

BE. The method of BA, BB or BD, wherein the primers comprise a primer that hybridizes to exon K15 of KIF5B and/or a primer that hybridizes to exon K22 of KIF5B.

BF. The method of BE, wherein the primer that hybridizes to exon K15 of KIF5B amplifies reverse-transcribed cDNA from a KIF5B-RET gene fusion variant comprising a K15R12 gene fusion and reverse-transcribed cDNA from a KIF5B-RET gene fusion variant comprising a K16R12 gene fusion and/or the primer that hybridizes to exon K22 of KIF5B amplifies reverse-transcribed cDNA from a KIF5B-RET gene fusion variant comprising a K22R12 gene fusion and reverse-transcribed cDNA from a KIF5B-RET gene fusion variant comprising a K23R12 gene fusion.

BG. The method of BA or BB, wherein one or more primers is/are detectably labeled.

BH. The method of BG, wherein detection of the one or more detectably labeled primers can be distinguished.

BI. The method of BB, wherein detecting the amplified cDNA comprises contacting the amplified cDNA with at least one probe under hybridizing conditions and detecting hybridization of the at least one probe to the amplified cDNA.

BJ. The method of BI, wherein at least one probe is a probe that hybridizes to a nucleotide sequence at or near a junction of a KIF5B-RET gene fusion comprising the 3′ end of exon K15 of KIF5B and the 5′ end of exon R12 of RET, a nucleotide sequence at or near a junction of a KIF5B-RET gene fusion comprising the 3′ end of exon K16 of KIF5B and the 5′ end of exon R12 of RET, a nucleotide sequence at or near a junction of a KIF5B-RET gene fusion comprising the 3′ end of exon K22 of KIF5B and the 5′ end of exon R12 of RET, or a nucleotide sequence at or near a junction of a KIF5B-RET gene fusion comprising the 3′ end of exon K23 of KIF5B and the 5′ end of exon R12 of RET.

BK. The method of BI or BJ, wherein detecting the amplified cDNA comprises contacting the amplified cDNA with at least two probes, wherein hybridization of each probe to the amplified cDNA is distinguishable.

BL. The method of BB, wherein (a) further comprises using primers for at least one IC gene, wherein the IC primers hybridize to the same or different exons of the at least one IC gene, and (b) further comprises detecting the amplified IC cDNA.

BM. The method of BL, wherein one or more IC primers is/are detectably labeled.

BN. The method of BM, wherein detection of the one or more detectably labeled IC primers can be distinguished.

BO. The method of BL, wherein detecting the amplified IC cDNA comprises contacting the amplified IC cDNA with at least one IC probe and/or at least one IC primer under hybridizing conditions and detecting hybridization of the at least one IC probe/primer to the amplified IC cDNA.

BP. The method of BO, wherein detecting the amplified IC cDNA comprises contacting the amplified IC cDNA with at least two IC probes/primers, wherein each IC probe/primer is specific for a different IC cDNA and hybridization of each IC probe/primer to the amplified IC cDNA is distinguishable.

BQ. A method of amplifying mRNA from fusion variants of the promyelocytic leukemia (PML) gene and the retinoic acid receptor alpha (RARα) gene in a sample of mRNA from a human, which method comprises:

(a) (i) obtaining cDNA, which has been reverse-transcribed from the sample of mRNA, or reverse-transcribing the sample of mRNA to cDNA and (ii) amplifying the reverse-transcribed cDNA using primers comprising primers for a group of PML-RARα gene fusion variants comprising at least two gene fusion variants selected from the group consisting of a P3R3 gene fusion, a P6aR3 gene fusion, and a P6bR3 gene fusion,

whereupon mRNA from PML-RARα gene fusion variants in a sample of mRNA from a human is amplified.

BR. The method of BQ, which further comprises:

(b) detecting the amplified cDNA or detecting and quantitating the amplified cDNA,

whereupon mRNA from PML-RARα gene fusion variants in a sample of mRNA from a human is detected.

BS. The method of BQ, wherein the reverse-transcribed cDNA is amplified by polymerase chain reaction in the presence of a mixture of dNTP, a buffer, a polymerase, and a divalent ion, while cycling between a temperature of about 58° C. to about 62° C. for about 30 seconds to about 40 seconds and a temperature of about 92° C. for about 30 seconds.

BT. The method of BQ or BR, wherein the primers comprise a primer that hybridizes to exon R3 of RARα.

BU. The method of BQ, BR or BT, wherein the primers comprise a primer that hybridizes to exon P3 of PML and/or a primer that hybridizes to exon 6a of PML.

BV. The method of BU, wherein the primer that hybridizes to exon 6a of PML amplifies reverse-transcribed cDNA from a PML-RARα gene fusion variant comprising a P6aR3 gene fusion and reverse-transcribed cDNA from a PML-RARα gene fusion variant comprising a P6bR3 gene fusion.

BW. The method of BQ or BR, wherein one or more primers is/are detectably labeled.

BX. The method of BW, wherein detection of the one or more detectably labeled primers can be distinguished.

BY. The method of BR, wherein detecting the amplified cDNA comprises contacting the amplified cDNA with at least one probe under hybridizing conditions and detecting hybridization of the at least one probe to the amplified cDNA.

BZ. The method of BY, wherein at least one probe is a probe that hybridizes to a nucleotide sequence at or near a junction of a PML-RARα gene fusion comprising the 3′ end of exon P3 of PML and the 5′ end of exon R3 of RARα, a nucleotide sequence at or near a junction of a PML-RARα gene fusion comprising the 3′ end of exon P6a of PML and the 5′ end of exon R3 of RARα, or a nucleotide sequence at or near a junction of a PML-RARα gene fusion comprising the 3′ end of exon P6b of PML and the 5′ end of exon R3 of RARα.

CA. The method of BY or BZ, wherein detecting the amplified cDNA comprises contacting the amplified cDNA with at least two probes, wherein hybridization of each probe to the amplified cDNA is distinguishable.

CB. The method of BR, wherein (a) further comprises using primers for at least one IC gene, wherein the IC primers hybridize to the same or different exons of the at least one IC gene, and (b) further comprises detecting the amplified IC cDNA.

CC. The method of CB, wherein one or more IC primers is/are detectably labeled.

CD. The method of CC, wherein detection of the one or more detectably labeled IC primers can be distinguished.

CE. The method of CB, wherein detecting the amplified IC cDNA comprises contacting the amplified IC cDNA with at least one IC probe and/or at least one IC primer under hybridizing conditions and detecting hybridization of the at least one IC probe/primer to the amplified IC cDNA.

CF. The method of CE, wherein detecting the amplified IC cDNA comprises contacting the amplified IC cDNA with at least two IC probes/primers, wherein each IC probe/primer is specific for a different IC cDNA and hybridization of each IC probe/primer to the amplified IC cDNA is distinguishable.

CG. A set of primers comprising at least two primers selected from the group consisting of a primer that hybridizes to exon e1 of BCR, a primer that hybridizes to exon b2 of BCR, and a primer that hybridizes to exon e19 of BCR, wherein the primer can be detectably labeled and/or wherein the set of primers is combined with at least one detectably labeled probe selected from the group consisting of a probe that hybridizes to a nucleotide sequence at or near a junction of a BCR-ABL gene fusion comprising the 3′ end of exon e1 of BCR and the 5′ end of exon a2 of ABL, a probe that hybridizes to a nucleotide sequence at or near a junction of a BCR-ABL gene fusion comprising the 3′ end of exon b2 of BCR and the 5′ end of exon a2 of ABL, a probe that hybridizes to a nucleotide sequence at or near a junction of a BCR-ABL gene fusion comprising the 3′ end of exon b3 of BCR and the 5′ end of exon a2 of ABL, and a probe that hybridizes to a nucleotide sequence at or near a junction of a BCR-ABL gene fusion comprising the 3′ end of exon e19 of BCR and the 5′ end of exon a2 of ABL.

CH. The set of primers of CG, wherein the primer that hybridizes to exon e1 of BCR comprises a nucleotide sequence selected from the group consisting of SEQ ID NOS: 1, 18, 19, 20, 21, and 22.

CI. The set of primers of CG, wherein the primer that hybridizes to exon b2 of BCR comprises a nucleotide sequence selected from the group consisting of SEQ ID NOS: 2 and 23.

CJ. The set of primers of CG, wherein the primer that hybridizes to exon e19 of BCR comprises a nucleotide sequence selected from the group consisting of SEQ ID NOS: 3 and 24.

CK. The set of primers of CG, which further comprises a primer that hybridizes to exon a2 of ABL.

CL. The set of primers of CK, wherein the primer that hybridizes to exon a2 of ABL comprises a nucleotide sequence selected from the group consisting of SEQ ID NOS: 4, 25, 26, and 27.

CM. A set of primers comprising at least two primers selected from the group consisting of a primer that hybridizes to exon E13 of EML4, a primer that hybridizes to exon E6a of EML4, a primer that hybridizes to exon Ebb of EML4, a primer that hybridizes to exon E14 of EML4, and a primer that hybridizes to exon E20 of EML4, wherein the primer can be detectably labeled and/or wherein the set of primers is combined with at least one detectably labeled probe selected from the group consisting of a probe that hybridizes to a nucleotide sequence at or near a junction of a EML4-ALK gene fusion comprising the 3′ end of exon E13 of EML4 and the 5′ end of exon A20 of ALK, a probe that hybridizes to a nucleotide sequence at or near a junction of a EML4-ALK gene fusion comprising the 3′ end of exon E6a of EML4 and the 5′ end of exon A20 of ALK, a probe that hybridizes to a nucleotide sequence at or near a junction of a EML4-ALK gene fusion comprising the 3′ end of exon Ebb of EML4 and the 5′ end of exon A20 of ALK, a probe that hybridizes to a nucleotide sequence at or near a junction of a EML4-ALK gene fusion comprising the 3′ end of exon E14 of EML4 and the 5′ end of exon A20 of ALK, and a probe that hybridizes to a nucleotide sequence at or near a junction of a EML4-AKL gene fusion comprising the 3′ end of exon E20 of EML4 and the 5′ end of exon A20 of ALK.

CN. The set of primers of CM, which further comprises a primer that hybridizes to exon A20 of ALK.

CO. A set of primers comprising at least two primers selected from the group consisting of a primer that hybridizes to exon K15 of KIF5B, a primer that hybridizes to exon K16 of KIF5B, a primer that hybridizes to exon K22 of KIF5B, and a primer that hybridizes to exon K23 of KIF5B, wherein the primer can be detectably labeled and/or wherein the set of primers is combined with at least one detectably labeled probe selected from the group consisting of a probe that hybridizes to a nucleotide sequence at or near a junction of a KIF5B-RET gene fusion comprising the 3′ end of exon K15 of KIF5B and the 5′ end of exon R12 of RET, a probe that hybridizes to a nucleotide sequence at or near a junction of a KIF5B-RET gene fusion comprising the 3′ end of exon K16 of KIF5B and the 5′ end of exon R12 of RET, a probe that hybridizes to a nucleotide sequence at or near a junction of a KIF5B-RET gene fusion comprising the 3′ end of exon K22 of KIF5B and the 5′ end of exon R12 of RET, and a probe that hybridizes to a nucleotide sequence at or near a junction of a KIF5B-RET gene fusion comprising the 3′ end of exon K23 of KIF5B and the 5′ end of exon R12 of RET.

CP. The set of primers of CO, which further comprises a primer that hybridizes to exon R12 of RET.

CQ. A set of primers comprising at least two primers selected from the group consisting of a primer that hybridizes to exon P3 of PML, a primer that hybridizes to exon 6a of PML, and a primer that hybridizes to exon 6b of PML, wherein the primer can be detectably labeled and/or wherein the set of primers is combined with at least one detectably labeled probe selected from the group consisting of a probe that hybridizes to a nucleotide sequence at or near a junction of a PML-RARα gene fusion comprising the 3′ end of exon P3 of PML and the 5′ end of exon R3 of RARα, a probe that hybridizes to a nucleotide sequence at or near a junction of a PML-RARα gene fusion comprising the 3′ end of exon P6a of PML and the 5′ end of exon R3 of RARα, and a probe that hybridizes to a nucleotide sequence at or near a junction of a PML-RARα gene fusion comprising the 3′ end of exon P6b of PML and the 5′ end of exon R3 of RARα.

CR. The set of primers of CQ, which further comprises a primer that hybridizes to exon R3 of RARα.

CS. A set of probes comprising at least two probes selected from the group consisting of a probe that hybridizes to a nucleotide sequence at or near a junction of a BCR-ABL gene fusion comprising the 3′ end of exon e1 of BCR and the 5′ end of exon a2 of ABL, a probe that hybridizes to a nucleotide sequence at or near a junction of a BCR-ABL gene fusion comprising the 3′ end of exon b2 of BCR and the 5′ end of exon a2 of ABL, a probe that hybridizes to a nucleotide sequence at or near a junction of a BCR-ABL gene fusion comprising the 3′ end of exon b3 of BCR and the 5′ end of exon a2 of ABL, and a probe that hybridizes to a nucleotide sequence at or near a junction of a BCR-ABL gene fusion comprising the 3′ end of exon e19 of BCR and the 5′ end of exon a2 of ABL.

CT. The set of probes of CS, wherein the probe that hybridizes to a nucleotide sequence at or near a junction of a BCR-ABL gene fusion comprising the 3′ end of exon e1 of BCR and the 5′ end of exon a2 of ABL comprises a nucleotide sequence selected from the group consisting of SEQ ID NOS: 5, 28, 29, 30, and a sequence complementary thereto.

CU. The set of probes of CS, wherein the probe that hybridizes to a nucleotide sequence at or near a junction of a BCR-ABL gene fusion comprising the 3′ end of exon b2 of BCR and the 5′ end of exon a2 of ABL comprises a nucleotide sequence selected from the group consisting of SEQ ID NOS: 6, 31, 32, and a sequence complementary thereto.

CV. The set of probes of CS, wherein the probe that hybridizes to a nucleotide sequence at or near a junction of a BCR-ABL gene fusion comprising the 3′ end of exon b3 of BCR and the 5′ end of exon a2 of ABL comprises a nucleotide sequence selected from the group consisting of SEQ ID NOS: 7, 33, 34, 35, and a sequence complementary thereto.

CW. The set of probes of CS, wherein the probe that hybridizes to a nucleotide sequence at or near a junction of a BCR-ABL gene fusion comprising the 3′ end of exon e19 of BCR and the 5′ end of exon a2 of ABL comprises a nucleotide sequence selected from the group consisting of SEQ ID NOS: 8, 36, and a sequence complementary thereto.

CX. A set of probes comprising at least two probes selected from the group consisting of a probe that hybridizes to a nucleotide sequence at or near a junction of a EML4-ALK gene fusion comprising the 3′ end of exon E13 of EML4 and the 5′ end of exon A20 of ALK, a probe that hybridizes to a nucleotide sequence at or near a junction of a EML4-ALK gene fusion comprising the 3′ end of exon E6a of EML4 and the 5′ end of exon A20 of ALK, a probe that hybridizes to a nucleotide sequence at or near a junction of a EML4-ALK gene fusion comprising the 3′ end of exon Ebb of EML4 and the 5′ end of exon A20 of ALK, a probe that hybridizes to a nucleotide sequence at or near a junction of a EML4-ALK gene fusion comprising the 3′ end of exon E14 of EML4 and the 5′ end of exon A20 of ALK, and a probe that hybridizes to a nucleotide sequence at or near a junction of a EML4-AKL gene fusion comprising the 3′ end of exon E20 of EML4 and the 5′ end of exon A20 of ALK.

CY. A set of probes comprising at least two probes selected from the group consisting of a probe that hybridizes to a nucleotide sequence at or near a junction of a KIF5B-RET gene fusion comprising the 3′ end of exon K15 of KIF5B and the 5′ end of exon R12 of RET, a probe that hybridizes to a nucleotide sequence at or near a junction of a KIF5B-RET gene fusion comprising the 3′ end of exon K16 of KIF5B and the 5′ end of exon R12 of RET, a probe that hybridizes to a nucleotide sequence at or near a junction of a KIF5B-RET gene fusion comprising the 3′ end of exon K22 of KIF5B and the 5′ end of exon R12 of RET, and a probe that hybridizes to a nucleotide sequence at or near a junction of a KIF5B-RET gene fusion comprising the 3′ end of exon K23 of KIF5B and the 5′ end of exon R12 of RET.

CZ. A set of probes comprising at least two probes selected from the group consisting of a probe that hybridizes to a nucleotide sequence at or near a junction of a PML-RARα gene fusion comprising the 3′ end of exon P3 of PML and the 5′ end of exon R3 of RARα, a probe that hybridizes to a nucleotide sequence at or near a junction of a PML-RARα gene fusion comprising the 3′ end of exon P6a of PML and the 5′ end of exon R3 of RARα, and a probe that hybridizes to a nucleotide sequence at or near a junction of a PML-RARα gene fusion comprising the 3′ end of exon P6b of PML and the 5′ end of exon R3 of RARα.

DA. A kit comprising:

(i) a set of primers comprising a primer that hybridizes to an exon of a first gene, which becomes contiguous with a variant exon of a second gene, and a primer for each of two or more variant exons of the second gene; and

(ii) instructions for a method of detecting mRNA from fusions of the first gene and the second gene in a sample of mRNA, which method comprises:
(a) (i) obtaining cDNA, which has been reverse-transcribed from the sample of mRNA, or reverse-transcribing the sample of mRNA to cDNA and (ii′) amplifying the reverse-transcribed cDNA using primers comprising a primer for an exon of the first gene, which becomes contiguous with a variant exon of the second gene, and a primer for each of two or more variant exons of the second gene, wherein one or more of the primers can be detectably labeled, and
(b) detecting the amplified cDNA or detecting and quantitating the amplified cDNA.

DB. The kit of DA, which further comprises a probe for each gene fusion variant that is not detected using a detectably labeled primer, wherein the probe hybridizes to a nucleotide sequence at or near a junction of a variant of a fusion between the first gene and the second gene comprising the 3′ end of an exon of the first gene and the 5′ end of an exon of the second gene.

DC. The kit of DA, wherein (ii) (a) further comprises using primers for at least one IC gene, wherein the IC primers hybridize to the same or different exons of the at least one IC gene, and (b) further comprises detecting the amplified IC cDNA.

DD. A kit comprising:

(i) a set of primers comprising at least two primers selected from the group consisting of a primer that hybridizes to exon e1 of BCR, a primer that hybridizes to exon b2 of BCR, and a primer that hybridizes to exon e19 of BCR; and

(ii) instructions for a method of detecting mRNA from fusions of the BCR gene and the ABL proto-oncogene in a sample of mRNA from a human, which method comprises:

(a) (i) obtaining cDNA, which has been reverse-transcribed from the sample of mRNA, or reverse-transcribing the sample of mRNA to cDNA and (ii) amplifying the reverse-transcribed cDNA using primers for a group of BCR-ABL gene fusion variants comprising at least two gene fusion variants selected from the group consisting of an e1a2 gene fusion, a b2a2 gene fusion, a b3a2 gene fusion, and an e19a2 gene fusion, wherein one or more of the primers can be detectably labeled, and

(b) detecting the amplified cDNA or detecting and quantitating the amplified cDNA.

DE. The kit of DD, which comprises a primer that hybridizes to exon a2 of ABL.

DF. The kit of DD, which further comprises a probe for each gene fusion variant that is not detected using a detectably labeled primer, wherein the probe hybridizes to a nucleotide sequence at or near a junction of a BCR-ABL gene fusion comprising the 3′ end of exon e1 of BCR and the 5′ end of exon a2 of ABL, a nucleotide sequence at or near a junction of a BCR-ABL gene fusion comprising the 3′ end of exon b2 of BCR and the 5′ end of exon a2 of ABL, a nucleotide sequence at or near a junction of a BCR-ABL gene fusion comprising the 3′ end of exon b3 of BCR and the 5′ end of exon a2 of ABL, or a nucleotide sequence at or near a junction of a BCR-ABL gene fusion comprising the 3′ end of exon e19 of BCR and the 5′ end of exon a2 of ABL.

DG. The kit of DD, wherein (ii) (a) further comprises using primers for at least one IC gene, wherein the primers hybridize to the same or different exons of the at least one IC gene, and (b) further comprises detecting the amplified IC cDNA.

DH. A kit comprising:

(i) a set of primers comprising a primer comprising a nucleotide sequence selected from the group consisting of SEQ ID NO: 1, 2, 3, 18, 19, 20, 21, 22, 23, and 24; and

(ii) instructions for a method of detecting mRNA from fusions of the BCR gene and the ABL proto-oncogene in a sample of mRNA from a human, which method comprises:

(a) (i) obtaining cDNA, which has been reverse-transcribed from the sample of mRNA, or reverse-transcribing the sample of mRNA to cDNA and (ii) amplifying the reverse-transcribed cDNA using primers for at least one BCR-ABL gene fusion variant selected from the group consisting of an e1a2 gene fusion, a b2a2 gene fusion, a b3a2 gene fusion, and an e19a2 gene fusion, wherein the primers can be detectably labeled, and

(b) detecting the amplified cDNA or detecting and quantitating the amplified cDNA.

DI. The kit of DH, which further comprises a primer comprising a nucleotide sequence selected from the group consisting of SEQ ID NOS: 4, 25, 26, and 27.

DJ. The kit of DH, which further comprises a probe for each gene fusion variant that is not detected using a detectably labeled primer, wherein the probe comprises a nucleotide sequence selected from the group consisting of SEQ ID NO: 5, 6, 7, 8, 28, 29, 30, 31, 32, 33, 34, 35, 36, and a sequence complementary thereto.

DK. The kit of DH, wherein (ii) (a) further comprises using primers for at least one IC gene, wherein the IC primers hybridize to the same or different exons of the at least one IC gene, and (b) further comprises detecting the amplified IC cDNA.

DL. A kit comprising:

(i) a set of primers comprising at least two primers selected from the group consisting of a primer that hybridizes to exon E13 of EML4, a primer that hybridizes to exon E6a of EML4, a primer that hybridizes to exon Ebb of EML4, a primer that hybridizes to exon E14 of EML4, and a primer that hybridizes to exon E20 of EML4; and

(ii) instructions for a method of detecting mRNA from fusions of the EML4 gene and the ALK gene in a sample of mRNA from a human, which method comprises:
(a) (i) obtaining cDNA, which has been reverse-transcribed from the sample of mRNA, or reverse-transcribing the sample of mRNA to cDNA and (ii) amplifying the reverse-transcribed cDNA using primers for a group of EML4-ALK gene fusion variants comprising at least two gene fusion variants selected from the group consisting of an E13A20 gene fusion, an E6aA20 gene fusion, an E6bA20 gene fusion, an E14A20 gene fusion, and an E20A20 gene fusion, wherein one or more of the primers can be detectably labeled, and
(b) detecting the amplified cDNA or detecting and quantitating the amplified cDNA.

DM. The kit of DL, which comprises a primer that hybridizes to exon A20 of ALK.

DN. The kit of DL, which further comprises a probe for each gene fusion variant that is not detected using a detectably labeled primer, wherein the probe hybridizes to a nucleotide sequence at or near a junction of a EML4-ALK gene fusion comprising the 3′ end of exon E13 of EML4 and the 5′ end of exon A20 of ALK, a nucleotide sequence at or near a junction of a EML4-ALK gene fusion comprising the 3′ end of exon E6a of EML4 and the 5′ end of exon A20 of ALK, a nucleotide sequence at or near a junction of a EML4-ALK gene fusion comprising the 3′ end of exon Ebb of EML4 and the 5′ end of exon A20 of ALK, a nucleotide sequence at or near a junction of a EML4-ALK gene fusion comprising the 3′ end of exon E14 of EML4 and the 5′ end of exon A20 of ALK, or a nucleotide sequence at or near a junction of a EML4-AKL gene fusion comprising the 3′ end of exon E20 of EML4 and the 5′ end of exon A20 of ALK.

DO. The kit of DL, wherein (ii) (a) further comprises using primers for at least one IC gene, wherein the IC primers hybridize to the same or different exons of the at least one IC gene, and (b) further comprises detecting the amplified IC cDNA.

DP. A kit comprising:

(i) a set of primers comprising at least two primers selected from the group consisting of a primer that hybridizes to exon K15 of KIF5B, a primer that hybridizes to exon K16 of KIF5B, a primer that hybridizes to exon K22 of KIF5B, and a primer that hybridizes to exon K23 of KIF5B; and

(ii) instructions for a method of detecting mRNA from fusions of the KIF5B gene and the RET gene in a sample of mRNA from a human, which method comprises:
(a) (i) obtaining cDNA, which has been reverse-transcribed from the sample of mRNA, or reverse-transcribing the sample of mRNA to cDNA and (ii) amplifying the reverse-transcribed cDNA using primers for a group of KIF5B-RET gene fusion variants comprising at least two gene fusion variants selected from the group consisting of a K15R12 gene fusion, a K16R12 gene fusion, a K22R12 gene fusion, and a K23R12 gene fusion, wherein one or more of the primers can be detectably labeled, and
(b) detecting the amplified cDNA or detecting and quantitating the amplified cDNA.

DQ. The kit of DP, which comprises a primer that hybridizes to exon R12 of RET.

DR. The kit of DP, which further comprises a probe for each gene fusion variant that is not detected using a detectably labeled primer, wherein the probe hybridizes to a nucleotide sequence at or near a junction of a KIF5B-RET gene fusion comprising the 3′ end of exon K15 of KIF5B and the 5′ end of exon R12 of RET, a nucleotide sequence at or near a junction of a KIF5B-RET gene fusion comprising the 3′ end of exon K16 of KIF5B and the 5′ end of exon R12 of RET, a nucleotide sequence at or near a junction of a KIF5B-RET gene fusion comprising the 3′ end of exon K22 of KIF5B and the 5′ end of exon R12 of RET, or a nucleotide sequence at or near a junction of a KIF5B-RET gene fusion comprising the 3′ end of exon K23 of KIF5B and the 5′ end of exon R12 of RET.

DS. The kit of DP, wherein (ii) (a) further comprises using primers for at least one IC gene, wherein the IC primers hybridize to the same or different exons of the at least one IC gene, and (b) further comprises detecting the amplified IC cDNA.

DT. A kit comprising:

(i) a set of primers comprising at least two primers selected from the group consisting of a primer that hybridizes to exon P3 of PML, a primer that hybridizes to exon 6a of PML, and a primer that hybridizes to exon 6b of PML; and

(ii) instructions for a method of detecting mRNA from fusions of the PML gene and the RARα gene in a sample of mRNA from a human, which method comprises:
(a) (i) obtaining cDNA, which has been reverse-transcribed from the sample of mRNA, or reverse-transcribing the sample of mRNA to cDNA and (ii) amplifying the reverse-transcribed cDNA using primers for a group of PML-RARα gene fusion variants comprising at least two gene fusion variants selected from the group consisting of a P3R3 gene fusion, a P6aR3 gene fusion, and a P6bR3 gene fusion, wherein one or more of the primers can be detectably labeled and
(b) detecting the amplified cDNA or detecting and quantitating the amplified cDNA.

DU. The kit of DT, which comprises a primer that hybridizes to exon R3 of RARα.

DV. The kit of DT, which further comprises a probe for each gene fusion variant that is not detected using a detectably labeled primer, wherein the probe hybridizes to a nucleotide sequence at or near a junction of a PML-RARα gene fusion comprising the 3′ end of exon P3 of PML and the 5′ end of exon R3 of RARα, a nucleotide sequence at or near a junction of a PML-RARα gene fusion comprising the 3′ end of exon P6a of PML and the 5′ end of exon R3 of RARα, or a nucleotide sequence at or near a junction of a PML-RARα gene fusion comprising the 3′ end of exon P6b of PML and the 5′ end of exon R3 of RARα.

DW. The kit of DT, wherein (ii) (a) further comprises using primers for at least one IC gene, wherein the IC primers hybridize to the same or different exons of the at least one IC gene, and (b) further comprises detecting the amplified IC cDNA.

DX. A method of detecting mRNA from variants of a fusion between a first gene and a second gene in a sample of mRNA, which method comprises:

(a) (i) obtaining cDNA, which has been reverse-transcribed from the sample of mRNA, or reverse-transcribing the sample of mRNA to cDNA and (ii) amplifying the reverse-transcribed cDNA using a primer for an exon of the first gene, which, upon translocation, is contiguous with a variant exon of the second gene, and a primer for each of two or more variant exons of the second gene, and

(b) detecting the amplified cDNA or detecting and quantitating the amplified cDNA,

whereupon mRNA from variants of a fusion between the first gene and the second gene in a sample of mRNA is detected.

DY. The method of DX, wherein one or more primers is/are detectably labeled.

DZ. The method of DY, wherein detection of the one or more detectably labeled primers can be distinguished.

EA. The method of DX, wherein detecting the amplified cDNA comprises contacting the amplified cDNA with at least one probe under hybridizing conditions and detecting hybridization of at least one probe to the amplified cDNA.

EB. The method of EA, wherein at least one probe is a probe that hybridizes to a nucleotide sequence at or near a junction of a variant of a fusion between the first gene and the second gene comprising the 3′ end of an exon of the first gene and the 5′ end of an exon of the second gene.

EC. The method of EA or EB, wherein detecting the amplified cDNA comprises contacting the amplified cDNA with at least two probes, wherein hybridization of each probe to the amplified cDNA is distinguishable.

ED. The method of DX, wherein (a) further comprises using primers for at least one IC gene, wherein the IC primers hybridize to the same or different exons of the at least one IC gene, and (b) further comprises detecting the amplified IC cDNA.

EE. The method of ED, wherein one or more IC primers is/are detectably labeled.

EF. The method of EE, wherein detection of the one or more detectably labeled IC primers can be distinguished.

EG. The method of EF, wherein detecting the amplified IC cDNA comprises contacting the amplified IC cDNA with at least one IC probe and/or at least one IC primer under hybridizing conditions and detecting hybridization of the at least one IC probe/primer to the amplified IC cDNA.

EH. The method of EG, wherein detecting the amplified IC cDNA comprises contacting the amplified IC cDNA with at least two IC probes/primers, wherein each IC probe/primer is specific for a different IC cDNA and hybridization of each IC probe/primer to the amplified IC cDNA is distinguishable.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation. In this regard, where certain terms are set forth under “Terms” and are otherwise defined, described, explained, or discussed elsewhere in the “Detailed Description,” all such definitions, descriptions, explanations, and discussions are intended to be attributed to such terms. There also is no intention in the use of such terms and expressions of excluding any equivalents of the features shown and described or portions thereof. Furthermore, while subheadings, e.g., “Terms,” are used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, it should be understood that, although the present invention has been specifically disclosed in the context of preferred embodiments and optional features, those skilled in the art may resort to modifications and variations of the concepts disclosed herein. Such modifications and variations are considered to be within the scope of the invention as defined by the appended claims.

What is claimed is:

1. A method of amplifying mRNA from variants of a fusion between a first gene and a second gene in a sample of mRNA, which method comprises:
(a) (i) obtaining cDNA, which has been reverse-transcribed from the sample of mRNA, or reverse-transcribing the sample of mRNA to cDNA and (ii) amplifying the reverse-transcribed cDNA using primers comprising a primer for an exon of the first gene, which becomes contiguous with a variant exon of the second gene, and a primer for each of two or more variant exons of the second gene,
wherein one or more primers can be detectably labeled,
whereupon mRNA from variants of a fusion between a first gene and a second gene in a sample of mRNA is amplified.

2. The method of claim 1, which further comprises:
(b) detecting the amplified cDNA or detecting and quantitating the amplified cDNA,
whereupon mRNA from variants of a fusion between the first gene and the second in a sample of mRNA is detected.

3. The method of claim 2, wherein detecting the amplified cDNA comprises contacting the amplified cDNA with at least one probe under hybridizing conditions and detecting hybridization of the at least one probe to the amplified cDNA.

4. The method of any of claim 1, 2 or 3, wherein the first gene and the second gene are the breakpoint cluster region (BCR) gene and the Abelson murine leukemia (ABL) proto-oncogene, the echinoderm microtubule-associated protein-like 4 (EML4) gene and the anaplastic lymphoma kinase (ALK) gene, the kinesin family member 5B (KIF5B) gene and the Ret (RET) proto-oncogene, or the promyelocytic leukemia (PML) gene and the retinoic acid receptor alpha (RARα) gene.

5. The method of claim 3, wherein at least one probe is a probe that hybridizes to a nucleotide sequence at or near a junction of a variant of a fusion between the first gene and the second gene comprising the 3′ end of an exon of the first gene and the 5′ end of an exon of the second gene.

6. The method of claim 4, wherein the primers comprise primers for a group of BCR-ABL gene fusion variants comprising at least two gene fusion variants selected from the group consisting of an e1a2 gene fusion, a b2a2 gene fusion, a b3a2 gene fusion, and an e19a2 gene fusion.

7. The method of claim 6, wherein the primers comprise (i) a primer that hybridizes to exon a2 of ABL and (ii) at least two of a primer that hybridizes to exon e1 of BCR, a primer that hybridizes to exon b2 of BCR, and a primer that hybridizes to exon e19 of BCR.

8. The method of claim 7, wherein the primer that hybridizes to exon b2 of BCR amplifies reverse-transcribed cDNA from a BCR-ABL gene fusion variant comprising a b2a2 gene fusion and reverse-transcribed cDNA from a BCR-ABL gene fusion variant comprising a b3a2 gene fusion.

9. The method of claim 6, wherein at least one probe is a probe that hybridizes to a nucleotide sequence at or near a junction of a BCR-ABL gene fusion comprising the 3′ end of exon e1 of BCR and the 5′ end of exon a2 of ABL, a nucleotide sequence at or near a junction of a BCR-ABL gene fusion comprising the 3′ end of exon b2 of BCR and the 5′ end of exon a2 of ABL, a nucleotide sequence at or near a junction of a BCR-ABL gene fusion comprising the 3′ end of exon b3 of BCR and the 5′ end of exon a2 of ABL, or a nucleotide sequence at or near a junction of a BCR-ABL gene fusion comprising the 3′ end of exon e19 of BCR and the 5′ end of exon a2 of ABL.

10. The method of claim 7, wherein the primer that hybridizes to exon a2 of ABL comprises a nucleotide sequence selected from the group consisting of SEQ ID NOs: 4, 25, 26, and 27, the primer that hybridizes to exon e1 of BCR comprises a nucleotide sequence selected from the group consisting of SEQ ID NOs: 1, 18, 19, 20, 21, and 22, the primer that hybridizes to exon b2 of BCR comprises a nucleotide sequence selected from the group consisting of SEQ ID NOs: 2 and 23, and/or the primer that hybridizes to exon e19 of BCR comprises a nucleotide sequence selected from the group consisting of SEQ ID NOs: 3 and 24.

11. The method of claim 9, wherein the probe that hybridizes to a nucleotide sequence at or near a junction of a BCR-ABL gene fusion comprising the 3′ end of exon e1 of BCR and the 5′ end of exon a2 of ABL comprises a nucleotide sequence selected from the group consisting of SEQ ID NOs: 5, 28, 29, 30, and a sequence complementary thereto, the probe that hybridizes to a nucleotide sequence at or near a junction of a BCR-ABL gene fusion comprising the 3′ end of exon b2 of BCR and the 5′ end of exon a2 of ABL comprises a nucleotide sequence selected from the group consisting of SEQ ID NOs: 6, 31, 32, and a sequence complementary thereto, the probe that hybridizes to a nucleotide sequence at or near a junction of a BCR-ABL gene fusion comprising the 3′ end of exon b3 of BCR and the 5′ end of exon a2 of ABL comprises a nucleotide sequence selected from the group consisting of SEQ ID NOs: 7, 33, 34, 35, and a sequence complementary thereto, and/or the probe that hybridizes to a nucleotide sequence at or near a junction of a BCR-ABL gene fusion comprising the 3′ end of exon e19 of BCR and the 5′ end of exon a2 of ABL comprises a nucleotide sequence selected from the group consisting of SEQ ID NOs: 8, 36, and a sequence complementary thereto.

12. The method of claim 4, wherein the primers comprise primers for a group of EML4-ALK gene fusion variants comprising at least two gene fusion variants selected from the group consisting of an E13A20 gene fusion, an E6aA20 gene fusion, an E6bA20 gene fusion, an E14A20 gene fusion, and an E20A20 gene fusion.

13. The method of claim 12, wherein the primers comprise (i) a primer that hybridizes to exon A20 of ALK and (ii) two or more of a primer that hybridizes to exon E13 of EML4, a primer that hybridizes to exon E6a of EML4, and a primer that hybridizes to exon E20 of EML4.

14. The method of claim 13, wherein the primer that hybridizes to exon E6a of EML4 amplifies reverse-transcribed cDNA from an EML4-ALK gene fusion variant comprising an E6a gene fusion and reverse-transcribed cDNA from an EML4-ALK gene fusion variant comprising an E6b gene fusion and/or the primer that hybridizes to exon E13 of EML4 amplifies reverse-transcribed cDNA from an EML4-ALK gene fusion variant comprising an E13 gene fusion and reverse-transcribed cDNA from an EML4-ALK gene fusion variant comprising an E14 gene fusion.

15. The method of claim 12, wherein at least one probe is a probe that hybridizes to a nucleotide sequence at or near a junction of an EML4-ALK gene fusion comprising the 3′ end of exon E13 of EML4 and the 5′ end of exon A20 of ALK, a nucleotide sequence at or near a junction of an EML4-ALK gene fusion comprising the 3′ end of exon E6a of EML4 and the 5′ end of exon A20 of ALK, a nucleotide sequence at or near a junction of an EML4-ALK gene fusion comprising the 3′ end of exon E6b of EML4 and the 5′ end of exon A20 of ALK, a nucleotide sequence at or near a junction of a EML4-ALK gene fusion comprising the 3′ end of exon E14 of EML4 and the 5′ end of exon A20 of ALK, or a nucleotide sequence at or near a junction of an EML4-ALK gene fusion comprising the 3′ end of exon E20 of EML4 and the 5′ end of exon A20 of ALK.

16. The method of claim 12, wherein (a)(ii) further comprises amplifying the reverse-transcribed cDNA using primers for at least one further EML4-ALK gene fusion variant selected from the group consisting of an E18A20 gene fusion, an E15A20 gene fusion, an E2A20 gene fusion, and an E17A20 gene fusion.

17. The method of claim 4, wherein the primers comprise primers for a group of KIF5B-RET gene fusion variants comprising at least two gene fusion variants selected from the group consisting of a K15R12 gene fusion, a K16R12 gene fusion, a K22R12 gene fusion, and a K23R12 gene fusion.

18. The method of claim 17, wherein the primers comprise (i) a primer that hybridizes to exon R12 of RET and (ii) a primer that hybridizes to exon K15 of KIF5B and/or a primer that hybridizes to exon K22 of KIF5B.

19. The method of claim 18, wherein the primer that hybridizes to exon K15 of KIF5B amplifies reverse-transcribed cDNA from a KIF5B-RET gene fusion variant comprising a K15R12 gene fusion and reverse-transcribed cDNA from a KIF5B-RET gene fusion variant comprising a K16R12 gene fusion and/or the primer that hybridizes to exon K22 of KIF5B amplifies reverse-transcribed cDNA from a KIF5B-RET gene fusion variant comprising a K22R12 gene fusion and reverse-transcribed cDNA from a KIF5B-RET gene fusion variant comprising a K23R12 gene fusion.

20. The method of claim 17, wherein at least one probe is a probe that hybridizes to a nucleotide sequence at or near a junction of a KIF5B-RET gene fusion comprising the 3′ end of exon K15 of KIF5B and the 5′ end of exon R12 of RET, a nucleotide sequence at or near a junction of a KIF5B-RET gene fusion comprising the 3′ end of exon K16 of KIF5B and the 5′ end of exon R12 of RET, a nucleotide sequence at or near a junction of a KIF5B-RET gene fusion comprising the 3′ end of exon K22 of KIF5B and the 5′ end of exon R12 of RET, or a nucleotide sequence at or near a junction of a KIF5B-RET gene fusion comprising the 3′ end of exon K23 of KIF5B and the 5′ end of exon R12 of RET.

21. The method of claim 4, wherein the primers comprise primers for a group of PML-RARα gene fusion variants comprising at least two gene fusion variants selected from the group consisting of a P3R3 gene fusion, a P6aR3 gene fusion, and a P6bR3 gene fusion.

22. The method of claim 21, wherein the primers comprise (i) a primer that hybridizes to exon R3 of RARα and (ii) a primer that hybridizes to exon P3 of PML and/or a primer that hybridizes to exon 6a of PML.

23. The method of claim 22, wherein the primer that hybridizes to exon 6a of PML amplifies reverse-transcribed cDNA from a PML-RARα gene fusion variant comprising a P6aR3 gene fusion and reverse-transcribed cDNA from a PML-RARα gene fusion variant comprising a P6bR3 gene fusion.

24. The method of claim 21, wherein at least one probe is a probe that hybridizes to a nucleotide sequence at or near a junction of a PML-RARα gene fusion comprising the 3′ end of exon P3 of PML and the 5′ end of exon R3 of RARα, a nucleotide sequence at or near a junction of a PML-RARα gene fusion comprising the 3′ end of exon P6a of PML and the 5′ end of exon R3 of RARα, or a nucleotide sequence at or near a junction of a PML-RARα gene fusion comprising the 3′ end of exon P6b of PML and the 5′ end of exon R3 of RARα.

25. A set of primers comprising at least two primers selected from the group consisting of a primer that hybridizes to exon e1 of BCR, a primer that hybridizes to exon b2 of BCR, and a primer that hybridizes to exon e19 of BCR, wherein the primer can be detectably labeled and/or wherein the set of primers is combined with at least one detectably labeled probe selected from the group consisting of a probe that hybridizes to a nucleotide sequence at or near a junction of a BCR-ABL gene fusion comprising the 3′ end of exon e1 of BCR and the 5′ end of exon a2 of ABL, a probe that hybridizes to a nucleotide sequence at or near a junction of a BCR-ABL gene fusion comprising the 3′ end of exon b2 of BCR and the 5′ end of exon a2 of ABL, a probe that hybridizes to a nucleotide sequence at or near a junction of a BCR-ABL gene fusion comprising the 3′ end of exon b3 of BCR and the 5′ end of exon a2 of ABL, and a probe that hybridizes to a nucleotide sequence at or near a junction of a BCR-ABL gene fusion comprising the 3′ end of exon e19 of BCR and the 5′ end of exon a2 of ABL.

26. The set of primers of claim 25, wherein the primer that hybridizes to exon e1 of BCR comprises a nucleotide sequence selected from the group consisting of SEQ ID NOs: 1, 18, 19, 20, 21, and 22, the primer that hybridizes to exon b2 of BCR comprises a nucleotide sequence selected from the group consisting of SEQ ID NOs: 2 and 23, and/or the primer that hybridizes to exon e19 of BCR comprises a nucleotide sequence selected from the group consisting of SEQ ID NOs: 3 and 24.

27. The set of primers of claim 25, which further comprises a primer that hybridizes to exon a2 of ABL.

28. The set of primers of claim 27, wherein the primer that hybridizes to exon a2 of ABL comprises a nucleotide sequence selected from the group consisting of SEQ ID NOs: 4, 25, 26, and 27.

29. A set of probes comprising at least two probes selected from the group consisting of a probe that hybridizes to a nucleotide sequence at or near a junction of a BCR-ABL gene fusion comprising the 3′ end of exon e1 of BCR and the 5′ end of exon a2 of ABL, a probe that hybridizes to a nucleotide sequence at or near a junction of a BCR-ABL gene fusion comprising the 3′ end of exon b2 of BCR and the 5′ end of exon a2 of ABL, a probe that hybridizes to a nucleotide sequence at or near a junction of a BCR-ABL gene fusion comprising the 3′ end of exon b3 of BCR and the 5′ end of exon a2 of ABL, and a probe that hybridizes to a nucleotide sequence at or near a junction of a BCR-ABL gene fusion comprising the 3′ end of exon e19 of BCR and the 5′ end of exon a2 of ABL.

30. The set of probes of claim 29, wherein the probe that hybridizes to a nucleotide sequence at or near a junction of a BCR-ABL gene fusion comprising the 3′ end of exon e1 of BCR and the 5′ end of exon a2 of ABL comprises a nucleotide sequence selected from the group consisting of SEQ ID NOs: 5, 28, 29, 30, and a sequence complementary thereto, the probe that hybridizes to a nucleotide sequence at or near a junction of a BCR-ABL gene fusion comprising the 3′ end of exon b2 of BCR and the 5′ end of exon a2 of ABL comprises a nucleotide sequence selected from the group consisting of SEQ ID NOs: 6, 31, 32, and a sequence complementary thereto, the probe that hybridizes to a nucleotide sequence at or near a junction of a BCR-ABL gene fusion comprising the 3′ end of exon b3 of BCR and the 5′ end of exon a2 of ABL comprises a nucleotide sequence selected from the group consisting of SEQ ID NOs: 7, 33, 34, 35, and a sequence complementary thereto, and/or the probe that hybridizes to a nucleotide sequence at or near a junction of a BCR-ABL gene fusion comprising the 3′ end of exon e19 of BCR and the 5′ end of exon a2 of ABL comprises a nucleotide sequence selected from the group consisting of SEQ ID NOs: 8, 36, and a sequence complementary thereto.

31. A kit comprising:
(i) a set of primers comprising a primer that hybridizes to an exon of a first gene, which becomes contiguous with a variant exon of a second gene, and a primer for each of two or more variant exons of the second gene; and
(ii) instructions for a method of detecting mRNA from fusions of the first gene and the second gene in a sample of mRNA, which method comprises:
(a) (i′) obtaining cDNA, which has been reverse-transcribed from the sample of mRNA, or reverse-transcribing the sample of mRNA to cDNA and (ii′) amplifying the reverse-transcribed cDNA using primers comprising a primer for an exon of the first gene, which becomes contiguous with a variant exon of the second gene, and a primer for each of two or more variant exons of the second gene, wherein one or more of the primers can be detectably labeled, and
(b) detecting the amplified cDNA or detecting and quantitating the amplified cDNA.

32. The kit of claim 31, which further comprises a probe for each gene fusion variant that is not detected using a detectably labeled primer, wherein the probe hybridizes to a nucleotide sequence at or near a junction of a variant of a fusion between the first gene and the second gene comprising the 3′ end of an exon of the first gene and the 5′ end of an exon of the second gene.

33. The kit of claim 31 or 32, wherein the first gene and the second gene are the BCR gene and the ABL proto-oncogene, the EML4 gene and the ALK gene, the KIF5B gene and the RET proto-oncogene, or the PML gene and the RARα gene.

34. The kit of claim 33, wherein the set of primers comprises (a) at least two primers selected from the group consisting of a primer that hybridizes to exon e1 of BCR, a primer that hybridizes to exon b2 of BCR, and a primer that hybridizes to exon e19 of BCR or (b) a primer comprising a nucleotide sequence selected from the group consisting of SEQ ID NOs: 1, 2, 3, 18, 19, 20, 21, 22, 23, and 24, and wherein the instructions are for a method of detecting mRNA from fusions of the BCR gene and the ABL proto-oncogene in a sample of mRNA from a human, which method comprises:
(a) (i) obtaining cDNA, which has been reverse-transcribed from the sample of mRNA, or reverse-transcribing the sample of mRNA to cDNA and (ii) amplifying the reverse-transcribed cDNA using primers for a group of BCR-ABL gene fusion variants comprising at least two gene fusion variants selected from the group consisting of an e1a2 gene fusion, a b2a2 gene fusion, a b3a2 gene fusion, and an e19a2 gene fusion, wherein one or more of the primers can be detectably labeled, and
(b) detecting the amplified cDNA or detecting and quantitating the amplified cDNA.

35. The kit of claim 34, wherein the set of primers further comprises (a) a primer that hybridizes to exon a2 of ABL or (b) a primer comprising a nucleotide sequence selected from the group consisting of SEQ ID NOs: 4, 25, 26, and 27.

36. The kit of claim 34, which further comprises a probe for each gene fusion variant that is not detected using a detectably labeled primer, wherein (a) the probe hybridizes to a nucleotide sequence at or near a junction of a BCR-ABL gene fusion comprising the 3′ end of exon e1 of BCR and the 5′ end of exon a2 of ABL, a nucleotide sequence at or near a junction of a BCR-ABL gene fusion comprising the 3′ end of exon b2 of BCR and the 5′ end of exon a2 of ABL, a nucleotide sequence at or near a junction of a BCR-ABL gene fusion comprising the 3′ end of exon b3 of BCR and the 5′ end of exon a2 of ABL, or a nucleotide sequence at or near a junction of a BCR-ABL gene fusion comprising the 3′ end of exon e19 of BCR and the 5′ end of exon a2 of ABL or (b) the probe comprises a nucleotide sequence selected from the group consisting of SEQ ID NOs: 5, 6, 7, 8, 28, 29, 30, 31, 32, 33, 34, 35, 36, and a sequence complementary thereto.
